(19) United States
(12) Patent Application Publication
(10) Pub. No.: US 2024/0309076 A1
(43) Pub. Date: Sep. 19, 2024

(54) TAU-SPECIFIC ANTIBODY GENE THERAPY COMPOSITIONS, METHODS AND USES THEREOF

(71) Applicants: REGENXBIO INC., Rockville, MD (US); NEURIMMUNE AG, Schlieren (CH)

(72) Inventors: Danielle Sliter, Rockville, MD (US); Joseph Bruder, Gaithersburg, MD (US); Devin McDougald, Boston, MA (US); Fabio Montrasio, Schlieren (CH); Jan Grimm, Schlieren (CH); Chunping Qiao, Chapel Hill, NC (US)

(21) Appl. No.: 18/259,734

(22) PCT Filed: Dec. 29, 2021
(86) PCT No.: PCT/US2021/065465

§ 371 Date: Jun. 28, 2023

Related U.S. Application Data
(60) Provisional application No. 63/131,660, filed on Dec. 29, 2020.
(60) Provisional application No. 63/139,496, filed on Jan. 20, 2021.

(30) Foreign Application Priority Data
Jan. 20, 2021 (EP) 21150810

(51) Int. Cl. C07K 16/18; A61K 39/00; A61P 25/28; C12N 15/86

(52) U.S. Cl. CPC C07K 16/18; A61P 25/28; C12N 15/86; A61K 2039/505; C07K 2317/34; C07K 2317/522; C07K 2317/565; C07K 2317/622; C07K 2317/64; C07K 2319/02; C12N 2750/14143

(57) ABSTRACT

The present invention relates to nucleic acid expression cassettes that are engineered to express Tau-specific antibodies. Vectors and methods of employing the expression cassettes containing novel Tau-specific antibodies are provided. The invention is particularly useful for delivery of transgenes to target CNS cells and confers desirable properties for CNS-directed gene therapy. Moreover, the invention relates to a novel method of engineering, and expressing Tau-specific antibody transgenes for example within CNS cells, and delivery of therapeutics for treating various tauopathic diseases and disorders.

### CROSS-REFERENCE TO RELATED APPLICATIONS

This application is a national stage filing under 35 U.S.C. § 371 of International Application No. PCT/US2021/65465, filed Dec. 29, 2021, which claims the benefit of priority to US Provisional Application Number 63,139,496 filed on Jan. 20, 2021 and US Provisional Application Number 63,131,660 filed on Dec. 29, 2020 and EP Application 21150810 filed on Jan. 8, 2021. The entire contents of the aforementioned applications are incorporated by reference in their entireties.

### 1. FIELD OF THE INVENTION

The present invention relates to Tau-specific human-derived monoclonal antibodies (“mAbs”) and Tau-binding fragments and variants thereof engineered to be delivered for therapeutic use by viral vectors, methods of making and employing the viral vectors and uses thereof. Use of the biotherapeutic proteins delivered to target cells by expression from viral vectors in the central nervous system (CNS) confers desirable properties for gene therapy. In particular, the invention provides nucleic acid regulatory elements operably linked to a heterologous gene (transgene) inserted into an expression cassette, such that the regulatory elements drive and control expression of an anti-Tau transgene in CNS cells. Provided are methods to target CNS tissues, in particular, with expression cassettes comprising the anti-Tau transgene, using viral vectors suitable for various routes of administration and use in the treatment of tauopathies. As such, compositions and methods are described for the long-term expression of a human therapeutic Tau-specific mAb or the antigen-binding fragment of a therapeutic Tau-specific mAb in the CNS.

### 2. BACKGROUND

Protein accumulation, modifications and aggregation are pathological aspects of numerous neurodegenerative diseases. Pathologically modified and aggregated Tau, including hyperphosphorylated Tau conformers, are an invariant hallmark of tauopathies and correlate with disease severity.

Tau is a microtubule-associated protein expressed in the central nervous system with a primary function to stabilize microtubules. There are six major isoforms of Tau expressed mainly in the adult human brain, which are derived from a single gene by alternative splicing. Under pathological conditions, the Tau protein becomes hyperphosphorylated, resulting in a loss of tubulin binding and destabilization of microtubules followed by the aggregation and deposition of Tau in pathogenic neurofibrillary tangles. There is compelling evidence that Tau hyperphosphorylation and the subsequent formation of higher order multimeric structures leads to neuronal dysfunction and death. For example, there is a strong correlation between the extent of tau pathology and the degree of dementia in Alzheimer's disease (AD) patients, and mutations within the tau gene are known to cause forms of frontotemporal lobar degeneration (FTLD) (Khanna et al. Alzheimers Dement. 10 (2016), 1051-1065). Further disorders related to Tau—collectively referred to as neurodegenerative tauopathies—are for example, Pick's disease (PiD) and corticobasal degeneration (CBD).

Thus, CNS expression of therapeutic proteins that bind to tau or otherwise ameliorate the deleterious effects of tau neurofibrillary tangles would be desirable. There remains a need for CNS-targeted gene expression and vectors that are therapeutic to subjects suffering from tau-related disorders.

### 3. SUMMARY OF THE INVENTION

Provided are embodiments as characterized in the claims, disclosed in the description and illustrated in the Examples and Figures further below. Thus, the present disclosure relates to expression of Tau-specific human-derived monoclonal antibodies in an expression cassette delivered by a viral vector. Tau-specific antibodies include Tau-binding fragments thereof, as well as synthetic variants and biotechnological derivatives of the antibodies exemplified herein (e.g., comprising the antigen binding domain of the Tau-specific human derived monoclonal antibodies described herein, including as depicted in FIG. 1A), that recognize the Tau protein, including pathologically hyperphosphorylated forms of Tau. The Tau specific human derived monoclonal antibodies expressed by the expression cassettes may bind pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD); and/or capture Tau and AD-associated Tau in an immunoprecipitation (IP) assay with brain extracts of synthetic phosphorylated peptide Tau pS202/pT205 or Tau pT212/pS214 using assays disclosed herein; and/or recognizes an epitope comprising the amino acid sequence 217-TPPTREPKKVA-227 (SEQ ID NO: 120) and 249-PMPDLKN-255 (SEQ ID NO: 121) or the phosphorylated Tau peptide pS202/pT205 having the amino acid sequence SGYSSPG(pS)PG(pT)PGSRSRT (SEQ ID NO: 122) or the phosphorylated Tau peptide pT212/pS214 having the amino acid sequence GTPGSRSR(pT)P(pS)LPTPPTR (SEQ ID NO: 123).

Provided are nucleic acid expression cassettes that comprise transgenes which encode the Tau-specific human-derived monoclonal antibodies, which transgenes are operably linked to one or more regulatory sequences that control expression of the transgene in human CNS tissue or in liver cells. The Tau-specific human derived monoclonal antibodies comprise, in embodiments, the CDRs of the heavy and light chain variable domains of NI-502.4P3, NI-502.31B6, or NI-502.8H1 or variants thereof interspersed with framework regions, or the heavy and light chain variable domains of NI-502.4P3, NI-502.31B6, or NI-502.8H1 (see Table 2 for amino acid sequences), as encoded, for example, by nucleotide sequences provided in Table 1, or variants thereof. In certain embodiments, the CDRs of the heavy and light chain variable domain of the Tau-specific human derived monoclonal antibodies NI-502.4P3, NI-502.31B6, or NI-502.8H1 comprise or consist of the amino acid sequences provided in Table 3 and are interspersed with framework regions. In certain embodiments, the nucleic acid expression cassette comprises a transgene which encodes an anti-Tau mAb or antigen-binding fragment thereof or a recombinant form incorporating an antigen-binding domain thereof, wherein the an anti-Tau mAb or antigen-binding fragment thereof or a recombinant form incorporating an antigen-binding domain thereof recognizes an epitope comprising the amino acid sequence 217-TPPTREPKKVA-227 (SEQ ID NO: 120) and 249-PMPDLKN-255 (SEQ ID NO: 121) or the phosphorylated Tau peptide pS202/pT205 having the amino acid sequence SGYSSPG(pS)PG(pT)PGSRSRT (SEQ ID NO: 122) or the phosphorylated Tau peptide pT212/pS214 having the amino acid sequence GTPGSRSR(pT)P(pS)LPTPPTR (SEQ ID NO: 123).

In an aspect, the Tau-specific human derived monoclonal antibodies encoded by the transgenes are full length antibodies, having a heavy chain with VH, C_{H}1, CH2, and CH3 domains and a light chain with VL and CL domains (see Table 2 for amino acid sequences for the VH and VL domains). In embodiments, the Tau-specific human derived monoclonal antibodies encoded by the transgenes disclosed herein comprise VH and VL domains having CDR1, CDR2 and CDR3 of the heavy chain and CDR1, CDR2, and CDR3 of the light chain of NI-502.4P3, NI-502.31B6, and NI-502.8H1 antibodies (see Table 3 for the amino acid sequences of CDRs) interspersed with human framework regions, and heavy chain constant domains (C_{H}1, CH2 and CH3) and light chain constant domains (CL) (see Table 6 for amino acid sequences of constant domains).

In other aspects, the Tau-specific human derived monoclonal antibody is an antigen binding fragment, including a Fab fragment, a F(ab′) fragment, or a F(ab′)₂ fragment. See for example, the expression cassette depicted in FIG. 1B. In embodiments, the antigen binding fragment comprises or consists of a heavy and light chain variable domain or variable domain comprising the heavy and light chain CDRs (interspersed in human framework regions) of one of the Tau-specific human derived monoclonal antibodies NI-502.4P3, NI-502.31B6, and NI-502.8H1 (see Table 2 and, for example, the CDR amino acid sequences of Table 3) and a C_{H}1 domain (in certain embodiments having no hinge sequence or including all or a portion of the hinge) in the heavy chain and a CL domain in the light chain (see Table 6 for constant domain sequences). In specific embodiments, the anti-Tau antibody comprises heavy and light chain CDRs (interspersed in human framework regions) of one of the Tau-specific human derived monoclonal antibodies NI-502.4P3, NI-502.31B6, and NI-502.8H1 comprise amino acid sequences provided in Table 3. In specific embodiments, the anti-Tau Fab comprises heavy and light chain CDRs (interspersed in human framework regions) of one of the Tau-specific human derived monoclonal antibodies NI-502.4P3, NI-502.31B6, and NI-502.8H1 and, in specific embodiments, comprise amino acid sequences of Fab fragment heavy and light chains as provided in Table 2 and 11G.

In aspects, the expression cassette encodes heavy chains and light chains with either an IRES or self cleaving linker, such as a Furin 2A linker, between the nucleotide sequences coding for the heavy chains and light chains, such that the heavy and light chains are expressed from the expression cassette as two separate polypeptides.

In other aspects, the expression cassettes encode recombinant forms incorporating an antigen-binding domain of a Tau-specific human derived monoclonal antibody, which include forms having a heavy chain variable domain and a light chain variable domain, for example, of NI-502.4P3, NI-502.31B6, and NI-502.8H1 antibodies (see Table 2 for amino acid sequences for the VH and VL domains and Table 3 for amino acid sequences of the CDR1, CDR2 and CDR3 of the heavy chain and CDR1, CDR2, and CDR3 of the light chain of NI-502.4P3, NI-502.31B6, and NI-502.8H1 antibodies) fused by a peptide bond, including linked by a flexible, non-cleavable linker, such as an scFv, a minibody, a diabody, or an ScFv-Fc. See, for example, the expression cassettes depicted in FIGS. 1B and 1C. The amino acid sequences of exemplary scFvs are provided in Table 11C, as encoded by nucleotide sequences for the components provided in Table 11D. The disclosed recombinant forms may further comprise an Fc domain or a CH3 domain (see Table 6 for amino acid sequences of Fc domains and CH3 domains) linked to the VH or VL by a hinge/linker sequence (see Table 11A). Amino acid sequences of exemplary scFv-Fcs are provided in Table 11B, as encoded by nucleotide sequences provided in Table 11E and 11F. Nucleotide sequences that encode exemplary Fabs are provided in Table 11G.

The expression cassettes disclosed herein encode heavy and light chains of the Tau-specific human derived monoclonal antibodies each having a signal or leader sequence at the N terminus appropriate for expression and secretion in human cells, particularly human CNS cells or liver cells. The single chain recombinant forms of the Tau-specific human derived monoclonal antibodies have a signal or leader sequence at the N terminus appropriate for expression and secretion in human cells, particularly human CNS cells or liver cells. Table 9 discloses signal or leader sequences that may be used in the disclosed constructs. In the expression cassettes, regulatory sequences are operably linked to the nucleotide sequences encoding the Tau-specific human derived monoclonal antibody. Such regulatory sequences include promoters, enhancers, introns, polyadenylation sequences, for example, as provided in Table 8.

Provided are artificial genomes comprising the expression cassettes described herein, which comprise AAV inverted terminal repeats flanking the expression cassette, in certain embodiments AAV2 ITRs.

In other aspects, provided are recombinant vectors, particularly, recombinant adeno-associated viruses (AAVs) comprising these artificial genomes.

In another aspect, provided are recombinant AAV vectors comprising transgenes encoding a Tau specific human antibody or antigen-binding fragment or recombinant form incorporating an antigen binding domain that binds the determined epitopes (disclosed in Table 4), that is, wherein the antigen-binding fragment or domain recognizes an epitope comprising the amino acid sequence 217-TPPTREPKKVA-227 (SEQ ID NO: 120) and 249-PMPDLKN-255 (SEQ ID NO: 121) or the phosphorylated Tau peptide pS202/pT205 having the amino acid sequence SGYSSPG(pS)PG(pT)PGSRSRT (SEQ ID NO: 122) or the phosphorylated Tau peptide pT212/pS214 having the amino acid sequence GTPGSRSR(pT)P(pS)LPTPPTR (SEQ ID NO: 123).

In another aspect, a method of treatment by delivery of an rAAV comprising an antibody (including antigen binding fragment and forms)-encoding nucleic acid expression cassette described herein are also provided. A method for treating a disease or disorder in a subject in need thereof comprising the administration of recombinant AAV particles comprising an expression cassette encoding Tau-specific human antibodies or antibody-binding fragments or variants thereof is provided.

Methods of producing the rAAVs, host cells for production of rAAVs and rAAVs produced by the methods are disclosed herein.

The embodiments disclosed herein are illustrated by way of examples infra describing the construction and function of gene cassettes engineered with antibodies and antigen-binding fragments thereof operably linked to regulatory elements designed for expression of the antibodies at Tau-associates sites of pathology, for example, but not limited to, the CNS.

### 3.1. Embodiments

1. A nucleic acid expression cassette, wherein the expression cassette comprises a transgene encoding a full-length or substantially full-length anti-Tau protein (anti-Tau) mAb or an antigen-binding fragment thereof, or a recombinant form incorporating an antigen-binding domain thereof, which
(i) binds pathological hyperphosphorylated Tau filaments in dystrophic neurites, neurofibrillary tangles and neuropil threads in an immunohistochemical (IHC) assay with brain tissue of patients with Alzheimer's Disease (AD), Progressive supranuclear palsy (PSP) and/or Pick's Disease (PiD); and/or (ii) captures Tau and AD-associated Tau in an immunoprecipitation (IP) assay with brain extracts of patients with AD;
wherein said transgene is operably linked to one or more regulatory sequences that promote expression of the transgene in human CNS tissue or liver cells.

2. The nucleic acid expression cassette of embodiment 1, wherein said anti-Tau protein mAb or antigen binding fragment thereof or recombinant form incorporating an antigen-binding domain thereof has an EC₅₀ of about 15 nM for Tau or of about 2 nM for the synthetic phosphorylated peptide Tau pS202/pT205 or Tau pT212/pS214.

3. The nucleic acid expression cassette of either of embodiment 1 or 2, which anti-Tau protein mAb or antigen binding fragment thereof or recombinant form incorporating an antigen-binding domain thereof, comprises a variable heavy (VH) chain domain comprising VH complementary determining regions (CDRs) 1, 2, and 3, and a variable light (VL) chain domain comprising VL CDRs 1, 2, and 3, optionally, wherein
(a) VH-CDR1 comprises the amino acid sequence of SEQ ID NO: 154 or a variant thereof, wherein the variant comprises one or two amino acid substitutions,
(b) VH-CDR2 comprises the amino acid sequence of SEQ ID NO: 155 or a variant thereof, wherein the variant comprises one or two amino acid substitutions,
(c) VH-CDR3 comprises the amino acid sequence of SEQ ID NO: 156 or a variant thereof, wherein the variant comprises one or two amino acid substitutions,
(d) VL-CDR1 comprises the amino acid sequence of SEQ ID NO: 157 or a variant thereof, wherein the variant comprises one or two amino acid substitutions,
(e) VL-CDR2 comprises the amino acid sequence of SEQ ID NO: 158 or a variant thereof, wherein the variant comprises one or two amino acid substitutions, and
(f) VL-CDR3 comprises the amino acid sequence of SEQ ID NO: 159 or a variant thereof, wherein the variant comprises one or two amino acid substitutions; or wherein
(g) VH-CDR1 comprises the amino acid sequence of SEQ ID NO: 160 or a variant thereof, wherein the variant comprises one or two amino acid substitutions,
(h) VH-CDR2 comprises the amino acid sequence of SEQ ID NO: 161 or a variant thereof, wherein the variant comprises one or two amino acid substitutions,
(i) VH-CDR3 comprises the amino acid sequence of SEQ ID NO: 162 or a variant thereof, wherein the variant comprises one or two amino acid substitutions,
(j) VL-CDR1 comprises the amino acid sequence of SEQ ID NO: 163 or a variant thereof, wherein the variant comprises one or two amino acid substitutions,
(k) VL-CDR2 comprises the amino acid sequence of SEQ ID NO: 164 or a variant thereof, wherein the variant comprises one or two amino acid substitutions, and
(l) VL-CDR3 comprises the amino acid sequence of SEQ ID NO: 165 or a variant thereof, wherein the variant comprises one or two amino acid substitutions; or wherein
(m) VH-CDR1 comprises the amino acid sequence of SEQ ID NO: 166 or a variant thereof, wherein the variant comprises one or two amino acid substitutions,
(n) VH-CDR2 comprises the amino acid sequence of SEQ ID NO: 167 or a variant thereof, wherein the variant comprises one or two amino acid substitutions,
(o) VH-CDR3 comprises the amino acid sequence of SEQ ID NO: 168 or a variant thereof, wherein the variant comprises one or two amino acid substitutions,
(p) VL-CDR1 comprises the amino acid sequence of SEQ ID NO: 169 or a variant thereof, wherein the variant comprises one or two amino acid substitutions,
(q) VL-CDR2 comprises the amino acid sequence of SEQ ID NO: 170 or a variant thereof, wherein the variant comprises one or two amino acid substitutions, and
(r) VL-CDR3 comprises the amino acid sequence of SEQ ID NO: 171 or a variant thereof, wherein the variant comprises one or two amino acid substitutions.

4. The nucleic acid expression cassette of any of embodiments 1 to 3, wherein the anti-Tau mAb or antigen-binding fragment thereof or recombinant form incorporating an antigen-binding domain thereof recognizes an epitope comprising the amino acid sequence 217-TPPTREPKKVA-227 (SEQ ID NO: 120) and 249-PMPDLKN-255 (SEQ ID NO: 121) or the phosphorylated Tau peptide pS202/pT205 having the amino acid sequence SGYSSPG(pS)PG(pT)PGSRSRT (SEQ ID NO: 122) or the phosphorylated Tau peptide pT212/pS214 having the amino acid sequence GTPGSRSR(pT)P(pS)LPTPPTR (SEQ ID NO: 123).

5. The nucleic acid expression cassette of any of embodiments 1 to 4, wherein the VH domain comprises an amino acid sequence of SEQ ID NO: 97 or a variant thereof having at least 90% sequence identity to the amino acid sequence of SEQ ID NO: 97 and the VL domain comprises an amino acid sequence of SEQ ID NO: 98 or a variant thereof having at least 90% sequence identity to the amino acid sequence of SEQ ID NO: 98; or the VH domain comprises an amino acid sequence of SEQ ID NO: 99 or a variant thereof having at least 90% sequence identity to the amino acid sequence of SEQ ID NO: 99 and the VL domain comprises an amino acid sequence of SEQ ID NO: 100 or a variant thereof having at least 90% sequence identity to the amino acid sequence of SEQ ID NO: 100; or the VH domain comprises an amino acid sequence of SEQ ID NO: 101 or a variant thereof having at least 90% sequence identity to the amino acid sequence of SEQ ID NO: 101 and the VL domain comprises an amino acid sequence of SEQ ID NO: 102 or a variant thereof having at least 90% sequence identity to the amino acid sequence of SEQ ID NO: 102.

6. The nucleic acid expression cassette of any of embodiments 1 to 5, wherein the nucleotide sequence encoding the VH domain comprises SEQ ID NO: 1 or a variant thereof having at least 90% sequence identity to the nucleotide sequence of SEQ ID NO: 1 and the nucleotide sequence encoding the VL domain comprises SEQ ID NO: 2 or a variant thereof having at least 90% sequence identity to the nucleotide sequence of SEQ ID NO: 2; or the nucleotide sequence encoding the VH domain comprises SEQ ID NO: 3 or a variant thereof having at least 90% sequence identity to the nucleotide sequence of SEQ ID NO: 3 and the nucleotide sequence encoding the VL domain comprises SEQ ID NO: 4 or a variant thereof having at least 90% sequence identity to the nucleotide sequence of SEQ ID NO: 4; or the nucleotide sequence encoding the VH domain comprises SEQ ID NO: 5 or a variant thereof having at least 90% sequence identity to the nucleotide sequence of SEQ ID NO: 5 and the nucleotide sequence encoding the VL domain comprises SEQ ID NO: 6 or a variant thereof having at least 90% sequence identity to the nucleotide sequence of SEQ ID NO: 6.

7. The nucleic acid expression cassette of any of embodiments 1 to 6, wherein the anti-Tau antigen binding fragment is a Fab fragment, a F(ab′) fragment, or a F(ab′)₂ fragment.

8. The nucleic acid expression cassette of any of embodiments 1 to 7, wherein the anti-Tau mAb or antigen-binding fragment thereof comprises a heavy chain comprising or consisting of the VH domain and a C_{H}1 domain comprising or consisting of an amino acid sequence of SEQ ID NO: 193 (IgG1), 194 (IgG2), or 195 (IgG4), and comprising a light chain comprising or consisting of the VL domain and a light chain constant region comprising an amino acid sequence of SEQ ID NO: 116 or 118.

9. The nucleic acid expression cassette of embodiment 8, wherein the heavy chain comprises or consists of an amino acid sequence of SEQ ID NO: 351 (IgG1), 352 (IgG2), or 353, (IgG4) and the light chain comprises an amino acid sequence of SEQ ID NO: 188 or 366, or wherein the heavy chain comprises or consists of an amino acid sequence of SEQ ID NO: 356 (IgG1), 357 (IgG2), or 358 (IgG4) and the light chain comprises an amino acid sequence of SEQ ID NO: 190 or 367, or wherein the heavy chain comprises or consists of an amino acid sequence of SEQ ID NO: 361 (IgG1), 362 (IgG2), or 363 (IgG4) and the light chain comprises an amino acid sequence of SEQ ID NO: 192 or 368.

10. The nucleic acid expression cassette of any of embodiments 1 to 7, wherein the anti-Tau mAb or antigen-binding fragment thereof comprises a heavy chain comprising or consisting of the VH domain and a C_{H}1 domain comprising or consisting of an amino acid sequence of SEQ ID NO: 193 (IgG1), 194 (IgG2), or 195 (IgG4) and part or all of the hinge region comprising or consisting of an amino acid sequence of SEQ ID NOs: 172-186 or 202-216 (Table 7), and comprising a light chain comprising a light chain constant region comprising an amino acid sequence of SEQ ID NO: 116 or 118.

11. The nucleic acid expression cassette of embodiment 10, wherein the heavy chain comprises or consists of an amino acid sequence of SEQ ID NO: 187 (IgG1), 354 (IgG2), or 355 (IgG4) and the light chain comprises an amino acid sequence of SEQ ID NO: 188 or 366, or wherein the heavy chain comprises or consists of an amino acid sequence of SEQ ID NO: 189 (IgG1), 359 (IgG2), or 360 (IgG4) and the light chain comprises an amino acid sequence of SEQ ID NO: 190 or 367, or wherein the heavy chain comprises or consists of an amino acid sequence of SEQ ID NO: 191 (IgG1), 364 (IgG2), or 365 (IgG4) and the light chain comprises an amino acid sequence of SEQ ID NO: 192 or 368.

12. The nucleic acid expression cassette of any of embodiments 1 to 6, wherein the anti-Tau mAb is a full length or substantially full length mAb.

13. The nucleic acid expression cassette of embodiment 12, wherein the anti-Tau mAb comprises a heavy chain constant region comprising an amino acid sequence of SEQ ID NO: 103, 105, or 107.

14. The nucleic acid expression cassette of embodiment 12 or 13, wherein the anti-Tau mAb comprises a heavy chain constant region which has one or more amino acid substitutions that reduce binding of the constant region to an FcγR or increase serum half-life.

15. The nucleic acid expression cassette of embodiment 14, wherein the heavy chain constant region comprises an amino acid sequence of SEQ ID NO: 109 or 110.

16. The nucleic acid expression cassette of any of embodiments 1 to 15 wherein the light chain comprises a light chain constant region of SEQ ID NO: 116 or 118.

17. The nucleic acid expression cassette of any of the embodiments 1 to 16, wherein the transgene comprises a VH domain amino acid sequence and a VL domain amino acid sequence that each have a signal sequence at the N-terminus, wherein the signal sequence is appropriate for expression and secretion in human cells.

18. The nucleic acid expression cassette of embodiment 17, wherein the signal sequence is appropriate for expression and secretion in human CNS tissue.

19. The nucleic acid expression cassette of embodiments 17 or 18, wherein said signal sequence is MYRMQLLLLIALSLALVTNS (SEQ ID NO: 87) or a signal sequence listed in Table 9.

20. The nucleic acid expression cassette of any of the embodiments of 1 to 19, wherein a nucleotide sequence encoding a furin 2A linker is in between the nucleotide sequence encoding the heavy chain and the nucleotide sequence encoding the light chain sequences, resulting in a construct with the structure: Leader—Heavy chain—Furin site—2A site—Leader—Light chain—PolyA.

21. The nucleic acid expression cassette of embodiment 20, wherein said furin 2A linker is RKRR(GSG)APVKQTLNFDLLKLAGDVESNPGP (SEQ ID NO: 138).

22. The nucleic acid expression cassette of any of the embodiments of 1 to 21, wherein the transgene comprises a nucleotide sequence encoding the heavy and light chain variable domains of an anti-Tau Fab fragment of Table 11G or the nucleic acid expression cassette has the nucleotide sequence of SEQ ID NO: 378, 384 or 390.

23. The nucleic acid expression cassette of embodiments 1 to 6, wherein the recombinant form incorporating an antigen-binding domain comprises the VH domain and the VL domain linked via a flexible, non-cleavable linker.

24. The nucleic acid expression cassette of embodiment 23 wherein the recombinant form incorporating the antigen-binding domain is an scFv, a minibody, a diabody or an scFv-Fc.

25. The nucleic acid expression cassette of either of embodiments 23 or 24, wherein the recombinant form incorporating the antigen-binding domain is an scFv and has an amino acid sequence of one of SEQ ID Nos: 297 to 305.

26. The nucleic acid expression cassette of any of embodiments 23 to 25, wherein the recombinant form incorporating the antigen-binding domain comprises an Fc domain or a CH3 domain fused to the VH or VL domain by a hinge/linker peptide.

27. The nucleic acid expression cassette of embodiment 26, wherein the hinge/linker peptide comprises an amino acid sequence of SEQ ID NO: 226 to 231.

28. The nucleic acid expression cassette of embodiment 26 or 27, wherein the recombinant form incorporating the antigen-binding domain comprises an Fc domain having an amino acid sequence of SEQ ID NO: 196, 197, or 198, or a CH3 domain having an amino acid sequence of SEQ ID NO: 199, 200, or 201.

29. The nucleic acid expression cassette of any of embodiments 26 to 28, wherein the recombinant form incorporating the antigen-binding domain comprises an amino acid sequence from Table 1 IF or of one of SEQ ID NOs: 234 to 296.

30. The nucleic acid expression cassette of embodiment 29 which comprises a nucleotide sequence of one of SEQ ID NOs: 317 to 340 or 345 to 350 or from Table 1 IF.

31. The nucleic acid expression cassette of embodiment 23, wherein two recombinant forms incorporating the antigen-binding domain are placed in tandem connected by a peptide linker.

32. The nucleic acid expression cassette of any of embodiments 23 to 31, wherein the recombinant form has a signal sequence at the N-terminus appropriate for expression and secretion in human cells.

33. The nucleic acid expression cassette of embodiment 32, wherein the signal or leader sequence is appropriate for expression and secretion in human CNS tissue.

34. The nucleic acid expression cassette of embodiment 32, wherein said signal sequence is MYRMQLLLLIALSLALVTNS (SEQ ID NO: 87) or a signal sequence listed in Table 9.

35. The nucleic acid expression cassette of any of embodiments 1 to 34, wherein the nucleic acid sequence encoding the anti-Tau mAb is codon optimized.

36. The nucleic acid expression cassette of any of embodiments 1 to 35, further comprising:
(1) AAV inverted terminal repeats that flank the expression cassette; and
(2) one or more regulatory control elements operably linked to the transgene, which regulatory control elements include a) a promoter, b) optionally an intron and c) a poly A signal.

37. The nucleic acid expression cassette of any of embodiments 1 to 36, which comprises a nucleotide sequence of SEQ ID NO: 7, 8, 9, 10, 11, 12, 13, 14 or 15 or in Table 11E, 11F or 11G.

38. The nucleic acid expression cassette of any of embodiments 1 to 37 which comprises regulatory element which is a constitutive promoter or a tissue specific promoter that directs expression of the transgene.

39. The nucleic acid expression cassette of embodiment 38 wherein said regulatory element is a CAG promoter, a CMV promoter, a Syn promoter, a GFAP promoter, a Mecp2 promoter, a hexaribonucleotide binding protein-3 (NeuN) promoter or a Cα2+/calmodulin-dependent protein kinase II (CamKII) promoter.

40. A recombinant viral vector comprising the nucleic acid expression cassette of any of embodiments 1 to 39.

41. The recombinant viral vector of embodiment 40 which is an adeno-associated virus (AAV) virus-based vector.

42. A recombinant viral vector, which comprises:
(a) a viral capsid that is at least 95% identical to the amino acid sequence of an AAV8 capsid (SEQ ID NO: 131), AAV9 capsid (SEQ ID NO: 132), AAVrh10 capsid (SEQ ID NO: 133), an AAV.PHP.B capsid (SEQ ID NO: 220), an AAV.PHP.eB capsid (SEQ ID NO: 219), an AAVrh20 capsid (SEQ ID NO: 134), an AAVrh39 capsid (SEQ ID NO: 341) or an AAVcy5 capsid; and
(b) an artificial genome comprising the expression cassette of any of embodiments 1 to 39.

43. The viral vector of embodiment 42, wherein the AAV capsid is AAV8 (SEQ ID NO: 132) or AAV9 (SEQ ID NO: 133).

44. The viral vector of embodiments 40 to 43, wherein the ITR sequences are derived from AAV2.

45. A pharmaceutical composition for use in the prophylactic or therapeutic treatment of a neurodegenerative tauopathy, Alzheimer's Disease (AD), Progressive supranuclear palsy (PSP) or Pick's Disease (PiD), in a human subject in need thereof, comprising an AAV vector having:
(a) a viral capsid that is at least 95% identical to the amino acid sequence of an AAV8 capsid (SEQ ID NO: 131), an AAV9 capsid (SEQ ID NO: 132), an AAV.PHP.B capsid (SEQ ID NO: 220), an AAV.PHP.eB capsid (SEQ ID NO: 219), AAVrh10 capsid (SEQ ID NO: 133), an AAVrh20 capsid (SEQ ID NO: 134), an AAVrh39 capsid (SEQ ID NO: 341) or an AAVcy5 capsid; and
(b) an artificial genome comprising a nucleic acid expression cassette of any of embodiments 1 to 39 flanked by AAV inverted terminal repeats (ITRs), wherein said AAV vector is formulated for administration to the subject, optionally wherein administration is intrathecal, intravenous, subcutaneous, intranasal, or intramuscular or to the cisterna magna.

46. A method for prophylactic or therapeutic treatment of a neurodegenerative tauopathy, Alzheimer's Disease (AD), Progressive supranuclear palsy (PSP) or Pick's Disease (PiD), in a human subject in need thereof, comprising delivering to the CNS of said human subject, a therapeutically effective amount of a substantially full-length or full-length anti-Tau mAb, or antigen-binding fragment thereof, or a recombinant form incorporating an antigen-binding domain thereof expressed from a nucleic acid expression cassette of any of embodiments 1 to 39 and produced by human CNS cells.

47. A method for prophylactic or therapeutic treatment of a neurodegenerative tauopathy, Alzheimer's Disease (AD), Progressive supranuclear palsy (PSP) and/or Pick's Disease (PiD), in a human subject in need thereof, comprising: administering to said subject a therapeutically effective amount of the recombinant viral vector of embodiments 40 to 44 or the pharmaceutical composition of embodiment 45.

48. The pharmaceutical composition or method for prophylactic or therapeutic treatment of a neurodegenerative tauopathy of any of embodiments 45 to 47, wherein the neurodegenerative tauopathy is selected from the group consisting of Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia and ischemic stroke.

49. The composition or method of any of embodiments 45 to 48, wherein the recombinant viral vector is administered intravenously, intrathecally, ICV or via the cisterna magna.

50. The composition or method of any of embodiments 45 to 49, wherein the mAb or antigen-binding fragment thereof is a hyperglycosylated mutant or wherein the Fc polypeptide of the mAb is glycosylated or aglycosylated.

51. The composition or method of embodiments 45 to 50 wherein the mAb or antigen-binding fragment thereof contains an alpha 2,6-sialylated glycan.

52. The composition or method of any of embodiments 45 to 50 wherein the mAb or antigen-binding fragment thereof is glycosylated but does not contain detectable NeuGc and/or α-Gal.

53. The composition or method of any of embodiments 45 to 50 wherein the mAb or antigen-binding fragment thereof contains a tyrosine sulfation.

54. A method of producing recombinant AAVs comprising:
(a) culturing a host cell containing:
   (i) an artificial genome comprising a cis expression cassette of any of embodiments 1 to 39 flanked by AAV ITRs
   (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
   (iii) sufficient adenovirus helper functions to permit replication and packaging of the artificial genome by the AAV capsid protein; and
(b) recovering recombinant AAV encapsidating the artificial genome from the cell culture.

55. The method of embodiment 54, wherein the transgene encodes a substantially full-length or full-length mAb or antigen binding fragment or a variant thereof that comprises the heavy and light chain variable domains of NI-502.4P3, NI-502.31B6, or NI-502.8H1.

56. The method of embodiment 54 or 55, wherein the AAV capsid protein is an AAV9 (SEQ ID NO: 131), AAV.PHP.B (SEQ ID NO: 220), AAV.PHP.eB (SEQ ID NO: 219), AAVrh10 (SEQ ID NO: 132), AAVrh20 (SEQ ID NO: 133), AAVrh39 (SEQ ID NO: 341), or AAVcy5 capsid protein.

57. The method of embodiment 54 to 56, wherein the transgene encodes a substantially full-length or full-length mAb, a single chain Fv fragment (scFv), a F(ab′) fragment, a F(ab) fragment, a F(ab′)₂ fragment, an scFv-Fc, a minibody, or a diabody.

58. A DNA molecule comprising the expression cassette of any one of embodiments 1 to 39.

59. The DNA molecule of embodiment 58 which is a plasmid.

60. A host cell comprising the DNA molecule of embodiment 58 or 59.

61. A host cell containing a nucleic acid comprising the expression cassette of any of embodiments 1 to 39 flanked by AAV ITRs.

62. A host cell comprising the DNA molecule of embodiment 58 or 59.

63. The host cell of embodiment 62 further containing a second nucleic acid comprising a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans.

64. A recombinant AAV produced by the method comprising:
(a) culturing a host cell containing:
   (i) an artificial genome comprising a cis expression cassette of any of embodiments 1 to 39 flanked by AAV ITRs
   (ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans;
   (iii) sufficient adenovirus helper functions to permit replication and packaging of the artificial genome by the AAV capsid protein; and
(b) recovering recombinant AAV encapsidating the artificial genome from the cell culture.

65. The recombinant AAV of embodiment 64, wherein the transgene encodes a substantially full-length or full-length mAb or antigen binding fragment or a variant thereof that comprises the heavy and light chain variable domains of NI-502.4P3, NI-502.31B6, or NI-502.8H1.

### 4. BRIEF DESCRIPTION OF THE FIGURES

FIGS. 1A-1F illustrate various molecular strategies for constructing gene therapy vectors. FIG. 1A illustrates various molecular formats containing therapeutic antigen-binding fragments, for example anti-Tau-binding fragments. FIG. 1B depicts schematics illustrating the construction of various rAAV vector genome (transgene) constructs, e.g. a vectorized Fab construct, a vectorized (full-length)IgG construct and a vectorized ScFv-Fc construct, containing an expression cassette encoding the heavy and light chains of the therapeutic mAb controlled by expression elements, and flanked by AAV ITRs. FIG. 1C depicts schematics illustrating the construction of further rAAV vector genome (transgene) constructs, e.g. a vectorized Minibody construct, a vectorized ScFv construct and a vectorized Diabody construct, containing an expression cassette encoding the heavy and light chains of the therapeutic mAb in various arrangements, including optional linkers, wherein transcription of the encoded mAbs are controlled by expression elements, and flanked by AAV ITRs. FIG. 1D provides the amino acid sequence of an exemplary transgene construct of the 4P3 antibody, such that the components are arranged to express a 4P3 Fab or IgG transgene. Various glycosylation and/or sulfation sites are depicted, illustrating that expression of the antibody transgene in human cells undergoes post-translational modification. FIG. 1E provides the amino acid sequence of an exemplary transgene construct of the 31B6 antibody, such that the components are arranged to express a 31B6 Fab or IgG transgene. Various glycosylation and/or sulfation sites are depicted, illustrating that expression of the antibody transgene in human cells undergoes post-translational modification. FIG. 1F provides the amino acid sequence of an exemplary transgene construct of the 8H1 antibody, such that the components are arranged to express a 8H1 Fab or IgG transgene. Various glycosylation and/or sulfation sites are depicted, illustrating that expression of the antibody transgene in human cells undergoes post-translational modification.

FIG. 2 (panels 1 to 5) depicts a Clustal Multiple Sequence Alignment of AAV capsids 1-9, rh.10, rh.20, rh.39, rh.74 (v1 and v2) and hu.37. The amino acid sequences of the AAV capsids are assigned SEQ ID NOs as follows: AAV1 is SEQ ID NO: 124; AAV2 is SEQ ID NO: 125; AAV3 is SEQ ID NO: 126; AAV4 is SEQ ID NO: 127; AAV5 is SEQ ID NO: 128; AAV6 is SEQ ID NO: 129; AAV7 is SEQ ID NO: 130; AAV8 is SEQ ID NO: 131; AAV9 is SEQ ID NO: 132; AAVrh.10 is SEQ ID NO: 133; AAVrh.20 is SEQ ID NO: 134; rh.74v1 is SEQ ID NO: 135; rh74.v2 is SEQ ID NO: 136; rh.39 is SEQ ID NO: 341 and hu.37 is SEQ ID NO: 137.

FIG. 3 depicts antibody protein expression in 293T cells as detected by Western Blot. Cells were transfected with cis plasmids each expressing the following antibodies: 43A11 IgG2a (isotype control mAb), 31B6 IgG2a (mAb 31B6 chimeric human/mouse, full-length), 4P3 IgG2a (mAb 4P3 chimeric human/mouse, full-length), 8H1 IgG2a (mAb 8H1 chimeric human/mouse, full-length), 31B6 IgG2a LALAPG (mAb 31B6 chimeric human/mouse, full-length having Fc mutations), 4P3 IgG2a LALAPG (mAb 4P3 chimeric human/mouse, full-length having Fc mutations), and 8H1 IgG2a LALAPG (mAb 8H1 chimeric human/mouse, full-length having Fc mutations). Note that mAb 43A11 (control), mAb 4P3, and mAb 8H1 contain a kappa light chain, and mAb 31B6 contains a lambda light chain.

FIGS. 4A and 4B depicts viral vector expression in 293T-AAVR cells transduced with Tau AGT vectors at 10e4 or 10e5 vector genome/cell. Supernatant was collected at 40 hours post-transduction and immunoblotted to confirm antibody expression. All antibodies were detected in supernatant. 43A11 (isotype control mAb with an IgG2a heavy chain constant domain), 31B6 (mAb 31B6 chimeric human/mouse, full-length, with a IgG2a heavy chain constant domain), 4P3 (mAb 4P3 chimeric human/mouse, full-length, with an IgG2a heavy chain constant domain), 8H1 IgG2a (mAb 8H1 chimeric human/mouse, full-length, with an IgG2a heavy chain constant domain), 31B6 LALAPG (mAb 31B6 chimeric human/mouse, full-length with an IgG2a heavy chain constant domain having Fc mutations), 4P3 LALAPG (mAb 4P3 chimeric human/mouse, full-length with an IgG2a heavy chain constant domain having Fc mutations having Fc mutations), and 8H1 IgG2a LALAPG (mAb 8H1 chimeric human/mouse, full-length with an IgG2a heavy chain constant domain having Fc mutations having Fc mutations). Note that mAb 43A11 (control), mAb 4P3, and mAb 8H1 contain a kappa light chain (A), and mAb 31B6 contains a lambda light chain (B).

FIG. 5 shows the results of antibody dose-dependent expression as detected in plasma following bilateral ICV administration of 4P3 IgG2a and 4P3 IgG2a LALAPG gene therapy vectors in P0/P1 pup mice at three different doses: 2.4e9 vg/side (low dose), 1.2e10 vg/side (mid dose), and 6e10 vg/side (high dose).

FIGS. 6A-6C. Antibody is detected following ICV administration of 4P3 IgG2a and 4P3 IgG2a LALAPG gene therapy vectors at three doses in various brain tissues (sandwich ELISA): cortex (A: Ab concentration in homogenate), hippocampus (B: Ab concentration in homogenate), and striatum (C: Ab concentration in homogenate), as depicted in the plots of antibody concentration (nM) quantitated in tissue homogenate and as a fraction of total protein (ng Ab per μg total protein).

FIG. 7. Antibody (4P3 IgG2a) levels (nM) detected in plasma following administration of AAV 4P3 IgG2a encoding gene therapy vector (mid dose level: 1.2e10 per side for total administration of 2.4e10) by intrahippocampal administration, intrastriatal administration or intraventricular administration to adult mice 4 weeks after dosing.

FIGS. 8A-8C: Antibody concentrations (ng Ab per μg total protein) determined by a sandwich ELISA mouse IgG2a capture assay in brain homogenates from mice 4 weeks after administration by intrahippocampal administration, intrastriatal administration, or intraventricular administration of the AAV 4P3 IgG2a encoding vector of cortex (A), hippocampus (B), and striatum (C)\.

FIGS. 9A-9C: Antibody concentrations (nM) determined by a sandwich ELISA mouse IgG2a capture assay in homogenates of cortex (A), hippocampus (B), and striatum (C) from C57BL/6 mice having been administered AAV9 vector controls or encoding anti-Tau vectored constructs described herein (see Table 15) bilaterally either intracerebroventricular (ICV) or, if indicated as “i.h.c”, intrahippocampal, at 4 weeks after administration.

FIGS. 10A-10C: Antibody concentrations (nM) determined by a sandwich ELISA mouse IgG2a capture assay in brain homogenates of cortex (A), hippocampus (B), and striatum (C) from C57BL/6 mice having been administered AAV9 vector controls or encoding anti-Tau vectored constructs described herein bilaterally either intrahippocampal or, if indicated as “icv”, intracerebroventricular, at 4 weeks after administration.

FIGS. 11A-11C: Antibody levels (pM) determined by a sandwich ELISA mouse IgG2a capture assay in brain homogenates of hippocampus (A), cortex (B) and striatum (C) from C57BL/6 mice having been administered AAV9 vector controls or encoding anti-Tau vectored constructs encoding the 4P3 IgG2a antibody under the control of the hSyn or GFAP promoters bilaterally intrahippocampally (1.2e10 vg/side) or 4P3 IgG2a antibody (as protein, not a gene therapy construct) intraperitoneally (i.p.) (30 mg/kg), each at 1 month and 3 months after administration (4P3 IgG2a antibody assessed only at one month).

FIGS. 12A-12C. Immunodepletion of seeding competent Tau from AD brain homogenate by anti-tau antibodies NI-502.4P3, NI-502.31B6 and NI-502.8H1. Tau aggregation in HEK293T Tau biosensor cells using AD brain homogenate from a selected donor that had been immunodepleted with increasing concentrations of NI-502.4P3 (A), NI-502.31B6 (B) or NI-502.8H1 (C) antibodies. The derived IC₅₀ values were 6.5, and 7.1 μg/mL, for donors NI-502.4P3 and NI-502.8H1, respectively. Due to the obtained curve fitting, no IC₅₀ value could be determined for NI-502.31B6. Data were fitted to a non-linear regression curve; each antibody concentration was tested in duplicate, error bars represent standard deviation. AD, Alzheimer's disease; FRET, fluorescence resonance energy transfer.

### 5. DETAILED DESCRIPTION

Provided, in part, are viral vectors and methods of treatment using vectors having transgenes encoding unique Tau-specific human-derived monoclonal antibodies and Tau-binding fragments thereof, as well as synthetic variants and biotechnological derivatives of the antibodies having the binding activity of the Tau-specific human-derived monoclonal antibodies. The invention thus provides viral vectors carrying the nucleotide sequences encoding antibodies and antigen-binding fragments thereof to the CNS, wherein the Tau-specific antibodies are expressed and bind pathological hyperphosphorylated Tau filaments in dystrophic neurites, neurofibrillary tangles and neuropil threads, as demonstrated in an immunohistochemical (IHC) assay with brain tissue of patients with Alzheimer's Disease (AD), Progressive supranuclear palsy (PSP) and/or Pick's Disease (PiD). The provided vectors deliver the antibodies by expression of transgenes by incorporating nucleotide sequences encoding the engineered antibodies, antigen-binding fragments or variants thereof described herein, into viral vectors, such as rAAVs, for use in therapy. The novel Tau-specific human-derived monoclonal antibody nucleic acids were engineered in expression cassettes including regulatory elements to provide antibody expression at the site of Tau-induced pathogenesis in order to re-stabilize microtubules or otherwise ameliorate the symptoms of pathogenic Tau. Ultimately, these viral vector designs may improve therapeutic efficacy by gene transfer of Tau-specific antibodies by providing stabile and persistent expression of the therapeutic product in the CNS.

### 5.1. Definitions

The term “viral vector” refers to a replication defective viral particle containing a nucleic acid transgene, in certain instances, a Tau-specific antibody transgene. In one embodiment, an expression cassette as described herein may be engineered onto a plasmid which is used for drug delivery or for production of a viral vector. Suitable viral vectors are preferably replication defective and selected from amongst those which target brain cells. Viral vectors may include any virus suitable for gene therapy, including but not limited to adenovirus; herpes virus; lentivirus; retrovirus; parvovirus, etc. However, for ease of understanding, the adeno-associated virus is referenced herein as an exemplary virus vector. A “replication-defective virus” or “viral vector” is a synthetic or recombinant viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the molecules required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral proteins required for replication.

The term “AAV” or “adeno-associated virus” refers to a Dependoparvovirus within the Parvoviridae genus of viruses. The AAV can be an AAV derived from a naturally occurring “wild-type” virus, an AAV derived from a rAAV genome packaged into a capsid comprising capsid proteins encoded by a naturally occurring cap gene and/or from a rAAV genome packaged into a capsid comprising capsid proteins encoded by a non-naturally occurring capsid cap gene. An example of the latter includes a rAAV having a capsid protein comprising a peptide insertion into or modification of the amino acid sequence of the naturally-occurring capsid.

The term “rAAV” refers to a “recombinant AAV.” In some embodiments, a recombinant AAV has an AAV genome in which part or all of the rep and cap genes have been replaced with heterologous sequences.

The term “rep-cap plasmid” refers to a plasmid that provides the viral rep and cap gene function and aids the production of AAVs from rAAV genomes lacking functional rep and/or the cap gene sequences.

The term “cap gene” refers to the nucleic acid sequences that encode capsid proteins that form or help form the capsid coat of the virus. For AAV, the capsid protein may be VP1, VP2, or VP3.

The term “rep gene” refers to the nucleic acid sequences that encode the non-structural proteins needed for replication and production of virus.

The term “expression cassette” or “nucleic acid expression cassette” refers to nucleic acid molecules that include the coding sequence for a gene of interest operably linked to one or more transcriptional control elements including, but not limited to promoters, enhancers and/or regulatory elements, introns and polyadenylation sequences. The enhancers and promoters typically function to direct (trans)gene expression in one or more desired cell types, tissues or organs. Polyadenylation sequences such as a bovine growth hormone (bGH) polyadenylation (polyA) site or a SV40 polyA site indicate the site of transcription termination.

The term “regulatory element” or “nucleic acid regulatory element” are non-coding nucleic acid sequences that control the transcription of neighboring genes. Cis regulatory elements typically regulate gene transcription by binding to transcription factors. This includes “composite nucleic acid regulatory elements” comprising more than one enhancer or promoter elements that regulate expression of transgene.

The term “operably linked” and “operably linked to” refers to nucleic acid sequences being linked and typically contiguous, or substantially contiguous, and, where necessary to join two protein coding regions, contiguous and in reading frame which functionally impact (increase, decrease or inhibit) or effect the expression of the other sequence, such as, for example a promoter sequence promoting transcription of the coding sequence of a gene of interest. However, since enhancers generally function when separated from the promoter by several kilobases and intronic sequences may be of variable lengths, some polynucleotide elements may be operably linked and still be functional while not directly contiguous with a downstream promoter and transgene.

The terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

“Percent (%) sequence identity” with respect to a reference polynucleotide or polypeptide sequence is referred to as the percentage of nucleic acids or amino acids in a candidate sequence that are identical to the nucleic acids or amino acids in the reference polynucleotide or polypeptide sequence, after aligning the sequences and introducing gaps, if necessary, to achieve the maximum percent sequence identity. Alignment for purposes of determining percent nucleic acid or amino acid sequence identity can be achieved in various ways that are well-known in the art, for example, using publicly available computer software such as BLAST, BLAST-2, or Megalign software. Using well-recognized and conventional methods, the appropriate parameters can be determined for aligning sequences, including any algorithms needed to achieve maximal alignment over the full length of the sequences being compared. For example, percent sequence identity values may be generated using the sequence comparison computer program BLAST. As an illustration, the percent sequence identity of a given nucleic acid or amino acid sequence, A, to, with, or against a given nucleic acid or amino acid sequence, B, (which can alternatively be phrased as a given nucleic acid or amino acid sequence, A that has a certain percent sequence identity to, with, or against a given nucleic acid or amino acid sequence, B) is calculated as follows:
100 multiplied by (the fraction X/Y),
where X is the number of nucleotides or amino acids scored as identical matches by a sequence alignment program (e.g., BLAST) in that program's alignment of A and B, and where Y is the total number of nucleic acids in B. It will be appreciated that where the length of nucleic acid or amino acid sequence A is not equal to the length of nucleic acid or amino acid sequence B, the percent sequence identity of A to B will not equal the percent sequence identity of B to A.

As used throughout, AAV “serotype” refers to an AAV having an immunologically distinct capsid, a naturally-occurring capsid, or an engineered capsid.

The terms “subject”, “host”, and “patient” are used interchangeably. As used herein, a subject is preferably a mammal such as a non-primate (e.g., cows, pigs, horses, cats, dogs, rats etc.) or a primate (e.g., monkey and human), in most cases, a human.

The terms “therapeutic protein” or “biotherapeutic protein” refer to any protein which is encoded by a transgene, and used in treating, managing, or ameliorating symptoms associated with a disease or disorder, where the disease or disorder is associated with a target molecule or function of the target molecule to be provided by or modulated by (including activated or increased by or inhibited by) the transgene product. Also, a “therapeutically effective amount” refers to the amount of protein, (e.g., amount of protein product expressed by the transgene) that provides at least one therapeutic benefit in the treatment or management of the target disease or disorder, following administration of a gene therapy to a subject suffering therefrom. Further, a therapeutically effective amount with respect to an antibody transgene of the invention means that amount of transgene product alone, or when in combination with other therapies, that provides at least one therapeutic benefit in the treatment or management of the disease or disorder.

The phrase “tissue-specific” or “tissue-directed” refers to targeting by capsid tropism or transcriptional targeting by use of specific promoters. Viral vector delivery or transduction of genome to specific cells or tissue is characteristic of a capsid's tropism, each capsid, particularly each rAAV capsid having a unique distribution in the body. For example, AAV serotype 9 (AAV9, SEQ ID NO: 132) has unique properties in the CNS, since AAV9 distribution spreads to much larger areas of the CNS after intra-CSF administration compared with other serotypes, however it may also reach the circulation and transduces the liver, providing a peripheral source of therapeutic protein (Samaranch, L. et al. Human Gene Therapy. Apr 2012; 23(4):382-389). In some instances, nucleic acid regulatory elements, such as promoters, drive the cell- or tissue-specific expression of a transgene, since promoters have adapted their activity in specific cells or tissue due to the interaction of such elements with the intracellular environment of such cells. Therefore tissue-specific expression may control the amount of expression in a given tissue. For example, the human synapsin 1 gene promoter (hSYN) provides neuronal-specific transgene expression. Transgene expression exclusively in astrocytes has been demonstrated by the full-length 2.2 bp glial fibrillary acidic protein (GFAP) promoter and its much shorter variant, the GfaABClD promoter, drove transgene expression exclusively in astrocytes. Other examples of promoters are described herein including those presented in Table 8.

### 5.2. Viral Vector Delivery of Tau-Specific Antibodies and Antigen-Binding Fragments and Variants Thereof

One aspect relates to nucleic acid sequences encoding Tau-specific mAbs, including antigen-binding fragments and forms thereof, and regulatory and structural elements that are arranged in an expression cassette to promote expression of the Tau-specific mAb for delivery to cells to create a permanent depot that continuously supplies the human Tau-specific mAb, e.g., human-glycosylated, transgene product.

Accordingly, the transgene may be any one of the genes or nucleic acids encoding the therapeutic proteins (e.g., containing the heavy and light chain variable domains of the Tau-specific antibodies) listed in, but not limited to, Table 1 for nucleotide sequences with amino acid sequences of the heavy and light chain variable domains (and Fab portions of the antibodies) in Table 2. Also provided are transgenes encoding anti-Tau-specific antibodies having the heavy chain CDRs of the VH of SEQ ID NO 97, 99 or 101 and the light chain CDRs of the VL of SEQ ID NO: 98, 100 or 102 (interspersed with appropriate framework, including human framework, regions) (CDR amino acid sequences provided in Table 3).

Provided are Tau-specific mAb heavy and light chain arrangements and nucleic acid regulatory elements that promote mAb transgene expression in CNS tissue. Exemplary nucleotide sequences encoding the individual heavy and light chain variable domains (and Fab fragments thereof) of Tau-specific antibodies are provided in Table 1. Exemplary promoter, enhancer and other elements for transgene expression and arrangement in an expression cassette are provided in Table 8.

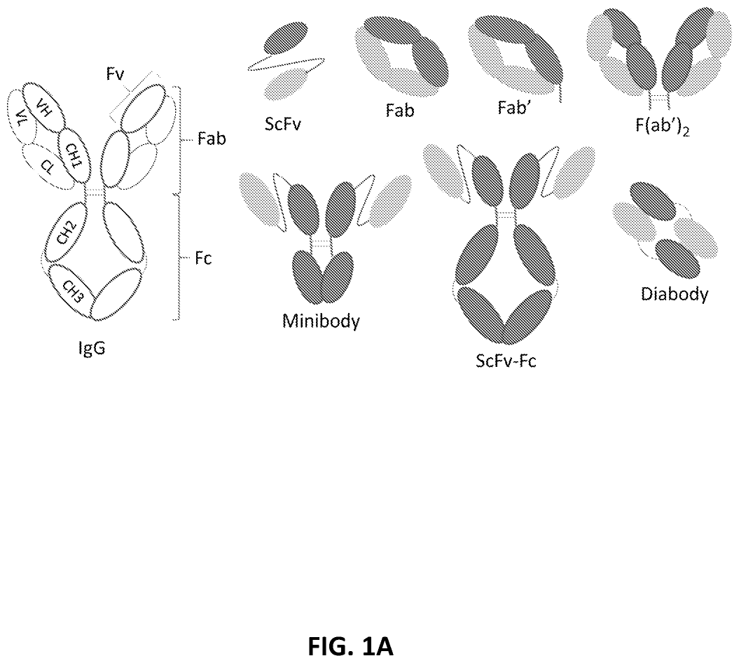

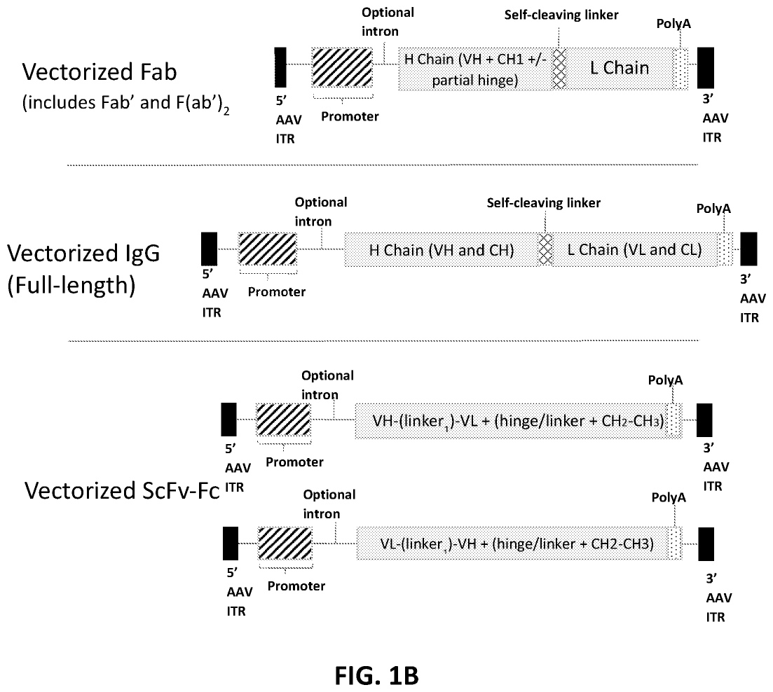

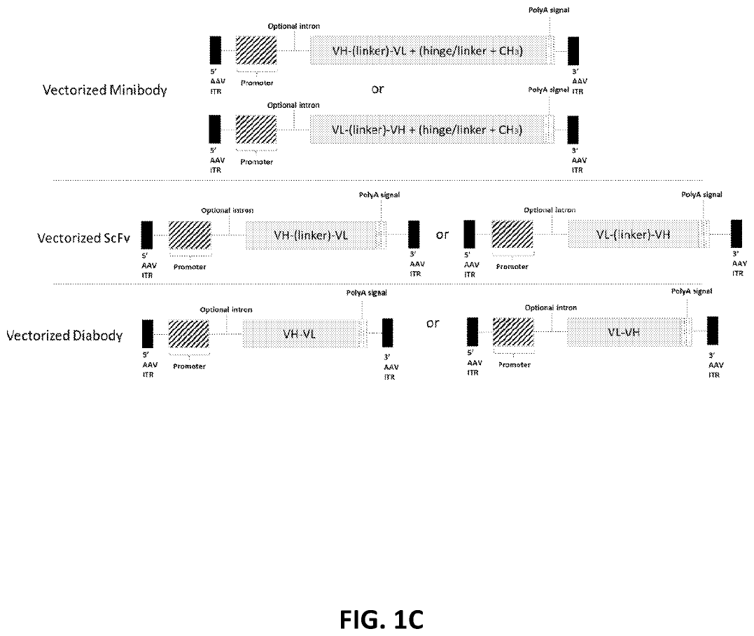

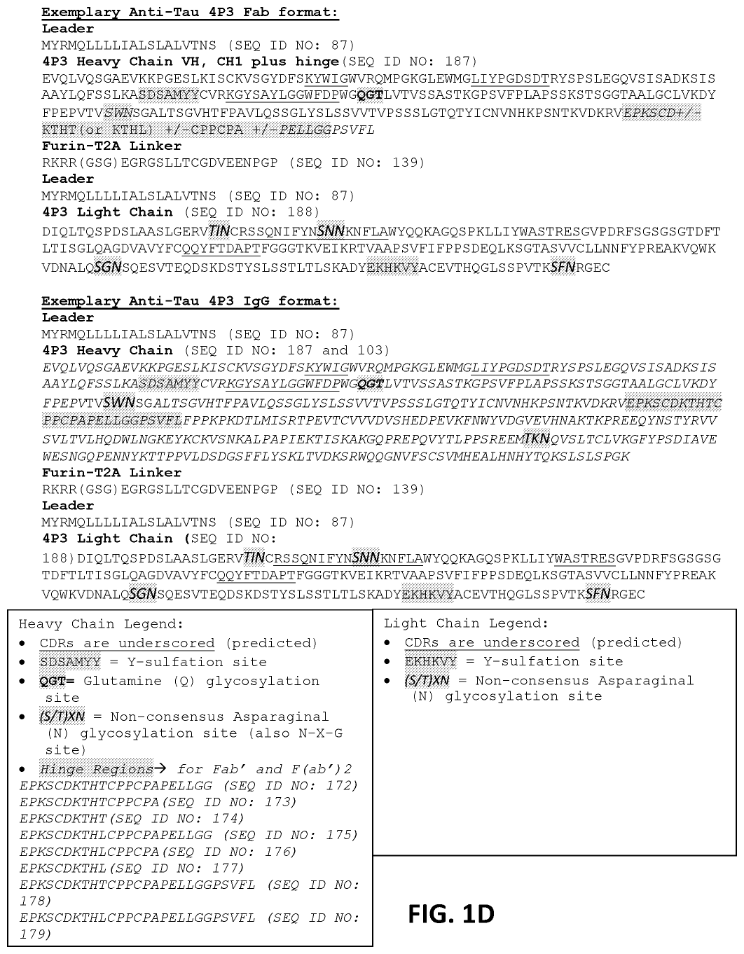

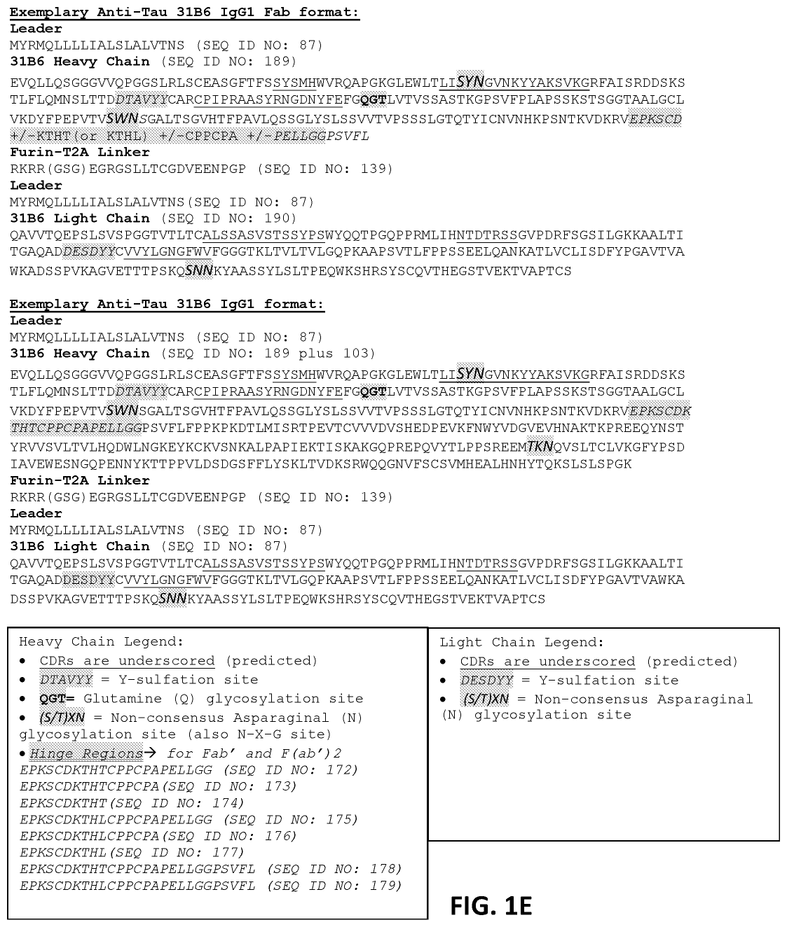

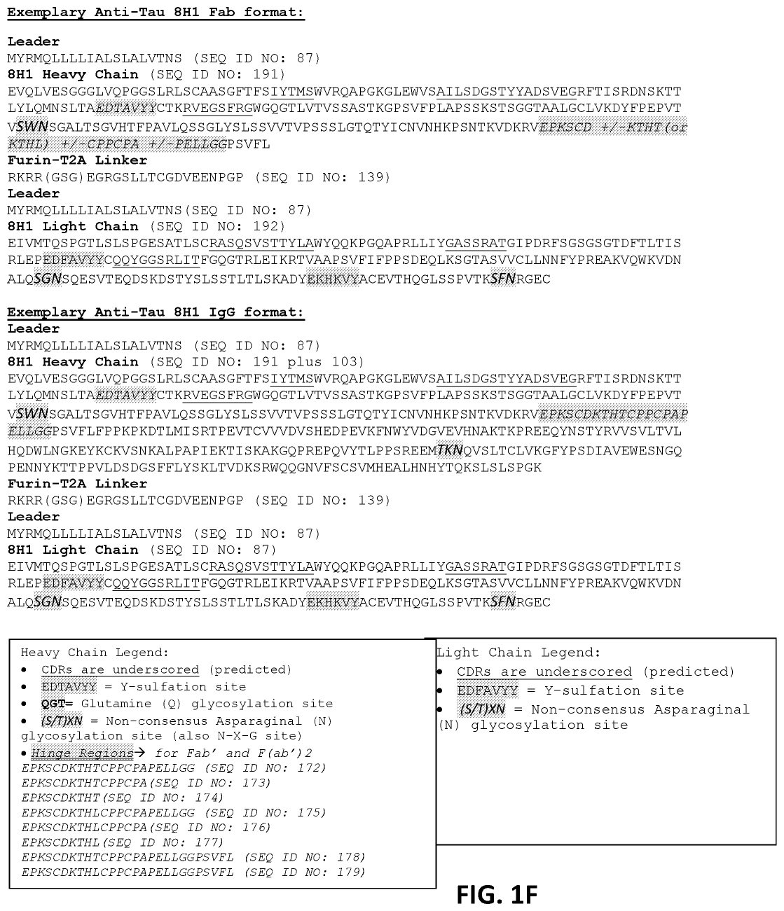

Provided are composite nucleic acid transgene sequences, or “vectored” antibody constructs, for expression of antibody or antigen fragment or variant thereof listed in, but not limited to, Table 2, Table 5 or Table 11G. Accordingly, the transgene may be any one of the transgenes or nucleic acids encoding the therapeutic antibodies comprising nucleic acid sequences encoding heavy and light chain variable domains, including, SEQ ID Nos: 1 and 2, or 3 and 4, or 5 and 6, respectively, for example, as set forth in Table 1. Exemplary vectored antibody expression cassettes for vectorized Fab and full-length IgGs, as well as for various mAb formats, are diagrammed in FIG. 1B and FIG. 1C with amino acid sequences in FIGS. 1D-1F and Tables 2, 5, 11A, 11B, 11C, 11F, and 11G and nucleotide sequences in Tables 1, 11D, 11E, 11F, and 11G.

### 5.2.1. Tau-Binding Molecules

The Tau-specific antigen-binding molecules described herein convey certain properties and the gene therapy constructs, compositions and methods preserves or enhances the antigen-binding molecules' therapeutic properties, as well as directs the therapeutic molecules to the site of pathological hyperphosphorylated Tau filaments.

The combinations of heavy and light chain sequences may be expressed by a multicistronic cassette while maintaining the therapeutic molecule's unique binding specificity. Alternatively, the heavy and light chain variable domain sequences may be expressed as a single polypeptide, for example as an scFv or an scFv-hinge-Fc, or other format, as described herein.

The human monoclonal anti-Tau antibodies described herein were identified in a complex antibody discovery process surprisingly yielding antibodies that bind different forms of Tau in brain tissues of patients suffering from tauopathies, in particular, the antibodies bind pathological hyperphosphorylated Tau filaments in dystrophic neurites, neurofibrillary tangles and neuropil threads, as determined in an immunohistochemical (IHC) assay with brain tissue of patients with Alzheimer's Disease (AD), Progressive supranuclear palsy (PSP) and/or Pick's Disease (PiD) (EP Application No. 20 217 601.2, filed Dec. 29, 2020, which is incorporated by reference herein in its entirety). Tables 1 and 2 present the nucleic acid sequence and encoded amino acid sequence of the VH and VL domains of these antibodies, respectively. Table 2 further includes the amino acid sequences of VH and VL domains fused to portions or all of the constant domain and/or hinge, as indicated. Table 3 presents the amino acid sequences of the heavy and light chain CDR1 CDR2 and CDR3 of the anti-Tau antibodies disclosed herein.

TABLE 1
Tau-specific Antigen-Binding Nucleic Acids

| Antibody | SEQ ID | |
|---|---|---|
| Chain Name | NO: | Nucleic Acid Sequence |
| NI-502.4P3-VH | 1 | gaggtgcagctggtgcagtctggagcagaggtgaagaagcccgggga |
| | | gtctctgaagatctcctgtaaggtttctggatacgactttagcaagt |
| | | actggatcggctgggtgcgccagatgcccgggaaaggcctggagtgg |
| | | atggggctcatctatcctggtgactctgataccagatatagtccatc |
| | | tttggaagggcaagtcagcatctcagccgacaagtccatcagcgccg |
| | | cctacttgcaattcagcagcctgaaggcctcggacagcgccatgtat |
| | | tattgtgtgaggaagggatatagtgcctaccttggagggtggttcga |
| | | cccctggggccagggaaccctggtcaccgtctcctcg |
| NI-502.4P3-VL | 2 | gacatccagttgacccagtctccagactccctggctgcgtctctggg |
| | | cgagagggtcaccatcaactgcaggtccagccagaatattttttaca |
| | | actccaacaataagaacttcttagcttggtatcagcagaaagcagga |
| | | cagtctcctaagttgcttatttactgggcttctacgcgggaatccgg |
| | | ggtccctgaccgattcagtggcagcggctctgggacagatttcactc |
| | | tcaccatcagcggcctgcaggctggagatgtggcagtttatttctgt |
| | | cagcaatattttactgatgctcccactttcggcggagggaccaaggt |
| | | ggagatcaaa |
| NI-502.31B6- | 3 | gaggtgcagctgttgcagtctgggggaggcgtggtccagcctggggg |
| VH | | gtccctgagactctcctgtgaagcctctggattcaccttcagttcct |
| | | attctatgcactgggtccgccaggctccaggcaaggggctggaatgg |
| | | ctgacacttatctcatataatggagtcaataagtattacgcaaagtc |
| | | cgtgaagggccgattcgccatctccagagacgattccaagagcacac |
| | | tgtttttgcaaatgaacagcctgacaactgacgacacggctgtatat |
| | | tactgtgcgagatgtccaatcccgcgggcggcgtcgtatagaaatgg |
| | | agataattatttcgaattcggccagggcaccctggtcaccgtctcct |
| | | cg |
| NI-502.31B6- | 4 | caggctgtggtgactcaggagccatcgctctcagtgtcccctggagg |
| VL | | gacagtcacactcacttgtgccctgagttctgcctcagtctctactt |
| | | cttcgtaccccagctggtaccagcagaccccaggccagcctccacgc |
| | | atgctcatccacaacacagacactcgctcttctggtgtccctgatcg |
| | | cttctctggctccatccttgggaagaaagctgccctcaccatcacgg |
| | | gggcccaggcagatgatgaatctgattactactgtgtggtatatttg |
| | | gggaatggcttttgggtgttcggcggagggaccaagctgaccgtcct |
| | | a |
| NI-502.8H1-VH | 5 | gaggtgcagctggtggagtctgggggaggcttggtgcagccgggggg |
| | | gtccctaagactctcctgtgcagcctccggattcacctttagcatct |
| | | ataccatgagctgggtccgccaggctccagggaaggggctggagtgg |
| | | gtctcagctattctcagtgatggtagcacatactatgcagactccgt |
| | | agagggccgattcaccatctccagagacaattccaagaccaccctgt |
| | | atctacaaatgaacagcctgacagccgaggacacggccgtatattat |
| | | tgtacaaaacgtgttgaggggagctttcgggggtggggccagggaac |
| | | cctggtcaccgtctcctcg |
| NI-502.8H1-VL | 6 | gaaattgtgatgacacagtctccaggcaccctgtctttgtctccagg |
| | | ggaaagcgccaccctctcctgcagggccagtcagagtgttagcacta |
| | | cctacttagcctggtaccagcagaaacctggccaggctcccaggctc |
| | | ctcatctatggtgcatccagcagggccactggcatcccagacagatt |
| | | cagtggcagtgggtctgggacagacttcactctcacaatcagcagac |
| | | tggagcctgaagattttgcagtgtattactgtcagcagtatggtggg |
| | | tcacgcttgatcaccttcggccaagggacacgactggagattaaa |

TABLE 2
Tau-specific Antibody Amino Acid Sequences

| | SEQ ID | Amino Acid Sequence |
|---|---|---|
| Name | NO: | (CDRs in bold) |
| NI-502.4P3 | 97 | EVQLVQSGAEVKKPGESLKISCKVSGYDFSKYWIGWVRQMPGKG |
| VH | | LEWMGLIYPGDSDTRYSPSLEGQVSISADKSISAAYLQFSSLKA |
| | | SDSAMYYCVRKGYSAYLGGWFDPWGQGTLVTVSS |
| NI-502.4P3 | 351 | EVQLVQSGAEVKKPGESLKISCKVSGYDFSKYWIGWVRQMPGKG |
| VH + C_{H}1(IgG1) | | LEWMGLIYPGDSDTRYSPSLEGQVSISADKSISAAYLQFSSLKA |
| | | SDSAMYYCVRKGYSAYLGGWFDPWGQGTLVTVSSASTKGPSVFP |
| | | LAPSSKSTSGGTAALGCLVKDYFPEPVTVSWNSGALTSGVHTFP |
| | | AVLQSSGLYSLSSVVTVPSSSLGTQTYICNVNHKPSNTKVDKRV |
| NI-502.4P3 | 352 | EVQLVQSGAEVKKPGESLKISCKVSGYDFSKYWIGWVRQMPGKG |
| VH + C_{H}1(IgG2) | | LEWMGLIYPGDSDTRYSPSLEGQVSISADKSISAAYLQFSSLKA |
| | | SDSAMYYCVRKGYSAYLGGWFDPWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSNFGTQTYTCNVDHKPSNTKVDKTV |
| NI-502.4P3 | 353 | EVQLVQSGAEVKKPGESLKISCKVSGYDFSKYWIGWVRQMPGKG |
| VH + C_{H}1(IgG4) | | LEWMGLIYPGDSDTRYSPSLEGQVSISADKSISAAYLQFSSLKA |
| | | SDSAMYYCVRKGYSAYLGGWFDPWGQGTLVTVSSASTKGPSVFP |
| | | LAPCSRSTSESTAALGCLVKDYFPEPVTVSWNSGALTSGVHTFP |
| | | AVLQSSGLYSLSSVVTVPSSSLGTKTYTCNVDHKPSNTKVDKRV |
| NI-502.4P3 VH + C_{H}1 + Hinge (IgG1) | 187 | 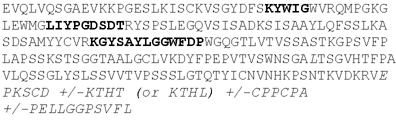 |
| NI-502.4P3 VH + C_{H}1 + Hinge (IgG2) | 354 | 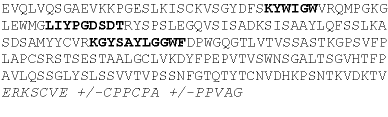 |
| NI-502.4P3 VH + C_{H}1 + Hinge (IgG4) | 355 | 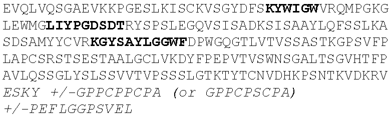 |
| NI-502.4P3 | 98 | DIQLTQSPDSLAASLGERVTINCRSSQNIFYNSNNKNFLAWYQQ |
| VL | | KAGQSPKLLIYWASTRESGVPDRFSGSGSGTDFTLTISGLQAGD |
| | | VAVYFCQQYFTDAPTFGGGTKVEIK |
| NI-502.4P3- | 188 | DIQLTQSPDSLAASLGERVTINCRSSQNIFYNSNNKNFLAWYQQ |
| VL + CL (kappa) | | KAGQSPKLLIYWASTRESGVPDRFSGSGSGTDFTLTISGLQAGD |
| | | VAVYFCQQYFTDAPTFGGGTKVEIKRTVAAPSVFIFPPSDEQLK |
| | | SGTASVVCLLNNFYPREAKVQWKVDNALQSGNSQESVTEQDSKD |
| | | STYSLSSTLTLSKADYEKHKVYACEVTHQGLSSPVTKSFNRGEC |
| NI-502.4P3- | 366 | DIQLTQSPDSLAASLGERVTINCRSSQNIFYNSNNKNFLAWYQQ |
| VL + CL | | KAGQSPKLLIYWASTRESGVPDRFSGSGSGTDFTLTISGLQAGD |
| (lambda) | | VAVYFCQQYFTDAPTFGGGTKVEIKGQPKAAPSVTLFPPSSEEL |
| | | QANKATLVCLISDFYPGAVTVAWKADSSPVKAGVETTTPSKQSN |
| | | NKYAASSYLSLTPEQWKSHRSYSCQVTHEGSTVEKTVAPTCS |
| NI-502.31B6 | 99 | EVQLLQSGGGVVQPGGSLRLSCEASGFTFSSYSMHWVRQAPGKG |
| VH | | LEWLTLISYNGVNKYYAKSVKGRFAISRDDSKSTLFLQMNSLTT |
| | | DDTAVYYCARCPIPRAASYRNGDNYFEFGQGTLVTVSS |
| NI-502.31B6 | 356 | EVQLLQSGGGVVQPGGSLRLSCEASGFTFSSYSMHWVRQAPGKG |
| VH + C_{H}1(IgG1) | | LEWLTLISYNGVNKYYAKSVKGRFAISRDDSKSTLFLQMNSLTT |
| | | DDTAVYYCARCPIPRAASYRNGDNYFEFGQGTLVTVSSASTKGP |
| | | SVFPLAPSSKSTSGGTAALGCLVKDYFPEPVTVSWNSGALTSGV |
| | | HTFPAVLQSSGLYSLSSVVTVPSSSLGTQTYICNVNHKPSNTKV |
| | | DKRV |
| NI-502.31B6 | 357 | EVQLLQSGGGVVQPGGSLRLSCEASGFTFSSYSMHWVRQAPGKG |
| VH + C_{H}1(IgG2) | | LEWLTLISYNGVNKYYAKSVKGRFAISRDDSKSTLFLQMNSLTT |
| | | DDTAVYYCARCPIPRAASYRNGDNYFEFGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSNFGTQTYTCNVDHKPSNTKV |
| | | DKTV |
| NI-502.31B6 | 358 | EVQLLQSGGGVVQPGGSLRLSCEASGFTFSSYSMHWVRQAPGKG |
| VH + C_{H}1(IgG4) | | LEWLTLISYNGVNKYYAKSVKGRFAISRDDSKSTLFLQMNSLTT |
| | | DDTAVYYCARCPIPRAASYRNGDNYFEFGQGTLVTVSSASTKGP |
| | | SVFPLAPCSRSTSESTAALGCLVKDYFPEPVTVSWNSGALTSGV |
| | | HTFPAVLQSSGLYSLSSVVTVPSSSLGTKTYTCNVDHKPSNTKV |
| | | DKRV |
| NI-502.31B6 VH + C_{H}1 + Hinge (IgG1) | 189 | 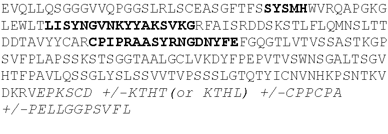 |
| NI-502.31B6 VH + C_{H}1 + Hinge (IgG2) | 359 | 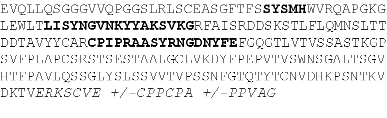 |
| NI-502.31B6 VH + C_{H}1 + Hinge (IgG4) | 360 | 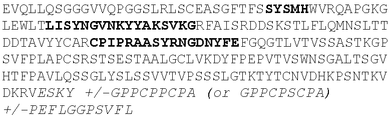 |
| NI-502.31B6 | 100 | QAVVTQEPSLSVSPGGTVTLTCALSSASVSTSSYPSWYQQTPGQ |
| VL | | PPRMLIHNTDTRSSGVPDRFSGSILGKKAALTITGAQADDESDY |
| | | YCVVYLGNGFWVFGGGTKLTVL |
| NI-502.31B6 | 190 | QAVVTQEPSLSVSPGGTVTLTCALSSASVSTSSYPSWYQQTPGQ |
| VL + CL | | PPRMLIHNTDTRSSGVPDRFSGSILGKKAALTITGAQADDESDY |
| (lambda) | | YCVVYLGNGFWVFGGGTKLTVLGQPKAAPSVTLFPPSSEELQAN |
| | | KATLVCLISDFYPGAVTVAWKADSSPVKAGVETTTPSKQSNNKY |
| | | AASSYLSLTPEQWKSHRSYSCQVTHEGSTVEKTVAPTCS |
| NI-502.31B6 | 367 | QAVVTQEPSLSVSPGGTVTLTCALSSASVSTSSYPSWYQQTPGQ |
| VL + CL (kappa) | | PPRMLIHNTDTRSSGVPDRFSGSILGKKAALTITGAQADDESDY |
| | | YCVVYLGNGFWVFGGGTKLTVLRTVAAPSVFIFPPSDEQLKSGT |
| | | ASVVCLLNNFYPREAKVQWKVDNALQSGNSQESVTEQDSKDSTY |
| | | SLSSTLTLSKADYEKHKVYACEVTHQGLSSPVTKSFNRGEC |
| NI-502.8H1 | 101 | EVQLVESGGGLVQPGGSLRLSCAASGFTFSIYTMSWVRQAPGKG |
| VH | | LEWVSAILSDGSTYYADSVEGRFTISRDNSKTTLYLQMNSLTAE |
| | | DTAVYYCTKRVEGSFRGWGQGTLVTVSS |
| NI-502.8H1- | 361 | EVQLVESGGGLVQPGGSLRLSCAASGFTFSIYTMSWVRQAPGKG |
| VH + C_{H}1(IgG1) | | LEWVSAILSDGSTYYADSVEGRFTISRDNSKTTLYLQMNSLTAE |
| | | DTAVYYCTKRVEGSFRGWGQGTLVTVSSASTKGPSVFPLAPSSK |
| | | STSGGTAALGCLVKDYFPEPVTVSWNSGALTSGVHTFPAVLQSS |
| | | GLYSLSSVVTVPSSSLGTQTYICNVNHKPSNTKVDKRV |
| NI-502.8H1- | 362 | EVQLVESGGGLVQPGGSLRLSCAASGFTFSIYTMSWVRQAPGKG |
| VH + C_{H}1(IgG2) | | LEWVSAILSDGSTYYADSVEGRFTISRDNSKTTLYLQMNSLTAE |
| | | DTAVYYCTKRVEGSFRGWGQGTLVTVSSASTKGPSVFPLAPCSR |
| | | STSESTAALGCLVKDYFPEPVTVSWNSGALTSGVHTFPAVLQSS |
| | | GLYSLSSVVTVPSSNFGTQTYTCNVDHKPSNTKVDKTV |
| NI-502.8H1- | 363 | EVQLVESGGGLVQPGGSLRLSCAASGFTFSIYTMSWVRQAPGKG |
| VH + C_{H}1(IgG4) | | LEWVSAILSDGSTYYADSVEGRFTISRDNSKTTLYLQMNSLTAE |
| | | DTAVYYCTKRVEGSFRGWGQGTLVTVSSASTKGPSVFPLAPCSR |
| | | STSESTAALGCLVKDYFPEPVTVSWNSGALTSGVHTFPAVLQSS |
| | | GLYSLSSVVTVPSSSLGTKTYTCNVDHKPSNTKVDKRV |
| NI-502.8H1- VH + C_{H}1 + Hinge (IgG1) | 191 | 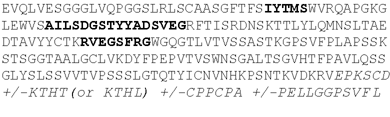 |
| NI-502.8H1- VH + C_{H}1 + Hinge (IgG2) | 364 | 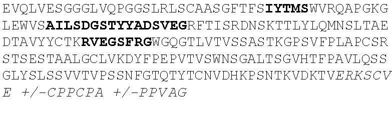 |
| NI-502.8H1- VH + C_{H}1 + Hinge (IgG4) | 365 | 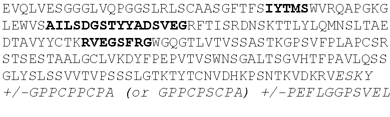 |
| NI-502.8H1 | 102 | EIVMTQSPGTLSLSPGESATLSCRASQSVSTTYLAWYQQKPGQA |
| VL | | PRLLIYGASSRATGIPDRFSGSGSGTDFTLTISRLEPEDFAVYY |
| | | CQQYGGSRLITFGQGTRLEIK |
| NI-502.8H1 | 192 | EIVMTQSPGTLSLSPGESATLSCRASQSVSTTYLAWYQQKPGQA |
| VL + CL | | PRLLIYGASSRATGIPDRFSGSGSGTDFTLTISRLEPEDFAVYY |
| (kappa) | | CQQYGGSRLITFGQGTRLEIKRTVAAPSVFIFPPSDEQLKSGTA |
| | | SVVCLLNNFYPREAKVQWKVDNALQSGNSQESVTEQDSKDSTYS |
| | | LSSTLTLSKADYEKHKVYACEVTHQGLSSPVTKSFNRGEC |
| NI-502.8H1 | 368 | EIVMTQSPGTLSLSPGESATLSCRASQSVSTTYLAWYQQKPGQA |
| VL + CL | | PRLLIYGASSRATGIPDRFSGSGSGTDFTLTISRLEPEDFAVYY |
| (lambda) | | CQQYGGSRLITFGQGTRLEIKGQPKAAPSVTLFPPSSEELQANK |
| | | ATLVCLISDFYPGAVTVAWKADSSPVKAGVETTTPSKQSNNKYA |
| | | ASSYLSLTPEQWKSHRSYSCQVTHEGSTVEKTVAPTCS |

TABLE 3
CDR sequences

| Antibody | CDR | SEQ ID NO: | Sequence |
|---|---|---|---|
| 4P3 | VH-CDR1 | 154 | KYWIG |
| | VH-CDR2 | 155 | LIYPGDSDT |
| | VH-CDR3 | 156 | KGYSAYLGGWFDP |
| | VL-CDR1 | 157 | RSSQNIFYNSNNKNFLA |
| | VL-CDR2 | 158 | WASTRES |
| | VL-CDR3 | 159 | QQYFTDAPT |
| 31B6 | VH-CDR1 | 160 | SYSMH |
| | VH-CDR2 | 161 | LISYNGVNKYYAKSVKG |
| | VH-CDR3 | 162 | CPIPRAASYRNGDNYFE |
| | VL-CDR1 | 163 | ALSSASVSTSSYPS |
| | VL-CDR2 | 164 | NTDTRSS |
| | VL-CDR3 | 165 | VVYLGNGFWV |
| 8H1 | VH-CDR1 | 166 | IYTMS |
| | VH-CDR2 | 167 | AILSDGSTYYADSVEG |
| | VH-CDR3 | 168 | RVEGSFRG |
| | VL-CDR1 | 169 | GASSRAT |
| | VL-CDR2 | 170 | RASQSVSTTYLA |
| | VL-CDR3 | 171 | QQYGGSRLIT |

Several anti-Tau antibodies against various epitopes of tau have been cloned and tested in IHC assays, only a few of which were found to bind Tau in brain tissue of patients with AD; see Table 4. Antibodies NI-502.4P3, NI-502.31B6, and NI-502.8H1 all bind in brain tissues of patients with AD, with PSP, and with PiD, as well as brain tissue extracts from transgenic mice hemizygous for Tau P301L (also known as FTDP-17 mutation) (Lewis, J. et al. 2000 Nature Genetics 25:402-405). Furthermore, said antibodies have been found to capture Tau and AD-associated Tau in an immunoprecipitation (IP) assay with brain extracts of patients with AD (Table 4).

TABLE 4
Binding properties of anti-Tau antibodies

| | | IHC | IP | |
|---|---|---|---|---|
| | | Human | Human | IHC |
| Antibody | Epitope | AD | AD | P301L |
| NI- | 217-TPPTREPKKVA-227 (SEQ ID NO: 120) and | + | + | + |
| 502.4P3 | 249-PMPDLKN-255 (SEQ ID NO: 121) | | | |
| (4P3) | | | | |
| NI- | pS202/pT205: SGYSSPG(pS)PG(pT)PGSRSRT | + | + | + |
| 502.31B6 | (SEQ ID NO: 122) | | | |
| (31B6) | | | | |
| NI- | pT212/pS214: GTPGSRSR(pT)P(pS)LPTPPTR | + | + | + |
| 502.8H1 | (SEQ ID NO: 123) | | | |
| (8H1) | | | | |

The binding specificity and EC50 of human-derived, Tau-specific antibodies were also determined by indirect ELISA (See Section 5.8, infra, for exemplary assay for determining the EC50). Antibody NI-502.4P3 specifically recognized the Tau protein with an EC50 of 15.0 nM. Antibody NI-502.31B6 specifically targeted the synthetic phosphorylated peptide Tau pS202/pT205 with an EC50 of 2.0 nM whereas antibody NI-502.8H1 specifically bound the synthetic phosphorylated peptide Tau pT212/pS214 with an EC50 of 2.2 nM. The Tau-specific antibodies were shown to specifically recognize human Tau. By “specifically recognizing tau”, “binding tau”, “antibody specific to/for tau” and “anti-tau antibody” means specifically, generally, and collectively, antibodies to the native form of tau, or aggregated or pathologically modified tau isoforms. Provided herein are human antibodies selective for full-length, pathologically phosphorylated and aggregated forms. Furthermore, specific recognition or binding to tau epitopes may be determined by well-known methods. One such method, Pepspot™ epitope mapping analysis, was used to map epitopes within the human Tau protein recognized by the Tau specific antibodies. Pepscan membrane loaded with over 100 linear 15-meric peptides having about 11 aa overlap between individual peptides covering the entire human Tau protein sequence were used. Overlapping amino acids between peptides being recognized by each test antibody elucidated the putative binding epitopes.

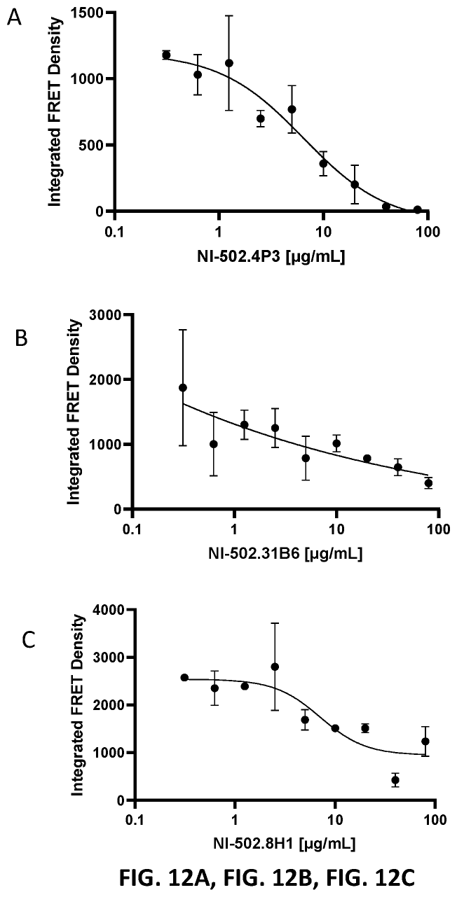

In addition, antibodies NI-502.4P3, NI-502.31B6 and NI-502.8H1 could be shown to deplete seeding-competent tau from AD homogenates; see FIGS. 12A to 12C.

Table 2 provides the amino acid sequence of the heavy and light chain variable domains of the anti-Tau mAbs. Table 2 further provides the amino acid sequences of the heavy and light chains of the Fab fragments-including the VH—C_{H}1 and VL-CL, or VH, C_{H}1 and at least a portion of the hinge region of the heavy chain and the VL and constant domain of the light chain (CL). In some embodiments, the antibodies of Table 2 further comprise a constant region, including a C_{H}1 domain or a CL domain, and optionally, for the heavy chains, an Fc domain (which includes the C_{H}2 and C_{H}3 domains of the heavy chain). In embodiments, the antibodies are full length antibodies having VH—C_{H}1-C_{H}2-C_{H}3 domains for the heavy chain and VL-CL for the light chain. In some embodiments, the constant region, including the Fc domain, of the antibody is selected from a constant domain or Fc domain from human IgG1, human IgG2, human IgG4, mouse IgG2a or mouse IgG2b, or a variant thereof (as described in Section 5.2.4, which amino acid sequences are provided in Table 6).

CDR sequences of the heavy and light chain variable domains in the sequences provided by Table 2 may be readily discerned by those skilled in the art. Accordingly, provided are anti-Tau antibodies having the CDRs of the heavy and light chain variable domains interspersed among human framework regions. Table 3 provides the exemplary amino acid sequences for the CDR1, CDR2 and CDR3 of the heavy chain variable domain and the CDR1, CDR2 and CDR3 of the light chain variable domain. It will be appreciated that these CDR sequences may be interspersed among variable domain framework sequences, particularly human framework sequences, to introduce the antigen binding domain of NI-502.4P3, NI-502.31B6, or NI-502.8H1 into an anti-Tau antibody as known by those in the art. Further, one, two, three, four or more amino acid substitutions may be made in the framework regions or the CDRs to improve binding efficiency using methods well known in the art.

Furthermore, sequence analysis, i.e. comparison of the human Tau sequence (NCBI Gene ID: 4137) with the mouse Tau sequence (NCBI Gene ID: 17762) revealed that the binding epitopes of antibody NI-502.4P3 are shared between human and murine Tau proteins, which makes it prudent to assume that antibody NI-502.4P3 also recognizes the murine Tau protein.

In addition, the epitopes of antibody NI-502.4P3 are located adjacent to and in the microtubule binding region (MTBR), respectively, which spans from residues 224-369 of Tau; see, e.g., Horie et al., Brain 144 (2021), 515-527. Antibodies binding an epitope in that upstream region of MTBR demonstrated a significant and selective ability to mitigate tau seeding and a reduction of inducing tau pathology in cellular and in vivo transgenic mice models seeded by human Alzheimer's disease brain extracts; see summary in Horie et al., (2021) and references cited therein.

### 5.2.2. Transgenes

The transgenes provided herein encode an anti-Tau mAb, particularly either as a full-length antibody or an antigen binding fragment thereof, e.g. a Fab or Fab′ fragment or a F(ab′)₂, or a synthetic or recombinant form incorporating an antigen-binding domain thereof (such as, for example, an scFv, minibody, diabody, nanobody, scFv-Fc). Exemplary structures of antigen-binding fragments and recombinant forms are depicted in FIG. 1A and schematics of exemplary transgenes are provided in FIGS. 1B and 1C. Exemplary nucleotide sequences that are encompassed by the disclosed transgenes and the amino acid sequences of the anti-Tau antibodies, antigen binding fragments and recombinant forms encoded by the transgenes can be found in Tables 2, 5, and 11A-11G.

In certain embodiments, provided are transgenes that encode the anti-Tau antibody, the anti-Tau antigen-binding fragment or other recombinant anti-Tau antigen-binding form which comprise the nucleotide sequences encoding the heavy and light chains of the variable regions of NI-502.4P3 (nucleotide sequences SEQ ID NOs. 1 and 2, respectively, see Table 1). The nucleotide sequences may be codon optimized for expression in human cells. The amino acid sequences of the heavy and light chain variable domains of NI-502.4P3 (also 4P3) are provided in Table 2, and, in particular, are SEQ ID NO: 97 (encoding the NI-502.4P3 heavy chain variable portion) and SEQ ID NO: 98 (encoding the NI-502.4P3 light chain variable portion). Exemplary transgene products are depicted in FIG. 1D. The transgene may encode heavy and light chain variable domain sequences that have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 87) or a signal sequence listed in Table 9.

In addition to the heavy and light chain variable domains, the heavy and light chains encoded by the transgene may also comprise C_{H}1 and CL domain sequences, and, in certain embodiments, the transgenes may comprise, at the C-terminus of the heavy chain C_{H}1 domain sequence, all or a portion of the hinge region (Table 7 for hinge sequences). The C_{H}1-domain may be an IgG1 C_{H}1 (SEQ ID NO: 193), IgG2 C_{H}1 (SEQ ID NO: 194) or IgG4 C_{H}1 (SEQ ID NOs: 195) domain. In some embodiments, the anti-Tau-antigen binding domain has or comprises a heavy chain variable domain of SEQ ID NO: 97, and a C_{H}1 of IgG1 (SEQ ID NO: 193), IgG2 (SEQ ID NO: 194) or IgG4 (SEQ ID NO: 195), or a variant thereof. In certain embodiments, the anti-Tau-antigen binding domain has a heavy chain domain comprising VH and C_{H}1, with the heavy chain consisting of or comprising an amino acid sequence of SEQ ID NOs: 351, 352, or 353 (Table 2) and a light chain consisting of or comprising a VL and a CL with an amino acid sequence of SEQ ID NO: 188 or 366.

In certain embodiments, the anti-Tau-antigen binding domain has a heavy chain variable domain of SEQ ID NO: 97 and a IgG1 C_{H}1 domain sequence (SEQ ID NO: 193) with additional hinge region sequence starting after the C-terminal valine (V), contains all or a portion of the amino acid sequence EP, EPKS, EPKS, EPKSCDKTHTCPPCPAPELLGG (SEQ ID NO: 172), EPKSCDKTHTCPPCPA(SEQ ID NO: 173), EPKSCDKTHT(SEQ ID NO: 174), EPKSCDKTHLCPPCPAPELLGG (SEQ ID NO: 175), EPKSCDKTHLCPPCPA(SEQ ID NO: 176), EPKSCDKTHL(SEQ ID NO: 177), EPKSCDKTHTCPPCPAPELLGGPSVFL (SEQ ID NO: 178) or EPKSCDKTHLCPPCPAPELLGGPSVFL (SEQ ID NO: 179) as set forth in FIG. 1D. In particular embodiments, the anti-Tau antigen binding domain has a heavy chain comprising a VH, C_{H}1 and all or a portion of the hinge domain, with the heavy chain consisting of or comprising an amino acid sequence of SEQ ID NO: 187 and a light chain consisting of or comprising a VL and a CL (for example a kappa CL or a lambda CL) with an amino acid sequence of SEQ ID NO: 188 or 366 (see FIG. 1D and Table 2 for heavy and light chain sequences).

In other embodiments, the anti-Tau-antigen binding domain has a heavy chain variable domain of SEQ ID NO: 97 and a IgG4 C_{H}1 domain sequence (e.g. SEQ ID NO: 195) with optionally additional hinge region sequence starting after the C-terminal valine (V), contains all or a portion of the amino acid sequence, ES, ESKY, ESKYGPPCPPCPAPEFLGG (SEQ ID NO: 180), and specifically, ESKYGPPCPPCPA (SEQ ID NO: 181), ESKYGPPCPSCPA (SEQ ID NO: 182), ESKYGPPCPSCPAPEFLGGPSVFL (SEQ ID NO: 183), or ESKYGPPCPPCPAPEFLGGPSVFL (SEQ ID NO: 184). In other embodiments, the anti-Tau-antigen binding domain has a heavy chain variable domain of SEQ ID NO: 97 and a IgG2 C_{H}1 domain sequence (e.g. SEQ ID NO: 194) with additional hinge region sequence starting after the C-terminal valine (V), contains all or a portion of the amino acid sequence ERKCCVECPPCPAPPVAG (SEQ ID NO: 185) or ERKCCVECPPCPA (SEQ ID NO: 186). See also Table 7 for a listing of the amino acid sequences of hinge regions.

In another embodiment, the transgenes comprise the amino acid sequences encoding the full length (or substantially full length) heavy and light chains of the antibody, that is, the heavy chain comprises the VH, C_{H}1 and hinge domain and, in addition, the Fc domain. The Fc domains that may be incorporated into the anti-Tau antibodies encoded by the transgenes described herein are as disclosed in Section 5.2.4, infra. The Fc domain which includes the C_{H}2 and C_{H}3 domains of the heavy chain may be an IgG1 (SEQ ID NO: 196), IgG2 (SEQ ID NO: 197) or IgG4 (SEQ ID NO: 198) Fc domain and may have one or more amino acid modifications that alter Fc effector function (including altered binding to one or more Fc receptors) or increase serum half-life (e.g. LALA-PG). In particular embodiments, the Fc domain has an amino acid sequence of SEQ ID NOs: 196, 197, or 198 (Table 6), or a mutant or variant thereof. Exemplary amino acid sequences of the entire constant region (SEQ ID NOs: 103, 105, 107, 109, or 110) comprising C_{H}1 domain, hinge region, C_{H}2 domain, and C_{H}3 domain are also provided in Table 6. Also provided are anti-Tau mAbs which have a murine constant region or Fc domain (for example an IgG2a Fc domain or the entire IgG2a constant region, SEQ ID NOs: 112 or 113) which may be useful for study in mouse models of disease.

In certain embodiments, the anti-Tau antigen-binding fragment transgene encodes an Tau antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 98. In certain embodiments, the anti-Tau antigen-binding fragment transgene encodes an Tau antigen-binding fragment comprising a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 97. In certain embodiments, the anti-Tau antigen-binding fragment transgene encodes an antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 98 and a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 97. In certain embodiments, the Tau antigen binding fragment comprises a heavy chain variable domain comprising an amino acid sequence of SEQ ID NO: 97 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1D and the sequences set forth in Table 3 (SEQ ID NOs: 154, 155, and 156, being the amino acid sequences of the VH-CDR1, VH-CDR2 and VH-CDR3, respectively). In certain embodiments, the Tau antigen binding fragment comprises a light chain comprising an amino acid sequence of SEQ ID NO: 98 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1D and the sequences set forth in Table 3 (SEQ ID NOs: 157, 158, and 159 being the amino acid sequence of VL-CDR1, VL-CDR2 and VL-CDR3, respectively). In certain embodiments, the anti-Tau antigen-binding fragment transgene encodes a hyperglycosylated NI-502.4P3 Fab, comprising a heavy chain and a light chain of SEQ ID NOs: 187 and 188, respectively, with one or more of the following mutations: L117N (heavy chain) and/or Q166N, Q166S, and/or E201N (light chain).

In certain embodiments, the anti-Tau antigen binding fragment transgene encodes an antigen binding fragment and comprises the nucleotide sequences encoding the six NI-502.4P3 CDRs, which may be determined readily by those skilled in the art and including those which are underlined in the heavy and light chain variable domain sequences of FIG. 1D (see also Table 3) which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof. In certain embodiments, the anti-Tau antigen-binding fragment transgene encodes an antigen-binding fragment and comprises the nucleotide sequences encoding the six NI-502.4P3 CDRs (comprising, for example, SEQ ID NOs: 154-159) which are underlined in the heavy and light chain variable domain sequences of FIG. 1D which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

Exemplary transgenes are provided that comprise nucleotide sequences encoding anti-Tau ScFvs or anti-Tau ScFv-Fcs include, for example, the nucleotide sequences of SEQ ID NOs:369-370. In some embodiments, the transgenes are present in artificial AAV genomes in which the transgene is flanked by ITR sequences, for example the nucleotide sequence of SEQ ID NO:369. Exemplary transgenes that encode an anti-Tau Fab include nucleotide sequences of SEQ ID NOs:378 to 379. In some embodiments, the transgenes are present in constructs providing an ITR to ITR cassette of SEQ ID NO:378.

The transgenes provided herein encode an anti-Tau mAb (FIG. 1D), particularly either as a full-length antibody or an antigen binding fragment thereof, e.g. a Fab or Fab′ fragment or a F(ab′)₂, or a synthetic or recombinant form incorporating an antigen-binding domain thereof (such as, for example, an scFv, minibody, diabody, nanobody, scFv-Fc). Exemplary structures of antigen-binding fragments and recombinant forms are depicted in FIG. 1A, exemplary transgenes are depicted in FIGS. 1B and 1C, and exemplary amino acid sequences as well as nucleotide sequences are provided in Tables 2, 5, and 11A-11G.

In certain embodiments, provided are transgenes that encode the anti-Tau antibody, the anti-Tau antigen-binding fragment or other recombinant anti-Tau antigen-binding form which comprises the nucleotide sequences encoding the heavy and light chains of the variable regions of NI-502.31B6 (31B6) (nucleotides sequences SEQ ID NOs. 3 and 4, respectively, see Table 1). The nucleotide sequences may be codon optimized for expression in human cells. The amino acid sequences of the heavy and light chain variable domains of NI-502.31B6 are provided in Table 2, and in particular, are SEQ ID NO: 99 (encoding the NI-502.31B6 heavy chain variable portion) and SEQ ID NO: 100 (encoding the NI-502.31B6 light chain variable portion). Exemplary transgene products are provided in FIG. 1E. The transgene may encode heavy and light chain variable domain sequences that have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 87) or a signal sequence listed in Table 9.

In addition to the heavy and light chain variable domains, the heavy and light chains encoded by the transgene may also comprise C_{H}1 and CL domain sequences, and, in certain embodiments, the transgenes may comprise, at the C-terminus of the heavy chain C_{H}1 domain sequence, all or a portion of the hinge region. The C_{H}1-domain may be an IgG1 (SEQ ID NO: 193), IgG2 (SEQ ID NO: 194) or IgG4 (SEQ ID NO: 195) C_{H}1 domain. In some embodiments, the anti-Tau-antigen binding domain has or comprises a heavy chain variable domain of SEQ ID NO: 99, and a C_{H}1 of IgG1 (SEQ ID NO: 193), IgG2 (SEQ ID NO: 194) or IgG4 (SEQ ID NO: 195), or a variant thereof. In certain embodiments, the anti-Tau-antigen binding domain has a heavy chain domain comprising VH and C_{H}1, with the heavy chain consisting of or comprising an amino acid sequence of SEQ ID NOs: 356 (IgG1), 357 (IgG2), or 358 (IgG4) (Table 2) and a light chain consisting of or comprising a VL and a CL with an amino acid sequence of SEQ ID NO: 190 or 367.

In certain embodiments, the anti-Tau-antigen binding domain has a heavy chain variable domain of SEQ ID NO: 99 and a IgG1 C_{H}1 domain sequence (SEQ ID NO: 193) with additional hinge region sequence starting after the C-terminal valine (V), contains all or a portion of the amino acid sequence EP, EPKS, EPKSCDKTHTCPPCPAPELLGG (SEQ ID NO: 172), EPKSCDKTHTCPPCPA (SEQ ID NO: 173), EPKSCDKTHT(SEQ ID NO: 174), EPKSCDKTHLCPPCPAPELLGG (SEQ ID NO: 175), EPKSCDKTHLCPPCPA(SEQ ID NO: 176), EPKSCDKTHL(SEQ ID NO: 177), EPKSCDKTHTCPPCPAPELLGGPSVFL (SEQ ID NO: 178) or EPKSCDKTHLCPPCPAPELLGGPSVFL (SEQ ID NO: 179) as set forth in FIG. 1E. In particular embodiments, the anti-Tau antigen binding domain has a heavy chain comprising a VH, C_{H}1 and all or a portion of the hinge domain, with the heavy chain consisting of or comprising an amino acid sequence of SEQ ID NO: 189 and a light chain consisting of or comprising a VL and a CL (for example a lambda CL) with an amino acid sequence of SEQ ID NO: 190 (see FIG. 1E and Table 2 for heavy and light chain amino acid sequences).

In other embodiments, the anti-Tau-antigen binding domain has a heavy chain variable domain of SEQ ID NO: 99 and a IgG4 C_{H}1 domain sequence (Table 6, SEQ ID NO: 195) with optionally additional hinge region sequence starting after the C-terminal valine (V), contains all or a portion of the amino acid sequence ESKYGPPCPPCPAPEFLGG (SEQ ID NO: 180), and specifically, ES, ESKY, ESKYGPPCPPCPA (SEQ ID NO: 181), ESKYGPPCPSCPA (SEQ ID NO: 182), ESKYGPPCPSCPAPEFLGGPSVFL (SEQ ID NO: 183), or ESKYGPPCPPCPAPEFLGGPSVFL (SEQ ID NO: 184). In other embodiments, the anti-Tau-antigen binding domain has a heavy chain variable domain of SEQ ID NO: 99 and a IgG2 C_{H}1 domain sequence (SEQ ID NO: 194) with additional hinge region sequence starting after the C-terminal valine (V), contains all or a portion of the amino acid sequence ERKCCVECPPCPAPPVAG (SEQ ID NO: 185) or ERKCCVECPPCPA (SEQ ID NO: 186). See also Table 7 for a listing of the amino acid sequences of hinge regions.

In another embodiment, the transgenes comprise the amino acid sequences encoding the full length (or substantially full length) heavy and light chains of the antibody, that is the heavy chain comprising the VH, C_{H}1, hinge and, in addition, the Fc domain at the C terminus of the heavy chain. The Fc domains that may be incorporated into the anti-Tau antibodies encoded by the transgenes described herein are as disclosed in Section 5.2.3.1, infra. The Fc domain may be an IgG1 (SEQ ID NO: 196), IgG2 (SEQ ID NO: 197) or IgG4 (SEQ ID NO: 198) Fc domain and may have one or more amino acid modifications that alter Fc effector function (including altered binding to one or more Fc receptors) or increase serum half-life. In particular embodiments, the Fc domain which includes the C_{H}2 and C_{H}3 domains of the heavy chain has an amino acid sequence of SEQ ID NOs: 196, 197, or 198 (Table 6), or a mutant or variant thereof. Exemplary amino acid sequences of the entire constant region (SEQ ID NOs: 103, 105, 107, 109, or 110) comprising C_{H}1 domain, hinge region, C_{H}2 domain, and C_{H}3 domain are also provided in Table 6. Also provided are anti-Tau mAbs which have a murine constant region of Fc domain (for example, an IgG2a Fc domain or the entire IgG2 constant region, SEQ ID NOs: 112 or 113) which may be useful for study in mouse models of disease.

In certain embodiments, the anti-Tau antigen-binding fragment transgene encodes an Tau antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 100. In certain embodiments, the anti-Tau antigen-binding fragment transgene encodes an Tau antigen-binding fragment comprising a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 99. In certain embodiments, the anti-Tau antigen-binding fragment transgene encodes an antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 100 and a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 99. In certain embodiments, the Tau antigen binding fragment comprises a heavy chain variable domain comprising an amino acid sequence of SEQ ID NO: 99 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1E and the sequences set forth in Table 3 (SEQ ID NOs: 160, 161, and 162, being the amino acid sequences of the VH-CDR1, VH-CDR2, and VH-CDR3, respectively)). In certain embodiments, the Tau antigen binding fragment comprises a light chain variable domain comprising an amino acid sequence of SEQ ID NO: 100 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1E and the sequences set forth in Table 3 (SEQ ID NOs: 163, 164, and 165, being the amino acid sequences of the VL-CDR1, VL-CDR2, and VL-CDR3, respectively)). In certain embodiments, the anti-Tau antigen-binding fragment transgene encodes a hyperglycosylated NI-502.31B6 Fab, comprising a heavy chain and a light chain of SEQ ID NOs: 189 and 190, respectively, with one or more of the following mutations: L121N (heavy chain) and/or E201N (light chain).

In certain embodiments, the anti-Tau antigen binding fragment transgene encodes an antigen binding fragment and comprises the nucleotide sequence encoding the six NI-502.31B6 CDRs, which may be determined readily by those skilled in the art and including those which are underlined in the heavy and light chain variable domain sequences of FIG. 1E (see also Table 3) which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof. In certain embodiments, the anti-Tau antigen-binding fragment transgene encodes an antigen-binding fragment and comprises the nucleotide sequences encoding the six NI-502.31B6 CDRs (comprising for example, SEQ ID NOs: 160-165) which are underlined in the heavy and light chain variable domain sequences of FIG. 1E which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

The transgenes provided herein encode an anti-Tau mAb (FIG. 1E), particularly either as a full-length antibody or an antigen binding fragment thereof, e.g. a Fab or Fab′ fragment or a F(ab′)₂, or a synthetic or recombinant form incorporating an antigen-binding domain thereof (such as, for example, an scFv, minibody, diabody, nanobody, scFv-Fc). Exemplary structures of antigen-binding fragments and recombinant forms are depicted in FIG. 1A and exemplary amino acid sequences as well as nucleotide sequences are provided in Tables 2, 5, and 11A-11G respectively.

Exemplary transgenes that encoding an anti-Tau ScFv-Fc include nucleotide sequences of SEQ ID NOs: 372 or 373. In some embodiments, the transgenes are present in constructs providing an ITR to ITR cassette of SEQ ID NO:372. Exemplary transgenes that represent an anti-Tau Fab or are nucleic acid sequences that encode an anti-Tau Fab are those sequences of SEQ ID NOs: 390 and 391. In some embodiments, the transgenes are present in constructs providing an ITR to ITR cassette with a nucleotide sequence of SEQ ID NO:390.

In certain embodiments, provided are transgenes that encode the anti-Tau antibody, the anti-Tau antigen-binding fragment or other recombinant anti-Tau antigen-binding form which comprises the nucleotide sequences encoding the heavy and light chains of the variable regions of NI-502.8H1 (8H1) (nucleotide sequences SEQ ID NOs. 5 and 6, respectively, see Table 1). The nucleotide sequences may be codon optimized for expression in human cells. The amino acid sequences of the heavy and light chain variable domains of NI-502.8H1 are provided in Table 2, and, in particular, are SEQ ID NO: 101 (encoding the NI-502.8H1 heavy chain variable portion) and SEQ ID NO: 102 (encoding the NI-502.8H1 light chain variable portion). The transgene may encode heavy and light chain variable domain sequences that have a signal or leader sequence at the N-terminus appropriate for expression and secretion in human cells, in particular, human CNS cells. The signal sequence may have the amino acid sequence of MYRMQLLLLIALSLALVTNS (SEQ ID NO: 87) or a signal sequence listed in Table 9.

In addition to the heavy and light chain variable domains, the heavy and light chains encoded by the transgene may also comprise C_{H}1 and CL domain sequences, and, in certain embodiments, the transgenes may comprise, at the C-terminus of the heavy chain C_{H}1 domain sequence, all or a portion of the hinge region (see Table 7 for hinge sequences). The C_{H}1-domain may be an IgG1 (SEQ ID NO: 193), IgG2 (SEQ ID NO: 194) or IgG4 (SEQ ID NO: 195) C_{H}1 domain. In some embodiments, the anti-Tau-antigen binding domain has or comprises a heavy chain variable domain of SEQ ID NO: 101, and a C_{H}1 of IgG1 (SEQ ID NO: 193), IgG2 (SEQ ID NO: 194) or IgG4 (SEQ ID NO: 195), or a variant thereof. In certain embodiments, the anti-Tau-antigen binding domain has a heavy chain domain comprising VH and C_{H}1, with the heavy chain consisting of or comprising an amino acid sequence of SEQ ID NOs: 361 (IgG1), 362 (IgG2), or 363 (IgG4) (Table 2) and a light chain consisting of or comprising a VL and a CL with an amino acid sequence of SEQ ID NO: 192 or 368.

In some embodiments, the anti-Tau-antigen binding domain has a heavy chain variable domain of SEQ ID NO: 101 and a IgG1 C_{H}1 domain sequence (SEQ ID NO: 193) with additional hinge region sequence starting after the C-terminal valine (V), contains all or a portion of the amino acid sequence EP, EPKS, EPKSCDKTHTCPPCPAPELLGG (SEQ ID NO: 172), EPKSCDKTHTCPPCPA(SEQ ID NO: 173), EPKSCDKTHT(SEQ ID NO: 174), EPKSCDKTHLCPPCPAPELLGG (SEQ ID NO: 175), EPKSCDKTHLCPPCPA(SEQ ID NO: 176), EPKSCDKTHL(SEQ ID NO: 177), EPKSCDKTHTCPPCPAPELLGGPSVFL (SEQ ID NO: 178) or EPKSCDKTHLCPPCPAPELLGGPSVFL (SEQ ID NO: 179) as set forth in FIG. 1F. In particular embodiments, the anti-Tau antigen binding domain has a heavy chain comprising a VH, C_{H}1 and all or a portion of the hinge domain with the heavy chain consisting of or comprising an amino acid sequence of SEQ ID NO: 191 and a light chain consisting of or comprising a VL and a CL (for example, a kappa CL) with an amino acid sequence of SEQ ID NO: 192 or 368 (see FIG. 1F and Table 2 for heavy and light chain sequences).

In other embodiments, the anti-Tau-antigen binding domain has a heavy chain variable domain of SEQ ID NO: 101 and a IgG4 C_{H}1 domain sequence (SEQ ID NOs: 195) with optionally additional hinge region sequence starting after the C-terminal valine (V), contains all or a portion of the amino acid sequence ESKYGPPCPPCPAPEFLGG (SEQ ID NO: 180), and specifically, ES, ESKY, ESKYGPPCPPCPA (SEQ ID NO: 181), ESKYGPPCPSCPA (SEQ ID NO: 182), ESKYGPPCPSCPAPEFLGGPSVFL (SEQ ID NO: 183), or ESKYGPPCPPCPAPEFLGGPSVFL (SEQ ID NO: 184). In other embodiments, the anti-Tau-antigen binding domain has a heavy chain variable domain of SEQ ID NO: 101 and a IgG2 C_{H}1 domain sequence (SEQ ID NO: 194) with additional hinge region sequence starting after the C-terminal valine (V), contains all or a portion of the amino acid sequence ERKCCVECPPCPAPPVAG (SEQ ID NO: 185) or ERKCCVECPPCPA (SEQ ID NO: 186). See also Table 7 for a listing of the amino acid sequences of hinge regions.

In another embodiment, the transgenes comprise the amino acid sequences encoding the full length (or substantially full length) heavy and light chains of the antibody, that is the heavy chain comprises the VH, C_{H}1 and hinge domain and, in addition, the Fc domain. The Fc domains that may be incorporated into the anti-Tau antibodies encoded by the transgenes described herein are as disclosed in Section 5.2.4, infra. The Fc domain which includes the C_{H}2 and C_{H}3 domains of the heavy chain may be an IgG1 (SEQ ID NO: 196), IgG2 (SEQ ID NO: 197) or IgG4 (SEQ ID NO: 198) Fc domain and may have one or more amino acid modifications that alter Fc effector function (including altered binding to one or more Fc receptors) or increase serum half-life. In particular embodiments, the Fc domain has an amino acid sequence of SEQ ID NOs: 196, 197, or 198 (Table 6), or a mutant or variant thereof. Exemplary amino acid sequences of the entire constant region (SEQ ID NOs: 103, 105, 107, 109, or 110) comprising C_{H}1 domain, hinge region, C_{H}2 domain, and C_{H}3 domain are also provided in Table 6. Also provided are anti-Tau mAbs which have a murine constant region of Fc domain (for example, an IgG2a Fc domain or the entire IgG2 constant region, SEQ ID NOs: 112 or 113) which may be useful for study in mouse models of disease.

In certain embodiments, the anti-Tau antigen-binding fragment transgene encodes an Tau antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 102. In certain embodiments, the anti-Tau antigen-binding fragment transgene encodes an Tau antigen-binding fragment comprising a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 101. In certain embodiments, the anti-Tau antigen-binding fragment transgene encodes an antigen-binding fragment comprising a light chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 102 and a heavy chain variable domain comprising an amino acid sequence that is at least 85%, 86%, 87%, 88%, 89%, 90%, 91%, 92%, 93%, 94%, 95%, 96%, 97%, 98% or 99% identical to the sequence set forth in SEQ ID NO: 101. In certain embodiments, the Tau antigen binding fragment comprises a heavy chain variable domain comprising an amino acid sequence of SEQ ID NO: 101 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1F and the sequences set forth in Table 3 (SEQ ID NOs: 166, 167, and 168, being the amino acid sequences of the VH-CDR1, VH-CDR2, and VH-CDR3, respectively)). In certain embodiments, the Tau antigen binding fragment comprises a light chain comprising an amino acid sequence of SEQ ID NO: 100 with 1, 2, 3, 4, 5, 6, 7, 8, 9, 10, 11, 12, 13, 14, 15 or more amino acid substitutions, insertions or deletions, and the substitutions, insertions or deletions are made, e.g., in the framework regions (e.g., those regions outside of the CDRs, which CDRs are underlined in FIG. 1F and the sequences set forth in Table 3 (SEQ ID NOs: 169, 170, and 171, being the amino acid sequences of the VL-CDR1, VL-CDR2, and VL-CDR3, respectively)). In certain embodiments, the anti-Tau antigen-binding fragment transgene encodes a hyperglycosylated NI-502.8H1 Fab, comprising a heavy chain and a light chain of SEQ ID NOs: 191 and 192, respectively, with one or more of the following mutations: L117N (heavy chain) and/or Q157N, Q157S, and/or E197N (light chain).

In certain embodiments, the anti-Tau antigen binding fragment transgene encodes an antigen binding fragment and comprises the nucleotide sequence encoding the six NI-502.8H1 CDRs which may be determined readily by those skilled in the art and including those which are underlined in FIG. 1F (see also Table 3), which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof. In certain embodiments, the anti-Tau antigen-binding fragment transgene encodes an antigen-binding fragment and comprises the nucleotide sequences encoding the six NI-502.8H1 CDRs (comprising, for example, SEQ ID NOs: 166-171) which are underlined in the heavy and light chain variable domain sequences of FIG. 1F which are spaced between framework regions, generally human framework regions, and associated with constant domains depending upon the form of the antigen-binding molecule, as is known in the art to form the heavy and/or light chain variable domain of an anti-Tau antibody or antigen-binding fragment thereof.

The transgenes provided herein encode an anti-Tau mAb (FIG. 1F), particularly either as a full-length antibody or an antigen binding fragment thereof, e.g. a Fab or Fab′ fragment or a F(ab′)₂, or a synthetic or recombinant form incorporating an antigen-binding domain thereof (such as, for example, an scFv, minibody, diabody, nanobody, scFv-Fc). Exemplary structures of antigen-binding fragments and recombinant forms are depicted in FIG. 1A and exemplary amino acid sequences as well as nucleotide sequences are provided in Tables 2, 5, and 11A-11G, respectively.

Exemplary transgenes that encode an anti-Tau Fab include nucleotide sequences of SEQ ID NOs: 384 and 385. In some embodiments, the transgenes are present in constructs providing an AAV artificial genome ITR to ITR cassette of SEQ ID NO: 384.

### 5.2.3. Vectored Antibody Constructs

The recombinant expression cassettes provided herein comprise the following components: (1) AAV inverted terminal repeats that flank the expression cassette; (2) regulatory control elements, which include a) a promoter, b) optionally an intron and c) a poly A signal; and (3) nucleic acid sequences coding for the heavy chain and light chain of a Tau-specific mAb or antigen-binding fragments thereof or a recombinant antigen-binding protein, such as the heavy and light chains of a full length antibody, a Fab fragment, an scFv, an scFv-Fc, or other recombinant antigen binding form as described herein. Nucleotide sequences of exemplary regulatory sequences are provided in Table 8. In some embodiments, the nucleic acid regulatory control element is operably linked to a composite transgene. The composite transgene comprises a heavy chain antibody sequence linked to a 5′ leader (signal) sequence, a linker (for example, an IRES or cleavable linker as disclosed herein), and a light chain antibody sequence linked to a 5′ leader (signal) sequence. In some embodiments, the heavy chain antibody sequence comprises a full or partial heavy chain constant region (e.g., comprising a heavy chain variable domain and optionally a C_{H}1 domain. In some embodiments, the light chain antibody sequence comprises a full or partial light chain constant region (e.g., comprising a light chain variable domain and optionally a light chain constant domain). The expression cassette may comprise any combination of one of the genes or nucleic acids, including one or more nucleic acids from Tables 1, 11D, 11E, 11F, or 11G, encoding the therapeutic proteins, with amino acid sequences listed in, but not limited to, Tables 2, 11A, 11B, or 11C. Exemplary expression cassettes are depicted in FIGS. 1B and C, and the amino acid sequences of expressed transgenes are provided in FIGS. 1D-1F.

In an aspect of the invention, various regulatory elements and combinations of elements, such as linkers (see Table 8 infra), were utilized to design and generate nucleic acid expression cassettes for expression of the composite antibody sequences, which are listed in Table 5 for the full length antibodies. See also Tables 11F and 11G for constructs encoding scFv-Fcs and Fab fragments.

TABLE 5
Composite Antibody Transgene Nucleic Acids

| | SEQ | |
|---|---|---|
| Name | ID NO: | Nucleic Acid Sequence |
| 4P3 IgG2a | 7 | ATGTACAGAATGCAGCTGCTGCTGCTCATTGCCCTGTCTCTGGCCCT |
| Leader- | | GGTCACCAATTCTGAGGTGCAGCTGGTGCAGTCTGGGGCTGAAGTGA |
| 4P3chim. HC- | | AGAAGCCTGGGGAGAGCCTGAAGATCAGCTGCAAAGTGTCTGGCTAT |
| furin/T2A- | | GACTTCAGCAAGTACTGGATTGGCTGGGTCAGACAGATGCCTGGCAA |
| Leader-4P3.LC | | AGGCCTGGAATGGATGGGACTGATCTACCCTGGGGACTCTGACACCA |
| (HC = mIgG2a) | | GATACAGCCCTAGCCTGGAAGGCCAGGTGTCCATCTCTGCTGACAAG |
| (LC = kappa) | | AGCATCTCTGCAGCCTACCTGCAGTTCAGCAGCCTGAAGGCCTCTGA |
| | | CTCTGCCATGTACTACTGTGTCAGAAAGGGCTACTCTGCCTACCTTG |
| | | GAGGATGGTTTGATCCTTGGGGCCAGGGCACACTGGTCACAGTGTCC |
| | | TCTGCTAAGACAACAGCCCCTTCTGTGTACCCTCTGGCTCCTGTGTG |
| | | TGGGGACACAACAGGATCCTCTGTGACACTGGGCTGCCTGGTCAAGG |
| | | GATACTTCCCTGAGCCAGTGACACTGACCTGGAACTCTGGATCTCTG |
| | | TCCTCTGGGGTGCACACATTCCCTGCTGTGCTGCAGTCTGACCTGTA |
| | | CACCCTGAGCAGCAGTGTGACAGTGACCAGCAGCACATGGCCCAGCC |
| | | AGAGCATCACCTGTAATGTGGCTCACCCTGCCAGCTCCACCAAGGTG |
| | | GACAAGAAGATTGAGCCTAGAGGCCCCACCATCAAGCCCTGTCCTCC |
| | | ATGCAAGTGTCCTGCTCCTAACCTGCTTGGAGGCCCCTCTGTGTTCA |
| | | TCTTCCCACCTAAGATCAAGGATGTGCTGATGATCTCTCTGAGCCCC |
| | | ATTGTGACCTGTGTGGTGGTGGATGTGTCTGAGGATGACCCTGATGT |
| | | GCAGATCTCTTGGTTTGTGAACAATGTGGAAGTGCACACAGCCCAGA |
| | | CACAGACCCACAGAGAGGACTACAACAGCACCCTGAGAGTGGTGTCT |
| | | GCCCTGCCTATCCAGCACCAGGACTGGATGAGTGGCAAAGAGTTCAA |
| | | GTGCAAAGTGAACAACAAGGACCTGCCTGCTCCAATTGAGAGAACCA |
| | | TCAGCAAGCCCAAGGGCTCTGTTAGAGCCCCTCAAGTGTATGTGCTG |
| | | CCTCCACCTGAGGAAGAGATGACCAAGAAACAAGTGACCCTGACATG |
| | | CATGGTCACAGACTTCATGCCTGAGGACATCTATGTGGAATGGACCA |
| | | ACAATGGCAAGACAGAGCTGAACTACAAGAACACTGAGCCTGTGCTG |
| | | GACTCTGATGGCAGCTACTTCATGTACAGCAAGCTGAGGGTTGAGAA |
| | | GAAGAACTGGGTTGAGAGAAACAGCTACAGCTGCTCTGTGGTGCATG |
| | | AGGGCCTGCACAATCACCACACCACCAAGAGCTTCAGCAGAACCCCT |
| | | GGCAAGAGAAAGAGAAGAGGCTCTGGAGAAGGCAGAGGCTCCCTGCT |
| | | GACATGTGGGGATGTTGAAGAGAATCCTGGGCCTATGTATAGGATGC |
| | | AACTGCTCCTCCTGATTGCTCTGAGCCTGGCTCTTGTGACCAACTCT |
| | | GACATCCAGCTGACACAGAGCCCTGACAGCCTGGCTGCTTCTCTGGG |
| | | AGAGAGAGTGACCATCAACTGCAGAAGCTCCCAGAACATCTTCTACA |
| | | ACAGCAACAACAAGAACTTCCTGGCCTGGTATCAGCAGAAGGCTGGC |
| | | CAGTCTCCTAAGCTGCTGATCTACTGGGCCAGCACCAGAGAATCTGG |
| | | GGTGCCAGATAGATTTTCTGGCAGTGGCTCTGGCACAGACTTCACCC |
| | | TGACAATCAGTGGACTGCAGGCTGGGGATGTTGCTGTGTACTTCTGC |
| | | CAGCAGTACTTCACAGATGCCCCTACCTTTGGTGGTGGCACCAAGGT |
| | | GGAAATCAAGAGGGCTGATGCTGCCCCTACAGTGTCTATCTTCCCAC |
| | | CTAGCTCTGAGCAGCTGACCAGTGGTGGTGCCTCTGTTGTGTGCTTC |
| | | CTGAACAACTTCTACCCCAAGGACATCAATGTGAAGTGGAAGATTGA |
| | | TGGCTCTGAGAGGCAGAATGGGGTGCTGAACAGCTGGACAGACCAGG |
| | | ACAGCAAGGACTCCACCTACAGCATGAGCAGCACACTGACCCTGACC |
| | | AAGGATGAGTATGAGAGGCACAACAGCTACACCTGTGAAGCCACACA |
| | | CAAGACCAGCACAAGCCCCATTGTGAAGTCCTTCAACAGAAATGAGT |
| | | GTTAA |
| 31B6 IgG2a | 8 | ATGTACAGAATGCAGCTGCTGCTGCTCATTGCCCTGTCTCTGGCCCT |
| Leader- | | GGTCACCAATTCTGAGGTGCAGCTGCTGCAGAGTGGTGGTGGTGTTG |
| 31B6.chim.HC- | | TGCAGCCTGGTGGCTCTCTGAGACTGTCTTGTGAAGCCTCTGGCTTC |
| furin/T2A- | | ACCTTCAGCAGCTACAGCATGCACTGGGTCAGACAGGCCCCTGGCAA |
| Leader-31B6.LC | | AGGACTGGAATGGCTGACCCTGATCAGCTACAATGGGGTCAACAAGT |
| (HC = mIgG2a) | | ACTATGCCAAGTCTGTGAAGGGCAGATTTGCCATCAGCAGAGATGAC |
| (LC = lambda) | | AGCAAGTCTACCCTGTTCCTGCAGATGAACAGCCTGACCACAGATGA |
| | | CACTGCTGTGTACTACTGTGCCAGATGTCCCATTCCTAGAGCTGCCA |
| | | GCTACAGAAATGGGGACAACTACTTTGAGTTTGGCCAGGGCACCCTG |
| | | GTCACAGTGTCCTCTGCTAAGACAACAGCCCCTTCTGTGTACCCTCT |
| | | GGCTCCTGTGTGTGGGGACACAACAGGATCCTCTGTGACACTGGGCT |
| | | GCCTGGTCAAGGGCTACTTTCCTGAGCCAGTGACACTGACCTGGAAC |
| | | TCTGGATCTCTGTCCTCTGGGGTGCACACATTCCCTGCTGTGCTGCA |
| | | GTCTGACCTGTACACCCTGAGCAGCAGTGTGACAGTGACCAGCAGCA |
| | | CATGGCCCAGCCAGAGCATCACCTGTAATGTGGCTCACCCTGCCAGC |
| | | TCCACCAAGGTGGACAAGAAGATTGAGCCTAGAGGCCCCACCATCAA |
| | | GCCCTGTCCTCCATGCAAGTGTCCTGCTCCTAACCTGCTTGGAGGCC |
| | | CCTCTGTGTTCATCTTCCCACCTAAGATCAAGGATGTGCTGATGATC |
| | | TCTCTGAGCCCCATTGTGACCTGTGTGGTGGTTGATGTGTCTGAGGA |
| | | TGACCCAGATGTGCAGATCTCTTGGTTTGTGAACAATGTGGAAGTGC |
| | | ACACAGCCCAGACACAGACCCACAGAGAGGACTACAACAGCACCCTG |
| | | AGAGTGGTGTCTGCCCTGCCTATCCAGCACCAGGACTGGATGTCTGG |
| | | CAAAGAGTTCAAGTGCAAAGTGAACAACAAGGACCTGCCTGCTCCAA |
| | | TTGAGAGAACCATCAGCAAGCCCAAGGGCTCTGTTAGAGCCCCTCAA |
| | | GTGTATGTGCTGCCTCCACCTGAGGAAGAGATGACCAAGAAACAAGT |
| | | GACCCTGACATGCATGGTCACTGACTTCATGCCTGAGGACATCTATG |
| | | TGGAATGGACCAACAATGGCAAGACAGAGCTGAACTACAAGAACACT |
| | | GAGCCTGTGCTGGACTCTGATGGCAGCTACTTCATGTACAGCAAGCT |
| | | GAGGGTTGAGAAGAAGAACTGGGTTGAGAGAAACAGCTACTCCTGCT |
| | | CTGTGGTGCATGAGGGCCTGCACAACCATCACACCACAAAGAGCTTC |
| | | AGCAGAACCCCAGGCAAGAGAAAGAGAAGAGGCTCTGGAGAAGGCAG |
| | | AGGCTCCCTGCTGACATGTGGGGATGTTGAAGAGAATCCTGGGCCTA |
| | | TGTATAGGATGCAACTGCTCCTCCTGATTGCTCTGAGCCTGGCTCTT |
| | | GTGACCAACTCTCAGGCTGTGGTCACCCAAGAGCCTAGCCTGTCTGT |
| | | GTCTCCTGGTGGCACAGTGACACTGACATGTGCCCTGTCCTCTGCCT |
| | | CTGTGTCTACCAGCAGCTACCCCAGCTGGTATCAGCAGACACCTGGC |
| | | CAGCCTCCAAGAATGCTGATCCACAACACAGACACCAGATCCTCTGG |
| | | GGTGCCAGACAGATTCTCTGGCAGCATCCTGGGCAAGAAGGCTGCCC |
| | | TGACAATCACTGGTGCCCAGGCTGATGATGAGTCTGACTACTACTGT |
| | | GTGGTGTACCTTGGCAATGGCTTCTGGGTGTTTGGTGGTGGCACCAA |
| | | GCTGACAGTGCTGGGCCAGCCTAAAAGCAGCCCCTCTGTGACACTGT |
| | | TCCCTCCAAGCTCTGAGGAACTGGAAACAAACAAGGCCACACTTGTG |
| | | TGCACCATCACAGACTTCTACCCTGGGGTTGTGACAGTGGACTGGAA |
| | | AGTGGATGGCACCCCTGTGACACAAGGCATGGAAACCACACAGCCCA |
| | | GCAAGCAGAGCAACAACAAGTACATGGCCAGCTCCTATCTGACCCTG |
| | | ACAGCCAGAGCCTGGGAGAGACACAGCTCTTACAGCTGCCAAGTGAC |
| | | CCATGAGGGCCACACAGTGGAAAAGAGCCTGAGCAGAGCTGACTGCT |
| | | CTTAA |
| 8H1 IgG2a | 9 | ATGTACAGAATGCAGCTGCTGCTGCTCATTGCCCTGTCTCTGGCCCT |
| Leader- | | GGTCACCAATTCTGAGGTGCAGCTGGTGGAAAGTGGTGGTGGACTGG |
| 8H1.chim.HC.- | | TTCAGCCTGGTGGCAGCCTGAGACTGTCTTGTGCTGCCTCTGGCTTC |
| furin/T2A- | | ACCTTCAGCATCTACACCATGAGCTGGGTCAGACAGGCTCCTGGCAA |
| Leader-8H1.LC | | AGGCCTGGAATGGGTGTCAGCTATCCTGTCTGATGGCAGCACCTACT |
| (HC = mIgG2a) | | ATGCTGACTCTGTGGAAGGCAGATTCACCATCAGCAGAGACAACAGC |
| (LC = kappa) | | AAGACCACACTGTACCTGCAGATGAACAGCCTGACAGCTGAGGACAC |
| | | TGCTGTGTACTACTGCACCAAGAGAGTGGAAGGCTCCTTCAGAGGCT |
| | | GGGGACAGGGAACACTGGTCACTGTGTCCTCTGCCAAGACAACTGCC |
| | | CCTTCTGTGTACCCTCTGGCTCCTGTGTGTGGGGACACAACAGGATC |
| | | CTCTGTGACACTGGGCTGCCTGGTCAAGGGCTACTTTCCTGAGCCAG |
| | | TGACACTGACCTGGAACTCTGGATCTCTGTCCTCTGGGGTGCACACA |
| | | TTCCCTGCTGTGCTGCAGTCTGACCTGTACACCCTGAGCAGCTCTGT |
| | | GACTGTGACCAGCAGCACATGGCCCAGCCAGAGCATCACCTGTAATG |
| | | TGGCTCACCCTGCCAGCTCCACCAAGGTGGACAAGAAGATTGAGCCT |
| | | AGAGGCCCCACCATCAAGCCCTGTCCTCCATGCAAGTGTCCTGCTCC |
| | | TAACCTGCTTGGAGGCCCCTCTGTGTTCATCTTCCCACCTAAGATCA |
| | | AGGATGTGCTGATGATCTCTCTGAGCCCCATTGTGACCTGTGTGGTG |
| | | GTTGATGTGTCTGAGGATGACCCTGATGTGCAGATCTCTTGGTTTGT |
| | | GAACAATGTGGAAGTGCACACAGCCCAGACACAGACCCACAGAGAGG |
| | | ACTACAACAGCACCCTGAGAGTGGTGTCTGCCCTGCCTATCCAGCAC |
| | | CAGGACTGGATGTCTGGCAAAGAGTTCAAGTGCAAAGTGAACAACAA |
| | | GGACCTGCCTGCTCCAATTGAGAGAACCATCTCCAAGCCTAAGGGCT |
| | | CTGTCAGGGCCCCTCAAGTGTATGTGCTGCCACCTCCTGAGGAAGAG |
| | | ATGACCAAGAAACAAGTGACCCTGACATGCATGGTCACAGACTTCAT |
| | | GCCTGAGGACATCTATGTGGAATGGACCAACAATGGCAAGACAGAGC |
| | | TGAACTACAAGAACACTGAGCCTGTGCTGGACTCTGATGGCTCCTAC |
| | | TTCATGTACAGCAAGCTGAGGGTTGAGAAGAAGAACTGGGTTGAGAG |
| | | AAACAGCTACAGCTGCTCTGTGGTGCATGAGGGCCTGCACAATCACC |
| | | ACACCACCAAGAGCTTCAGCAGAACCCCTGGCAAGAGAAAGAGAAGA |
| | | GGCTCTGGAGAAGGCAGAGGCTCCCTGCTGACATGTGGGGATGTTGA |
| | | AGAGAATCCTGGGCCTATGTATAGGATGCAACTGCTCCTCCTGATTG |
| | | CTCTGAGCCTGGCTCTTGTGACCAACTCTGAGATTGTGATGACACAG |
| | | AGCCCTGGCACACTGTCTCTGAGCCCTGGGGAATCTGCCACACTGAG |
| | | CTGTAGAGCCAGCCAGTCTGTGTCTACCACCTACCTGGCCTGGTATC |
| | | AGCAGAAGCCTGGACAGGCTCCCAGACTGCTGATCTATGGGGCCTCT |
| | | TCTAGAGCCACAGGCATCCCTGACAGATTTTCTGGCAGTGGCTCTGG |
| | | CACAGACTTCACCCTGACCATCAGCAGACTGGAACCTGAGGACTTTG |
| | | CTGTGTACTACTGCCAGCAGTATGGTGGCAGCAGGCTGATCACATTT |
| | | GGCCAGGGCACAAGACTGGAAATCAAGAGGGCTGATGCTGCCCCTAC |
| | | AGTGTCTATCTTCCCACCTAGCTCTGAGCAGCTGACCAGTGGTGGTG |
| | | CCTCTGTTGTGTGCTTCCTGAACAACTTCTACCCCAAGGACATCAAT |
| | | GTGAAGTGGAAGATTGATGGCTCTGAGAGGCAGAATGGGGTGCTGAA |
| | | CAGCTGGACAGACCAGGACAGCAAGGACTCCACCTACAGCATGAGCA |
| | | GCACCCTGACACTGACCAAGGATGAGTATGAGAGGCACAACAGCTAC |
| | | ACCTGTGAAGCCACACACAAGACCAGCACAAGCCCCATTGTGAAGTC |
| | | CTTCAACAGAAATGAGTGTTAA |
| 4P3 IgG2a | 10 | ATGTACAGAATGCAGCTGCTGCTGCTCATTGCCCTGTCTCTGGCCCT |
| LALAPG | | GGTCACCAATTCTGAGGTGCAGCTGGTGCAGTCTGGGGCTGAAGTGA |
| Leader- | | AGAAGCCTGGGGAGAGCCTGAAGATCAGCTGCAAAGTGTCTGGCTAT |
| 4P3chim.HC. | | GACTTCAGCAAGTACTGGATTGGCTGGGTCAGACAGATGCCTGGCAA |
| LALAPGmut- | | AGGCCTGGAATGGATGGGACTGATCTACCCTGGGGACTCTGACACCA |
| furin/T2A- | | GATACAGCCCTAGCCTGGAAGGCCAGGTGTCCATCTCTGCTGACAAG |
| Leader-4P3.LC | | AGCATCTCTGCAGCCTACCTGCAGTTCAGCAGCCTGAAGGCCTCTGA |
| (HC = mut | | CTCTGCCATGTACTACTGTGTCAGAAAGGGCTACTCTGCCTACCTTG |
| mIgG2a) | | GAGGATGGTTTGATCCTTGGGGCCAGGGCACACTGGTCACAGTGTCC |
| (LC = kappa) | | TCTGCTAAGACAACAGCCCCTTCTGTGTACCCTCTGGCTCCTGTGTG |
| | | TGGGGACACAACAGGATCCTCTGTGACACTGGGCTGCCTGGTCAAGG |
| | | GATACTTCCCTGAGCCAGTGACACTGACCTGGAACTCTGGATCTCTG |
| | | TCCTCTGGGGTGCACACATTCCCTGCTGTGCTGCAGTCTGACCTGTA |
| | | CACCCTGAGCAGCAGTGTGACAGTGACCAGCAGCACATGGCCCAGCC |
| | | AGAGCATCACCTGTAATGTGGCTCACCCTGCCAGCTCCACCAAGGTG |
| | | GACAAGAAGATTGAGCCTAGAGGCCCCACCATCAAGCCCTGTCCTCC |
| | | ATGCAAGTGTCCTGCTCCTAACGCTGCCGGAGGCCCCTCTGTGTTCA |
| | | TCTTCCCACCTAAGATCAAGGATGTGCTGATGATCTCTCTGAGCCCC |
| | | ATTGTGACCTGTGTGGTGGTGGATGTGTCTGAGGATGACCCTGATGT |
| | | GCAGATCTCTTGGTTTGTGAACAATGTGGAAGTGCACACAGCCCAGA |
| | | CACAGACCCACAGAGAGGACTACAACAGCACCCTGAGAGTGGTGTCT |
| | | GCCCTGCCTATCCAGCACCAGGACTGGATGAGTGGCAAAGAGTTCAA |
| | | GTGCAAAGTGAACAACAAGGACCTGGGCGCTCCAATTGAGAGAACCA |
| | | TCAGCAAGCCCAAGGGCTCTGTTAGAGCCCCTCAAGTGTATGTGCTG |
| | | CCTCCACCTGAGGAAGAGATGACCAAGAAACAAGTGACCCTGACATG |
| | | CATGGTCACAGACTTCATGCCTGAGGACATCTATGTGGAATGGACCA |
| | | ACAATGGCAAGACAGAGCTGAACTACAAGAACACTGAGCCTGTGCTG |
| | | GACTCTGATGGCAGCTACTTCATGTACAGCAAGCTGAGGGTTGAGAA |
| | | GAAGAACTGGGTTGAGAGAAACAGCTACAGCTGCTCTGTGGTGCATG |
| | | AGGGCCTGCACAATCACCACACCACCAAGAGCTTCAGCAGAACCCCT |
| | | GGCAAGAGAAAGAGAAGAGGCTCTGGAGAAGGCAGAGGCTCCCTGCT |
| | | GACATGTGGGGATGTTGAAGAGAATCCTGGGCCTATGTATAGGATGC |
| | | AACTGCTCCTCCTGATTGCTCTGAGCCTGGCTCTTGTGACCAACTCT |
| | | GACATCCAGCTGACACAGAGCCCTGACAGCCTGGCTGCTTCTCTGGG |
| | | AGAGAGAGTGACCATCAACTGCAGAAGCTCCCAGAACATCTTCTACA |
| | | ACAGCAACAACAAGAACTTCCTGGCCTGGTATCAGCAGAAGGCTGGC |
| | | CAGTCTCCTAAGCTGCTGATCTACTGGGCCAGCACCAGAGAATCTGG |
| | | GGTGCCAGATAGATTTTCTGGCAGTGGCTCTGGCACAGACTTCACCC |
| | | TGACAATCAGTGGACTGCAGGCTGGGGATGTTGCTGTGTACTTCTGC |
| | | CAGCAGTACTTCACAGATGCCCCTACCTTTGGTGGTGGCACCAAGGT |
| | | GGAAATCAAGAGGGCTGATGCTGCCCCTACAGTGTCTATCTTCCCAC |
| | | CTAGCTCTGAGCAGCTGACCAGTGGTGGTGCCTCTGTTGTGTGCTTC |
| | | CTGAACAACTTCTACCCCAAGGACATCAATGTGAAGTGGAAGATTGA |
| | | TGGCTCTGAGAGGCAGAATGGGGTGCTGAACAGCTGGACAGACCAGG |
| | | ACAGCAAGGACTCCACCTACAGCATGAGCAGCACACTGACCCTGACC |
| | | AAGGATGAGTATGAGAGGCACAACAGCTACACCTGTGAAGCCACACA |
| | | CAAGACCAGCACAAGCCCCATTGTGAAGTCCTTCAACAGAAATGAGT |
| | | GTTAA |
| 31B6 IgG2a | 11 | ATGTACAGAATGCAGCTGCTGCTGCTCATTGCCCTGTCTCTGGCCCT |
| LALAPG | | GGTCACCAATTCTGAGGTGCAGCTGCTGCAGAGTGGTGGTGGTGTTG |
| Leader- | | TGCAGCCTGGTGGCTCTCTGAGACTGTCTTGTGAAGCCTCTGGCTTC |
| 31B6.chim.HC. | | ACCTTCAGCAGCTACAGCATGCACTGGGTCAGACAGGCCCCTGGCAA |
| LALAPGmut- | | AGGACTGGAATGGCTGACCCTGATCAGCTACAATGGGGTCAACAAGT |
| furin/T2A- | | ACTATGCCAAGTCTGTGAAGGGCAGATTTGCCATCAGCAGAGATGAC |
| Leader-31B6.LC | | AGCAAGTCTACCCTGTTCCTGCAGATGAACAGCCTGACCACAGATGA |
| (HC = mut | | CACTGCTGTGTACTACTGTGCCAGATGTCCCATTCCTAGAGCTGCCA |
| mIgG2a) | | GCTACAGAAATGGGGACAACTACTTTGAGTTTGGCCAGGGCACCCTG |
| (LC = lambda) | | GTCACAGTGTCCTCTGCTAAGACAACAGCCCCTTCTGTGTACCCTCT |
| | | GGCTCCTGTGTGTGGGGACACAACAGGATCCTCTGTGACACTGGGCT |
| | | GCCTGGTCAAGGGCTACTTTCCTGAGCCAGTGACACTGACCTGGAAC |
| | | TCTGGATCTCTGTCCTCTGGGGTGCACACATTCCCTGCTGTGCTGCA |
| | | GTCTGACCTGTACACCCTGAGCAGCAGTGTGACAGTGACCAGCAGCA |
| | | CATGGCCCAGCCAGAGCATCACCTGTAATGTGGCTCACCCTGCCAGC |
| | | TCCACCAAGGTGGACAAGAAGATTGAGCCTAGAGGCCCCACCATCAA |
| | | GCCCTGTCCTCCATGCAAGTGTCCTGCTCCTAACGCTGCCGGAGGCC |
| | | CCTCTGTGTTCATCTTCCCACCTAAGATCAAGGATGTGCTGATGATC |
| | | TCTCTGAGCCCCATTGTGACCTGTGTGGTGGTTGATGTGTCTGAGGA |
| | | TGACCCAGATGTGCAGATCTCTTGGTTTGTGAACAATGTGGAAGTGC |
| | | ACACAGCCCAGACACAGACCCACAGAGAGGACTACAACAGCACCCTG |
| | | AGAGTGGTGTCTGCCCTGCCTATCCAGCACCAGGACTGGATGTCTGG |
| | | CAAAGAGTTCAAGTGCAAAGTGAACAACAAGGACCTGGGAGCTCCAA |
| | | TTGAGAGAACCATCAGCAAGCCCAAGGGCTCTGTTAGAGCCCCTCAA |
| | | GTGTATGTGCTGCCTCCACCTGAGGAAGAGATGACCAAGAAACAAGT |
| | | GACCCTGACATGCATGGTCACTGACTTCATGCCTGAGGACATCTATG |
| | | TGGAATGGACCAACAATGGCAAGACAGAGCTGAACTACAAGAACACT |
| | | GAGCCTGTGCTGGACTCTGATGGCAGCTACTTCATGTACAGCAAGCT |
| | | GAGGGTTGAGAAGAAGAACTGGGTTGAGAGAAACAGCTACTCCTGCT |
| | | CTGTGGTGCATGAGGGCCTGCACAACCATCACACCACAAAGAGCTTC |
| | | AGCAGAACCCCAGGCAAGAGAAAGAGAAGAGGCTCTGGAGAAGGCAG |
| | | AGGCTCCCTGCTGACATGTGGGGATGTTGAAGAGAATCCTGGGCCTA |
| | | TGTATAGGATGCAACTGCTCCTCCTGATTGCTCTGAGCCTGGCTCTT |
| | | GTGACCAACTCTCAGGCTGTGGTCACCCAAGAGCCTAGCCTGTCTGT |
| | | GTCTCCTGGTGGCACAGTGACACTGACATGTGCCCTGTCCTCTGCCT |
| | | CTGTGTCTACCAGCAGCTACCCCAGCTGGTATCAGCAGACACCTGGC |
| | | CAGCCTCCAAGAATGCTGATCCACAACACAGACACCAGATCCTCTGG |
| | | GGTGCCAGACAGATTCTCTGGCAGCATCCTGGGCAAGAAGGCTGCCC |
| | | TGACAATCACTGGTGCCCAGGCTGATGATGAGTCTGACTACTACTGT |
| | | GTGGTGTACCTTGGCAATGGCTTCTGGGTGTTTGGTGGTGGCACCAA |
| | | GCTGACAGTGCTGGGCCAGCCTAAAAGCAGCCCCTCTGTGACACTGT |
| | | TCCCTCCAAGCTCTGAGGAACTGGAAACAAACAAGGCCACACTTGTG |
| | | TGCACCATCACAGACTTCTACCCTGGGGTTGTGACAGTGGACTGGAA |
| | | AGTGGATGGCACCCCTGTGACACAAGGCATGGAAACCACACAGCCCA |
| | | GCAAGCAGAGCAACAACAAGTACATGGCCAGCTCCTATCTGACCCTG |
| | | ACAGCCAGAGCCTGGGAGAGACACAGCTCTTACAGCTGCCAAGTGAC |
| | | CCATGAGGGCCACACAGTGGAAAAGAGCCTGAGCAGAGCTGACTGCT |
| | | CTTAA |
| 8H1 IgG2a | 12 | ATGTACAGAATGCAGCTGCTGCTGCTCATTGCCCTGTCTCTGGCCCT |
| LALAPG | | GGTCACCAATTCTGAGGTGCAGCTGGTGGAAAGTGGTGGTGGACTGG |
| Leader- | | TTCAGCCTGGTGGCAGCCTGAGACTGTCTTGTGCTGCCTCTGGCTTC |
| 8H1.chim.HC. | | ACCTTCAGCATCTACACCATGAGCTGGGTCAGACAGGCTCCTGGCAA |
| LALAPGmut- | | AGGCCTGGAATGGGTGTCAGCTATCCTGTCTGATGGCAGCACCTACT |
| furin/T2A- | | ATGCTGACTCTGTGGAAGGCAGATTCACCATCAGCAGAGACAACAGC |
| Leader-8H1.LC | | AAGACCACACTGTACCTGCAGATGAACAGCCTGACAGCTGAGGACAC |
| (HC = mut | | TGCTGTGTACTACTGCACCAAGAGAGTGGAAGGCTCCTTCAGAGGCT |
| mIgG2a) | | GGGGACAGGGAACACTGGTCACTGTGTCCTCTGCCAAGACAACTGCC |
| (LC = kappa) | | CCTTCTGTGTACCCTCTGGCTCCTGTGTGTGGGGACACAACAGGATC |
| | | CTCTGTGACACTGGGCTGCCTGGTCAAGGGCTACTTTCCTGAGCCAG |
| | | TGACACTGACCTGGAACTCTGGATCTCTGTCCTCTGGGGTGCACACA |
| | | TTCCCTGCTGTGCTGCAGTCTGACCTGTACACCCTGAGCAGCTCTGT |
| | | GACTGTGACCAGCAGCACATGGCCCAGCCAGAGCATCACCTGTAATG |
| | | TGGCTCACCCTGCCAGCTCCACCAAGGTGGACAAGAAGATTGAGCCT |
| | | AGAGGCCCCACCATCAAGCCCTGTCCTCCATGCAAGTGTCCTGCTCC |
| | | TAACGCCGCTGGAGGCCCCTCTGTGTTCATCTTCCCACCTAAGATCA |
| | | AGGATGTGCTGATGATCTCTCTGAGCCCCATTGTGACCTGTGTGGTG |
| | | GTTGATGTGTCTGAGGATGACCCTGATGTGCAGATCTCTTGGTTTGT |
| | | GAACAATGTGGAAGTGCACACAGCCCAGACACAGACCCACAGAGAGG |
| | | ACTACAACAGCACCCTGAGAGTGGTGTCTGCCCTGCCTATCCAGCAC |
| | | CAGGACTGGATGTCTGGCAAAGAGTTCAAGTGCAAAGTGAACAACAA |
| | | GGACCTGGGAGCTCCAATTGAGAGAACCATCTCCAAGCCTAAGGGCT |
| | | CTGTCAGGGCCCCTCAAGTGTATGTGCTGCCACCTCCTGAGGAAGAG |
| | | ATGACCAAGAAACAAGTGACCCTGACATGCATGGTCACAGACTTCAT |
| | | GCCTGAGGACATCTATGTGGAATGGACCAACAATGGCAAGACAGAGC |
| | | TGAACTACAAGAACACTGAGCCTGTGCTGGACTCTGATGGCTCCTAC |
| | | TTCATGTACAGCAAGCTGAGGGTTGAGAAGAAGAACTGGGTTGAGAG |
| | | AAACAGCTACAGCTGCTCTGTGGTGCATGAGGGCCTGCACAATCACC |
| | | ACACCACCAAGAGCTTCAGCAGAACCCCTGGCAAGAGAAAGAGAAGA |
| | | GGCTCTGGAGAAGGCAGAGGCTCCCTGCTGACATGTGGGGATGTTGA |
| | | AGAGAATCCTGGGCCTATGTATAGGATGCAACTGCTCCTCCTGATTG |
| | | CTCTGAGCCTGGCTCTTGTGACCAACTCTGAGATTGTGATGACACAG |
| | | AGCCCTGGCACACTGTCTCTGAGCCCTGGGGAATCTGCCACACTGAG |
| | | CTGTAGAGCCAGCCAGTCTGTGTCTACCACCTACCTGGCCTGGTATC |
| | | AGCAGAAGCCTGGACAGGCTCCCAGACTGCTGATCTATGGGGCCTCT |
| | | TCTAGAGCCACAGGCATCCCTGACAGATTTTCTGGCAGTGGCTCTGG |
| | | CACAGACTTCACCCTGACCATCAGCAGACTGGAACCTGAGGACTTTG |
| | | CTGTGTACTACTGCCAGCAGTATGGTGGCAGCAGGCTGATCACATTT |
| | | GGCCAGGGCACAAGACTGGAAATCAAGAGGGCTGATGCTGCCCCTAC |
| | | AGTGTCTATCTTCCCACCTAGCTCTGAGCAGCTGACCAGTGGTGGTG |
| | | CCTCTGTTGTGTGCTTCCTGAACAACTTCTACCCCAAGGACATCAAT |
| | | GTGAAGTGGAAGATTGATGGCTCTGAGAGGCAGAATGGGGTGCTGAA |
| | | CAGCTGGACAGACCAGGACAGCAAGGACTCCACCTACAGCATGAGCA |
| | | GCACCCTGACACTGACCAAGGATGAGTATGAGAGGCACAACAGCTAC |
| | | ACCTGTGAAGCCACACACAAGACCAGCACAAGCCCCATTGTGAAGTC |
| | | CTTCAACAGAAATGAGTGTTAA |
| 4P3 IgG1 | 13 | ATGTACAGAATGCAGCTGCTGCTGCTCATTGCCCTGTCTCTGGCCCT |
| Leader- | | GGTCACCAATTCTGAAGTGCAGCTGGTGCAGTCTGGGGCTGAAGTGA |
| 4P3hum.HC- | | AGAAGCCTGGGGAGAGCCTGAAGATCAGCTGCAAAGTGTCTGGCTAT |
| furin/T2A- | | GACTTCAGCAAGTACTGGATTGGCTGGGTCAGACAGATGCCTGGCAA |
| Leader-4P3.LC | | AGGCCTGGAATGGATGGGCCTGATCTACCCTGGGGACTCTGACACCA |
| (HC = hIgG1) | | GATACAGCCCTAGCCTGGAAGGCCAGGTGTCCATCTCTGCTGACAAG |
| (LC = kappa) | | AGCATCTCTGCAGCCTACCTGCAGTTCAGCAGCCTGAAGGCCTCTGA |
| | | CTCTGCCATGTACTACTGTGTCAGAAAGGGCTACTCTGCCTACCTTG |
| | | GAGGATGGTTTGATCCTTGGGGCCAGGGCACACTGGTTACAGTGTCC |
| | | TCTGCCAGCACAAAGGGCCCCTCTGTTTTTCCACTGGCTCCCAGCAG |
| | | CAAGTCCACCAGTGGTGGTACAGCTGCCCTGGGCTGTCTGGTCAAGG |
| | | ATTACTTCCCTGAGCCTGTGACTGTGTCCTGGAACTCTGGTGCTCTG |
| | | ACCTCTGGGGTGCACACCTTTCCAGCTGTGCTGCAGTCCTCTGGCCT |
| | | GTACTCTCTGTCCTCTGTGGTCACAGTGCCTAGCTCTAGCCTGGGCA |
| | | CCCAGACCTACATCTGCAATGTGAACCACAAGCCTAGCAACACCAAG |
| | | GTGGACAAGAGAGTGGAACCCAAGAGCTGTGACAAGACCCACACCTG |
| | | TCCTCCATGTCCTGCTCCAGAACTGCTTGGAGGCCCTTCTGTGTTCC |
| | | TGTTTCCTCCAAAGCCTAAGGACACCCTGATGATCAGCAGAACCCCT |
| | | GAAGTGACCTGTGTGGTGGTGGATGTGTCCCATGAGGACCCAGAAGT |
| | | GAAGTTCAATTGGTATGTGGATGGGGTTGAAGTGCACAATGCCAAGA |
| | | CCAAGCCTAGAGAGGAACAGTACAACAGCACCTACAGAGTGGTGTCT |
| | | GTGCTGACAGTGCTGCACCAGGACTGGCTGAATGGCAAAGAGTACAA |
| | | GTGCAAGGTGTCCAACAAGGCCCTGCCTGCTCCTATTGAGAAAACCA |
| | | TCAGCAAGGCCAAGGGCCAGCCAAGAGAACCCCAGGTTTACACCCTG |
| | | CCACCTAGCAGAGAAGAGATGACCAAGAATCAGGTGTCCCTGACCTG |
| | | CCTGGTTAAGGGCTTCTACCCCTCTGACATTGCTGTGGAATGGGAGA |
| | | GCAATGGCCAGCCTGAGAACAACTACAAGACAACCCCTCCTGTGCTG |
| | | GACTCTGATGGCTCATTCTTCCTGTACAGCAAGCTGACTGTGGACAA |
| | | GTCCAGATGGCAGCAGGGAAATGTGTTCAGCTGCTCTGTGATGCATG |
| | | AGGCCCTGCACAACCACTACACCCAGAAAAGTCTGTCTCTGTCCCCT |
| | | GGCAAGAGAAAGAGAAGAGGCTCTGGAGAAGGCAGAGGCTCCCTGCT |
| | | GACATGTGGGGATGTTGAAGAGAATCCTGGGCCTATGTATAGGATGC |
| | | AACTGCTCCTCCTGATTGCTCTGAGCCTGGCTCTTGTGACCAACTCT |
| | | GATATCCAGCTGACACAGAGCCCTGACAGCCTGGCTGCTTCTCTGGG |
| | | AGAGAGAGTGACCATCAACTGCAGAAGCTCCCAGAACATCTTCTACA |
| | | ACTCCAACAACAAGAACTTCCTGGCCTGGTATCAGCAAAAGGCTGGA |
| | | CAGAGCCCCAAGCTGCTGATCTACTGGGCCTCTACCAGAGAGTCTGG |
| | | GGTCCCAGACAGATTCTCTGGCTCTGGATCTGGCACAGACTTCACCC |
| | | TGACCATCAGTGGACTGCAGGCTGGGGATGTTGCTGTGTACTTCTGC |
| | | CAGCAGTACTTCACAGATGCCCCTACCTTTGGTGGTGGCACAAAGGT |
| | | GGAAATCAAGAGAACAGTGGCTGCCCCATCTGTGTTCATCTTCCCAC |
| | | CATCTGATGAACAGCTGAAGTCTGGCACTGCCTCTGTTGTGTGCCTG |
| | | CTGAACAACTTCTACCCTAGAGAAGCCAAGGTGCAGTGGAAAGTGGA |
| | | TAATGCCCTCCAGTCTGGCAATAGCCAAGAGTCAGTGACAGAGCAGG |
| | | ACTCCAAGGATTCCACCTACAGCCTGAGCAGCACCCTGACACTGAGC |
| | | AAGGCTGACTATGAGAAGCACAAAGTGTATGCCTGTGAAGTGACACA |
| | | CCAGGGACTGAGCAGCCCAGTGACCAAGAGCTTCAACAGGGGAGAGT |
| | | GCTGA |
| 31B6 IgG1 | 14 | ATGTACAGGATGCAGCTGCTGCTGCTCATTGCCCTGTCTCTGGCCCT |
| Leader- | | GGTCACCAATTCTGAAGTGCAGCTTCTGCAGTCTGGTGGTGGTGTTG |
| 31B6.hum.HC- | | TGCAGCCTGGTGGCTCTCTGAGACTGTCTTGTGAAGCCTCTGGCTTC |
| furin/T2A- | | ACCTTCAGCAGCTACAGCATGCACTGGGTTAGACAGGCCCCTGGCAA |
| Leader-31B6.LC | | AGGCCTGGAATGGCTGACCCTGATCAGCTACAATGGGGTCAACAAGT |
| (HC = hIgG1) | | ACTATGCCAAGTCTGTGAAGGGCAGATTTGCCATCAGCAGAGATGAC |
| (LC = lambda) | | AGCAAGAGCACCCTGTTTCTGCAGATGAACAGCCTGACCACAGATGA |
| | | CACAGCAGTGTACTACTGTGCCAGATGTCCCATTCCTAGAGCTGCCA |
| | | GCTACAGAAATGGGGACAACTACTTTGAGTTTGGCCAGGGCACACTG |
| | | GTCACAGTGTCCTCTGCCTCTACAAAGGGCCCCTCTGTGTTTCCTCT |
| | | GGCTCCTAGCAGCAAGTCCACCAGTGGTGGTACAGCTGCCCTGGGCT |
| | | GTCTGGTCAAGGATTACTTCCCTGAGCCTGTGACTGTGTCCTGGAAT |
| | | TCTGGGGCTCTGACCTCTGGGGTGCACACCTTTCCAGCTGTGCTGCA |
| | | GTCCTCTGGCCTGTACTCTCTGTCCTCTGTGGTTACAGTGCCAAGCA |
| | | GCTCTCTGGGCACCCAGACCTACATCTGCAATGTGAACCACAAGCCT |
| | | AGCAACACCAAGGTGGACAAGAGAGTGGAACCCAAGAGCTGTGACAA |
| | | GACCCACACCTGTCCTCCATGTCCTGCTCCAGAACTGCTTGGAGGCC |
| | | CATCAGTGTTTCTGTTCCCTCCAAAGCCTAAGGACACCCTGATGATC |
| | | AGCAGAACCCCTGAAGTGACCTGTGTGGTGGTGGATGTGTCCCATGA |
| | | GGACCCAGAAGTGAAGTTCAATTGGTATGTGGATGGGGTTGAAGTGC |
| | | ACAATGCCAAGACCAAGCCTAGAGAGGAACAGTACAACAGCACCTAC |
| | | AGAGTGGTGTCTGTGCTGACAGTGCTGCACCAGGACTGGCTGAATGG |
| | | CAAAGAGTACAAGTGCAAGGTGTCCAACAAGGCCCTGCCTGCTCCTA |
| | | TTGAGAAAACCATCAGCAAGGCCAAGGGCCAGCCAAGAGAACCCCAG |
| | | GTTTACACCCTGCCACCTAGCAGAGAAGAGATGACCAAGAACCAGGT |
| | | GTCCCTGACCTGCCTGGTTAAGGGCTTCTACCCCTCTGACATTGCTG |
| | | TGGAATGGGAGAGCAATGGCCAGCCTGAGAACAACTACAAGACAACC |
| | | CCTCCTGTGCTGGACTCTGATGGCTCATTCTTCCTGTACAGCAAGCT |
| | | GACTGTGGACAAGTCCAGATGGCAGCAGGGAAATGTGTTCAGCTGCT |
| | | CTGTGATGCATGAGGCCCTGCACAACCACTACACCCAGAAAAGTCTG |
| | | AGTCTGAGCCCTGGCAAGAGAAAGAGAAGAGGCTCTGGAGAAGGCAG |
| | | AGGCAGCCTGCTGACATGTGGGGATGTTGAAGAGAATCCTGGGCCTA |
| | | TGTATAGAATGCAACTGCTCCTCCTGATTGCTCTGAGCCTGGCTCTG |
| | | GTTACCAACAGCCAGGCTGTGGTCACACAAGAGCCCAGCCTGTCTGT |
| | | GTCTCCTGGTGGAACAGTGACCCTGACTTGTGCCCTCTCTTCTGCCT |
| | | CTGTGTCCACAAGCAGCTACCCCAGCTGGTATCAGCAGACACCTGGC |
| | | CAGCCACCTAGAATGCTGATCCACAACACTGACACCAGAAGCTCTGG |
| | | GGTCCCAGACAGATTCTCTGGCAGCATCCTGGGAAAGAAGGCTGCCC |
| | | TGACAATCACTGGTGCCCAGGCTGATGATGAGTCTGACTACTATTGT |
| | | GTGGTGTACCTTGGCAATGGCTTTTGGGTGTTCGGTGGTGGCACCAA |
| | | ACTGACTGTGCTGGGACAGCCTAAAGCTGCCCCTTCTGTGACACTGT |
| | | TCCCACCTAGCTCTGAGGAACTGCAGGCCAACAAAGCCACACTTGTG |
| | | TGCCTGATCTCTGACTTCTACCCTGGGGCTGTGACAGTGGCTTGGAA |
| | | GGCTGATAGCAGCCCTGTGAAGGCTGGGGTTGAGACAACCACACCTA |
| | | GCAAGCAGAGCAACAACAAATATGCTGCCTCCAGCTATCTGTCTCTG |
| | | ACCCCTGAGCAGTGGAAGTCCCACAGATCCTACAGCTGTCAAGTGAC |
| | | CCATGAGGGCTCCACAGTGGAAAAGACAGTGGCCCCAACAGAGTGCA |
| | | GCTGA |
| 8H1 IgG1 | 15 | ATGTACAGAATGCAGCTGCTGCTGCTCATTGCCCTGTCTCTGGCCCT |
| Leader- | | GGTCACCAATTCTGAAGTGCAGCTGGTGGAAAGTGGTGGTGGACTGG |
| 8H1.hum.HC | | TTCAGCCTGGTGGCAGCCTGAGACTGTCTTGTGCTGCCTCTGGCTTC |
| furin/T2A- | | ACCTTCAGCATCTACACCATGAGCTGGGTCAGACAGGCCCCTGGCAA |
| Leader-8H1.LC | | AGGACTGGAATGGGTGTCAGCCATCCTGTCTGATGGCAGCACCTACT |
| (HC = hIgG1) | | ATGCTGACTCTGTGGAAGGCAGATTCACCATCAGCAGAGACAACAGC |
| (LC = kappa) | | AAGACCACACTGTACCTGCAGATGAACAGCCTGACAGCTGAGGACAC |
| | | AGCAGTGTACTACTGCACCAAGAGAGTGGAAGGCTCCTTCAGAGGCT |
| | | GGGGCCAGGGAACACTGGTTACAGTGTCCTCTGCCAGCACAAAGGGC |
| | | CCCTCTGTTTTTCCACTGGCTCCCAGCAGCAAGAGCACCAGTGGTGG |
| | | AACAGCTGCCCTGGGCTGTCTGGTCAAGGATTACTTCCCTGAGCCTG |
| | | TGACTGTGTCCTGGAACTCTGGTGCACTGACCTCTGGGGTGCACACC |
| | | TTTCCAGCTGTGCTGCAGTCCTCTGGCCTGTACTCTCTGTCCTCTGT |
| | | GGTCACAGTGCCTAGCTCTAGCCTGGGCACCCAGACCTACATCTGCA |
| | | ATGTGAACCACAAGCCTAGCAACACCAAGGTGGACAAAAGAGTGGAA |
| | | CCCAAGAGCTGTGACAAGACCCACACCTGTCCTCCATGTCCTGCTCC |
| | | AGAACTGCTTGGAGGCCCTTCTGTGTTCCTGTTTCCTCCAAAGCCTA |
| | | AGGACACCCTGATGATCAGCAGAACCCCTGAAGTGACCTGTGTGGTG |
| | | GTTGATGTGTCCCATGAGGACCCAGAAGTGAAGTTCAATTGGTATGT |
| | | GGATGGGGTTGAAGTGCACAATGCCAAGACCAAGCCTAGAGAGGAAC |
| | | AGTACAACTCCACCTACAGAGTGGTGTCTGTGCTGACAGTGCTGCAC |
| | | CAGGACTGGCTGAATGGCAAAGAGTACAAGTGCAAGGTGTCCAACAA |
| | | GGCCCTGCCTGCTCCTATTGAGAAAACCATCTCCAAGGCCAAGGGCC |
| | | AGCCAAGAGAACCCCAGGTTTACACCCTGCCACCTAGCAGAGAAGAG |
| | | ATGACCAAGAACCAGGTGTCCCTGACCTGCCTGGTTAAGGGCTTCTA |
| | | CCCCTCTGACATTGCTGTGGAATGGGAGAGCAATGGCCAGCCTGAAA |
| | | ACAACTACAAGACAACCCCTCCTGTGCTGGACTCAGATGGCTCATTC |
| | | TTCCTGTACAGCAAGCTGACTGTGGACAAGTCCAGGTGGCAGCAGGG |
| | | AAATGTGTTCAGCTGCTCTGTGATGCATGAGGCCCTGCACAACCACT |
| | | ACACCCAGAAAAGTCTGAGTCTGAGCCCTGGCAAGAGAAAGAGAAGA |
| | | GGCTCTGGAGAAGGCAGAGGCTCCCTGCTGACATGTGGGGATGTTGA |
| | | AGAGAATCCTGGGCCTATGTATAGGATGCAACTGCTCCTCCTGATTG |
| | | CTCTGAGCCTGGCTCTTGTGACCAACTCTGAGATTGTGATGACACAG |
| | | AGCCCAGGCACACTGTCACTGTCTCCAGGGGAATCTGCCACTCTGAG |
| | | CTGTAGAGCCAGCCAGTCTGTGTCTACCACCTACCTGGCCTGGTATC |
| | | AGCAAAAGCCTGGACAGGCTCCCAGACTGCTGATCTATGGGGCCTCT |
| | | TCTAGAGCCACAGGCATCCCTGACAGATTTTCTGGCAGTGGCTCTGG |
| | | CACAGACTTCACCCTGACCATCTCTAGACTGGAACCTGAGGACTTTG |
| | | CTGTCTACTACTGCCAGCAGTATGGTGGCTCCAGACTGATCACCTTT |
| | | GGCCAGGGCACCAGACTGGAAATCAAGAGAACAGTGGCTGCCCCATC |
| | | TGTGTTCATCTTCCCACCATCTGATGAGCAGCTCAAGTCTGGCACTG |
| | | CCTCTGTTGTGTGCCTGCTGAACAACTTCTACCCTAGAGAAGCCAAG |
| | | GTGCAGTGGAAGGTTGACAATGCCCTGCAGTCTGGCAATAGCCAAGA |
| | | ATCTGTGACAGAGCAGGACTCCAAGGATAGCACCTACAGCCTGTCTA |
| | | GCACCCTGACACTGAGCAAGGCTGACTATGAGAAGCACAAAGTGTAT |
| | | GCCTGTGAAGTGACACACCAGGGCCTGAGCAGCCCAGTGACCAAGAG |
| | | CTTCAATAGAGGGGAGTGCTGA |

Composite nucleic acid sequences that are incorporated into expression cassettes are engineered to express various formats of an antibody. See FIGS. 1B and 1C.—For dimeric antibody formats, gene therapy constructs are designed such that both the heavy and light chains (or fragments thereof making up an antigen-binding fragment) are expressed. The coding sequences for the heavy and light chains can be engineered in a single construct in which the heavy and light chains are separated by a cleavable linker or IRES so that separate heavy and light chain polypeptides are expressed. In certain embodiments, the composite nucleic acid sequences encode for a Fab, Fab′ or F(ab′)₂. In certain embodiments, the construct expresses, from the N-terminus to C-terminus, NH2-leader or localization sequence—VL (optionally CL)-cleavable linker-leader or localization sequence—VH (optionally C_{H}1 and all or a portion of the hinge)-COOH, or NH2-leader or localization sequence—VH (optionally C_{H}1 and all or a portion of the hinge)—cleavable linker-leader or localization sequence—VL (optionally CL)-COOH. In some embodiments, the construct expresses, from the N-terminus to C-terminus, NH2—VL (optionally CL)-cleavable linker-VH (optionally C_{H}1 and all or a portion of the hinge)-COOH, or NH2-VH-cleavable linker-VL-COOH. In certain embodiments the full length heavy and light chains of the antibody are expressed. In some embodiments, the construct expresses, from the N-terminus to C-terminus, NH2—VL-CL-cleavable linker-VH+HC—COOH, or NH2—VH+HC-cleavable linker-VL-CL-COOH. In other embodiments, the construct expresses, from the N-terminus to C-terminus, NH2—leader or localization sequence-VL-cleavable linker-leader or localization sequence-VH+HC≡COOH, or NH2-leader or localization sequence—VH+HC—cleavable linker-leader or localization sequence—VL-CL-COOH.

Gene therapy constructs are also designed such that the antibody is expressed as a single chain format comprising at least one heavy chain variable region and at least one light chain variable region (such as scFv). In some embodiments, the construct expresses a scFv in which the heavy and light chain variable domains are connected via a flexible, non-cleavable linker. In some embodiments, the construct expresses two tandem scFv molecules (taFv). In certain embodiments, the construct expresses, from the N-terminus to C-terminus, NH2-VL-linker-VH—COOH or NH2-VH-linker-VL-COOH. In other embodiments, the construct expresses, from the N-terminus to C-terminus, NH2-leader or localization sequence-VL-linker-VH—COOH or NH2—leader or localization sequence-VH-linker-VL-COOH. In certain embodiments, a taFv is expressed from a single-gene construct encoding two scFv in tandem connected by a peptide linker. In some embodiments, the construct expresses, from the N-terminus to the C-terminus, NH2-leader or localization sequence-VH(A)-linker(>12)-VL(A)-peptide linker-VH(B)-linker(>12)-VL(B)—COOH. The amino acid sequences of the components of the scFv proteins expressed are provided in Table 11A. The proteins encoded by exemplary constructs are provided in Tables 11B (scFv-Fcs) and 11C (scFvs), and may be encoded by the nucleotide sequences provided in Table 11D (encoding components of the scFvs and scFv-Fcs) and 11E and 11F (encoding scFv-Fc constructs) and Table 11G (encoding Fab constructs).

In certain embodiments, the scFv is fused to the hinge and Fc regions of immunoglobulins (scFv-Fc) or to the third constant domain of an IgG (scFv-C_{H}3, also known as a minibody), with or without a hinge region. In intact IgG, the upper hinge contains a cysteine residue which is incorporated into a disulfide bond with the C-terminal cysteine of the kappa light chain. Since kappa light chain is absent from scFv-Fc fusion proteins, the upper hinge can be truncated, the cysteine mutated to another amino acid such as serine or proline, or the “native” sequence can be maintained, in which case it is possible that the extra cysteine residues could form an additional interchain disulfide bond. In some embodiments, the construct expresses from the N-terminus to C-terminus, NH2-leader or localization sequence-VH-linker-VL-hinge region-C_{H}2-C_{H}3-COOH. In other embodiments, the construct expresses from the N-terminus to the C-terminus, NH2-leader or localization sequence-VH-linker-VL-hinge region-C_{H}3-COOH. In some embodiments, the configuration of VH and VL is reversed. For example, in one embodiment, the construct expresses from the N-terminus to C-terminus, NH2-leader or localization sequence-VL-linker-VH-hinge region-C_{H}3-COOH. Exemplary scFv-Fc proteins encoded by the expression constructs are provided in Table 11B, as encoded by exemplary nucleotide sequences provided in Table 11E and Table 11F.

In some embodiments, the construct expresses a scFv-Fc-scFv antibody. In one embodiment, the construct expresses, from the N-terminus to the C-terminus, NH2-leader or localization sequence-VH(A)-linker-VL(A)-hinge region-constant C_{H}2 and constant C_{H}3 regions of an IgG-spacer-VH(B)-linker(VL(B)—COOH (see Pohl SC et al, (2012), “A Cassette Vector System for the Rapid Cloning and Production of Bispecific Tetravalent Antibodies.”).

In some embodiments, the construct expresses a single antibody fragment consisting of a single monomeric variable antibody domain (“nanobody”). In embodiments, the single antibody fragment is a heavy chain variable domain or a light chain variable domain. In one embodiment, the construct expresses, from the N-terminus to the C-terminus, NH2-leader or localization sequence-VH—COOH. In another embodiment, the construct expresses, from the N-terminus to the C-terminus, NH2-leader or localization sequence-VL-COOH.

Gene therapy constructs are also designed such that the antibody is expressed as diabody or triabody. Diabodies are stable non-covalent scFv dimers produced by reducing the length of the intra scFv peptide linkers to less than 8 amino acid residues. This prohibits the VH and VL domains of a single chain from associating with each other to form a functional scFv, as the VH and VL domains have a high affinity to each other. The most stable conformation is a non-covalent dimer in which the VH and VL domain from one scFv pairs with the VH and VL domain of a second scFv to form a functional structure with two binding pockets. In some embodiments, the construct expresses from the N-terminus to C-terminus, NH2-leader or localization sequence-VH-linker with less than 9 amino acid residues-VL-COOH. In some embodiments, the intra-scFv linker length is further reduced to less than 5 amino acid residues leading to the formation of a non-covalent tripod-shaped timer (“tribody”). In some embodiments, two polypeptide chains are expressed with either the arrangement VH(A)-linker-VL(B) and VH(B)-linker-VL(A) (HL configuration) or VL(A)-linker(<=9)-VH(B) and VL(B)-linker(<=9)-VH(A) (LH configuration), respectively, to create a bispecific diabody. In some embodiments, two polypeptide chains are expressed using a furin 2A-based or IRES-based bicistronic cassette carrying both diabody chain A and diabody chain B. In preferred embodiments, the diabody is a single chain diabody (scDb), wherein the linker connecting the two chains has the same length as used for the generation of scFv molecules (e.g. ˜15). In some embodiments, the construct expresses, from the N-terminus to the C-terminus, NH2-leader or localization sequence-VH(A)-linker(<=9)-VL(B)-peptide linker-VH(B)-linker(<=9)-VL(A)-COOH. In some embodiments, the construct expresses, from the N-terminus to the C-terminus, NH2-leader or localization sequence-VH(A)-linker(<=9)-VL(A)-peptide linker(<12)-VH(B)-linker(<=9)-VL(B)—COOH. In some embodiments, the diabody or scDb is fused to a C_{H}3 or Fc region. In some embodiments, the Db—C_{H}3 or DB-Fc fusion protein is obtained by fusing the C_{H}3 domain or the Fc region (including the hinge region) to one of the two different chains (“di-diabodies”).

Gene therapy constructs are also designed such that the antibody is expressed as divalent molecules that are produced through the genetic fusion of an scFv molecule and a C_{H}3 domain of a human IgG molecule. The presence of C_{H}3 domains in minibodies leads to dimerization of two scFv-C_{H}3 fusion proteins to yield the (scFv-C_{H}3)₂ minibody structure. In some embodiments, the construct expresses, from the N-terminus to the C-terminus, NH2-leader or localization sequence-VL-linker(>12)-VH—C_{H}3 domain-COOH.

### Furin-2A Linkers

In other embodiments, the viral vectors provided herein encode the heavy and light chains (either full length, or variable domain, or variable domain and one constant domain, such as the C_{H}1 domain or the CL domain) of the anti-Tau antibodies described herein separated by an IRES or a cleavable linker such as the self-cleaving 2A and 2A-like peptides, with or without upstream furin cleavage sites, e.g. Furin/2A linkers, such as furin/F2A (F/F2A) or furin/T2A (F/T2A) linkers (Fang et al., 2005, Nature Biotechnology 23: 584-590, Fang, 2007, Mol Ther 15: 1153-9, and Chang, J. et al, MAbs 2015, 7(2):403-412, each of which is incorporated by reference herein in its entirety). For example, a furin/2A linker may be incorporated into an expression cassette to separate the heavy and light chain coding sequences, resulting in a construct with the structure:

Leader—Heavy chain—Furin site—2A site—Leader—Light chain—PolyA.

A 2A site or 2A-like site, such as an F2A site comprising the amino acid sequence RKRR(GSG)APVKQTLNFDLLKLAGDVESNPGP (SEQ ID NO: 138) or a T2A site comprising the amino acid sequence RKRR(GSG)EGRGSLLTCGDVEENPGP (SEQ ID NO: 139), is self-processing, resulting in “cleavage” between the final G and P amino acid residues.

Several linkers, with or without an upstream flexible Gly-Ser-Gly (GSG) linker sequence (SEQ ID NO: 140), that could be used include but are not limited to:

T2A:
(SEQ ID NO: 141)
(GSG)EGRGSLLTCGDVEENPGP;
P2A:
(SEQ ID NO: 142)
(GSG)ATNFSLLKQAGDVEENPGP;
E2A:
(SEQ ID NO: 143)
(GSG)QCTNYALLKLAGDVESNPGP;
F2A:
(SEQ ID NO: 144)
(GSG)APVKQTLNFDLLKLAGDVESNPGP

(see also, e.g., Szymczak, et al., 2004, Nature Biotechnol 22(5):589-594, and Donnelly, et al., 2001, J Gen Virol, 82:1013-1025, each of which is incorporated herein by reference). Exemplary nucleotide sequences encoding different parts of the linker are described in Table 8.

In certain embodiments an additional proteolytic cleavage site, e.g., a furin cleavage site, is incorporated into the expression construct adjacent to the self-processing cleavage site (e.g., 2A or 2A-like sequence), thereby providing a means to remove additional amino acids that remain following cleavage by the self-processing cleavage sequence. Without being bound to any one theory, a peptide bond is skipped when the ribosome encounters the 2A sequence in the open reading frame, resulting in the termination of translation, or continued translation of the downstream sequence (the light chain). This self-processing sequence results in a string of additional amino acids at the end of the C-terminus of the heavy chain. However, such additional amino acids can then be cleaved by host cell Furin at the furin cleavage site(s), e.g., located immediately prior to the 2A site and after the heavy chain sequence, and further cleaved by carboxypeptidases. The resultant heavy chain may have one, two, three, or more additional amino acids included at the C-terminus, or it may not have such additional amino acids, depending on the sequence of the Furin linker used and the carboxypeptidase that cleaves the linker in vivo (See, e.g., Fang et al., 17 Apr. 2005, Nature Biotechnol. Advance Online Publication; Fang et al., 2007, Molecular Therapy 15(6):1153-1159; Luke, 2012, Innovations in Biotechnology, Ch. 8, 161-186). Furin linkers that may be used comprise a series of four basic amino acids, for example, RKRR (SEQ ID NO: 145), RRRR (SEQ ID NO: 146), RRKR (SEQ ID NO: 147), or RKKR (SEQ ID NO: 148). Once this linker is cleaved by a carboxypeptidase, additional amino acids may remain, such that an additional zero, one, two, three or four amino acids may remain on the C-terminus of the heavy chain, for example, R, RR, RK, RKR, RRR, RRK, RKK, RKRR (SEQ ID NO: 145), RRRR (SEQ ID NO: 146), RRKR (SEQ ID NO: 147), or RKKR (SEQ ID NO: 148). In certain embodiments, once the linker is cleaved by a carboxypeptidase, no additional amino acids remain. In certain embodiments, 0.5% to 1%, 1% to 2%, 5%, 10%, 15%, or 20% of the antibody, e.g., antigen-binding fragment, population produced by the constructs for use in the methods described herein has one, two, three, or four amino acids remaining on the C-terminus of the heavy chain after cleavage. In certain embodiments, the furin linker has the sequence R-X-K/R-R, such that the additional amino acids on the C-terminus of the heavy chain are R, RX, RXK, RXR, RXKR, or RXRR, where X is any amino acid, for example, alanine (A). In certain embodiments, no additional amino acids may remain on the C-terminus of the heavy chain.

### Flexible Peptide Linkers

In some embodiments, a single construct can be engineered to encode both the heavy and light chains or fragments thereof that participate in antigen binding (e.g. the heavy and light chain variable domains) separated by a flexible peptide linker such as those in a scFv. These linkers can also link an scFv to an Fc domain to form an scFv-Fc. A flexible peptide linker can be composed of flexible residues like glycine and serine so that the adjacent heavy chain and light chain domains are free to move relative to one another. Commonly used flexible linkers have sequences consisting primarily of stretches of four Gly and one Ser residue (“GS” linker), an example of the most widely used flexible linker having the sequence of (Gly-Gly-Gly-Gly-Ser)n (GGGGS or G4S; SEQ ID NO: 149). By adjusting the copy number “n”, the length of this GS linker can be optimized to achieve appropriate separation of the functional domains, or to maintain necessary inter-domain interactions. Examples include, but are not limited to (Gly-Gly-Gly-Gly-Ser)2 (SEQ ID NO: 150), (Gly-Gly-Gly-Gly-Ser)3 (SEQ ID NO: 151), (Gly-Gly-Gly-Gly-Ser)4 (SEQ ID NO: 152), and (Gly-Gly-Gly-Gly-Ser)5 (SEQ ID NO: 153). Besides the GS linkers, many other flexible linkers have been designed for recombinant fusion proteins (Chen, X. et al, Adv Drug Deliv Rev. 2013 Oct. 15; 65(10): 1357-1369). See, e.g., Table 11A.

The construct may be arranged such that the heavy chain variable domain is at the N-terminus of the scFv, followed by the linker and then the light chain variable domain. Alternatively, the construct may be arranged such that the light chain variable domain is at the N-terminus of the scFv, followed by the linker and then the heavy chain variable domain. That is, the components may be arranged as NH2-VL-linker-VH—COOH or NH2-VH-linker-VL-COOH.

In certain embodiments, an expression cassette described herein is contained within a viral vector with a restraint on the size of the polynucleotide(s) therein. In certain embodiments, the expression cassette is contained within an AAV virus-based vector. Due to the size restraints of certain vectors, the vector may or may not accommodate the coding sequences for the full heavy and light chains of the therapeutic antibody but may accommodate the coding sequences of the heavy and light chains of antigen binding fragments, such as the heavy and light chains of a Fab or F(ab′)₂ fragment or an scFv. In particular, the AAV vectors described herein may accommodate a transgene of approximately 4.7-up to theoretically 5.2 kilobases. For constructs such as those in FIGS. 1B and 1C that contain the CAG or CB7 promoter, rabbit β-globin polyA signal, and ITRs, the therapeutic antibody encoded may be more than 750 amino acids. Substitution of smaller expression elements would permit the expression of larger protein products, such as full-length therapeutic antibodies.

### 5.2.4. Constant Region Modifications

In certain embodiments, the transgenes encode full length or substantially full length heavy and light chains that associate to form a full length or intact antibody. (“Substantially intact” or “substantially full length” refers to a mAb having a heavy chain sequence that is at least 95% identical to the full-length heavy chain mAb amino acid sequence and a light chain sequence that is at least 95% identical to the full-length light chain mAb amino acid sequence). Accordingly, the transgenes comprise nucleotide sequences (Table 1, SEQ ID NOs: 1-6) that encode, for example, the light and heavy chains variable domains, that may make up, with the C_{H}1 and all or a portion of the hinge domain and the CL domain, Fab fragments, e.g., FIGS. 1D-1F (Table 2, SEQ ID NOs: 97-102 for the heavy and light chain variable domain amino acid sequences and 187-192 for the amino acid sequences of the Fab portion of the heavy and light chain)), including the hinge region of the heavy chain (Table 7) and C-terminal of the heavy chain of the Fab fragment, an Fc domain peptide (Table 6). Table 6 provides the amino acid sequences of the IgG1 (SEQ ID NO: 196), IgG2 (SEQ ID NO: 197), and IgG4 (SEQ ID NO: 198) Fc domain (C_{H}2 and C_{H}3), IgG1 (SEQ ID NO: 103, 110 (PG-LALA form)), IgG2 (SEQ ID NO: 105), and IgG4 (SEQ ID NO: 107, 109 (S228P form)) constant domains (C_{H}1, hinge region, C_{H}2, and C_{H}3), and IgG1 (SEQ ID NO: 199), IgG2 (SEQ ID NO: 200), and IgG4 (SEQ ID NO: 201) C_{H}3 domains, which may be utilized for the therapeutic antibodies described herein. As detailed, the transgene may comprise a nucleotide sequence encoding the Fc polypeptide for the therapeutic antibody linked to the nucleotide sequence encoding the heavy chain Fab fragment at the C terminus of the hinge region as provided in section 5.2.1 and FIGS. 1D-1F. The transgene may also comprise a nucleotide sequence encoding the C_{H}3 domain of the Fc polypeptide for the therapeutic antibody linked to the nucleotide sequence encoding the heavy chain Fab fragment at the C terminus of the hinge region as provided in section 5.2.1 or may include both the C_{H}2 and C_{H}3 domain (an Fc domain) linked to the C terminus of the hinge domain.

TABLE 6
Exemplary Heavy and Light chain constant region sequences

| | SEQ | |
|---|---|---|
| Structure | ID | Sequence |
| Human IgG1 | 193 | ASTKGPSVFPLAPSSKSTSGGTAALGCLVKDYFPEPVTVSWNSGALTSG |
| C_{H}1 domain | | VHTFPAVLQSSGLYSLSSVVTVPSSSLGTQTYICNVNHKPSNTKVDKRV |
| amino acid | | |
| sequence | | |
| Human IgG1- | 196 | FPPKPKDTLMISRTPEVTCVVVDVSHEDPEVKFNWYVDGVEVHNAKTKP |
| Fc (CH2 and | | REEQYNSTYRVVSVLTVLHQDWLNGKEYKCKVSNKALPAPIEKTISKAK |
| CH3) domain | | GQPREPQVYTLPPSREEMTKNQVSLTCLVKGFYPSDIAVEWESNGQPEN |
| amino acid | | NYKTTPPVLDSDGSFFLYSKLTVDKSRWQQGNVFSCSVMHEALHNHYTQ |
| sequence | | KSLSLSPGK |
| Human IgG1 | 199 | QPREPQVYTLPPSREEMTKNQVSLTCLVKGFYPSDIAVEWESNGQPENN |
| CH3 domain | | YKTTPPVLDSDGSFFLYSKLTVDKSRWQQGNVFSCSVMHEALHNHYTQK |
| amino acid | | SLSLSPGK |
| sequence | | |
| Human IgG1- | 103 | ASTKGPSVFPLAPSSKSTSGGTAALGCLVKDYFPEPVTVSWNSGALTSG |
| constant domain | | VHTFPAVLQSSGLYSLSSVVTVPSSSLGTQTYICNVNHKPSNTKVDKRV |
| amino acid | | EPKSCDKTHTCPPCPAPELLGGPSVFLFPPKPKDTLMISRTPEVTCVVV |
| sequence | | DVSHEDPEVKFNWYVDGVEVHNAKTKPREEQYNSTYRVVSVLTVLHQDW |
| (C_{H}1-hinge- | | LNGKEYKCKVSNKALPAPIEKTISKAKGQPREPQVYTLPPSREEMTKNQ |
| CH2-CH3) | | VSLTCLVKGFYPSDIAVEWESNGQPENNYKTTPPVLDSDGSFFLYSKLT |
| | | VDKSRWQQGNVFSCSVMHEALHNHYTQKSLSLSPGK |
| | | C_{H}1 domain is in italics. Hinge is underlined. |
| Human IgG1- | 104 | GCCAGCACAAAGGGCCCCTCTGTTTTTCCACTGGCTCCCAGCAGCAAGA |
| constant domain | | GCACCAGTGGTGGAACAGCTGCCCTGGGCTGTCTGGTCAAGGATTACTT |
| exemplary | | CCCTGAGCCTGTGACTGTGTCCTGGAACTCTGGTGCACTGACCTCTGGG |
| nucleotide | | GTGCACACCTTTCCAGCTGTGCTGCAGTCCTCTGGCCTGTACTCTCTGT |
| sequence | | CCTCTGTGGTCACAGTGCCTAGCTCTAGCCTGGGCACCCAGACCTACAT |
| (C_{H}1-hinge- | | CTGCAATGTGAACCACAAGCCTAGCAACACCAAGGTGGACAAAAGAGTG |
| CH2-CH3) | | GAACCCAAGAGCTGTGACAAGACCCACACCTGTCCTCCATGTCCTGCTC |
| | | CAGAACTGCTTGGAGGCCCTTCTGTGTTCCTGTTTCCTCCAAAGCCTAA |
| | | GGACACCCTGATGATCAGCAGAACCCCTGAAGTGACCTGTGTGGTGGTT |
| | | GATGTGTCCCATGAGGACCCAGAAGTGAAGTTCAATTGGTATGTGGATG |
| | | GGGTTGAAGTGCACAATGCCAAGACCAAGCCTAGAGAGGAACAGTACAA |
| | | CTCCACCTACAGAGTGGTGTCTGTGCTGACAGTGCTGCACCAGGACTGG |
| | | CTGAATGGCAAAGAGTACAAGTGCAAGGTGTCCAACAAGGCCCTGCCTG |
| | | CTCCTATTGAGAAAACCATCTCCAAGGCCAAGGGCCAGCCAAGAGAACC |
| | | CCAGGTTTACACCCTGCCACCTAGCAGAGAAGAGATGACCAAGAACCAG |
| | | GTGTCCCTGACCTGCCTGGTTAAGGGCTTCTACCCCTCTGACATTGCTG |
| | | TGGAATGGGAGAGCAATGGCCAGCCTGAAAACAACTACAAGACAACCCC |
| | | TCCTGTGCTGGACTCAGATGGCTCATTCTTCCTGTACAGCAAGCTGACT |
| | | GTGGACAAGTCCAGGTGGCAGCAGGGAAATGTGTTCAGCTGCTCTGTGA |
| | | TGCATGAGGCCCTGCACAACCACTACACCCAGAAAAGTCTGAGTCTGAG |
| | | CCCTGGCAAG |
| Human IgG2 | 194 | ASTKGPSVFPLAPCSRSTSESTAALGCLVKDYFPEPVTVSWNSGALTSG |
| CH1 domain | | VHTFPAVLQSSGLYSLSSVVTVPSSNFGTQTYTCNVDHKPSNTKVDKTV |
| Human IgG2- | 197 | FPPKPKDTLMISRTPEVTCVVVDVSHEDPEVQFNWYVDGVEVHNAKTKP |
| Fc (CH2 and | | REEQFNSTFRVVSVLTVVHQDWLNGKEYKCKVSNKGLPAPIEKTISKTK |
| CH3) domain | | GQPREPQVYTLPPSREEMTKNQVSLTCLVKGFYPSDISVEWESNGQPEN |
| amino acid | | NYKTTPPMLDSDGSFFLYSKLTVDKSRWQQGNVFSCSVMHEALHNHYTQ |
| sequence | | KSLSLSPGK |
| Human IgG2 | 200 | GQPREPQVYTLPPSREEMTKNQVSLTCLVKGFYPSDISVEWESNGQPEN |
| CH3 domain | | NYKTTPPMLDSDGSFFLYSKLTVDKSRWQQGNVFSCSVMHEALHNHYTQ |
| amino acid | | KSLSLSPGK |
| sequence | | |
| Human IgG2- | 105 | ASTKGPSVFPLAPCSRSTSESTAALGCLVKDYFPEPVTVSWNSGALTSG |
| constant domain | | VHTFPAVLQSSGLYSLSSVVTVPSSNFGTQTYTCNVDHKPSNTKVDKTV |
| amino acid | | ERKCCVECPPCPAPPVAGPSVFLFPPKPKDTLMISRTPEVTCVVVDVSH |
| sequence | | EDPEVQFNWYVDGVEVHNAKTKPREEQFNSTFRVVSVLTVVHQDWLNGK |
| (C_{H}1-hinge- | | EYKCKVSNKGLPAPIEKTISKTKGQPREPQVYTLPPSREEMTKNQVSLT |
| CH2-CH3) | | CLVKGFYPSDISVEWESNGQPENNYKTTPPMLDSDGSFFLYSKLTVDKS |
| | | RWQQGNVFSCSVMHEALHNHYTQKSLSLSPGK |
| | | C_{H}1 domain is in italics. Hinge is underlined. |
| Human IgG2- | 106 | GCGAGCACCAAAGGCCCGAGCGTGTTTCCGCTGGCGCCGTGCAGCCGCA |
| constant domain | | GCACCAGCGAAAGCACCGCGGCGCTGGGCTGCCTGGTGAAAGATTATTT |
| exemplary | | TCCGGAACCGGTGACCGTGAGCTGGAACAGCGGCGCGCTGACCAGCGGC |
| nucleotide | | GTGCATACCTTTCCGGCGGTGCTGCAGAGCAGCGGCCTGTATAGCCTGA |
| sequence | | GCAGCGTGGTGACCGTGCCGAGCAGCAACTTTGGCACCCAGACCTATAC |
| (C_{H}1-hinge- | | CTGCAACGTGGATCATAAACCGAGCAACACCAAAGTGGATAAAACCGTG |
| CH2-CH3) | | GAACGCAAATGCTGCGTGGAATGCCCGCCGTGCCCGGCGCCGCCGGTGG |
| | | CGGGCCCGAGCGTGTTTCTGTTTCCGCCGAAACCGAAAGATACCCTGAT |
| | | GATTAGCCGCACCCCGGAAGTGACCTGCGTGGTGGTGGATGTGAGCCAT |
| | | GAAGATCCGGAAGTGCAGTTTAACTGGTATGTGGATGGCGTGGAAGTGC |
| | | ATAACGCGAAAACCAAACCGCGCGAAGAACAGTTTAACAGCACCTTTCG |
| | | CGTGGTGAGCGTGCTGACCGTGGTGCATCAGGATTGGCTGAACGGCAAA |
| | | GAATATAAATGCAAAGTGAGCAACAAAGGCCTGCCGGCGCCGATTGAAA |
| | | AAACCATTAGCAAAACCAAAGGCCAGCCGCGCGAACCGCAGGTGTATAC |
| | | CCTGCCGCCGAGCCGCGAAGAAATGACCAAAAACCAGGTGAGCCTGACC |
| | | TGCCTGGTGAAAGGCTTTTATCCGAGCGATATTAGCGTGGAATGGGAAA |
| | | GCAACGGCCAGCCGGAAAACAACTATAAAACCACCCCGCCGATGCTGGA |
| | | TAGCGATGGCAGCTTTTTTCTGTATAGCAAACTGACCGTGGATAAAAGC |
| | | CGCTGGCAGCAGGGCAACGTGTTTAGCTGCAGCGTGATGCATGAAGCGC |
| | | TGCATAACCATTATACCCAGAAAAGCCTGAGCCTGAGCCCGGGCAAATA |
| | | A |
| Human IgG4 | 195 | ASTKGPSVFPLAPCSRSTSESTAALGCLVKDYFPEPVTVSWNSGALTSG |
| C_{H}1 domain | | VHTFPAVLQSSGLYSLSSVVTVPSSSLGTKTYTCNVDHKPSNTKVDKRV |
| amino acid | | |
| sequence | | |
| Human IgG4 | 201 | QPREPQVYTLPPSQEEMTKNQVSLTCLVKGFYPSDIAVEWESNGQPENN |
| CH3 domain | | YKTTPPVLDSDGSFFLYSRLTVDKSRWQEGNVFSCSVMHEALHNHYTQK |
| | | SLSLSLGK |
| Human IgG4- | 198 | FPPKPKDTLMISRTPEVTCVVVDVSQEDPEVQFNWYVDGVEVHNAKTKP |
| Fc (CH2 and | | REEQFNSTYRVVSVLTVLHQDWLNGKEYKCKVSNKGLPSSIEKTISKAK |
| CH3) domain | | GQPREPQVYTLPPSQEEMTKNQVSLTCLVKGFYPSDIAVEWESNGQPEN |
| amino acid | | NYKTTPPVLDSDGSFFLYSRLTVDKSRWQEGNVFSCSVMHEALHNHYTQ |
| sequence | | KSLSLSLGK |
| Human IgG4- | 107 | ASTKGPSVFPLAPCSRSTSESTAALGCLVKDYFPEPVTVSWNSGALTSG |
| constant amino | | VHTFPAVLQSSGLYSLSSVVTVPSSSLGTKTYTCNVDHKPSNTKVDKRV |
| acid sequence | | ESKYGPPCPSCPAPEFLGGPSVFLFPPKPKDTLMISRTPEVTCVVVDVS |
| (C_{H}1-hinge- | | QEDPEVQFNWYVDGVEVHNAKTKPREEQFNSTYRVVSVLTVLHQDWLNG |
| CH2-CH3) | | KEYKCKVSNKGLPSSIEKTISKAKGQPREPQVYTLPPSQEEMTKNQVSL |
| | | TCLVKGFYPSDIAVEWESNGQPENNYKTTPPVLDSDGSFFLYSRLTVDK |
| | | SRWQEGNVFSCSVMHEALHNHYTQKSLSLSLGK |
| | | C_{H}1 domain is in italics. Hinge is underlined. |
| Human IgG4- | 108 | GCCTCCACCAAGGGCCCATCGGTCTTCCCCCTGGCGCCCTGCTCCAGGA |
| constant domain | | GCACCTCCGAGAGCACAGCGGCCCTGGGCTGCCTGGTCAAGGACTACTT |
| exemplary | | CCCCGAACCGGTGACGGTGTCGTGGAACTCAGGCGCCCTGACCAGCGGC |
| nucleotide | | GTGCACACCTTCCCGGCTGTCCTACAGTCCTCAGGACTCTACTCCCTCA |
| sequence | | GCAGCGTGGTGACCGTGCCCTCCAGCAGCTTGGGCACGAAGACCTACAC |
| (C_{H}1-hinge- | | CTGCAATGTAGATCACAAGCCCAGCAACACCAAGGTGGACAAGAGAGTT |
| CH2-CH3) | | GAGTCCAAATATGGTCCCCCGTGCCCATCATGCCCAGCACCTGAATTCC |
| | | TGGGGGGACCATCAGTCTTCCTGTTCCCCCCAAAACCCAAGGACACCCT |
| | | CATGATCTCCCGGACCCCTGAGGTCACGTGCGTGGTGGTGGACGTGAGC |
| | | CAGGAAGACCCCGAGGTCCAGTTCAACTGGTACGTGGATGGCGTGGAGG |
| | | TGCATAATGCCAAGACAAAGCCGCGGGAGGAGCAGTTCAACAGCACGTA |
| | | CCGTGTGGTCAGCGTCCTCACCGTCGTGCACCAGGACTGGCTGAACGGC |
| | | AAGGAGTACAAGTGCAAGGTCTCCAACAAAGGCCTCCCGTCCTCCATCG |
| | | AGAAAACCATCTCCAAAGCCAAAGGGCAGCCCCGAGAGCCACAGGTGTA |
| | | CACCCTGCCCCCATCCCAGGAGGAGATGACCAAGAACCAGGTCAGCCTG |
| | | ACCTGCCTGGTCAAAGCCTTCTACCCCAGCGACATCGCCGTGGAGTGGG |
| | | AGAGCAATGGGCAGCCGGAGAACAACTACAAGACCACGCCTCCCGTGCT |
| | | GGACTCCGACGGCTCCTTCTTCCTCTACAGCAGGCTAACCGTGGACAAG |
| | | AGCAGGTGGCAGGAGGGGAATGTCTTCTCATGCTCCGTGATGCATGAGG |
| | | CTCTGCACAACCACTACACGCAGAAGAGCCTCTCCCTGTCTCTGGGTAA |
| | | A |
| Human IgG4 | 109 | ASTKGPSVFPLAPCSRSTSESTAALGCLVKDYFPEPVTVSWNSGALTSG |
| constant domain | | VHTFPAVLQSSGLYSLSSVVTVPSSSLGTKTYTCNVDHKPSNTKVDKRV |
| S228P amino | | ESKYGPPCPPCPAPEFLGGPSVFLFPPKPKDTLMISRTPEVTCVVVDVS |
| acid sequence | | QEDPEVQFNWYVDGVEVHNAKTKPREEQFNSTYRVVSVLTVLHQDWLNG |
| (C_{H}1-hinge- | | KEYKCKVSNKGLPSSIEKTISKAKGQPREPQVYTLPPSQEEMTKNQVSL |
| CH2-CH3) | | TCLVKGFYPSDIAVEWESNGQPENNYKTTPPVLDSDGSFFLYSRLTVDK |
| | | SRWQEGNVFSCSVMHEALHNHYTQKSLSLSLGK |
| | | C_{H}1 domain is in italics. Hinge is underlined. |
| | | S228P is shown in bold. |
| Human IgG1- | 110 | ASTKGPSVFPLAPSSKSTSGGTAALGCLVKDYFPEPVTVSWNSGALTSG |
| constant | | VHTFPAVLQSSGLYSLSSVVTVPSSSLGTQTYICNVNHKPSNTKVDKKV |
| domain-LALA- | | EPKSCDKTHTCPPCPAPEAAGGPSVFLFPPKPKDTLMISRTPEVTCVVV |
| PG-amino acid | | DVSHEDPEVKFNWYVDGVEVHNAKTKPREEQYNSTYRVVSVLTVLHQDW |
| sequence | | LNGKEYKCKVSNKALGAPIEKTISKAKGQPREPQVYTLPPSRDELTKNQ |
| (C_{H}1-hinge- | | VSLTCLVKGFYPSDIAVEWESNGQPENNYKTTPPVLDSDGSFFLYSKLT |
| CH2-CH3) | | VDKSRWQQGNVFSCSVMHEALHNHYTQKSLSLSPGK |
| | | C_{H}1 domain is in italics. Hinge is underlined. |
| | | L234A/L235A/P329G are shown in bold. |
| Human IgG1- | 111 | GCGAGCACCAAAGGCCCGAGCGTGTTTCCGCTGGCGCCGAGCAGCAAAA |
| constant | | GCACCAGCGGCGGCACCGCGGCGCTGGGCTGCCTGGTGAAAGATTATTT |
| domain-LALA- | | TCCGGAACCGGTGACCGTGAGCTGGAACAGCGGCGCGCTGACCAGCGGC |
| PG-exemplary | | GTGCATACCTTTCCGGCGGTGCTGCAGAGCAGCGGCCTGTATAGCCTGA |
| nucleotide | | GCAGCGTGGTGACCGTGCCGAGCAGCAGCCTGGGCACCCAGACCTATAT |
| sequence | | TTGCAACGTGAACCATAAACCGAGCAACACCAAAGTGGATAAAAAAGTG |
| (C_{H}1-hinge- | | GAACCGAAAAGCTGCGATAAAACCCATACCTGCCCGCCGTGCCCGGCGC |
| CH2-CH3) | | CGGAAGCGGCGGGCGGCCCGAGCGTGTTTCTGTTTCCGCCGAAACCGAA |
| | | AGATACCCTGATGATTAGCCGCACCCCGGAAGTGACCTGCGTGGTGGTG |
| | | GATGTGAGCCATGAAGATCCGGAAGTGAAATTTAACTGGTATGTGGATG |
| | | GCGTGGAAGTGCATAACGCGAAAACCAAACCGCGCGAAGAACAGTATAA |
| | | CAGCACCTATCGCGTGGTGAGCGTGCTGACCGTGCTGCATCAGGATTGG |
| | | CTGAACGGCAAAGAATATAAATGCAAAGTGAGCAACAAAGCGCTGGGCG |
| | | CGCCGATTGAAAAAACCATTAGCAAAGCGAAAGGCCAGCCGCGCGAACC |
| | | GCAGGTGTATACCCTGCCGCCGAGCCGCGATGAACTGACCAAAAACCAG |
| | | GTGAGCCTGACCTGCCTGGTGAAAGGCTTTTATCCGAGCGATATTGCGG |
| | | TGGAATGGGAAAGCAACGGCCAGCCGGAAAACAACTATAAAACCACCCC |
| | | GCCGGTGCTGGATAGCGATGGCAGCTTTTTTCTGTATAGCAAACTGACC |
| | | GTGGATAAAAGCCGCTGGCAGCAGGGCAACGTGTTTAGCTGCAGCGTGA |
| | | TGCATGAAGCGCTGCATAACCATTATACCCAGAAAAGCCTGAGCCTGAG |
| | | CCCGGGCAAA |
| Mouse IgG2a- | 112 | AKTTAPSVYPLAPVCGDTTGSSVTLGCLVKGYFPEPVTLTWNSGSLSSG |
| constant domain | | VHTFPAVLQSDLYTLSSSVTVTSSTWPSQSITCNVAHPASSTKVDKKIE |
| amino acid | | PRGPTIKPCPPCKCPAPNLLGGPSVFIFPPKIKDVLMISLSPIVTCVVV |
| sequence | | DVSEDDPDVQISWFVNNVEVHTAQTQTHREDYNSTLRVVSALPIQHQDW |
| (C_{H}1-hinge- | | VTLTCMVTDFMPEDIYVEWTNNGKTELNYKNTEPVLDSDGSYFMYSKLR |
| CH2-CH3) | | VEKKNWVERNSYSCSVVHEGLHNHHTTKSFSRTPGK |
| | | C_{H}1 is in italics. Hinge region is underlined. |
| Mouse IgG2a- | 113 | GCTAAGACAACAGCCCCTTCTGTGTACCCTCTGGCTCCTGTGTGTGGGG |
| constant domain | | ACACAACAGGATCCTCTGTGACACTGGGCTGCCTGGTCAAGGGCTACTT |
| exemplary | | TCCTGAGCCAGTGACACTGACCTGGAACTCTGGATCTCTGTCCTCTGGG |
| nucleotide | | GTGCACACATTCCCTGCTGTGCTGCAGTCTGACCTGTACACCCTGAGCA |
| sequence | | GCAGTGTGACAGTGACCAGCAGCACATGGCCCAGCCAGAGCATCACCTG |
| (C_{H}1-hinge- | | TAATGTGGCTCACCCTGCCAGCTCCACCAAGGTGGACAAGAAGATTGAG |
| CH2-CH3) | | CCTAGAGGCCCCACCATCAAGCCCTGTCCTCCATGCAAGTGTCCTGCTC |
| | | CTAACCTGCTTGGAGGCCCCTCTGTGTTCATCTTCCCACCTAAGATCAA |
| | | GGATGTGCTGATGATCTCTCTGAGCCCCATTGTGACCTGTGTGGTGGTT |
| | | GATGTGTCTGAGGATGACCCAGATGTGCAGATCTCTTGGTTTGTGAACA |
| | | ATGTGGAAGTGCACACAGCCCAGACACAGACCCACAGAGAGGACTACAA |
| | | CAGCACCCTGAGAGTGGTGTCTGCCCTGCCTATCCAGCACCAGGACTGG |
| | | ATGTCTGGCAAAGAGTTCAAGTGCAAAGTGAACAACAAGGACCTGCCTG |
| | | CTCCAATTGAGAGAACCATCAGCAAGCCCAAGGGCTCTGTTAGAGCCCC |
| | | TCAAGTGTATGTGCTGCCTCCACCTGAGGAAGAGATGACCAAGAAACAA |
| | | GTGACCCTGACATGCATGGTCACTGACTTCATGCCTGAGGACATCTATG |
| | | TGGAATGGACCAACAATGGCAAGACAGAGCTGAACTACAAGAACACTGA |
| | | GCCTGTGCTGGACTCTGATGGCAGCTACTTCATGTACAGCAAGCTGAGG |
| | | GTTGAGAAGAAGAACTGGGTTGAGAGAAACAGCTACTCCTGCTCTGTGG |
| | | TGCATGAGGGCCTGCACAACCATCACACCACAAAGAGCTTCAGCAGAAC |
| | | CCCAGGCAAG |
| Mouse IgG2a- | 114 | AKTTAPSVYPLAPVCGDTTGSSVTLGCLVKGYFPEPVTLTWNSGSLSSG |
| constant domain | | VHTFPAVLQSDLYTLSSSVTVTSSTWPSQSITCNVAHPASSTKVDKKIE |
| LALA-PG- | | PRGPTIKPCPPCKCPAPNAAGGPSVFIFPPKIKDVLMISLSPIVTCVVV |
| amino acid | | DVSEDDPDVQISWFVNNVEVHTAQTQTHREDYNSTLRVVSALPIQHQDW |
| sequence | | MSGKEFKCKVNNKDLGAPIERTISKPKGSVRAPQVYVLPPPEEEMTKKQ |
| | | VTLTCMVTDFMPEDIYVEWTNNGKTELNYKNTEPVLDSDGSYFMYSKLR |
| | | VEKKNWVERNSYSCSVVHEGLHNHHTTKSFSRTPGK |
| | | C_{H}1 domain is in italics. Hinge is underlined. |
| | | LALA-PG variants are shown in bold. |
| Mouse IgG2a- | 115 | GCCAAGACAACTGCCCCTTCTGTGTACCCTCTGGCTCCTGTGTGTGGGG |
| constant domain | | ACACAACAGGATCCTCTGTGACACTGGGCTGCCTGGTCAAGGGCTACTT |
| LALA-PG- | | TCCTGAGCCAGTGACACTGACCTGGAACTCTGGATCTCTGTCCTCTGGG |
| exemplary | | GTGCACACATTCCCTGCTGTGCTGCAGTCTGACCTGTACACCCTGAGCA |
| nucleotide | | GCTCTGTGACTGTGACCAGCAGCACATGGCCCAGCCAGAGCATCACCTG |
| sequence | | TAATGTGGCTCACCCTGCCAGCTCCACCAAGGTGGACAAGAAGATTGAG |
| | | CCTAGAGGCCCCACCATCAAGCCCTGTCCTCCATGCAAGTGTCCTGCTC |
| | | CTAACGCCGCTGGAGGCCCCTCTGTGTTCATCTTCCCACCTAAGATCAA |
| | | GGATGTGCTGATGATCTCTCTGAGCCCCATTGTGACCTGTGTGGTGGTT |
| | | GATGTGTCTGAGGATGACCCTGATGTGCAGATCTCTTGGTTTGTGAACA |
| | | ATGTGGAAGTGCACACAGCCCAGACACAGACCCACAGAGAGGACTACAA |
| | | CAGCACCCTGAGAGTGGTGTCTGCCCTGCCTATCCAGCACCAGGACTGG |
| | | ATGTCTGGCAAAGAGTTCAAGTGCAAAGTGAACAACAAGGACCTGGGAG |
| | | CTCCAATTGAGAGAACCATCTCCAAGCCTAAGGGCTCTGTCAGGGCCCC |
| | | TCAAGTGTATGTGCTGCCACCTCCTGAGGAAGAGATGACCAAGAAACAA |
| | | GTGACCCTGACATGCATGGTCACAGACTTCATGCCTGAGGACATCTATG |
| | | TGGAATGGACCAACAATGGCAAGACAGAGCTGAACTACAAGAACACTGA |
| | | GCCTGTGCTGGACTCTGATGGCTCCTACTTCATGTACAGCAAGCTGAGG |
| | | GTTGAGAAGAAGAACTGGGTTGAGAGAAACAGCTACAGCTGCTCTGTGG |
| | | TGCATGAGGGCCTGCACAATCACCACACCACCAAGAGCTTCAGCAGAAC |
| | | CCCTGGCAAG |
| Kappa light | 116 | RTVAAPSVFIFPPSDEQLKSGTASVVCLLNNFYPREAKVQWKVDNALQS |
| chain C_{L} amino | | GNSQESVTEQDSKDSTYSLSSTLTLSKADYEKHKVYACEVTHQGLSSPV |
| acid sequence | | TKSFNRGEC |
| Kappa light | 117 | AGAACAGTGGCTGCCCCATCTGTGTTCATCTTCCCACCATCTGATGAGC |
| chain C_{L} | | AGCTCAAGTCTGGCACTGCCTCTGTTGTGTGCCTGCTGAACAACTTCTA |
| exemplary | | CCCTAGAGAAGCCAAGGTGCAGTGGAAGGTTGACAATGCCCTGCAGTCT |
| nucleotide | | GGCAATAGCCAAGAATCTGTGACAGAGCAGGACTCCAAGGATAGCACCT |
| sequence | | ACAGCCTGTCTAGCACCCTGACACTGAGCAAGGCTGACTATGAGAAGCA |
| | | CAAAGTGTATGCCTGTGAAGTGACACACCAGGGCCTGAGCAGCCCAGTG |
| | | ACCAAGAGCTTCAATAGAGGGGAGTGC |
| Lambda light | 118 | GQPKSSPSVTLFPPSSEELETNKATLVCTITDFYPGVVTVDWKVDGTPV |
| chain C_{L} amino | | TQGMETTQPSKQSNNKYMASSYLTLTARAWERHSSYSCQVTHEGHTVEK |
| acid sequence | | SLSRADCS |
| Lambda light | 119 | GGCCAGCCTAAAAGCAGCCCCTCTGTGACACTGTTCCCTCCAAGCTCTG |
| chain C_{L} | | AGGAACTGGAAACAAACAAGGCCACACTTGTGTGCACCATCACAGACTT |
| exemplary | | CTACCCTGGGGTTGTGACAGTGGACTGGAAAGTGGATGGCACCCCTGTG |
| nucleotide | | ACACAAGGCATGGAAACCACACAGCCCAGCAAGCAGAGCAACAACAAGT |
| sequence | | ACATGGCCAGCTCCTATCTGACCCTGACAGCCAGAGCCTGGGAGAGACA |
| | | CAGCTCTTACAGCTGCCAAGTGACCCATGAGGGCCACACAGTGGAAAAG |
| | | AGCCTGAGCAGAGCTGACTGCTCT |

Based on the degeneracy of the genetic code, it is understood that the nucleotide sequences provided in Table 6 are exemplary and non-limiting. In some embodiments, the expressed heavy chain constant region is selected from a sequence in Table 6 having a nucleic acid sequence of SEQ ID NOs: 104, 106, 108, 111, 113, or 115 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of 104, 106, 108, 111, 113, or 115 and that has the biologic activity of the constant region.

In some embodiments, the antibody light chain constant region or domain is selected from a sequence in Table 6 having a nucleic acid sequence of SEQ ID NOs: 117 or 119 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 117, or 119 and that has the biologic activity of the constant region.

In certain embodiments, a viral vector of the disclosure incorporates a heavy chain constant region and/or a light chain constant region selected from Table 6 having a nucleic acid sequence of SEQ ID NOs: 104, 106, 108, 111, 113, 115, 117, or 119 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 104, 106, 108, 111, 113, 115, 117, or 119 and that has the biologic activity of the constant region.

In other embodiments, the immunoglobulin constant regions are engineered to provide “effectorless” function. In some embodiments, the anti-Tau antibodies have an IgG4 or IgG2 isotype constant region, such that antibodies having an Fc domain of the IgG4 or IgG2 isotype exhibit reduced effector function as compared to antibodies having an Fc domain of the IgG1 isotype. In some embodiments, the effectorless Fc domain is an aglycosylated IgG1, IgG2, or IgG4 Fc domain that has a substitution at residue 297 or 299 to alter the glycosylation site at 297 such that the Fc domain exhibits reduced ADCC or other effector activity. Amino acid numbering of immunoglobulin constant regions described throughout the present disclosure is according to the EU index as in Kabat et al. (1991, NIH Publication 91-3242, National Technical Information Services, Springfield, Va., which is hereby incorporated by reference). In some embodiments, amino acids at positions 234, 235, 329 of the IgG1 constant region are modified (or mutated) in order to reduce effector function, also known as Fc function. As such, the L234A, L235A, P329G (LALA-PG) variant eliminates complement binding and fixation as well as Fc-7 dependent antibody-dependent cell-mediated cytotoxity (ADCC) in both murine IgG2a and human IgG1. Other non-limiting Fc modifications are described herein.

In certain embodiments, the transgenes encode full length or substantially full length heavy and light chains that associate to form a full length or intact antibody. “Substantially full length” refers to a mAb having a heavy chain sequence that includes the hinge region of the heavy chain and C-terminal (C_{H}1, SEQ ID NOs: 193-195) of the heavy chain (Fab fragment), and all or part of an Fc domain (C_{H}2 and/or C_{H}3, SEQ ID NOs: 196, 197, or 198). Table 6 provides the amino acid sequences of the Fc polypeptides for certain of the therapeutic antibodies described herein.

The term “Fc region” refers to a dimer of two “Fc polypeptides” (or “Fc domains”), each “Fc polypeptide” comprising the heavy chain constant region of an antibody excluding the first constant region immunoglobulin domain. In some embodiments, an “Fc region” includes two Fc polypeptides linked by one or more disulfide bonds, chemical linkers, or peptide linkers. “Fc polypeptide” refers to at least the last two constant region immunoglobulin domains of IgA, IgD, and IgG, or the last three constant region immunoglobulin domains of IgE and IgM and may also include part or all of the flexible hinge N-terminal to these domains. For IgG, e.g., “Fc polypeptide” (or “Fc domain”) comprises immunoglobulin domains Cgamma2 (Cγ2, often referred to as C_{H}2 domain) and Cgamma3 (Cγ3, also referred to as C_{H}3 domain) and may or may not include a portion of the lower part of the hinge domain between Cgamma1 (Cγ1, also referred to as C_{H}1 domain) and C_{H}2 domain. Although the boundaries of the Fc polypeptide may vary, the human IgG heavy chain Fc polypeptide may comprise residues starting at T223 or C226 or P230, to its carboxyl-terminus, wherein the numbering is according to the EU index as in Kabat et al. (1991, NIH Publication 91-3242, National Technical Information Services, Springfield, Va.). In other words, the Fc polypeptide (or Fc domain) may or may not comprise some portion of the hinge domain. Depending on the application, the hinge portion, or an engineered or chimeric hinge thereof, may be useful for flexibility or association (dimerization) of the Fc polypeptides. For IgA, e.g., Fc polypeptide comprises immunoglobulin domains Calpha2 (Cα2) and Calpha3 (Cα3) and may include the lower part of the hinge between Calpha1 (Cα1) and Cα2.

In certain embodiments, the Fc polypeptide corresponds to the Fc polypeptide of any immunoglobulin isotype. In still other embodiments, the Fc polypeptide is an IgG Fc polypeptide. The Fc polypeptide may be from the IgG1, IgG2, or IgG4, or may be an IgG3 Fc domain, depending, for example, upon the desired effector activity of the therapeutic antibody. In some embodiments, the engineered heavy chain constant region (C_{H}), which includes the Fc domain, is chimeric. As such, a chimeric C_{H} region combines C_{H} domains derived from more than one immunoglobulin isotype and/or subtype. For example, the chimeric (or hybrid) C_{H} region comprises part or all of an Fc region from IgG, IgA and/or IgM. In other examples, the chimeric C_{H} region comprises part or all a C_{H}2 domain derived from a human IgG1, human IgG2, or human IgG4 molecule, combined with part or all of a C_{H}3 domain derived from a human IgG1 (SEQ ID NO: 199), human IgG2 (SEQ ID NO: 200), or human IgG4 (SEQ ID NO: 201) molecule. In other embodiments, the chimeric C_{H} region contains a chimeric hinge region. Various hinge region sequences are set forth in Table 7. Also provided are embodiments in which 1, 2, 3, 4, 5, 6, 7 or 8 amino acids of the N-terminal sequence of the hinge is included, for example, EP, EPKS, EPKSCD, ES, ESKY, or ESKYGP.

The transgene may encode a Fab fragment having the nucleotide sequences encoding all or a portion of the hinge region with the amino acid sequences provided in Table 7, but not including the portion of the hinge region on the heavy chain that forms interchain di-sulfide bonds (e.g., the portion containing the sequence CPPCPA (SEQ ID NO: 202)). Heavy chain Fab domain sequences that do not contain a CPPCP (SEQ ID NO: 203) sequence of the hinge region at the C-terminus will not form intrachain disulfide bonds and, thus, will form Fab fragments with the corresponding light chain Fab domain sequences, whereas those heavy chain Fab domain sequences with a portion of the hinge region at the C-terminus containing the sequence CPPCP (SEQ ID NO: 203) will form intrachain disulfide bonds and, thus, will form Fab₂ fragments. For example, in some embodiments, the transgene may encode a scFv comprising a light chain variable domain and a heavy chain variable domain connected by a flexible linker in between (where the heavy chain variable domain may be either at the N-terminal end or the C-terminal end of the scFv), and optionally, may further comprise a Fc polypeptide (e.g., IgG1, IgG2, IgG3, or IgG4) on the C-terminal end of the heavy chain. Alternatively, in other embodiments, the transgene may encode F(ab′)₂ fragments comprising a nucleotide sequence that encodes the light chain and the heavy chain sequence that includes at least the sequence CPPCA (SEQ ID NO: 204) of the hinge region, as depicted in FIGS. 1D-1F, which depict various portions of the hinge region that may be included at the C-terminus of the heavy chain sequence. Pre-existing anti-hinge antibodies (AHA) may cause immunogenicity and reduce efficacy. Thus, in certain embodiments, for the IgG1 isotype, C-terminal ends with D221 or ends with a mutation T225L or with L242 can reduce binding to AHA. (See, e.g., Brezski, 2008, J Immunol 181: 3183-92 and Kim, 2016, 8: 1536-1547). For IgG2, the risk of AHA is lower since the hinge region of IgG2 is not as susceptible to enzymatic cleavage required to generate endogenous AHA. See e.g. Brezski 2011, MAbs 3: 558-567).

TABLE 7
Hinge Regions

| SEQ ID NO: | Sequence |
|---|---|
| 203 | CPPCP |
| 202 | CPPCPA |
| 204 | CPPCA |
| 172 | EPKSCDKTHTCPPCPAPELLGG |
| 175 | EPKSCDKTHLCPPCPAPELLGG |
| 177 | EPKSCDKTHL |
| 174 | EPKSCDKTHT |
| 173 | EPKSCDKTHTCPPCPA |
| 176 | EPKSCDKTHLCPPCPA |
| 178 | EPKSCDKTHTCPPCPAPELLGGPSVFL |
| 179 | EPKSCDKTHLCPPCPAPELLGGPSVFL |
| 205 | EPKSCDKTHTCPPCPAPEAAGG |
| 206 | EPKSCDKTHTCPPCPAPEAAGGPSVFL |
| 207 | EPKSCDKTHLCPPCPAPEAAGGPSVFL |
| 208 | ERKSCVECPPCPAPPVAG |
| 209 | ERKSCVECPPCPA |
| 180 | ESKYGPPCPPCPAPEFLGG |
| 210 | ESKYGPPCPSCPAPEFLGG |
| 181 | ESKYGPPCPPCPA |
| 182 | ESKYGPPCPSCPA |
| 183 | ESKYGPPCPSCPAPEFLGGPSVFL |
| 184 | ESKYGPPCPPCPAPEFLGGPSVFL |
| 185 | ERKCCVECPPCPAPPVAG |
| 186 | ERKCCVECPPCPA |
| 211 | EPKSCDKTHTCPPCPAPELAGA |
| 212 | EPKSCDKTHTCPPCPAPELAGAPSVFL |
| 213 | EPKSCDKTHLCPPCPAPELAGAPSVFL |
| 214 | EPKSCDKTHTCPPCPAPEFEGG |
| 215 | EPKSCDKTHTCPPCPAPEFEGGPSVFL |
| 216 | EPKSCDKTHLCPPCPAPEFEGGPSVFL |

In some embodiments, the recombinant vectors encode therapeutic antibodies comprising an engineered (mutant) Fc region, e.g. engineered Fc regions of an IgG constant region. Modifications to an antibody constant region, Fc region or Fc fragment of an IgG antibody may alter one or more effector functions such as Fc receptor binding or neonatal Fc receptor (FcRn) binding and thus half-life, CDC activity, ADCC activity, and/or ADPC activity, compared to a corresponding antibody having a wild-type IgG constant region, or an IgG heavy chain constant region without the recited modification(s). Accordingly, in some embodiments, the antibody may be engineered to provide an antibody constant region, Fc region or Fc fragment of an IgG antibody that exhibits altered binding (as compared to a reference or wild-type constant region without the recited modification(s)) to one or more Fc receptors (e.g., FcγRI, FcγRIIA, FcγRIIB, FcγRIIIA, FcγRIIIB, FcγRIV, or FcRn receptor). In some embodiments, the antibody an antibody constant region, Fc region or Fc fragment of an IgG antibody that exhibits a one or more altered effector functions such as CDC, ADCC, or ADCP activity, compared to a corresponding antibody having a wild-type IgG constant region, or an IgG constant without the recited modification(s).

“Effector function” refers to a biochemical event that results from the interaction of an antibody Fc region with an Fc receptor or ligand. Effector functions include FcγR-mediated effector functions such as ADCC and ADCP and complement-mediated effector functions such as CDC.

An “effector cell” refers to a cell of the immune system that expresses one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and T cells, and may be from any organism including but not limited to humans, mice, rats, rabbits, and monkeys.

“ADCC” or “antibody dependent cell-mediated cytotoxicity” refers to the cell-mediated reaction wherein nonspecific cytotoxic effector (immune) cells that express FcγRs recognize bound antibody on a target cell and subsequently cause lysis of the target cell.

“ADCP” or “antibody dependent cell-mediated phagocytosis” refers to the cell-mediated reaction wherein nonspecific cytotoxic effector (immune) cells that express FcγRs recognize bound antibody on a target cell and subsequently cause phagocytosis of the target cell.

“CDC” or “complement-dependent cytotoxicity” refers to the reaction wherein one or more complement protein components recognize bound antibody on a target cell and subsequently cause lysis of the target cell.

In some embodiments, the modifications of the Fc domain include, but are not limited to, the following modifications and combinations thereof, with reference to EU numbering of an IgG constant region (Kabat et al, supra): 233, 234, 235, 236, 237, 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In certain embodiments, the Fc region comprises an amino acid addition, deletion, or substitution of one or more of amino acid residues 251-256, 285-290, 308-314, 385-389, and 428-436 of the IgG. In some embodiments, 251-256, 285-290, 308-314, 385-389, and 428-436 (EU numbering of Kabat) is substituted with histidine, arginine, lysine, aspartic acid, glutamic acid, serine, threonine, asparagine, or glutamine. In some embodiments, a non-histidine residue is substituted with a histidine residue. In some embodiments, a histidine residue is substituted with a non-histidine residue.

Enhancement of FcRn binding by an antibody having an engineered Fc leads to preferential binding of the affinity-enhanced antibody to FcRn as compared to antibody having wild-type Fc, and thus leads to a net enhanced recycling of the FcRn-affinity-enhanced antibody, which results in further increased antibody half-life. An enhanced recycling approach allows highly effective targeting and clearance of antigens, including e.g. “high titer” circulating antigens, such as C5, cytokines, or bacterial or viral antigens.

Provided in certain embodiments are modified constant region, Fc region or Fc fragment of an IgG antibody with enhanced binding to FcRn in serum as compared to a wild-type Fc region (without engineered modifications). In some instances, antibodies, e.g. IgG antibodies, are engineered to bind to FcRn at a neutral pH, e.g., at or above pH 7.4, to enhance pH-dependence of binding to FcRn as compared to a wild-type Fc region (without engineered modifications). In some instances, antibodies, e.g. IgG antibodies, are engineered to exhibit enhanced binding (e.g. increased affinity or K_{D}) to FcRn in endosomes (e.g., at an acidic pH, e.g., at or below pH 6.0) relative to a wild-type IgG and/or reference antibody binding to FcRn at an acidic pH, as well as in comparison to binding to FcRn in serum (e.g., at a neutral pH, e.g., at or above pH 7.4). Provided are antibodies with an engineered antibody constant region, Fc region or Fc fragment of an IgG antibody that exhibits an improved serum or resident tissue half-life, compared to a corresponding antibody having a wild-type IgG constant region, or an IgG constant without the recited modification(s);

Non-limiting examples of such Fc modifications include, e.g., a modification at position 250 (e.g., E or Q); 250 and 428 (e.g., L or F); 252 (e.g., LN/Y/W or T), 254 (e.g., S or T), and 256 (e.g., S/R/Q/E/D or T); or a modification at position 428 and/or 433 (e.g., H/L/R/S/P/Q or K) and/or 434 (e.g., H/F or Y); or a modification at position 250 and/or 428; or a modification at position 307 or 308 (e.g., 308F, V308F), and 434. In one embodiment, the modification comprises a 428L (e.g., M428L) and 434S (e.g., N434S) modification; a 428L, 2591 (e.g., V2591), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P) (EU numbering; see FIG. 23).

In some embodiments, the Fc region can be a mutant form such as hIgG1 Fc including M252 mutations, e.g. M252Y and S254T and T256E (“YTE mutation”) exhibit enhanced affinity for human FcRn (Dall'Acqua, et al., 2002, J Immunol 169:5171-5180) and subsequent crystal structure of this mutant antibody bound to hFcRn resulting in the creation of two salt bridges (Oganesyan, et al. 2014, JBC 289(11): 7812-7824). Antibodies having the YTE mutation have been administered to monkeys and humans, and have significantly improved pharmacokinetic properties (Haraya, et al., 2019, Drug Metabolism and Pharmacokinetics, 34(1):25-41).

In some embodiments, modifications to one or more amino acid residues in the Fc region may reduce half-life in systemic circulation (serum), however result in improved retainment in tissues (e.g. in the eye) by disabling FcRn binding (e.g. H435A, EU numbering of Kabat) (Ding et al., 2017, MAbs 9:269-284; and Kim, 1999, Eur J Immunol 29:2819).

In some embodiments, the Fc domain may be engineered to activate all, some, or none of the normal Fc effector functions, without affecting the Fc polypeptide's (e.g. antibody's) desired pharmacokinetic properties. Fc polypeptides having altered effector function may be desirable as they may reduce unwanted side effects, such as activation of effector cells, by the therapeutic protein.

Methods to alter or even ablate effector function may include mutation(s) or modification(s) to the hinge region amino acid residues of an antibody. For example, IgG Fc domain mutants comprising 234A, 237A, and 238S substitutions, according to the EU numbering system, exhibit decreased complement dependent lysis and/or cell mediated destruction. Deletions and/or substitutions in the lower hinge, e.g. where positions 233-236 within a hinge domain (EU numbering) are deleted or modified to glycine, have been shown in the art to significantly reduce ADCC and CDC activity.

In certain embodiments, the Fc domain is an aglycosylated Fc domain that has a substitution at residue 297 or 299 to alter the glycosylation site at 297 such that the Fc domain is not glycosylated. Such aglycosylated Fc domains may have reduced ADCC or other effector activity.

Non-limiting examples of proteins comprising mutant and/or chimeric C_{H} regions having altered effector functions, and methods of engineering and testing mutant antibodies, are described in the art, e.g. K. L. Amour, et al., Eur. J. Immunol. 1999, 29:2613-2624; Lazar et al., Proc. Natl. Acad. Sci. USA 2006, 103:4005; US Patent Application Publication No. 20070135620A1 published Jun. 14, 2007; US Patent Application Publication No. 20080154025 A1, published Jun. 26, 2008; US Patent Application Publication No. 20100234572 A1, published Sep. 16, 2010; US Patent Application Publication No. 20120225058 A1, published Sep. 6, 2012; US Patent Application Publication No. 20150337053 A1, published Nov. 26, 2015; International Publication No. WO20/16161010A2 published Oct. 6, 2016; U.S. Pat. No. 9,359,437, issued Jun. 7, 2016; and U.S. Pat. No. 10,053,517, issued Aug. 21, 2018, all of which are each herein incorporated by reference.

The C-terminal lysines (-K) conserved in the heavy chain genes of all human IgG subclasses are generally absent from antibodies circulating in serum—the C-terminal lysines are cleaved off in circulation, resulting in a heterogeneous population of circulating IgGs (van den Bremer et al., 2015, mAbs 7:672-680). In the vectored constructs for full length mAbs, the DNA encoding the C-terminal lysine (-K) or glycine-lysine (-GK) of the Fe terminus can be deleted to produce a more homogeneous antibody product in situ. (See, Hu et al., 2017 Biotechnol. Prog. 33: 786-794 which is incorporated by reference herein in its entirety).

### 5.2.5. Regulatory Elements

Enhancers, acting in cis, are nucleic acid elements that may enhance, or strongly stimulate, transcription of the antibody gene of interest, usually when bound by transcription factors. Enhancers may be upstream or downstream of the operably linked gene, and may even be thousands of base pairs away. Enhancer sequences may be in forward or reverse orientation, and still be active. In still other instances, the optimal expression of the antibody of interest may require the presence of one or more introns.

In other embodiments, the expression cassettes comprise a polyadenylation (polyA) site downstream of the coding region of the transgene. Any polyA site that signals termination of transcription and directs the synthesis of a polyA tail is suitable for use in AAV vectors of the present disclosure. Exemplary polyA signals are derived from, but not limited to, the following: the SV40 late gene, the rabbit β-globin gene the bovine growth hormone (BPH) gene, the human growth hormone (hGH) gene, the synthetic polyA (SPA) site, and the bovine growth hormone (bGH) gene. See, e.g., Powell and Rivera-Soto, 2015, Discov. Med., 19(102):49-57 and Table 8.

Introns appear to affect multiple aspects of gene expression, for ex. transcription, polyadenylation, translational efficiency, mRNA decay and even RNA polymerase processivity. Synergistic interactions may exist between splicing and polyadenylation functions and may contribute to more efficient 3′ end processing. The presence of an intron is optional, especially where there are space restraints in a given expression cassette, protein expression may be increased by intron addition.

TABLE 8
Exemplary regulatory, linker and cassette sequences

| | SEQ | |
|---|---|---|
| | ID | |
| Name | NO: | Nucleic Acid Sequence |
| CAG | 16 | GACATTGATTATTGACTAGTTATTAATAGTAATCAATTACGGGGTC |
| (1725 bp) | | ATTAGTTCATAGCCCATATATGGAGTTCCGCGTTACATAACTTACG |
| | | GTAAATGGCCCGCCTGGCTGACCGCCCAACGACCCCCGCCCATTGA |
| | | CGTCAATAATGACGTATGTTCCCATAGTAACGCCAATAGGGACTTT |
| | | CCATTGACGTCAATGGGTGGAGTATTTACGGTAAACTGCCCACTTG |
| | | GCAGTACATCAAGTGTATCATATGCCAAGTACGCCCCCTATTGACG |
| | | TCAATGACGGTAAATGGCCCGCCTGGCATTATGCCCAGTACATGAC |
| | | CTTATGGGACTTTCCTACTTGGCAGTACATCTACGTATTAGTCATC |
| | | GCTATTACCATGGTCGAGGTGAGCCCCACGTTCTGCTTCACTCTCC |
| | | CCATCTCCCCCCCCTCCCCACCCCCAATTTTGTATTTATTTATTTT |
| | | TTAATTATTTTGTGCAGCGATGGGGGCGGGGGGGGGGGGGGGGCGC |
| | | GCGCCAGGCGGGGCGGGGCGGGGCGAGGGGCGGGGCGGGGCGAGGC |
| | | GGAGAGGTGCGGCGGCAGCCAATCAGAGCGGCGCGCTCCGAAAGTT |
| | | TCCTTTTATGGCGAGGCGGCGGCGGCGGCGGCCCTATAAAAAGCGA |
| | | AGCGCGCGGCGGGCGGGAGTCGCTGCGCGCTGCCTTCGCCCCGTGC |
| | | CCCGCTCCGCCGCCGCCTCGCGCCGCCCGCCCCGGCTCTGACTGAC |
| | | CGCGTTACTCCCACAGGTGAGCGGGCGGGACGGCCCTTCTCCTCCG |
| | | GGCTGTAATTAGCGCTTGGTTTAATGACGGCTTGTTTCTTTTCTGT |
| | | GGCTGCGTGAAAGCCTTGAGGGGCTCCGGGAGGGCCCTTTGTGCGG |
| | | GGGGAGCGGCTCGGGGGGTGCGTGCGTGTGTGTGTGCGTGGGGAGC |
| | | GCCGCGTGCGGCTCCGCGCTGCCCGGCGGCTGTGAGCGCTGCGGGC |
| | | GCGGCGCGGGGCTTTGTGCGCTCCGCAGTGTGCGCGAGGGGAGCGC |
| | | GGCCGGGGGCGGTGCCCCGCGGTGCGGGGGGGGCTGCGAGGGGAAC |
| | | AAAGGCTGCGTGCGGGGTGTGTGCGTGGGGGGGTGAGCAGGGGGTG |
| | | TGGGCGCGTCGGTCGGGCTGCAACCCCCCCTGCACCCCCCTCCCCG |
| | | AGTTGCTGAGCACGGCCCGGCTTCGGGTGCGGGGCTCCGTACGGGG |
| | | CGTGGCGCGGGGCTCGCCGTGCCGGGCGGGGGGTGGCGGCAGGTGG |
| | | GGGTGCCGGGCGGGGGGGGGCCGCCTCGGGCCGGGGAGGGCTCGGG |
| | | GGAGGGGCGCGGCGGCCCCCGGAGCGCCGGCGGCTGTCGAGGCGCG |
| | | GCGAGCCGCAGCCATTGCCTTTTATGGTAATCGTGCGAGAGGGCGC |
| | | AGGGACTTCCTTTGTCCCAAATCTGTGCGGAGCCGAAATCTGGGAG |
| | | GCGCCGCCGCACCCCCTCTAGCGGGCGCGGGGCGAAGCGGTGCGGC |
| | | GCCGGCAGGAAGGAAATGGGCGGGGAGGGCCTTCGTGCGTCGCCGC |
| | | GCCGCCGTCCCCTTCTCCCTCTCCAGCCTCGGGGCTGTCCGCGGGG |
| | | GGACGGCTGCCTTCGGGGGGGACGGGGCAGGGCGGGGTTCGGCTTC |
| | | TGGCGTGTGACCGGCGGCTCTAGAGCCTCTGCTAACCATGTTCATG |
| | | CCTTCTTCTTTTTCCTACAGCTCCTGGGCAACGTGCTGGTTATTGT |
| | | GCTGTCTCATCATTTTGGCAAAG |
| CMV Enhancer | 17 | GACATTGATTATTGACTAGTTATTAATAGTAATCAATTACGGGGTC |
| (380 bp) | | ATTAGTTCATAGCCCATATATGGAGTTCCGCGTTACATAACTTACG |
| | | GTAAATGGCCCGCCTGGCTGACCGCCCAACGACCCCCGCCCATTGA |
| | | CGTCAATAATGACGTATGTTCCCATAGTAACGCCAATAGGGACTTT |
| | | CCATTGACGTCAATGGGTGGAGTATTTACGGTAAACTGCCCACTTG |
| | | GCAGTACATCAAGTGTATCATATGCCAAGTACGCCCCCTATTGACG |
| | | TCAATGACGGTAAATGGCCCGCCTGGCATTATGCCCAGTACATGAC |
| | | CTTATGGGACTTTCCTACTTGGCAGTACATCTACGTATTAGTCATC |
| | | GCTATTACCATG |
| Chimeric intron | 18 | GTAAGTATCAAGGTTACAAGACAGGTTTAAGGAGACCAATAGAAAC |
| (ß-globin/Ig | | TGGGCTTGTCGAGACAGAGAAGACTCTTGCGTTTCTGATAGGCACC |
| Intron) | | TATTGGTCTTACTGACATCCACTTTGCCTTTCTCTCCACAG |
| VH4 intron | 217 | GTGAGTATCTCAGGGATCCAGACATGGGGATATGGGAGGTGC |
| | | CTCTGATCCCAGGGCTCACTGTGGGTCTCTCTGTTCACAG |
| SV40 intron | 218 | GTAAGTTTAGTCTTTTTGTCTTTTATTTCAGGTCCCGGATCCGGTG |
| | | GTGGTGCAAATCAAAGAACTGCTCCTCAGTGGATGTTGCCTTTACT |
| | | TCTAG |
| Kozak sequence | 342 | GCCACC |
| CB/CBA | 19 | CGCGTGGTACCTCTGGTCGTTACATAACTTACGGTAAATGGCCCGC |
| promoter | | CTGGCTGACCGCCCAACGACCCCGCCCATTGACGTCAATAATGACG |
| (594 bp) | | TATGTTCCCATAGTAACGCCAATAGGGACTTTCCATTGACGTCAAT |
| | | GGGTGGAGTATTTACGGTAAACTGCCCACTTGGCAGTACATCAAGT |
| | | GTATCATATGCCAAGTACGCCCCCTATTGACGTCAATGACGGTAAA |
| | | TGGCCCGCCTGGCATTATGCCCAGTACATGACCTTATGGGACTTTC |
| | | CTACTTGGCAGTACATCTACTCGAGGCCACGTTCTGCTTCACTCTC |
| | | CCCATCTCCCCCCCCTCCCCACCCCCAATTTTGTATTTATTTATTT |
| | | TTTAATTATTTTGTGCAGCGATGGGGGGGGGGGGGGGGGGGGGGGG |
| | | GGCGCGCGCCAGGCGGGGCGGGGCGGGGCGAGGGGCGGGGCGGGGC |
| | | GAGGCGGAGAGGTGCGGCGGCAGCCAATCAGAGCGGCGCGCTCCGA |
| | | AAGTTTCCTTTTATGGCGAGGCGGCGGCGGCGGCGGCCCTATAAAA |
| | | AGCGAAGCGCGCGGCGGGCGGGAGCGGGATCAGCCACCGCGG |
| UbC promoter | 20 | TATATAAGGACGCGCCGGGTGTGGCACAGCTAGTTCCGTCGCAGCC |
| | | GGGATTTGGGTCGCAGTTCTTGTTTGTGGATCGCTGTGATCGTCAC |
| | | TTGGTGAGTAGCGGGCTGCTGGGCTGGCCGGGGCTTTCGTGGCCGC |
| | | CGGGCCGCTCGGTGGGACGGAGGCGTGTGGAGAGACCGCCAAGGGC |
| | | TGTAGTCTGGGTCCGCGAGCAAGGTTGCCCTGAACTGGGGGTTGGG |
| | | GGGAGCGCAGCAAAATGGCGGCTGTTCCCGAGTCTTGAATGGAAGA |
| | | CGCTTGTGAGGCGGGCTGTGAGGTCGTTGAAACAAGGTGGGGGGCA |
| | | TGGTGGGCGGCAAGAACCCAAGGTCTTGAGGCCTTCGCTAATGCGG |
| | | GAAAGCTCTTATTCGGGTGAGATGGGCTGGGGCACCATCTGGGGAC |
| | | CCTGACGTGAAGTTTGTCACTGACTGGAGAACTCGGTTTGTCGTCT |
| | | GTTGCGGGGGCGGCAGTTATGGCGGTGCCGTTGGGCAGTGCACCCG |
| | | TACCTTTGGGAGCGCGCGCCCTCGTCGTGTCGTGACGTCACCCGTT |
| | | CTGTTGGCTTATAATGCAGGGTGGGGCCACCTGCCGGTAGGTGTGC |
| | | GGTAGGCTTTTCTCCGTCGCAGGACGCAGGGTTCGGGCCTAGGGTA |
| | | GGCTCTCCTGAATCGACAGGCGCCGGACCTCTGGTGAGGGGAGGGA |
| | | TAAGTGAGGCGTCAGTTTCTCTGGTCGGTTTTATGTACCTATCTTC |
| | | TTAAGTAGCTGAAGCTCCGGTTTTGAACTATGCGCTCGGGGTTGGC |
| | | GAGTGTGTTTTGTGAAGTTTTTTAGGCACCTTTTGAAATGTAATCA |
| | | TTTGGGTCAATATGTAATTTTCAGTGTTAGACTAGTAAATTGTCCG |
| | | CTAAATTCTGGCCGTTTTTGGCTTTTTTGTTAGAC |
| UbC Enhancer | 21 | GGCCTCCGCGCCGGGTTTTGGCGCCTCCCGCGGGCGCCCCCCTCCT |
| | | CACGGCGAGCGCTGCCACGTCAGACGAAGGGCGCAGCGAGCGTCCT |
| | | GATCCTTCCGCCCGGACGCTCAGGACAGCGGCCCGCTGCTCATAAG |
| | | ACTCGGCCTTAGAACCCCAGTATCAGCAGAAGGACATTTTAGGACG |
| | | GGACTTGGGTGACTCTAGGGCACTGGTTTTCTTTCCAGAGAGCGGA |
| | | ACAGGCGAGGAAAAGTAGTCCCTTCTCGGCGATTCTGCGGAGGGAT |
| | | CTCCGTGGGGCGGTGAACGCCGATGAT |
| hSyn-1 promoter | 22 | AGTGCAAGTGGGTTTTAGGACCAGGATGAGGCGGGGTGGGGGTGCC |
| (448 bp) | | TACCTGACGACCGACCCCGACCCACTGGACAAGCACCCAACCCCCA |
| | | TTCCCCAAATTGCGCATCCCCTATCAGAGAGGGGGAGGGGAAACAG |
| | | GATGCGGCGAGGCGCGTGCGCACTGCCAGCTTCAGCACCGCGGACA |
| | | GTGCCTTCGCCCCCGCCTGGCGGCGCGCGCCACCGCCGCCTCAGCA |
| | | CTGAAGGCGCGCTGACGTCACTCGCCGGTCCCCCGCAAACTCCCCT |
| | | TCCCGGCCACCTTGGTCGCGTCCGCGCCGCCGCCGGCCCAGCCGGA |
| | | CCGCACCACGCGAGGCGCGAGATAGGGGGGCACGGGCGCGACCATC |
| | | TGCGCTGCGGCGCCGGCGACTCAGCGCTGCCTCAGTCTGCGGTGGG |
| | | CAGCGGAGGAGTCGTGTCGTGCCTGAGAGCGCAG |
| hSyn-2 promoter | 23 | CTGCAGAGGGCCCTGCGTATGAGTGCAAGTGGGTTTTAGGACCAGG |
| (449 bp) | | ATGAGGCGGGGTGGGGGTGCCTACCTGACGACCGACCCCGACCCAC |
| | | TGGACAAGCACCCAACCCCCATTCCCCAAATTGCGCATCCCCTATC |
| | | AGAGAGGGGGAGGGGAAACAGGATGCGGCGAGGCGCGTGCGCACTG |
| | | CCAGCTTCAGCACCGCGGACAGTGCCTTCGCCCCCGCCTGGCGGCG |
| | | CGCGCCACCGCCGCCTCAGCACTGAAGGCGCGCTGACGTCACTCGC |
| | | CGGTCCCCCGCAAACTCCCCTTCCCGGCCACCTTGGTCGCGTCCGC |
| | | GCCGCCGCCGGCCCAGCCGGACCGCACCACGCGAGGCGCGAGATAG |
| | | GGGGGCACGGGCGCGACCATCTGCGCTGCGGCGCCGGCGACTCAGC |
| | | GCTGCCTCAGTCTGC |
| hSyn-3 promoter | 24 | CTGCAGAGGGCCCTGCGTATGAGTGCAAGTGGGTTTTAGGACCAGG |
| (469 bp) | | ATGAGGCGGGGTGGGGGTGCCTACCTGACGACCGACCCCGACCCAC |
| | | TGGACAAGCACCCAACCCCCATTCCCCAAATTGCGCATCCCCTATC |
| | | AGAGAGGGGGAGGGGAAACAGGATGCGGCGAGGCGCGTGCGCACTG |
| | | CCAGCTTCAGCACCGCGGACAGTGCCTTCGCCCCCGCCTGGCGGCG |
| | | CGCGCCACCGCCGCCTCAGCACTGAAGGCGCGCTGACGTCACTCGC |
| | | CGGTCCCCCGCAAACTCCCCTTCCCGGCCACCTTGGTCGCGTCCGC |
| | | GCCGCCGCCGGCCCAGCCGGACCGCACCACGCGAGGCGCGAGATAG |
| | | GGGGGCACGGGCGCGACCATCTGCGCTGCGGCGCCGGCGACTCAGC |
| | | GCTGCCTCAGTCTGCGGTGGGCAGCGGAGGAGTCGTGTCGTGCCTG |
| | | AGAGCGCAGGGCGCGCC |
| hSyn-4 promoter | 25 | CTGCAGAGGGCCCTGCGTATGAGTGCAAGTGGGTTTTAGGACCAGG |
| 461 bp) | | ATGAGGCGGGGTGGGGGTGCCTACCTGACGACCGACCCCGACCCAC |
| | | TGGACAAGCACCCAACCCCCATTCCCCAAATTGCGCATCCCCTATC |
| | | AGAGAGGGGGAGGGGAAACAGGATGCGGCGAGGCGCGTGCGCACTG |
| | | CCAGCTTCAGCACCGCGGACAGTGCCTTCGCCCCCGCCTGGCGGCG |
| | | CGCGCCACCGCCGCCTCAGCACTGAAGGCGCGCTGACGTCACTCGC |
| | | CGGTCCCCCGCAAACTCCCCTTCCCGGCCACCTTGGTCGCGTCCGC |
| | | GCCGCCGCCGGCCCAGCCGGACCGCACCACGCGAGGCGCGAGATAG |
| | | GGGGGCACGGGCGCGACCATCTGCGCTGCGGCGCCGGCGACTCAGC |
| | | GCTGCCTCAGTCTGCCAATTGCAGCGGAGGAGTCGTGTCGTGCCTG |
| | | AGAGCGCAG |
| hSyn-5 promoter | 26 | CCTGCAGGGCCCACTAGTATCTGCAGAGGGCCCTGCGTATGAGTGC |
| (571 bp) | | AAGTGGGTTTTAGGACCAGGATGAGGCGGGGTGGGGGTGCCTACCT |
| | | GACGACCGACCCCGACCCACTGGACAAGCACCCAACCCCCATTCCC |
| | | CAAATTGCGCATCCCCTATCAGAGAGGGGGAGGGGAAACAGGATGC |
| | | GGCGAGGCGCGTGCGCACTGCCAGCTTCAGCACCGCGGACAGTGCC |
| | | TTCGCCCCCGCCTGGCGGCGCGCGCCACCGCCGCCTCAGCACTGAA |
| | | GGCGCGCTGACGTCACTCGCCGGTCCCCCGCAAACTCCCCTTCCCG |
| | | GCCACCTTGGTCGCGTCCGCGCCGCCGCCGGCCCAGCCGGACCGCA |
| | | CCACGCGAGGCGCGAGATAGGGGGGCACGGGCGCGACCATCTGCGC |
| | | TGCGGCGCCGGCGACTCAGCGCTGCCTCAGTCTGCGGTGGGCAGCG |
| | | GAGGAGTCGTGTCGTGCCTGAGAGCGCAGCTGTGCTCCTGGGCACC |
| | | GCGCAGTCCGCCCCCGCGGCTCCTGGCCAGACCACCCCTAGGACCC |
| | | CCTGCCCCAAGTCGCAGCC |
| Mecp2 promoter | 27 | AGCTGAATGGGGTCCGCCTCTTTTCCCTGCCTAAACAGACAGGAAC |
| | | TCCTGCCAATTGAGGGCGTCACCGCTAAGGCTCCGCCCCAGCCTGG |
| | | GCTCCACAACCAATGAAGGGTAATCTCGACAAAGAGCAAGGGGTGG |
| | | GGCGCGGGCGCGCAGGTGCAGCAGCACACAGGCTGGTCGGGAGGGC |
| | | GGGGCGCGACGTCTGCCGTGCGGGGTCCCGGCATCGGTTGCGCGC |
| hGFAP promoter | 28 | GTCTGCAAGCAGACCTGGCAGCATTGGGCTGGCCGCCCCCCAGGGC |
| | | CTCCTCTTCATGCCCAGTGAATGACTCACCTTGGCACAGACACAAT |
| | | GTTCGGGGTGGGCACAGTGCCTGCTTCCCGCCGCACCCCAGCCCCC |
| | | CTCAAATGCCTTCCGAGAAGCCCATTGAGTAGGGGGCTTGCATTGC |
| | | ACCCCAGCCTGACAGCCTGGCATCTTGGGATAAAAGCAGCACAGCC |
| | | CCCTAGGGGCTGCCCTTGCTGTGTGGCGCCACCGGCGGTGGAGAAC |
| | | AAGGCTCTATTCAGCCTGTGCCCAGGAAAGGGGATCAGGGGATGCC |
| | | CAGGCATGGACAGTGGGTGGCAGGGGGGGAGAGGAGGGCTGTCTGC |
| | | TTCCCAGAAGTCCAAGGACACAAATGGGTGAGGGGACTGGGCAGGG |
| | | TTCTGACCCTGTGGGACCAGAGTGGAGGGCGTAGATGGACCTGAAG |
| | | TCTCCAGGGACAACAGGGCCCAGGTCTCAGGCTCCTAGTTGGGCCC |
| | | AGTGGCTCCAGCGTTTCCAAACCCATCCATCCCCAGAGGTTCTTCC |
| | | CATCTCTCCAGGCTGATGTGTGGGAACTCGAGGAAATAAATCTCCA |
| | | GTGGGAGACGGAGGGGTGGCCAGGGAAACGGGGCGCTGCAGGAATA |
| | | AAGACGAGCCAGCACAGCCAGCTCATGCGTAACGGCTTTGTGGAGC |
| | | TGTCAAGGCCTGGTCTCTGGGAGAGAGGCACAGGGAGGCCAGACAA |
| | | GGAAGGGGTGACCTGGAGGGACAGATCCAGGGGCTAAAGTCCTGAT |
| | | AAGGCAAGAGAGTGCCGGCCCCCTCTTGCCCTATCAGGACCTCCAC |
| | | TGCCACATAGAGGCCATGATTGACCCTTAGACAAAGGGCTGGTGTC |
| | | CAATCCCAGCCCCCAGCCCCAGAACTCCAGGGAATGAATGGGCAGA |
| | | GAGCAGGAATGTGGGACATCTGTGTTCAAGGGAAGGACTCCAGGAG |
| | | TCTGCTGGGAATGAGGCCTAGTAGGAAATGAGGTGGCCCTTGAGGG |
| | | TACAGAACAGGTTCATTCTTCGCCAAATTCCCAGCACCTTGCAGGC |
| | | ACTTACAGCTGAGTGAGATAATGCCTGGGTTATGAAATCAAAAAGT |
| | | TGGAAAGCAGGTCAGAGGTCATCTGGTACAGCCCTTCCTTCCCTTT |
| | | TTTTTTTTTTTTTTTTTTTGTGAGACAAGGTCTCTCTCTGTTGCCC |
| | | AGGCTGGAGTGGCGCAAACACAGCTCACTGCAGCCTCAACCTACTG |
| | | GGCTCAAGCAATCCTCCAGCCTCAGCCTCCCAAAGTGCTGGGATTA |
| | | CAAGCATGAGCCACCCCACTCAGCCCTTTCCTTCCTTTTTAATTGA |
| | | TGCATAATAATTGTAAGTATTCATCATGGTCCAACCAACCCTTTCT |
| | | TGACCCACCTTCCTAGAGAGAGGGTCCTCTTGATTCAGCGGTCAGG |
| | | GCCCCAGACCCATGGTCTGGCTCCAGGTACCACCTGCCTCATGCAG |
| | | GAGTTGGCGTGCCCAGGAAGCTCTGCCTCTGGGCACAGTGACCTCA |
| | | GTGGGGTGAGGGGAGCTCTCCCCATAGCTGGGCTGCGGCCCAACCC |
| | | CACCCCCTCAGGCTATGCCAGGGGGTGTTGCCAGGGGCACCCGGGC |
| | | ATCGCCAGTCTAGCCCACTCCTTCATAAAGCCCTCGCATCCCAGGA |
| | | GCGAGCAGAGCCAGAGCAT |
| Rat NSE/RU5′ | 29 | AGCTCTGAGCTCCTCCTCTGCTCGCCCAATCCTTCCAACCCCCTAT |
| promoter | | GGTGGTATGGCTGACACAGAAAATGTCTGCTCCTGTATGGGACATT |
| (1743 bp) | | TGCCCCTCTTCTCCAAATATAAGACAGGATGAGGCCTAGCTTTTGC |
| | | TGCTCCAAAGTTTTAAAAGAACACATTGCACGGCATTTAGGGACTC |
| | | TAAAGGGTGGAGGAGGAATGAGGGAATTGCATCATGCCAAGGCTGG |
| | | TCCTCATCCATCACTGCTTCCAGGGCCCAGAGTGGCTTCCAGGAAG |
| | | TATTCTTACAAAGGAAGCCCGATCTGTAGCTAACACTCAGAGCCCA |
| | | TTTTCCTGCGTTAACCCCTCCCGACCTCATATACAGGAGTAACATG |
| | | ATCAGTGACCTGGGGGAGCTGGCCAAACTGCGGGACCTGCCCAAGC |
| | | TGAGGGCCTTGGTGCTGCTGGACAACCCCTGTGCCGATGAGACTGA |
| | | CTACCGCCAGGAGGCCCTGGTGCAGATGGCACACCTAGAGCGCCTA |
| | | GACAAAGAGTACTATGAGGACGAGGACCGGGCAGAAGCTGAGGAGA |
| | | TCCGACAGAGGCTGAAGGAGGAACAGGAGCAAGAACTCGACCCGGA |
| | | CCAAGACATGGAACCGTACCTCCCGCCAACTTAGTGGCTCCTCTAG |
| | | CCTGCAGGGACAGTAAAGGTGATGGCAGGAAGGCAGCCCCCGGAGG |
| | | TCAAAGGCTGGGCACGCGGGAGGAGAGGCCAGAGTCAGAGGCTGCG |
| | | GGTATCTCAGATATGAAGGAAAGATGAGAGAGGCTCAGGAAGAGGT |
| | | AAGAAAAGACACAAGAGACCAGAGAAGGGAGAAGAATTAGAGAGGG |
| | | AGGCAGAGGACCGCTGTCTCTACAGACATAGCTGGTAGAGACTGGG |
| | | AGGAAGGGATGAACCCTGAGCGCATGAAGGGAAGGAGGTGGCTGGT |
| | | GGTATATGGAGGATGTAGCTGGGCCAGGGAAAAGATCCTGCACTAA |
| | | AAATCTGAAGCTAAAAATAACAGGACACGGGGTGGAGAGGCGAAAG |
| | | GAGGGCAGAGTGAGGCAGAGAGACTGAGAGGCCTGGGGATGTGGGC |
| | | ATTCCGGTAGGGCACACAGTTCACTTGTCTTCTCTTTTTCCAGGAG |
| | | GCCAAAGATGCTGACGTCAAGAACTCATAATACCCCAGTGGGGACC |
| | | ACCGCATTCATAGCCCTGTTACAAGAAGTGGGAGATGTTCCTTTTT |
| | | GTCCCAGACTGGAAATCCGTTACATCCCGAGGCTCAGGTTCTGTGG |
| | | TGGTCATCTCTGTGTGGCTTGTTCTGTGGGCCTACCTAAAGTCCTA |
| | | AGCACAGCTCTCAAGCAGATCCGAGGCGACTAAGATGCTAGTAGGG |
| | | GTTGTCTGGAGAGAAGAGCCGAGGAGGTGGGCTGTGATGGATCAGT |
| | | TCAGCTTTCAAATAAAAAGGCGTTTTTATATTCTGTGTCGAGTTCG |
| | | TGAACCCCTGTGGTGGGCTTCTCCATCTGTCTGGGTTAGTACCTGC |
| | | CACTATACTGGAATAAGGGGACGCCTGCTTCCCTCGAGTTGGCTGG |
| | | ACAAGGTTATGAGCATCCGTGTACTTATGGGGTTGCCAGCTTGGTC |
| | | CTGGATCGCCCGGGCCCTTCCCCCACCCGTTCGGTTCCCCACCACC |
| | | ACCCGCGCTCGTACGTGCGTCTCCGCCTGCAGCTCTTGACTCATCG |
| | | GGGCCCCCGGGTCACATGCGCTCGCTCGGCTCTATAGGCGCCGCCC |
| | | CCTGCCCACCCCCCGCCCGCGCTGGGAGCCGCAGCCGCCGCCACTC |
| | | CTGCTCTCTCTGCGCCGAAGCTTCGAGGGGCTCGCATCTCTCCTTC |
| | | ACGCGCCCGCCGCCCTACCTGAGGCCGCCATCCACGCCGGTTGAGT |
| | | CGCGTTCTGCCGCCTCCCGCCTGTGGTGCCTCCTGAACTGCGTCCG |
| | | CCGTCTAGGTAAGTTTAAAGCTCAGGTCGAGACCGGGCCTTTGTCC |
| | | GGCGCTCCCTTGGAGCCTACCTAGACTCAGCCGGCTCTCCACGCTT |
| | | TGCCTGACCCTGCTTGCTCAACTCTACGTCTTTGTTTCGTTTTCTG |
| | | TTCTGCGCCGTTACAGATC |
| NeuN | 30 | GAGGAGGAGGAGAGAGACCGGGAGGGCGCCCGGGAGGCAGGGCGCG |
| (H. sapiens) | | CGCACACTCCGAGG |
| NG_053112.1 | | |
| CamKII | 31 | GATGCTGACGAAGGCTCGCGAGGCTGTGAGCAGCCACAGTGCCCTG |
| (α; H. sapiens) | | CTCAGAAGCCCCGG |
| NM_171825 | | |
| CamKII | 32 | ACTTGTGGACTAAGTTTGTTCGCATCCCCTTCTCCAACCCCCTCAG |
| (α; M. musculus) | | TACATCACCCTGGGGGAACAGGGTCCACTTGCTCCTGGGCCCACAC |
| 7625-7988: | | AGTCCTGCAGTATTGTGTATATAAGGCCAGGGCAAAGAGGAGCAGG |
| GenBank | | TTTTAAAGTGAAAGGCAGGCAGGTGTTGGGGAGGCAGTTACCGGGG |
| AJ222796 | | CAACGGGAACAGGGCGTTTCGGAGGTGGTTGCCATGGGGACCTGGA |
| (364 bp) | | TGCTGACGAAGGCTCGCGAGGCTGTGAGCAGCCACAGTGCCCTGCT |
| Hioki et al* | | CAGAAGCCCCAAGCTCGTCAGTCAAGCCGGTTCTCCGTTTGCACTC |
| | | AGGAGCACGGGCAGGCGAGTGGCCCCTAGTTCTGGGGGCAGC |
| CamKII | 33 | GTCTCCCGCGCCCGCGCCCGTGTCGCCGCCGTGCCCGCGAGCGGGA |
| (β1; H. sapiens) | | GCCGGAGTCGCCGC |
| NM_172084 | | |
| CamKII | 34 | CGTGTGCAGATGCAGGGCGCCGGTGCCCTGCGGGTGCGGGTGCAGG |
| (β2; H. sapiens) | | AGCAGCGTGTGCAG |
| NM_172084 | | |
| CamKII | 35 | CCCCACGCCACCCTTTCTGGTCATCTCCCCTCCCGCCCCGCCCCTG |
| (δ; H. sapiens) | | CGCACACTCCCTCG |
| NM_172115 | | |
| CamKII | 36 | TCTCCCCGGTAAAGTCTCGCGGTGCTGCCGGGCTCAGCCCCGTCTC |
| (γ; H. sapiens) | | CTCCTCTTGCTCCC |
| NM_172171 | | |
| NSE/ENO2 | 37 | CGCCTCCTCCGCCCGCCGCCCGGGAGCCGCAGCCGCCGCCGCCACT |
| (isoform 1; H. | | GCCACTCCCGCTCT |
| sapiens) | | |
| NM_001975 | | |
| NSE/ENO2 | 38 | TGGGTGCCCCCACCCTTCCCCCATCCTCCTCCCTTCCCCACTCCAC |
| (isoform 2; H. | | CCTCGTCGGTCCCC |
| sapiens) | | |
| NM_001975 | | |
| PDGFB | 39 | AAAAAAAAAAAAAAAGCCCACCCTCCAGCCTCGCTGCAAAGAGAAA |
| (isoform 1; H. | | ACCGGAGCAGCCGC |
| sapiens) | | |
| NM_033016 | | |
| PDGFB | 40 | TCTCGCACTCTCCCTTCTCCTTTATAAAGGCCGGAACAGCTGAAAG |
| (isoform 2; H. | | GGTGGCAACTTCTC |
| sapiens) | | |
| NM_033016 | | |
| PDGFB | 41 | GCCGCGTCCACCTGTCGGCCGGGCCCAGCCGAGCGCGCAGCGGGCA |
| (isoform 3; H. | | CGCCGCGCGCGCGG |
| sapiens) | | |
| NM_033016 | | |
| VGluT1/SLC17A7 | 42 | GCGCCCCGCCCCCGGCGCTGAGTCCTGTGACAGCCCCCGGGCCGCC |
| (H. sapiens) | | TGCACTTGCAGCCT |
| NM_020309 | | |
| VGluT2/SLC17A6 | 43 | AAAGAAGAGTCCCCTATTCCTGAAACTTACTCTGTCCGTGGTGCTG |
| (isoform 1; H. | | AAACATTGTACCGA |
| sapiens) | | |
| NM_020346 | | |
| VGluT2/SLC17A6 | 44 | CGTCCTCAAAGAGCAGCAAGCCTTCTCCATCTTAATTTGACTCTAC |
| (isoform 2; H. | | CGCAGAGCAGACTT |
| sapiens) | | |
| NM_020346 | | |
| VGluT2/SLC17A6 | 45 | ATGCAGCTATTCTGTTGTATTCTCATTCTCACTCTCCCTCCCTTCT |
| (isoform 3; H. | | CTCACTCTCACTCT |
| sapiens) | | |
| NM_020346 | | |
| VGluT3/SLC17A8 | 46 | CATGTTAGCGTCCCCAGCTGCAGCCCAGGGAGGGAGAGAGGCTGCG |
| (H. sapiens) | | CTCAGTCTGAGAGT |
| NM_001145288 | | |
| SST | 47 | TGACGTCAGAGAGAGAGTTTAAAACAGAGGGAGACGGTTGAGAGCA |
| (isoform 1; H. | | CACAAGCCGCTTTA |
| sapiens) | | |
| NM_001048 | | |
| SST | 48 | GAGTGAAAATAAAAGATTGTATAAATCGTGGGGCATGTGGAATTGT |
| (isoform 2; H. | | GTGTGCCTGTGCGT |
| sapiens) | | |
| NM_001048 | | |
| NPY | 49 | GCCGCGGCGAGGAAGCTCCATAAAAGCCCTGTCGCGACCCGCTCTC |
| (H. sapiens) | | TGCACCCCATCCGC |
| NM_000905 | | |
| VIP | 50 | CAGTCCTAAGTATAAGCCCTATAAAATGATGGGCTTTGAAATGCTG |
| (isoform 1; H. | | GTCAGGGTAGAGTG |
| sapiens) | | |
| NM_194435 | | |
| VIP | 51 | TTTTCCATTAATGTTTTCAGACTGCTGTTGACCACAGGTAACTGAA |
| (isoform 2; H. | | ATCATGGAAAGAGA |
| sapiens) | | |
| NM_194435 | | |
| PV | 52 | TGGTCATATGAGCAGAAATGATGAGAAAAGCACTTTTTAATCTTTT |
| (isoform 1; H. | | CGCACTTGCTCTGC |
| sapiens) | | |
| NM_002854 | | |
| PV | 53 | AATAGCCAGAGCAGAAGCCTATATAGGTGGCCATCCCACCTCCAGG |
| (isoform 2; H. | | CTCACTTCCCGACA |
| sapiens) | | |
| NM_002854 | | |
| PV | 54 | CAGCGCTCAGATTTTGCAGCATAAATTTGCATCCAGGACAGACCAG |
| (isoform 3; H. | | AGCAGAGGCTGAGG |
| sapiens) | | |
| NM_002854 | | |
| GAD65/GAD2 | 55 | GCACGCACGCGCGCGCAGGGCCAAGCCCGAGGCAGCTCGCCCGCAG |
| (isoform 1; H. | | CTCGCACTCGCAGG |
| sapiens) | | |
| NM_001134366 | | |
| GAD65/GAD2 | 56 | CCCGCCTCTGGCTCGCCCGAGGACGCGCTGGCACGCCTCCCACCCC |
| (isoform 2 H. | | CTCACTCTGACTCC |
| sapiens) | | |
| NM_001134366 | | |
| GAD65/GAD2 | 57 | CACTGGGCTCCCTTTCCCTCAAATGCTCTGGGGCTCTCCGCGCTTT |
| (isoform 3; H. | | CCTGAGTCCGGGCT |
| sapiens) | | |
| NM_001134366 | | |
| GAD65/GAD2 | 58 | CACAGAAAACTCCTCTGGGCCACGCTTCCCGCCTCGCCGAGGTCTC |
| (isoform 4; H. | | CCCAGTCTGCCCCT |
| sapiens) | | |
| NM_001134366 | | |
| GAD67/GAD1 | 59 | CTCTGCCCCCGCCTACCCCGGAGCCGTGCAGCCGCCTCTCCGAATC |
| (isoform 1 H. | | TCTCTCTTCTCCTG |
| sapiens) | | |
| NM_013445 | | |
| GAD67/GAD1 | 60 | CTGGATTTATAATCGCCCTATAAAGCTCCAGAGGCGGTCAGGCACC |
| (isoform 2; H. | | TGCAGAGGAGCCCC |
| sapiens) | | |
| NM_013445 | | |
| DRD1 | 61 | GGGACGCGCGGGCGGGGTGGGCTGTGCCCCGCGGGAACCCCGCCGG |
| (H. sapiens) | | CCTGTGCGCTTGCTG |
| NM_000794 | | |
| DRD2 | 62 | CTCCCTCCCGCGCTCCCCGCGCTCGGGCGCCGCAGAGCTGTCCAGC |
| (isoform 1; H. | | TTCAGTGCCGAACC |
| sapiens) | | |
| NM_016574 | | |
| DRD2 | 63 | GTACTGGTGTACAAGGACAAGGTGACTTTTTTTCTTTTCCCAGATT |
| (isoform 2; H. | | GAAAGGGCCAAAGA |
| sapiens) | | |
| NM_016574 | | |
| C1ql2 | 64 | CCTCCGCCGCTCAGCCCCGGACTCCTTACGTCAGGGTAGCGGGGTC |
| (H. sapiens) | | CCCCCTCCGCGCGG |
| NM_182528 | | |
| POMC | 65 | CCAGGAGAGCTCGGCAAGTATATAAGGACAGAGGAGCGCGGGACCA |
| (H. sapiens) | | AGCGGCGGCGAAGG |
| NM_001319204 | | |
| PROX1 | 66 | TTCCTTCAGCTGTGTCTTAAAGTAAATCTTGTTGTGGAGCGGAGCC |
| (isoform 1; H. | | CTCAGCTGAGGGAG |
| sapiens) | | |
| NM_002763 | | |
| PROX1 | 67 | GTAAGTATCTTCTTCTTCCCCTCGTGAGTCCCTCCCCTTTTCCAGA |
| (isoform 2) | | ATCACTTGCACTGT |
| NM_002763 | | |
| MAP1B | 68 | GGGGCGGAGCGGAGACAGTACCTTCGGAGATAATCCTTTCTCCTGC |
| (isoform 1; H. | | CGCAGTGGAGAGGA |
| sapiens) | | |
| NM_005909 | | |
| MAP1B | 69 | CCCTGCCTAGTCTCCATATAAAAGCGGCGCCGCCTCCCCGCCCTCT |
| (isoform 2; H. | | CTCACTCCCCGCTC |
| sapiens) | | |
| NM_005909 | | |
| MAP1B | 70 | GGGCGGCCCAGCCCCAGGTTACGTCGTCCCCAGAAAGAATCTGGCC |
| (isoform 3; H. | | AACAGTCTGGCCGT |
| sapiens) | | |
| NM_005909 | | |
| Tα-1/TUBA1A | 71 | GGCAGGTTCTCTTACATCGACCGCTTAAGAGTCGCGCTGTAAGAAG |
| (isoform 1; H. | | CAACACCTCCTCCTCGCCTCCGCCATCCACCCGGCAGCCGCGAAGC |
| sapiens) | | AGCAACC |
| NM_006009 | | |
| CK8 | 72 | CCACTACGGGTTTAGGCTGCCCATGTAAGGAGGCAAGGCCTGGGGA |
| | | CACCCGAGATGCCTGGTTATAATTAACCCAGACATGTGGCTGCCCC |
| | | CCCCCCCCCCAACACCTGCTGCCTCTAAAAATAACCCTGTCCCTGG |
| | | TGGATCCCACTACGGGTTTAGGCTGCCCATGTAAGGAGGCAAGGCC |
| | | TGGGGACACCCGAGATGCCTGGTTATAATTAACCCAGACATGTGGC |
| | | TGCCCCCCCCCCCCCCAACACCTGCTGCCTCTAAAAATAACCCTGT |
| | | CCCTGGTGGATCCCACTACGGGTTTAGGCTGCCCATGTAAGGAGGC |
| | | AAGGCCTGGGGACACCCGAGATGCCTGGTTATAATTAACCCAGACA |
| | | TGTGGCTGCCCCCCCCCCCCCCAACACCTGCTGCCTCTAAAAATAA |
| | | CCCTGTCCCTGGTGGATCCCCTGCATGCGAAGATCTTCGAACAAGG |
| | | CTGTGGGGGACTGAGGGCAGGCTGTAACAGGCTTGGGGGCCAGGGC |
| | | TTATACGTGCCTGGGACTCCCAAAGTATTACTGTTCCATGTTCCCG |
| | | GCGAAGGGCCAGCTGTCCCCCGCCAGCTAGACTCAGCACTTAGTTT |
| | | AGGAACCAGTGAGCAAGTCAGCCCTTGGGGCAGCCCATACAAGGCC |
| | | ATGGGGCTGGGCAAGCTGCACGCCTGGGTCCGGGGTGGGCACGGTG |
| | | CCCGGGCAACGAGCTGAAAGCTCATCTGCTCTCAGGGGCCCCTCCC |
| | | TGGGGACAGCCCCTCCTGGCTAGTCACACCCTGTAGGCTCCTCTAT |
| | | ATAACCCAGGGGCACAGGGGCTGCCCTCATTCTACCACCACCTCCA |
| | | CAGCACAGACAGACACTCAGGAGCCAGCCAGCGTCGA |
| mU1a | 73 | ATGGAGGCGGTACTATGTAGATGAGAATTCAGGAGCAAACTGGGAA |
| | | AAGCAACTGCTTCCAAATATTTGTGATTTTTACAGTGTAGTTTTGG |
| | | AAAAACTCTTAGCCTACCAATTCTTCTAAGTGTTTTAAAATGTGGG |
| | | AGCCAGTACACATGAAGTTATAGAGTGTTTTAATGAGGCTTAAATA |
| | | TTTACCGTAACTATGAAATGCTACGCATATCATGCTGTTCAGGCTC |
| | | CGTGGCCACGCAACTCATACT |
| EF-1α | 74 | GGGCAGAGCGCACATCGCCCACAGTCCCCGAGAAGTTGGGGGGAGG |
| | | GGTCGGCAATTGAACGGGTGCCTAGAGAAGGTGGCGCGGGGTAAAC |
| | | TGGGAAAGTGATGTCGTGTACTGGCTCCGCCTTTTTCCCGAGGGTG |
| | | GGGGAGAACCGTATATAAGTGCAGTAGTCGCCGTGAACGTTCTTTT |
| | | TCGCAACGGGTTTGCCGCCAGAACACAG |
| TBG | 75 | GGGCTGGAAGCTACCTTTGACATCATTTCCTCTGCGAATGCATGTA |
| | | TAATTTCTACAGAACCTATTAGAAAGGATCACCCAGCCTCTGCTTT |
| | | TGTACAACTTTCCCTTAAAAAACTGCCAATTCCACTGCTGTTTGGC |
| | | CCAATAGTGAGAACTTTTTCCTGCTGCCTCTTGGTGCTTTTGCCTA |
| | | TGGCCCCTATTCTGCCTGCTGAAGACACTCTTGCCAGCATGGACTT |
| | | AAACCCCTCCAGCTCTGACAATCCTCTTTCTCTTTTGTTTTACATG |
| | | AAGGGTCTGGCAGCCAAAGCAATCACTCAAAGTTCAAACCTTATCA |
| | | TTTTTTGCTTTGTTCCTCTTGGCCTTGGTTTTGTACATCAGCTTTG |
| | | AAAATACCATCCCAGGGTTAATGCTGGGGTTAATTTATAACTAAGA |
| | | GTGCTCTAGTTTTGCAATACAGGACATGCTATAAAAATGGAAAGAT |
| ApoE.hAAT | 76 | AGGCTCAGAGGCACACAGGAGTTTCTGGGCTCACCCTGCCCCCTTC |
| | | CAACCCCTCAGTTCCCATCCTCCAGCAGCTGTTTGTGTGCTGCCTC |
| | | TGAAGTCCACACTGAACAAACTTCAGCCTACTCATGTCCCTAAAAT |
| | | GGGCAAACATTGCAAGCAGCAAACAGCAAACACACAGCCCTCCCTG |
| | | CCTGCTGACCTTGGAGCTGGGGCAGAGGTCAGAGACCTCTCTGGGC |
| | | CCATGCCACCTCCAACATCCACTCGACCCCTTGGAATTTCGGTGGA |
| | | GAGGAGCAGAGGTTGTCCTGGCGTGGTTTAGGTAGTGTGAGAGGGG |
| | | TACCCGGGGATCTTGCTACCAGTGGAACAGCCACTAAGGATTCTGC |
| | | AGTGAGAGCAGAGGGCCAGCTAAGTGGTACTCTCCCAGAGACTGTC |
| | | TGACTCACGCCACCCCCTCCACCTTGGACACAGGACGCTGTGGTTT |
| | | CTGAGCCAGGTACAATGACTCCTTTCGGTAAGTGCAGTGGAAGCTG |
| | | TACACTGCCCAGGCAAAGCGTCCGGGCAGCGTAGGCGGGCGACTCA |
| | | GATCCCAGCCAGTGGACTTAGCCCCTGTTTGCTCCTCCGATAACTG |
| | | GGGTGACCTTGGTTAATATTCACCAGCAGCCTCCCCCGTTGCCCCT |
| | | CTGGATCCACTGCTTAAATACGGACGAGGACAGGGCCCTGTCTCCT |
| | | CAGCTTCAGGCACCACCACTGACCTGGGACAGT |
| pTetbidi | 343 | TTACCCGGGGAGCATGTCAAGGTCAAAATCGTCAAGAGCGTCAGCA |
| | | GGCAGCATATCAAGGTCAAAGTCGTCAAGGGCATCGGCTGGGAGCA |
| | | TGTCTAAGTCAAAATCGTCAAGGGCGTCGGTCGGCCCGCCGCTTTC |
| | | GCACTTTAGCTGTTTCTCCAGGCCACATATGATTAGTTCCAGGCCG |
| | | AAAAGGAAGGCAGGTTCGGCTCCCTGCCGGTCGAACAGCTCAATTG |
| | | CTTGTTTCAGAAGTGGGGGCATAGAATCGGTGGTAGGTGTCTCTCT |
| | | TTCCTCTTTTGCTACTTGATGCTCCTGTTCCTCCAATACGCAGCCC |
| | | AGTGTAAAGTGGCCCACGGCGGACAGAGCGTACAGTGCGTTCTCCA |
| | | GGGAGAAGCCTTGCTGACACAGGAACGCGAGCTGATTTTCCAGGGT |
| | | TTCGTACTGTTTCTCTGTTGGGCGGGTGCCGAGATGCACTTTAGCC |
| | | CCGTCGCGATGTGAGAGGAGAGCACAGCGGTATGACTTGGCGTTGT |
| | | TCCGCAGAAAGTCTTGCCATGACTCGCCTTCCAGGGGGCAGGAGTG |
| | | GGTATGATGCCTGTCCAGCATCTCGATTGGCAGGGCATCGAGCAGG |
| | | GCCCGCTTGTTCTTCACGTGCCAGTACAGGGTAGGCTGCTCAACTC |
| | | CCAGCTTTTGAGCGAGTTTCCTTGTCGTCAGGCCTTCGATACCGAC |
| | | TCCATTGAGTAATTCCAGAGCAGAGTTTATGATTTTGCTCTTGTCC |
| | | AGTCTAGACAT |
| Furin-T2A | 77 | AGAAAGAGAAGAGGCTCTGGAGAAGGCAGAGGCTCCCTGCTGACAT |
| | | GTGGGGATGTTGAAGAGAATCCTGGGCCT |
| Furin | 78 | AGAAAGAGAAGA |
| Furin-GSG | 79 | AGAAAGAGAAGAGGCTCTGGA |
| Linker | | |
| GSG | 80 | GGCTCTGGA |
| (GSG)₂ | 81 | GGCTCTGGAGGCTCTGGA |
| GGGGS | 82 | GGCGGAGGAGGCTCT |
| (G4S) | | |
| T2A | 83 | GAAGGCAGAGGCTCCCTGCTGACATGTGGGGATGTTGAAGAGAATC |
| | | CTGGGCCT |
| Rabbit ß-globin | 84 | GATCTTTTTCCCTCTGCCAAAAATTATGGGGACATCATGAAGCCCC |
| poly A | | TTGAGCATCTGACTTCTGGCTAATAAAGGAAATTTATTTTCATTGC |
| (signal in bold | | AATAGTGTGTTGGAATTTTTTGTGTCTCTCACTCG |
| type) | | |
| ß-globin PolyA | 85 | ATAAAGGAAATTTATTTTCATTGCAATAGTGTGTTGGAATTTTTTG |
| signal | | TGTCTCTCA |
| IL-2 leader | 86 | ATGTACAGAATGCAGCTGCTGCTGCTCATTGCCCTGTCTCTGGCCC |
| peptide v1 | | TGGTCACCAATTCT |

Ubiquitous, cell-type-specific, tissue-specific, developmental stage-specific, and conditional promoters, for example, drug-responsive promoters (e.g. tetracycline-responsive promoters) are all promoters potentially useful for Tau-specific antibody expression. The list provided (Table 8) is non-limiting and many promoters are well known in the art. In some embodiments, a portion or fragment of the promoter is included in the cis-acting plasmid or cassette. The portion or fragment of the promoter may be the transcriptionally active portion or fragment. Cell- and tissue specific promoter elements may be particularly suitable for expressing antibodies targeting Tau.

Exemplary promoters that are useful for the expression of the disclosed Tau-specific antibodies in mammalian cells (transduced with viral vector) include ubiquitous promoters such as, e.g., a phosphoglycerate kinase (PKG) promoter, CAG (composite of the (CMV) cytomegalovirus enhancer the chicken beta actin promoter (CBA) and the rabbit beta globin intron), the SV40 early promoter, murine mammary tumor virus LTR promoter; adenovirus major late promoter (Ad MLP); a herpes simplex virus (HSV) promoter, a CMV promoter such as the CMV immediate early promoter region (CMV-IE), rous sarcoma virus (RSV) promoter, and U6 promoter. For the purpose of driving cell-type specific expression of antibody sequences disclosed herein, cell-type specific promoters may be used. For example, neuron-specific expression of the antibodies can be conferred using neuronal-specific promoters, such as, e.g., a human synapsin 1 (hSyn) promoter (SEQ ID NO: 22), methyl CpG-binding protein 2 (Mecp2, SEQ ID NO: 27), GFAPNSE/RU5′ (SEQ ID NO: 28), hexaribonucleotide binding protein-3 (NeuN) promoter (SEQ ID NO: 30), Cα2+/calmodulin-dependent protein kinase II (CamKII) promoter (SEQ ID NOs: 31-36, Wang, L., Bai, J., & Hu, Y. (2007). Identification of the RA response element and transcriptional silencer in human αCaMKII promoter. Molecular Biology Reports, 35(1), 37-44), tubulin alpha I (Ta-1) promoter (SEQ ID NO: 71), neuron-specific enolase (NSE) promoter (SEQ ID NO: 29), platelet-derived growth factor beta chain (PDGFβ) promoter (SEQ ID NOs: 39-41), vesicular glutamate transporter (VGLUT) promoter (SEQ ID NOs: 42-46), somatostatin (SST) promoter (SEQ ID NO: 47), neuropeptide Y (NPY) promoter (SEQ ID NO: 49), vasoactive intestinal peptide (VIP) promoter (SEQ ID NOs: 50, 51), parvalbumin (PV) promoter (SEQ ID NOs: 42-54), glutamate decarboxylase (GAD65 or GAD67) promoter (SEQ ID Nos; 55-60), promoter of Dopamine-1 receptor (DRD1, SEQ ID NO: 61) and Dopamine-2 receptor (DRD2, SEQ ID NOs: 62, 63), microtubule-associated protein 1B (MAP1B, SEQ ID NOs: 68-70), complement component 1 q subcomponent-like 2 (Clq12) promoter (SEQ ID NO: 64), pro-opiomelanocortin (POMC) promoter (SEQ ID NO: 65), and prospero homeobox protein 1 (PROX1) promoter (SEQ ID NOs: 66, 67). Promoters suitable for driving polynucleotide expression specifically in astrocytes include Glial fibrillary acidic protein (GFAP)(Griffin, J M, et al. Gene Therapy (2019) 26:198-210), or variants thereof, e.g. GfaABC1D promoter, and ALDH1L1 (Koh, W. et al. Exp Neurobiol. 2017 December; 26(6):350-361). Promoters suitable for driving polynucleotide expression specifically in DG cells of the hippocampus include the Clql2, POMC, and PROX1 promoters. Synthetic promoters, hybrid promoters, and the like may also be used in conjunction with the methods and compositions disclosed herein. In addition, sequences derived from non-viral genes, such as the murine metallothionein gene, will also find use herein.

Such promoter sequences are commercially available from various sources, e.g., Stratagene (San Diego, CA) or InvivoGen (San Diego, CA), or may be engineered using standard molecular biology techniques. Exemplary promoter sequences suitable for use in expression vectors (e.g., plasmid or viral vector, such as, e.g., an AAV or a lentiviral vector) are provided in Table 8 above. Inducible promoters have been described, and provide regulatable transgene expression, including in the brain, utilizing, e.g. doxycycline-inducible viral vectors (Chtarto et al., Methods & Clinical Development (2016) 5, 16027; doi:10.1038/mtm.2016.27, SEQ ID NO: 343).

In some embodiments, a viral vector of the disclosure incorporates a promoter sequence. In a particular example, the promoter is a promoter selected from Table 8 having a nucleic acid sequence of SEQ ID NOs: 16, 19, 20, or 21 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 16, 19, 20, or 21.

In certain embodiments, a viral vector of the disclosure incorporates a CNS-specific promoter sequence. In a particular example, the CNS-specific promoter is a promoter selected from Table 8 having a nucleic acid sequence of SEQ ID NOs: 26, 27, 28, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, or 71 or a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 26, 27, 28, 39, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 71, or 71. In another particular example, the CNS-specific promoter is a promoter selected from Table 8 comprising a nucleic acid sequence of SEQ ID NOs: 31, 32, 33, 34, 35, or 36 or comprising a variant thereof having at least 70% (e.g., at least 70%, 75%, 80%, 85%, 90%, 91%, 92%, 93%, 94%, 95%, 96%, 97%, 98%, 99% or more) sequence identity to the nucleic acid sequence of SEQ ID NOs: 31, 32, 33, 34, 35, or 36.

### 5.2.6. Signal Peptides

In certain embodiments, the vectors provided herein comprise components encoding signal peptides that modulate protein delivery. In certain embodiments, the viral vectors provided herein comprise nucleotides sequences encoding one or more signal peptides linked to the heavy and/or light chains being expressed. When the heavy and light chains are expressed as two polypeptides, each should have a signal peptide fused to its N terminus whereas if the anti-Tau binding protein is expressed as one polypeptide (for example as an scFv or scFv-Fc), then the signal peptide should be fused to the N terminus of that anti-Tau binding protein. Signal peptides may also be referred to herein as “leader sequences” or “leader peptides”. In certain embodiments, the signal peptides allow for the transgene product to achieve the proper packaging (e.g., glycosylation) in the cell. In certain embodiments, the signal peptides allow for the transgene product to achieve the proper localization in the cell. In certain embodiments, the signal peptides allow for the transgene product to achieve secretion from the cell.

There are two general approaches to select a signal sequence for protein production in a gene therapy context or in cell culture. One approach is to use a signal peptide from proteins homologous to the protein being expressed. For example, a human antibody signal peptide may be used to express IgGs in CHO or other cells. Another approach is to identify signal peptides optimized for the particular host cells used for expression. Signal peptides may be interchanged between different proteins or even between proteins of different organisms, but usually the signal sequences of the most abundant secreted proteins of that cell type are used for protein expression. For example, the signal peptide of human albumin, the most abundant protein in plasma, was found to substantially increase protein production yield in CHO cells. However, certain signal peptides may retain function and exert activity after being cleaved from the expressed protein as “post-targeting functions”. Thus, in certain embodiments, the signal peptide is selected from signal peptides of the most abundant proteins secreted by the cells used for expression to avoid the post-targeting functions. In a certain embodiment, the signal sequence is fused to both the heavy and light chain sequences. An exemplary sequence is MYRMQLLLLIALSLALVTNS (SEQ ID NO: 87) (see FIGS. 1D-F and Table 9). Alternatively, signal sequences that are appropriate for expression of the mAb or Fab in CNS tissue are provided in Table 9, respectively, below.

TABLE 9
Signal peptides suitable for expression in CNS tissue

| Signal Peptide | SEQ ID | |
|---|---|---|
| Origin | NO: | Sequence |
| Mutant IL2 signal | 87 | MYRMQLLLLIALSLALVINS |
| peptide | | |
| VEGF-A signal | 88 | MNFLLSWVHWSLALLLYLHHAKWSQA |
| peptide | | |
| Fibulin-1 signal | 89 | MERAAPSRRVPLPLLLLGGLALLAAGVDA |
| peptide | | |
| Vitronectin signal | 90 | MAPLRPLLILALLAWVALA |
| peptide | | |
| Complement Factor | 91 | MRLLAKIICLMLWAICVA |
| H signal peptide | | |
| Opticin signal | 92 | MRLLAFLSLLALVLQETGT |
| peptide | | |
| Albumin signal | 93 | MKWVTFISLLFLESSAYS |
| peptide | | |
| Chymotrypsinogen | 94 | MAFLWLLSCWALLGTTFG |
| signal peptide | | |
| Interleukin-2 signal | 95 | MYRMQLLSCIALILALVINS |
| peptide | | |
| Trypsinogen-2 | 96 | MNLLLILTFVAAAVA |
| signal peptide | | |

### 5.3. CNS Delivery

The antibody gene therapies stem from the surprisingly discovered several antibodies with unique binding specificities, e.g. binding Tau in the brain tissue of patients with Alzheimer's Disease (AD), Progressive supranuclear palsy (PSP) as well as Prick's Disease (PiD) as evidenced by capturing Tau and AD-associated Tau in an immunoprecipitation assay with brain extracts of patients with AD. The antibody gene therapies described herein are suitable for administration to the CNS, or are administered systemically with ensuing blood-brain-barrier crossing of the rAAV encapsidating the antibody gene, or crossing of the therapeutic antibody expressed by rAAV transduced cells outside the CNS.

Accordingly, provided are methods of administration to the CNS, e.g. intracerebroventricular (ICV), intracisternal (IC), including direct injection into the cisterna magna, lumbar intrathecal (IT) or intraparenchymal administration, and following transduction, the vector's production of the protein product is enhanced by an expression cassette employing engineered CNS-specific nucleic acid regulatory elements. In other examples, the rAAV vector may cross the blood brain barrier if provided systemically by intravenous, intramuscular, and/or intra-peritoneal administration. In other embodiments, the rAAV is administered such that it is delivered to the liver where it transduces liver cells generating a depot of cells producing the anti-Tau binding mAbs and secreting them into the circulation where they are then delivered to the CNS.

To further enhance transduction of target cells to express the antibody, particularly brain cells, the rAAV genome cassette comprises CNS-specific, such as neuron-specific promoters and/or enhancers. Exemplary CNS-specific promoters are provided in Table 8

### 5.4. Cellular Expression of Transgene

Another aspect of the present invention relates to nucleic acid expression cassettes comprising cell-specific promoters, e.g. CNS cell-specific promoters. Combinations of promoter and enhancer sequences may improve transgene expression while maintaining cellular and tissue specificity. Because the nucleic acid sequences encoding Tau-specific mAbs, and regulatory and structural elements, will be provided and delivered to cells as an expression cassette, the target cells become a permanent depot that continuously expresses, or supplies, the human mAb, e.g., human-glycosylated, transgene product. Thus, upon viral vector transduction, the Tau-specific mAb becomes a product of the target cells within the target tissue. Cells of the CNS, such as neurons and astrocytes (glial cells), are also secretory cells capable of expressing a heterologous gene and secreting into the extracellular space (Merienne, N. et al, 2013, Front Cell Neurosci, 7:106; Drinkut A, et al, 2012, Mol Ther. 20:534-43; Griffin, J M, et al. 2019, Gene Therapy, 26:198-210).

The molecular format of the antibody gene, engineering the antibody structure and that of the expression cassette elements, determines the mechanistic nature of the gene and its interaction in and outside of the cell. It has been shown that different forms of antibody, either whole antibodies or single chain variable fragments (scFv), scFv-IgG (also known as scFv-Fc) or other, can be successfully delivered to the CNS for various indications (Elmer, M., et al, Dec. 30, 2019, PLoS ONE 14(12): e0226245; Hay, C. E. et al, 2018 PLoS ONE 13(6): e0200060; Hay, C. E. et al, 2020 J Pharmacol Exp Ther 374(1):16-23).

Tauopathies usually come along with hyperphosphorylated tau as intracellular neurofibrillary tangles. Thus, in one embodiment, it may be beneficial to use recombinant Fab (rFab) and scFvs of the anti-tau antibody, which might more readily penetrate a cell membrane. For example, Krishnaswamy et al., J Neurosci. 34 (2014), 16835-16850 describe the use of smaller antibody fragments that bind to tau as being attractive as ligands for in vivo imaging and showed that peripheral injection of scFvs resulted in a strong in vivo brain signal in transgenic tauopathy mice, but not in wild-type or amyloid-β plaque mice. Furthermore, Nisbet et al. Brain 140 (2017), 1220-1230 demonstrated that intravenous administration of anti-tau scFvs to transgenic mice reduced anxiety-like behavior and tau hyperphosphorylation.

Intrabodies (iAbs) are recombinant antibodies engineered to be intracellularly expressed and also provide a therapeutic tool for targeting intracellular proteins (Southwell, et al. 2009, The Journal of Neuroscience, 29(43):13589-13602; Khoshnan, et al. 2002, Proc Natl Acad Sci USA 99:1002-1007). IAbs may also target proteins to particular cellular compartments using localization sequences (Marschall and Dubel, 2016, Comput Struct Biotechnol J. 14:304-308; Lecerf J M, et al. 2001, Proc Natl Acad Sci USA 98:4764-4769).

Immunotherapy approaches using different antibody formats such as scFv, single-domain antibody fragments (VHHs or sdAbs), bispecific antibodies, intrabodies and nanobodies have shown therapeutic efficacy in several animal models of Alzheimer's disease (AD), Parkinson disease (PD), dementia with Lewy bodies (DLB), frontotemporal dementia (FTD), Huntington disease (HD), transmissible spongiform encephalopathies (TSEs) and multiple sclerosis (MS). It has been demonstrated that recombinant antibody fragments may neutralize toxic extra- and intracellular misfolded proteins involved in the pathogenesis neurodegenerative diseases and thus represent a promising tool for the development of antibody-based immunotherapeutics for those diseases; see review of Manoutcharian et al., Curr Neuropharmacol 15 (2017), 779-788.

The perceived advantages of using small Fab and scFv engineered antibody formats which lack the effector function include more efficient cell membrane penetration and minimizing the risk of triggering inflammatory side reactions. Furthermore, besides scFv and single-domain antibodies retain the binding specificity of full-length antibodies, they can be expressed as single genes and intracellularly in mammalian cells as intrabodies, with the potential for alteration of the folding, interactions, modifications, or subcellular localization of their targets; see for review, e.g., Miller and Messer, Molecular Therapy 12 (2005), 394-401.

### 5.5. Transgene Post-Translational Modifications

Another aspect of the present invention relates to Tau-specific antibodies having human cell-specific post-translational modifications, whereas the presence of an post-translational modifications of the expressed protein may alter several properties of the therapeutic protein, including increase expressionist pharmacokinetics in vivo. The amino acid sequence (primary sequence) of Tau-specific mAbs disclosed herein each may comprise at least one site at which N-glycosylation or tyrosine sulfation takes place for glycosylation and/or sulfation positions within the amino acid sequences of the variable regions as well as constant regions of the therapeutic antibodies. Post-translational modification is known to occur in the Fc domain of full-length antibodies, particularly at residue N297 (by EU numbering).

Alternatively, mutations may be introduced into the Fc domain to alter the glycosylation site at residue N297 (EU numbering), in particular substituting another amino acid for the asparagine at 297 or the threonine at 299 to remove the glycosylation site resulting in an aglycosylated Fc domain.

### 5.5.1. N-Glycosylation

### Reverse Glycosylation Sites

The canonical N-glycosylation sequence is known in the art to be Asn-X-Ser(or Thr), wherein X can be any amino acid except Pro. However, it recently has been demonstrated that asparagine (Asn) residues of human antibodies can be glycosylated in the context of a reverse consensus motif, Ser(or Thr)-X-Asn, wherein X can be any amino acid except Pro. See Valliere-Douglass et al., 2009, J. Biol. Chem. 284:32493-32506; and Valliere-Douglass et al., 2010, J. Biol. Chem. 285:16012-16022. As disclosed herein, certain Tau-specific mAbs disclosed herein comprise such reverse consensus sequences.

### Non-Consensus Glycosylation Sites

In addition to reverse N-glycosylation sites, it recently has been demonstrated that glutamine (Gln) residues of human antibodies can be glycosylated in the context of a non-consensus motif, Gln-Gly-Thr. See Valliere-Douglass et al., 2010, J. Biol. Chem. 285:16012-16022. Certain Tau-specific mAbs disclosed herein may comprise such non-consensus sequences. In addition, O-glycosylation comprises the addition of N-acetyl-galactosamine to serine or threonine residues by the enzyme. It has been demonstrated that amino acid residues present in the hinge region of antibodies can be O-glycosylated. The possibility of O-glycosylation confers another advantage to the therapeutic antibodies provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J. Bacteriol. 189:8088-8098.)

### Engineered N-Glycosylation Sites

In certain embodiments, a nucleic acid encoding a Tau-specific mAb is modified to include 1, 2, 3, 4, 5, 6, 7, 8, 9, 10, or more N-glycosylation sites (including the canonical N-glycosylation consensus sequence, reverse N-glycosylation site, and non-consensus N-glycosylation sites) than would normally be associated with the Tau-specific mAb (e.g., relative to the number of N-glycosylation sites associated with the Tau-specific mAb in its unmodified state). In certain embodiments, introduction of glycosylation sites is accomplished by insertion of N-glycosylation sites (including the canonical N-glycosylation consensus sequence, reverse N-glycosylation site, and non-consensus N-glycosylation sites) anywhere in the primary structure of the antigen-binding fragment, so long as said introduction does not impact binding of the antibody or antigen-binding fragment to its antigen. Introduction of glycosylation sites can be accomplished by, e.g., adding new amino acids to the primary structure of the antigen-binding fragment, or the antibody from which the antigen-binding fragment is derived (e.g., the glycosylation sites are added, in full or in part), or by mutating existing amino acids in the antigen-binding fragment, or the antibody from which the antigen-binding fragment is derived, in order to generate the N-glycosylation sites (e.g., amino acids are not added to the antigen-binding fragment/antibody, but selected amino acids of the antigen-binding fragment/antibody are mutated so as to form N-glycosylation sites). Those of skill in the art will recognize that the amino acid sequence of a protein can be readily modified using approaches known in the art, e.g., recombinant approaches that include modification of the nucleic acid sequence encoding the protein.

In a specific embodiment, a Tau-specific mAb or antigen-binding fragment is modified such that, when expressed in mammalian cells, such as CNS cells, it can be hyperglycosylated. See Courtois et al., 2016, mAbs 8:99-112 which is incorporated by reference herein in its entirety.

### 5.5.2 N-Glycosylation of Fully Human Post-Translationally Modified mAbs and Antigen-Binding Fragments

Unlike small molecule drugs, biologics usually comprise a mixture of many variants with different modifications or forms that could have a different potency, pharmacokinetics, and/or safety profile. It is not essential that every molecule produced either in the gene therapy or protein therapy approach be fully glycosylated and sulfated. Rather, the population of glycoproteins produced should have sufficient glycosylation (including 2,6-sialylation) and sulfation to demonstrate efficacy. The goal of gene therapy treatment provided herein can be, for example, to slow or arrest the progression of a disease or abnormal condition or to reduce the severity of one or more symptoms associated with the disease or abnormal condition.

When Tau-specific mAb is expressed in a human cell, the N-glycosylation sites of the antigen-binding fragment can be glycosylated with various different glycans. N-glycans of antigen-binding fragments and the Fc domain have been characterized in the art. For example, Bondt et al., 2014, Mol. & Cell. Proteomics 13.11:3029-3039 (incorporated by reference herein in its entirety for its disclosure of Fab-associated N-glycans) characterizes glycans associated with Fabs, and demonstrates that Fab and Fc portions of antibodies comprise distinct glycosylation patterns, with Fab glycans being high in galactosylation, sialylation, and bisection (e.g., with bisecting GlcNAc) but low in fucosylation with respect to Fc glycans. Like Bondt, Huang et al., 2006, Anal. Biochem. 349:197-207 (incorporated by reference herein in its entirety for its disclosure of Fab-associated N-glycans) found that most glycans of Fabs are sialylated. However, in the Fab of the antibody examined by Huang (which was produced in a murine cell background), the identified sialic residues were N-Glycolylneuraminic acid (“Neu5Gc” or “NeuGc”) (which is not natural to humans) instead of N-acetylneuraminic acid (“Neu5Ac,” the predominant human sialic acid). In addition, Song et al., 2014, Anal. Chem. 86:5661-5666 (incorporated by reference herein in its entirety for its disclosure of Fab-associated N-glycans) describes a library of N-glycans associated with commercially available antibodies.

Glycosylation of the Fc domain has been characterized and is a single N-linked glycan at asparagine 297 (EU numbering). The glycan plays an integral structural and functional role, impacting antibody effector function, such as binding to Fc receptor (see, for example, Jennewein and Alter, 2017, Trends In Immunology 38:358 for a discussion of the role of Fc glycosylation in antibody function). Removal of the Fc region glycan almost completely ablates effector function (Jennewien and Alter at 362). The composition of the Fc glycan has been shown to impact effector function, for example hypergalactosylation and reduction in fucosylation have been shown to increase ADCC activity while sialylation correlates with anti-inflammatory effects (Id. at 364). Disease states, genetics and even diet can impact the composition of the Fc glycan in vivo. For recombinantly expressed antibodies, the glycan composition can differ significantly by the type of host cell used for recombinant expression and strategies are available to control and modify the composition of the glycan in therapeutic antibodies recombinantly expressed in cell culture, such as CHO, to alter effector function (see, for example, US 2014/0193404 by Hansen et al.). Accordingly, the Tau-specific mAbs provided herein may advantageously have a glycan at N297 that is more like the native, human glycan composition than antibodies expressed in non-human host cells.

Importantly, when the Tau-specific mAbs are expressed in human cells, the need for in vitro production in prokaryotic host cells (e.g., E. coli) or eukaryotic host cells (e.g., CHO cells or NSO cells) is circumvented. Instead, as a result of the methods described herein, N-glycosylation sites of the Tau-specific mAbs are advantageously decorated with glycans relevant to and beneficial to treatment of humans. Such an advantage is unattainable when CHO cells, NSO cells, or E. coli are utilized in antibody/antigen-binding fragment production, because e.g., CHO cells (1) do not express 2,6 sialyltransferase and thus cannot add 2,6 sialic acid during N-glycosylation; (2) can add Neu5Gc as sialic acid instead of Neu5Ac; and (3) can also produce an immunogenic glycan, the α-Gal antigen, which reacts with anti-α-Gal antibodies present in most individuals, which at high concentrations can trigger anaphylaxis; and because (4) E. coli does not naturally contain components needed for N-glycosylation.

Assays for determining the glycosylation pattern of antibodies, including antigen-binding fragments are known in the art. For example, hydrazinolysis can be used to analyze glycans. First, polysaccharides are released from their associated protein by incubation with hydrazine (the Ludger Liberate Hydrazinolysis Glycan Release Kit, Oxfordshire, UK can be used). The nucleophile hydrazine attacks the glycosidic bond between the polysaccharide and the carrier protein and allows release of the attached glycans. N-acetyl groups are lost during this treatment and have to be reconstituted by re-N-acetylation. Glycans may also be released using enzymes such as glycosidases or endoglycosidases, such as PNGase F and Endo H, which cleave cleanly and with fewer side reactions than hydrazines. The free glycans can be purified on carbon columns and subsequently labeled at the reducing end with the fluorophor 2-amino benzamide. The labeled polysaccharides can be separated on a GlycoSep-N column (GL Sciences) according to the HPLC protocol of Royle et al, Anal Biochem 2002, 304(1):70-90. The resulting fluorescence chromatogram indicates the polysaccharide length and number of repeating units. Structural information can be gathered by collecting individual peaks and subsequently performing MS/MS analysis. Thereby the monosaccharide composition and sequence of the repeating unit can be confirmed and additionally in homogeneity of the polysaccharide composition can be identified. Specific peaks of low or high molecular weight can be analyzed by MALDI-MS/MS and the result used to confirm the glycan sequence. Each peak in the chromatogram corresponds to a polymer, e.g., glycan, consisting of a certain number of repeat units and fragments, e.g., sugar residues, thereof. The chromatogram thus allows measurement of the polymer, e.g., glycan, length distribution. The elution time is an indication for polymer length, while fluorescence intensity correlates with molar abundance for the respective polymer, e.g., glycan. Other methods for assessing glycans associated with antigen-binding fragments include those described by Bondt et al., 2014, Mol. & Cell. Proteomics 13.11:3029-3039, Huang et al., 2006, Anal. Biochem. 349:197-207, and/or Song et al., 2014, Anal. Chem. 86:5661-5666.

Homogeneity or heterogeneity of the glycan patterns associated with antibodies (including antigen-binding fragments), as it relates to both glycan length or size and numbers glycans present across glycosylation sites, can be assessed using methods known in the art, e.g., methods that measure glycan length or size and hydrodynamic radius. HPLC, such as size exclusion, normal phase, reversed phase, and anion exchange HPLC, as well as capillary electrophoresis, allows the measurement of the hydrodynamic radius. Higher numbers of glycosylation sites in a protein lead to higher variation in hydrodynamic radius compared to a carrier with less glycosylation sites. However, when single glycan chains are analyzed, they may be more homogenous due to the more controlled length. Glycan length can be measured by hydrazinolysis, SDS PAGE, and capillary gel electrophoresis. In addition, homogeneity can also mean that certain glycosylation site usage patterns change to a broader/narrower range. These factors can be measured by Glycopeptide LC-MS/MS.

In certain embodiments, the human post-translationally modified mAbs, or antigen binding fragments thereof, also do not contain detectable NeuGc and/or α-Gal. By “detectable NeuGc” or “detectable α-Gal” or “does not contain or does not have NeuGc or α-Gal” means herein that the human post-translationally modified mAb or antigen-binding fragment, does not contain NeuGc or α-Gal moieties detectable by standard assay methods known in the art. For example, NeuGc may be detected by HPLC according to Hara et al., 1989, “Highly Sensitive Determination of N-Acetyl-and N-Glycolylneuraminic Acids in Human Serum and Urine and Rat Serum by Reversed-Phase Liquid Chromatography with Fluorescence Detection.” J. Chromatogr., B: Biomed. 377, 111-119, which is hereby incorporated by reference for the method of detecting NeuGc. Alternatively, NeuGc may be detected by mass spectrometry. The α-Gal may be detected using an ELISA, see, for example, Galili et al., 1998, “A sensitive assay for measuring α-Gal epitope expression on cells by a monoclonal anti-Gal antibody.” Transplantation. 65(8):1129-32, or by mass spectrometry, see, for example, Ayoub et al., 2013, “Correct primary structure assessment and extensive glyco-profiling of cetuximab by a combination of intact, middle-up, middle-down and bottom-up ESI and MALDI mass spectrometry techniques.” Landes Bioscience. 5(5):699-710. See also the references cited in Platts-Mills et al., 2015, “Anaphylaxis to the Carbohydrate Side-Chain Alpha-gal” Immunol Allergy Clin North Am. 35(2): 247-260.

### Benefits of N-Glycosylation

N-glycosylation confers numerous benefits on the Tau-specific mAbs described herein. Such benefits are unattainable by production of antigen-binding fragments in E. coli, because E. coli does not naturally possess components needed for N-glycosylation. Further, some benefits are unattainable through antibody production in, e.g., CHO cells (or murine cells such as NSO cells), because CHO cells lack components needed for addition of certain glycans (e.g., 2,6 sialic acid and bisecting GlcNAc) and because either CHO or murine cell lines add N-N-Glycolylneuraminic acid (“Neu5Gc” or “NeuGc”) which is not natural to humans (and potentially immunogenic), instead of N-Acetylneuraminic acid (“Neu5Ac”) the predominant human sialic acid. See, e.g., Dumont et al., 2015, Crit. Rev. Biotechnol. 36(6):1110-1122; Huang et al., 2006, Anal. Biochem. 349:197-207 (NeuGc is the predominant sialic acid in murine cell lines such as SP2/0 and NSO); and Song et al., 2014, Anal. Chem. 86:5661-5666, each of which is incorporated by reference herein in its entirety). Moreover, CHO cells can also produce an immunogenic glycan, the α-Gal antigen, which reacts with anti-α-Gal antibodies present in most individuals, which at high concentrations can trigger anaphylaxis. See, e.g., Bosques, 2010, Nat. Biotech. 28:1153-1156. The human glycosylation pattern of the Tau-specific mAbs described herein should reduce immunogenicity of the transgene product and improve efficacy.

While non-canonical glycosylation sites usually result in low level glycosylation (e.g., 1-5%) of the antibody population, the functional benefits may be significant (See, e.g., van de Bovenkamp et al., 2016, J. Immunol. 196:1435-1441). For example, Fab glycosylation may affect the stability, half-life, and binding characteristics of an antibody. To determine the effects of Fab glycosylation on the affinity of the antibody for its target, any technique known to one of skill in the art may be used, for example, enzyme linked immunosorbent assay (ELISA), or surface plasmon resonance (SPR). To determine the effects of Fab glycosylation on the half-life of the antibody, any technique known to one of skill in the art may be used, for example, by measurement of the levels of radioactivity in the blood or organs in a subject to whom a radiolabeled antibody has been administered. To determine the effects of Fab glycosylation on the stability, for example, levels of aggregation or protein unfolding, of the antibody, any technique known to one of skill in the art may be used, for example, differential scanning calorimetry (DSC), high performance liquid chromatography (HPLC), e.g., size exclusion high performance liquid chromatography (SEC-HPLC), capillary electrophoresis, mass spectrometry, or turbidity measurement.

The presence of sialic acid on Tau-specific mAbs used in the methods described herein can impact clearance rate of Tau-specific mAbs. Accordingly, sialic acid patterns of a Tau-specific mAb can be used to generate a therapeutic having an optimized clearance rate. Methods of assessing antigen-binding fragment clearance rate are known in the art. See, e.g., Huang et al., 2006, Anal. Biochem. 349:197-207.

In another specific embodiment, a benefit conferred by N-glycosylation is reduced aggregation. Occupied N-glycosylation sites can mask aggregation prone amino acid residues, resulting in decreased aggregation. Such N-glycosylation sites can be native to an antigen-binding fragment used herein or engineered into an antigen-binding fragment used herein, resulting in Tau-specific mAb that is less prone to aggregation when expressed, e.g., expressed in human cells. Methods of assessing aggregation of antibodies are known in the art. See, e.g., Courtois et al., 2016, mAbs 8:99-112 which is incorporated by reference herein in its entirety.

In another specific embodiment, a benefit conferred by N-glycosylation is reduced immunogenicity. Such N-glycosylation sites can be native to an antigen-binding fragment used herein or engineered into an antigen-binding fragment used herein, resulting in Tau-specific mAb that is less prone to immunogenicity when expressed, e.g., expressed in human retinal cells, human CNS cells, human liver cells or human muscle cells.

In another specific embodiment, a benefit conferred by N-glycosylation is protein stability. N-glycosylation of proteins is well-known to confer stability on them, and methods of assessing protein stability resulting from N-glycosylation are known in the art. See, e.g., Sola and Griebenow, 2009, J Pharm Sci., 98(4): 1223-1245.

In another specific embodiment, a benefit conferred by N-glycosylation is altered binding affinity. It is known in the art that the presence of N-glycosylation sites in the variable domains of an antibody can increase the affinity of the antibody for its antigen. See, e.g., Bovenkamp et al., 2016, J. Immunol. 196:1435-1441. Assays for measuring antibody binding affinity are known in the art. See, e.g., Wright et al., 1991, EMBO J. 10:2717-2723; and Leibiger et al., 1999, Biochem. J. 338:529-538.

### 5.5.3 Tyrosine Sulfation

Tyrosine sulfation occurs at tyrosine (Y) residues with glutamate (E) or aspartate (D) within +5 to −5 position of Y, and where position −1 of Y is a neutral or acidic charged amino acid, but not a basic amino acid, e.g., arginine (R), lysine (K), or histidine (H) that abolishes sulfation. Surprisingly, the Tau-specific mAbs described herein may comprise tyrosine sulfation sites.

Importantly, tyrosine-sulfated antigen-binding fragments cannot be produced in E. coli, which naturally does not possess the enzymes required for tyrosine-sulfation. Further, CHO cells are deficient for tyrosine sulfation-they are not secretory cells and have a limited capacity for post-translational tyrosine-sulfation. See, e.g., Mikkelsen & Ezban, 1991, Biochemistry 30: 1533-1537. Advantageously, the methods provided herein call for expression of Tau-specific mAbs in human cells that are secretory and have capacity for tyrosine sulfation.

Tyrosine sulfation is advantageous for several reasons. For example, tyrosine-sulfation of the antigen-binding fragment of therapeutic antibodies against targets has been shown to dramatically increase avidity for antigen and activity. See, e.g., Loos et al., 2015, PNAS 112: 12675-12680, and Choe et al., 2003, Cell 114: 161-170. Assays for detection tyrosine sulfation are known in the art. See, e.g., Yang et al., 2015, Molecules 20:2138-2164.

### 5.5.4 O-Glycosylation

O-glycosylation comprises the addition of N-acetyl-galactosamine to serine or threonine residues by the enzyme. It has been demonstrated that amino acid residues present in the hinge region of antibodies can be O-glycosylated. In certain embodiments, the Tau-specific mAbs comprise all or a portion of their hinge region, and thus are capable of being O-glycosylated when expressed in human cells. The possibility of O-glycosylation confers another advantage to the Tau-specific mAbs provided herein, as compared to, e.g., antigen-binding fragments produced in E. coli, again because the E. coli naturally does not contain machinery equivalent to that used in human O-glycosylation. (Instead, O-glycosylation in E. coli has been demonstrated only when the bacteria is modified to contain specific O-glycosylation machinery. See, e.g., Farid-Moayer et al., 2007, J Bacteriol. 189:8088-8098.) O-glycosylated HuGlyFab, by virtue of possessing glycans, shares advantageous characteristics with N-glycosylated Tau-specific mAbs (as discussed above).

Other modifications may be employed to alter the transgene sequence and subsequently the expressed protein and its ability to be glycosylated or sulfated, and the like, and are well known to the skilled person.

### 5.5.5. Human Post Translationally Modified Antibodies

The production of the anti-Tau human post-translationally modified mAb or human post-translationally modified Fab, should result in a “biobetter” molecule for the treatment accomplished via gene therapy—e.g., by administering a viral vector or other DNA expression construct encoding the anti-Tau human post-translationally modified Fab or human post-translationally modified mAb, intrathecally, particularly intracisternal or lumbar administration, or intravenous administration to human subjects (patients) diagnosed with or having one or more symptoms of AD, PSP, and/or PiD, to create a permanent depot in the CNS that continuously supplies the fully-human post-translationally modified, e.g., human-glycosylated, sulfated transgene product produced by transduced CNS cells.

The cDNA construct for the anti-Tau human post-translationally modified mAb or anti-Tau human post-translationally modified Fab should include a signal peptide that ensures proper co- and post-translational processing (glycosylation and protein sulfation) by the transduced CNS cells. For example, the signal sequence may be MYRMQLLLLIALSLALVTNS (SEQ ID NO: 87).

As an alternative, or an additional treatment to gene therapy, the anti-Tau human post-translationally modified mAb or human post-translationally modified Fab can be produced in human cell lines by recombinant DNA technology, and administered to patients diagnosed with Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia and ischemic stroke, or for whom therapy for Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia and ischemic stroke is considered appropriate.

In some embodiments, the anti-Tau human post-translationally modified mAb or antigen-binding fragment thereof has heavy and light chains with the amino acid sequences of the heavy and light chain Fab portions of NI-502.4P3 as set forth in FIG. 1D (with non-consensus asparagine (N) glycosylation sites highlighted in grey, glutamine (Q) glycosylation sites highlighted in bold, and Y-sulfation sites highlighted in grey and italic) has glycosylation, particularly a 2,6-sialylation, at one or more of the amino acid positions Q114 and/or N164 of the heavy chain (SEQ ID NO: 187) or N22, N35, N164 and/or N216 of the light chain (SEQ ID NO: 188). Alternatively or in addition to, the human post-translationally modified mAb or antigen binding-fragment thereof with the heavy and light chain variable domain sequences of NI-502.4P3 has a sulfation group at Y94 and/or Y95 of the heavy chain (SEQ ID NO: 187) and/or Y198 of the light chain (SEQ ID NO: 188).

In some embodiments, the anti-Tau human post-translationally modified mAb or antigen-binding fragment thereof has heavy and light chains with the amino acid sequences of the heavy and light chain Fab portions of NI-502.31B6 as set forth in FIG. 1E (with non-consensus asparagine (N) glycosylation sites highlighted in grey, glutamine (Q) glycosylation sites highlighted in bold, and Y-sulfation sites highlighted in grey and italic) has glycosylation, particularly a 2,6-sialylation, at one or more of the amino acid positions N54, Q118 and/or N168 of the heavy chain (SEQ ID NO: 189) or N174 of the light chain (SEQ ID NO: 190). Alternatively or in addition to, the human post-translationally modified mAb or antigen binding-fragment thereof with the heavy and light chain variable domain sequences of NI-502.31B6 has a sulfation group at Y94 and/or Y95 of the heavy chain (SEQ ID NO: 189) and/or Y88 and/or Y89 of the light chain (SEQ ID NO: 190).

In some embodiments, the anti-Tau human post-translationally modified mAb or antigen-binding fragment thereof has heavy and light chains with the amino acid sequences of the heavy and light chain Fab portions of NI-502.8H1 as set forth in FIG. 1F (with non-consensus asparagine (N) glycosylation sites highlighted in grey, glutamine (Q) glycosylation sites highlighted in bold, and Y-sulfation sites highlighted in grey and italic) has glycosylation, particularly a 2,6-sialylation, at one or more of the amino acid positions Q114 and/or N164 of the heavy chain (SEQ ID NO: 191) or N160 and/or N212 of the light chain (SEQ ID NO: 192). Alternatively or in addition to, the HuPTM mAb or antigen binding-fragment thereof with the heavy and light chain variable domain sequences of NI-502.8H1 has a sulfation group at Y94 and/or Y95 of the heavy chain (SEQ ID NO: 191) and/or Y87 and/or Y88 and/or Y194 of the light chain (SEQ ID NO: 192).

In other embodiments, the anti-Tau human post-translationally modified mAb or antigen-binding fragment thereof does not contain any detectable (e.g., as detected by assays known in the art, for example, those described in section 5.5.2, infra) NeuGc moieties and/or does not contain any detectable (e.g., as detected by assays known in the art, for example, those described in section 5.5.2, infra) alpha-Gal moieties. In certain embodiments, the human post-translationally modified mAb is a full length or substantially full length mAb with an Fc region.

In certain embodiments, the human post-translationally modified mAb or Fab is therapeutically effective and is at least 0.5%, 1% or 2% 2,6 sialylated and/or sulfated and may be at least 5%, 10% or even 50% or 100% glycosylated 2,6 sialylation and/or sulfated. The goal of gene therapy treatment provided herein is to slow or arrest the progression of Alzheimer's disease, amyotrophic lateral sclerosis/parkinsonism-dementia complex, argyrophilic grain dementia, British type amyloid angiopathy, cerebral amyloid angiopathy, corticobasal degeneration, Creutzfeldt-Jakob disease, dementia pugilistica, diffuse neurofibrillary tangles with calcification, Down's syndrome, frontotemporal dementia, frontotemporal dementia with parkinsonism linked to chromosome 17, frontotemporal lobar degeneration, Gerstmann-Straussler-Scheinker disease, Hallervorden-Spatz disease, inclusion body myositis, multiple system atrophy, myotonic dystrophy, Niemann-Pick disease type C, non-Guamanian motor neuron disease with neurofibrillary tangles, Pick's disease, postencephalitic parkinsonism, prion protein cerebral amyloid angiopathy, progressive subcortical gliosis, progressive supranuclear palsy, subacute sclerosing panencephalitis, Tangle only dementia, multi-infarct dementia and ischemic stroke, particularly cognitive impairment, gross or fine motor skill impairment, or vision impairment. Efficacy may be monitored by measuring a reduction in plaque formation and/or an improvement in cognitive function, with motor skills, or with vision or a reduction in the decline in cognitive function, motor skills, or vision.

### 5.6. Vector Formats

### 5.6.1. ssAAV

In one embodiment, an expression cassette for use in an AAV vector is provided. In certain embodiments, a single-stranded AAV (ssAAV) may be used. The AAV genome is packaged as a linear ssDNA molecule with the palindromic inverted terminal repeat (ITR) sequences which form dsDNA hairpin structures at each end. These serve as replication origins during productive infection and as priming sites for host-cell DNA polymerase to begin synthesis of a complementary strand.

In that embodiment, the AAV expression cassette includes at least one AAV inverted terminal repeat (ITR) sequence. In another embodiment, the expression cassette comprises 5′ ITR sequences and 3′ ITR sequences. In one embodiment, the 5′ and 3′ ITRs flank the codon optimized nucleic acid sequence that encodes the transgene. Thus, as described herein, an AAV expression cassette is meant to describe an expression cassette as described above flanked on its 5′ end by a 5′AAV inverted terminal repeat sequence (ITR) and on its 3′ end by a 3′ AAV ITR. Thus, this rAAV genome contains the minimal sequences required to package the expression cassette into an AAV viral particle, i.e., the AAV 5′ and 3′ ITRs. The AAV ITRs may be obtained from the ITR sequences of any AAV, such as described herein. These ITRs may be of the same AAV origin as the capsid employed in the resulting recombinant AAV, or of a different AAV origin (to produce an AAV pseudotype). In one embodiment, the ITR sequences from AAV2, or the deleted version thereof (ΔITR), are used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, the AAV vector genome comprises an AAV 5′ ITR, the TPP1 coding sequences and any regulatory sequences, and an AAV 3′ ITR. However, other configurations of these elements may be suitable. A shortened version of the 5′ ITR, termed ΔITR, has been described in which the D-sequence and terminal resolution site (trs) are deleted. In other embodiments, the full-length AAV 5′ and 3′ ITRs are used. Each rAAV genome can be then introduced into a production plasmid.

### 5.6.2. ScAAV

In certain embodiments, a self-complementary vector, e.g., scAAV, may be used (see, e.g., Wu, 2007, Human Gene Therapy, 18(2):171-82, McCarty et al, 2001, Gene Therapy, Vol 8, Number 16, Pages 1248-1254; and U.S. Pat. Nos. 6,596,535; 7,125,717; and 7,456,683, each of which is incorporated herein by reference in its entirety). “Self-complementary AAV” refers a plasmid or vector having an expression cassette in which a coding region carried by a recombinant AAV nucleic acid sequence has been designed to form an intra-molecular double-stranded DNA template. Unlike ssDNA genomes, the scAAV genome is not subject to host-cell DNA polymerase and does not require synthesis of a complementary strand. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

### 5.7. Vectors for Gene Delivery

Viral vectors or other DNA expression constructs encoding a human Tau-specific mAb or antigen-binding fragment thereof, particularly a human glycosylated mAb or a hyperglycosylated derivative of a human Tau-specific mAb antigen-binding fragment are provided herein. The viral vectors and other DNA expression constructs provided herein include any suitable method for delivery of a transgene to a target cell. The means of delivery of a transgene include viral vectors, liposomes, other lipid-containing complexes, other macromolecular complexes, synthetic modified mRNA, unmodified mRNA, small molecules, non-biologically active molecules (e.g., gold particles), polymerized molecules (e.g., dendrimers), naked DNA, plasmids, phages, transposons, cosmids, or episomes. In some embodiments, the vector is a targeted vector, e.g., a vector targeted to CNS cells, or liver cells.

### 5.7.1. Viral Vectors

Viral vectors include adenovirus, adeno-associated virus (AAV, e.g., AAV8, AAV9, AAVrh10, AAV.PHP, AAV.PHP.eB), lentivirus, helper-dependent adenovirus, herpes simplex virus, poxvirus, hemagglutinin virus of Japan (HVJ), alphavirus, vaccinia virus, and retrovirus vectors. Retroviral vectors include murine leukemia virus (MLV) and human immunodeficiency virus (HIV)-based vectors. Alphavirus vectors include semliki forest virus (SFV) and sindbis virus (SIN). In certain embodiments, the viral vectors provided herein are recombinant viral vectors. In certain embodiments, the viral vectors provided herein are altered such that they are replication-deficient in humans. In certain embodiments, the viral vectors are hybrid vectors, e.g., an AAV vector placed into a “helpless” adenoviral vector. In certain embodiments, provided herein are viral vectors comprising a viral capsid from a first virus and viral envelope proteins from a second virus. In other embodiments, the second virus is vesicular stomatitus virus (VSV). In still other embodiments, the envelope protein is VSV-G protein.

In certain embodiments, the viral vectors used in the methods described herein are adenovirus based viral vectors. A recombinant adenovirus vector may be used to transfer in the transgene encoding the Tau-specific mAb or antigen-binding fragment thereof. The recombinant adenovirus can be a first-generation vector, with an E1 deletion, with or without an E3 deletion, and with the expression cassette inserted into either deleted region. The recombinant adenovirus can be a second-generation vector, which contains full or partial deletions of the E2 and E4 regions. A helper-dependent adenovirus retains only the adenovirus inverted terminal repeats and the packaging signal (phi). The transgene is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

In certain embodiments, the viral vectors used in the methods described herein are lentivirus based viral vectors. A recombinant lentivirus vector may be used to transfer in the transgene encoding the Tau-specific mAb antigen binding fragment. Four plasmids are used to make the construct: Gag/pol sequence containing plasmid, Rev sequence containing plasmids, Envelope protein containing plasmid (e.g., VSV-G), and Cis plasmid with the packaging elements and the anti-Tau antigen-binding fragment gene.

For lentiviral vector production, the four plasmids are co-transfected into cells (e.g., HEK293 based cells), whereby polyethylenimine or calcium phosphate can be used as transfection agents, among others. The lentivirus is then harvested in the supernatant (lentiviruses need to bud from the cells to be active, so no cell harvest needs/should be done). The supernatant is filtered (0.45 μm) and then magnesium chloride and benzonase added. Further downstream processes can vary widely, with using TFF and column chromatography being the most GMP compatible ones. Others use ultracentrifugation with/without column chromatography. Exemplary protocols for production of lentiviral vectors may be found in Lesch et al., 2011, “Production and purification of lentiviral vector generated in 293T suspension cells with baculoviral vectors,” Gene Therapy 18:531-538, and Ausubel et al., 2012, “Production of CGMP-Grade Lentiviral Vectors,” Bioprocess Int. 10(2):32-43, both of which are incorporated by reference herein in their entireties.

In a specific embodiment, a vector for use in the methods described herein is one that encodes a mAb or mAb antigen binding fragment, such that, upon introduction of the vector into a relevant cell, a glycosylated and/or tyrosine sulfated variant of the mAb or mAb antigen binding fragment is expressed by the cell.

Other viral vectors may be used, including but not limited to lentiviral vectors; vaccinia viral vectors, or non-viral expression vectors referred to as “naked DNA” constructs. Expression of the transgene can be controlled by constitutive or tissue-specific expression control elements.

### 5.7.2. Recombinant AAV

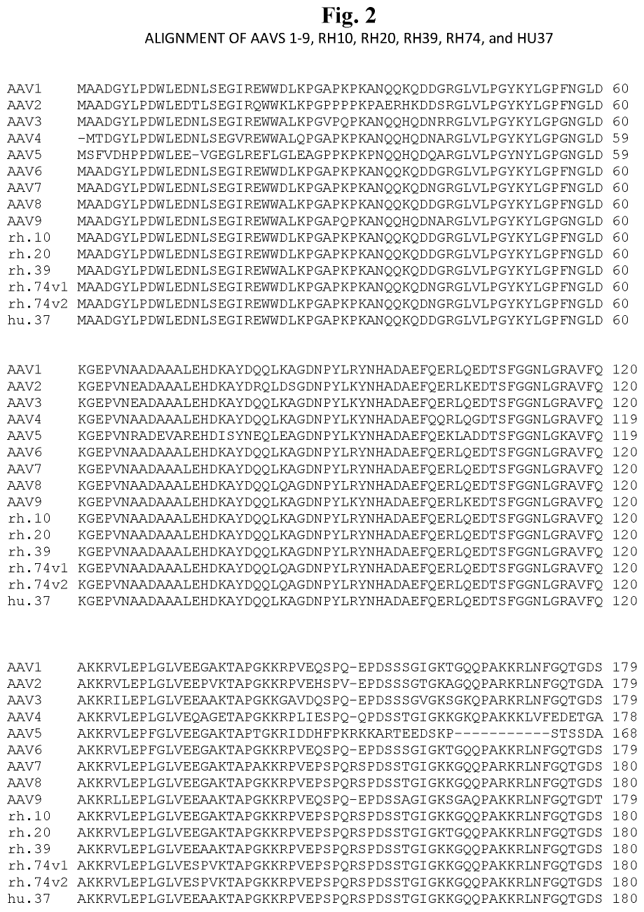

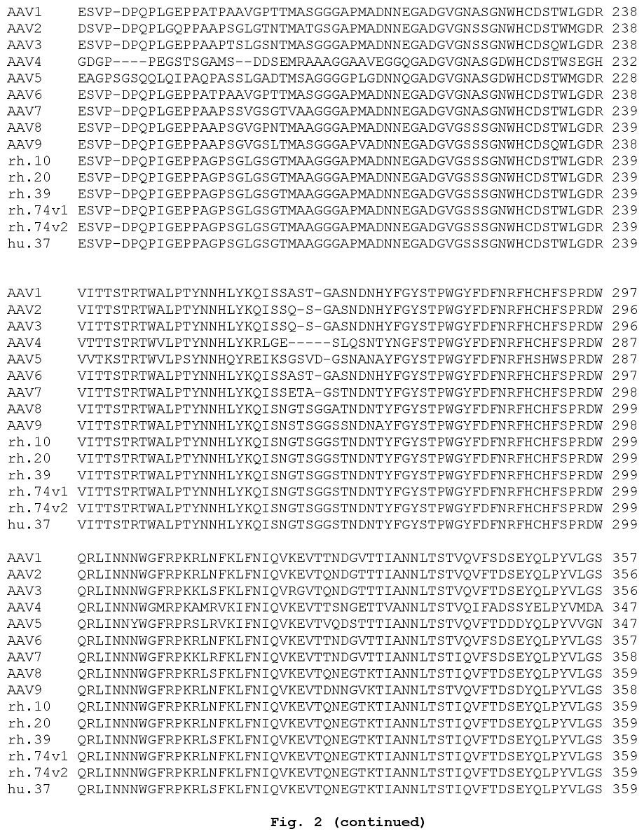

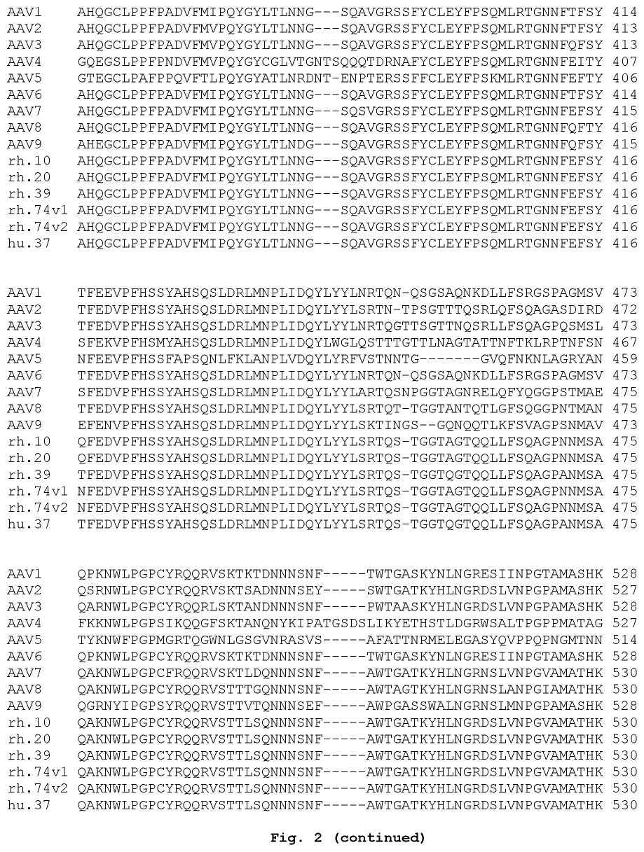

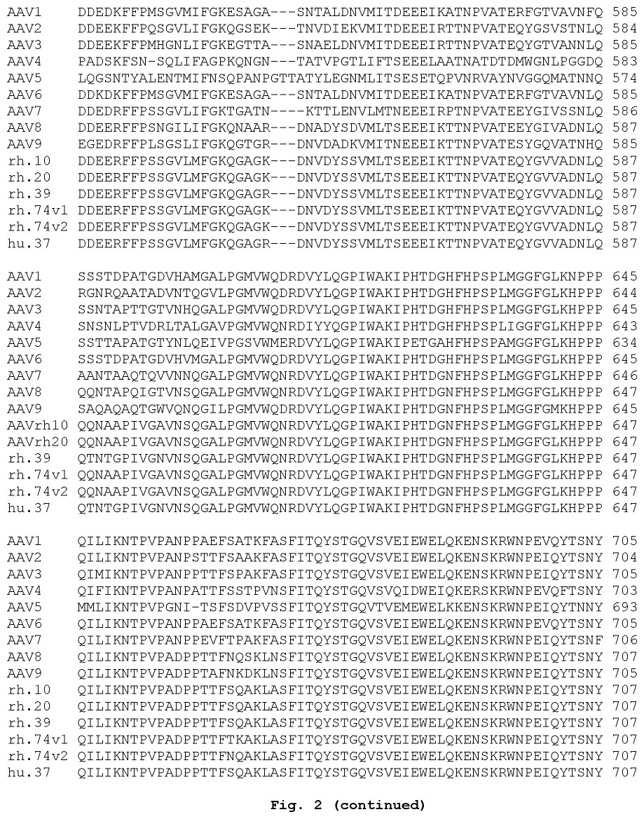

In certain embodiments, the viral vectors provided herein are AAV based viral vectors. In certain embodiments, the AAV-based vectors provided herein do not encode the AAV rep gene (required for replication) and/or the AAV cap gene (required for synthesis of the capsid proteins) (the rep and cap proteins may be provided by the packaging cells in trans). Multiple AAV serotypes have been identified. In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV.PHP.B, AAV.PHPeB, AAV10, AAV11, AAV.rh74v1, AAV.rh74v2, AAV.hu37, AAVrh10, AAVrh20, or AAVrh39. In certain embodiments, AAV based vectors provided herein comprise components from one or more of AAV8, AAV9, AAV.PHP.B, AAV.PHPeB, or AAVrh10 serotypes. Provided are viral vectors in which the capsid protein is a variant of the, AAV9 capsid protein (SEQ ID NO: 132), AAV.PHP.B (SEQ ID NO: 220), AAV.PHP.eB (SEQ ID NO: 219), or AAVrh10 capsid protein (SEQ ID NO: 133), and the capsid protein is e.g., at least 95%, 96%, 97%, 98%, 99% or 99.9% identical to the amino acid sequence of the AAV9 capsid protein (SEQ ID NO: 132), AAV.PHP.B (SEQ ID NO: 220), AAV.PHP.eB (SEQ ID NO: 219), or AAVrh10 capsid protein (SEQ ID NO: 133), while retaining the biological function of the native capsid. In certain embodiments, the encoded AAV capsid has the sequence of a wild-type capsid with 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions and retaining the biological function of the wild-type capsid, for example AAV9, AAV.PHP.eB, AAV.PHP.B, or AAVrh10 capsid. FIG. 2 provides a comparative alignment of the amino acid sequences of the capsid proteins of different AAV serotypes with potential amino acids that may be substituted at certain positions in the aligned sequences based upon the comparison in the row labeled SUBS. Accordingly, in certain embodiments, the AAV vector comprises an AAV1, AAV2, AAV3, AAV4, AAV5, AAV6, AAV7, AAV8, AAV9, AAV.PHP.B, AAV.PHPeB, AAV10, AAV11, AAV.rh74v1, AAV.rh74v2, AAV.hu37,AAVrh10, AAVrh20, or AAVrh39capsid variant that has 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acid substitutions that are not present at that position in the native AAV capsid sequence.

In certain embodiments, the AAV that is used in the compositions and methods described herein is Anc80 or Anc80L65, as described in Zinn et al., 2015, Cell Rep. 12(6): 1056-1068, which is incorporated by reference in its entirety. In certain embodiments, the AAV that is used in the methods described herein is AAV2.7m8 and/or comprises one of the following amino acid insertions: LGETTRP (or LALGETTRP), as described in U.S. Pat. Nos. 9,193,956; 9,458,517; and 9,587,282 and US patent application publication no. 2016/0376323, and International Publication WO 2018/075798, each of which is incorporated herein by reference in its entirety. In certain embodiments, the AAV that is used in the methods described herein is any AAV disclosed in U.S. Pat. No. 9,585,971, such as AAV-PHP.B (see Table 10 below). In certain embodiments, the AAV that is used in the methods described herein is any AAV disclosed in the International Patent Application Nos. PCT/US2019/032387 and PCT/US2019/055756, such as VOY101 (SEQ ID NO: 221), VOY201 (SEQ ID NO: 222), VOY701 (SEQ ID NO: 223), VOY801 (SEQ ID NO: 224), VOY1101 (SEQ ID NO: 225) (see Table 10 below).

In certain embodiments, the AAV used in the compositions and methods described herein is an AAV2/Rec2 or AAV2/Rec3 vector, which have hybrid capsid sequences derived from AAV8 capsids and capsids of serotypes cy5, rh20 or rh39 as described in Charbel Issa et al., 2013, PLoS One 8(4): e60361, which is incorporated by reference herein for these vectors. In certain embodiments, the AAV that is used in the methods described herein is an AAV disclosed in any of the following patents and patent applications, each of which is incorporated herein by reference in its entirety: U.S. Pat. Nos. 7,906,111; 8,524,446; 8,999,678; 8,628,966; 8,927,514; 8,734,809; 9,284,357; 9,409,953; 9,169,299; 9,193,956; 9,458,517; and 9,587,282 US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. AAV9-based, and AAVrh10-based viral vectors are used in certain of the methods described herein. Nucleotide sequences of AAV based viral vectors and methods of making recombinant AAV and AAV capsids are taught, for example, in U.S. Pat. No. 7,282,199 B2, U.S. Pat. No. 7,790,449 B2, U.S. Pat. No. 8,318,480 B2, U.S. Pat. No. 8,962,332 B2 and International Patent Application No. PCT/EP2014/076466, each of which is incorporated herein by reference in its entirety. In one aspect, provided herein are AAV (e.g., AAV9 or AAVrh10)-based viral vectors encoding a transgene. The amino acid sequences of AAV capsids, including AAV9 and AAVrh10 are provided in FIG. 2 and Table 10 (SEQ ID NOs: 124-133 and 219-225).

TABLE 10
Amino Acid Sequences of AAV Capsids

SEQ
ID

| Capsid | NO: | Amino Acid Sequence |
|---|---|---|
| AAV.PHP.eB | 219 | MAADGYLPDW LEDNLSEGIR EWWALKPGAP QPKANQQHQD NARGLVLPGY |
| (California | | KYLGPGNGLD KGEPVNAADA AALEHDKAYD QQLKAGDNPY LKYNHADAEF |
| Institute of | | QERLKEDTSF GGNLGRAVFQ AKKRLLEPLG LVEEAAKTAP GKKRPVEQSP |
| Technology-Chan | | QEPDSSAGIG KSGAQPAKKR LNFGQTGDTE SVPDPQPIGE PPAAPSGVGS |
| et al 2017, Nat | | LTMASGGGAP VADNNEGADG VGSSSGNWHC DSQWLGDRVI TTSTRTWALP |
| Neurosci | | TYNNHLYKQI SNSTSGGSSN DNAYFGYSTP WGYFDFNRFH CHFSPRDWQR |
| 20(8): 1172-1179) | | LINNNWGFRP KRLNFKLFNI QVKEVTDNNG VKTIANNLTS TVQVFTDSDY |
| | | QLPYVLGSAH EGCLPPFPAD VEMIPQYGYL TLNDGSQAVG RSSFYCLEYF |
| | | PSQMLRTGNN FQFSYEFENV PFHSSYAHSQ SLDRLMNPLI DQYLYYLSRT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSDGTL AVPFKAQAQT |
| | | GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK |
| | | RWNPEIQYTS NYYKSNNVEF AVNTEGVYSE PRPIGTRYLT RNL |
| AAV.PHP.B | 220 | MAADGYLPDW LEDNLSEGIR EWWALKPGAP QPKANQQHQD NARGLVLPGY |
| (California | | KYLGPGNGLD KGEPVNAADA AALEHDKAYD QQLKAGDNPY LKYNHADAEF |
| Institute of | | QERLKEDTSF GGNLGRAVFQ AKKRLLEPLG LVEEAAKTAP GKKRPVEQSP |
| Technology | | QEPDSSAGIG KSGAQPAKKR LNFGQTGDTE SVPDPQPIGE PPAAPSGVGS |
| GenBank entry: | | LTMASGGGAP VADNNEGADG VGSSSGNWHC DSQWLGDRVI TTSTRTWALP |
| ALU85156.1- | | TYNNHLYKQI SNSTSGGSSN DNAYFGYSTP WGYFDFNRFH CHFSPRDWQR |
| Deverman et al | | LINNNWGFRP KRLNFKLFNI QVKEVTDNNG VKTIANNLTS TVQVFTDSDY |
| 2016 Nature | | QLPYVLGSAH EGCLPPFPAD VFMIPQYGYL TLNDGSQAVG RSSFYCLEYF |
| Biotech 34(2): | | PSQMLRTGNN FQFSYEFENV PFHSSYAHSQ SLDRLMNPLI DQYLYYLSRT |
| 204-9) | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSAQTL AVPFKAQAQT |
| | | GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK |
| | | RWNPEIQYTS NYYKSNNVEF AVNTEGVYSE PRPIGTRYLT RNL |
| VOY101 | 221 | MAADGYLPDW LEDNLSEGIR EWWALKPGAP QPKANQQHQD NARGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSND NAYFGYSTPW GYFDFNRFHC HFSPRDWQRL |
| | | INNNWGFRPK RLNFKLFNIQ VKEVTDNNGV KTIANNLTST VQVFTDSDYQ |
| | | LPYVLGSAHE GCLPPFPADV FMIPQYGYLT LNDGSQAVGR SSFYCLEYFP |
| | | SQMLRTGNNF QFSYEFENVP FHSSYAHSQS LDRLMNPLID QYLYYLSKTI |
| | | NGSGQNQQTL KFSVAGPSNM AVQGRNYIPG PSYRQQRVST TVTQNNNSEF |
| | | AWPGASSWAL NGRNSLMNPG PAMASHKEGE DRFFPLSGSL IFGKQGTGRD |
| | | NVDADKVMIT NEEEIKTTNP VATESYGQVA TNHQSDGTLA VPFKAQAQTG |
| | | WVQNQGILPG MVWQDRDVYL QGPIWAKIPH TDGNFHPSPL MGGFGMKHPP |
| | | PQILIKNTPV PADPPTAFNK DKLNSFITQY STGQVSVEIE WELQKENSKR |
| | | WNPEIQYTSN YYKSNNVEFA VNTEGVYSEP RPIGTRYLTR NL |
| VOY201 | 222 | MAADGYLPDW LEDNLSEGIR EWWALKPGAP QPKANQQHQD NARGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY |
| | | QLPYVLGSAH EGCLPPFPAD VFMIPQYGYL TLNDGSQAVG RSSFYCLEYF |
| | | PSQMLRTGNN FQFSYEFENV PFHSSYAHSQ SLDRLMNPLI DQYLYYLSKT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSAQTL AVPFKAQAQT |
| | | GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK |
| | | RWNPEIQYTS NYYKSNNVEF AVNTEGVYSE PRPIGTRYLT RNL |
| VOY701 | 223 | MAADGYLPDW LEDNLSEGIR EWWALKPGAP QPKANQQHQD NARGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY |
| | | QLPYVLGSAH EGCLPPFPAD VFMIPQYGYL TLNDGSQAVG RSSFYCLEYF |
| | | PSQMLRTGNN FQFSYEFENV PFHSSYAHSQ SLDRLMNPLI DQYLYYLSRT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSESTL AVPFKAQAQT |
| | | GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK |
| | | RWNPEIQYTS NYYKSNNVEF AVNTEGVYSEP RPIGTRYLTRN L |
| VOY801 | 224 | MAADGYLPDW LEDNLSEGIR EWWALKPGAP QPKANQQHQD NARGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDENRFH CHFSPRDWQR |
| | | LINNNWGFRP KRLNFKLEN IQVKEVTDN NGVKTIANN LTSTVQVFT |
| | | DSDYQLPYV LGSAHEGCL PPFPADVFM IPQYGYLTL NDGSQAVGR |
| | | SSFYCLEYF PSQMLRTGN NFQFSYEFE NVPFHSSYA HSQSLDRLM |
| | | NPLIDQYLY YLSRTINGS GQNQQTLKF SVAGPSNMA VQGRNYIPG |
| | | PSYRQQRVS TTVTQNNNS EFAWPGASS WALNGRNSL MNPGPAMAS |
| | | HKEGEDRFF PLSGSLIFG KQGTGRDNV DADKVMITN EEEIKTTNP |
| | | VATESYGQV ATNHQSGGT LAVPFKAQA QTGWVQNQG ILPGMVWQD |
| | | RDVYLQGPI WAKIPHTDG NFHPSPLMG GFGMKHPPP QILIKNTPV |
| | | PADPPTAFN KDKLNSFIT QYSTGQVSV EIEWELQKE NSKRWNPEI |
| | | QYTSNYYKS NNVEFAVNTE GVYSEPRPIG TRYLTRNL |
| VOY1101 | 225 | MAADGYLPDW LEDNLSEGIR EWWALKPGAP QPKANQQHQD NARGLVLPGY |
| | | KYLGPGNGLD KGEPVNAADA AALEHDKAYD QQLKAGDNPY LKYNHADAEF |
| | | QERLKEDTSF GGNLGRAVFQ AKKRLLEPLG LVEEAAKTAP GKKRPVEQSP |
| | | QEPDSSAGIG KSGAQPAKKR LNFGQTGDTE SVPDPQPIGE PPAAPSGVGS |
| | | LTMASGGGAP VADNNEGADG VGSSSGNWHC DSQWLGDRVI TTSTRTWALP |
| | | TYNNHLYKQI SNSTSGGSSN DNAYFGYSTP WGYFDFNRFH CHFSPRDWQR |
| | | LINNNWGFRP KRLNFKLFNI QVKEVTDNNG VKTIANNLTS TVQVFTDSDY |
| | | QLPYVLGSAH EGCLPPFPAD VFMIPQYGYL TLNDGSQAVG RSSFYCLEYF |
| | | PSQMLRTGNN FQFSYEFENV PFHSSYAHSQ SLDRLMNPLI DQYLYYLSRT |
| | | INGSGQNQQT LKFSVAGPSN MAVQGRNYIP GPSYRQQRVS TTVTQNNNSE |
| | | FAWPGASSWA LNGRNSLMNP GPAMASHKEG EDRFFPLSGS LIFGKQGTGR |
| | | DNVDADKVMI TNEEEIKTTN PVATESYGQV ATNHQSDGTL ATPFKAQAQT |
| | | GWVQNQGILP GMVWQDRDVY LQGPIWAKIP HTDGNFHPSP LMGGFGMKHP |
| | | PPQILIKNTP VPADPPTAFN KDKLNSFITQ YSTGQVSVEI EWELQKENSK |
| | | RWNPEIQYTS NYYKSNNVEF AVNTEGVYSEP RPIGTRYLTR NL |

Another aspect of the present invention relates to the genetic engineering of tandem nucleic acid regulatory elements and incorporating these nucleic acid sequences in a vector expression system. In one embodiment, the vector is a viral vector, including but not limited to recombinant adeno-associated viral (rAAV) vectors (e.g. Gao G., et al 2003 Proc. Natl. Acad. Sci. U.S.A. 100(10):6081-6086), lentiviral vectors (e.g. Matrai, J, et al. 2011, Hepatology 53, 1696-707), retroviral vectors (e.g. Axelrod, J H, et al. 1990. Proc Natl Acad Sci USA; 87, 5173-7), adenoviral vectors (e.g. Brown et al., 2004 Blood 103, 804-10), herpes-simplex viral vectors (Marconi, P. et al. Proc Natl Acad Sci USA. 1996 93(21): 11319-11320; Baez, M V, et al. Chapter 19—Using Herpes Simplex Virus Type 1-Based Amplicon Vectors for Neuroscience Research and Gene Therapy of Neurologic Diseases, Ed.: Robert T. Gerlai, Molecular-Genetic and Statistical Techniques for Behavioral and Neural Research, Academic Press, 2018: Pages 445-477), and retrotransposon-based vector systems (e.g. Soifer, 2004, Current Gene Therapy 4(4):373-384). In another embodiment, the vector is a non-viral vector. rAAV vectors have limited packaging capacity of the vector particles (i.e. approximately 4.7 kb), constraining the size of the transgene expression cassette to obtain functional vectors (Jiang et al., 2006 Blood. 108:107-15). The length of the transgene and the length of the regulatory nucleic acid sequences comprising promoter(s) with or without composite enhancer elements are taken into consideration when selecting a regulatory region suitable for a particular mAb transgene and the target tissue.

Another aspect of the present invention relates to a viral vector comprising an expression cassette comprising a nucleic acid regulatory element, such as a promoter with or without an enhancer element, operably linked to a mAb transgene. In some embodiments, the expression cassette comprises the nucleic acid sequence of SEQ ID NOs: 7, 8, 9, 10, 11, 12, 13, 14, or 15, or a variant thereof having 95%, 96%, 97%, 98% or 99% identity thereof, which encodes an anti-Tau antibody. Also included are nucleic acid sequences found in Tables 1, 5, 11D, 11E, 11F, or 11G or a variant thereof having 95%, 96%, 97%, 98% or 99% identity thereof, which encodes an anti-Tau antibody, antigen binding domain thereof or recombinant form thereof.

In another aspect, the expression cassettes are suitable for packaging in an AAV capsid, as such the cassette comprises (1) AAV inverted terminal repeats (ITRs) flank the expression cassette; (2) regulatory control elements, a) promoter/enhancers, including but not limited to any one of the promoters as in Table 8, b) a poly A signal, and c) optionally an intron; and (3) a transgene providing (e.g., coding for) at least one of the heavy chain or light chain of an anti-Tau mAb.

In certain embodiments, the transgene has a heavy and light chain variable domain as encoded by the nucleotide sequences of Table 1 or has an amino acid sequence of the heavy and light chain variable domains or Fab portions as in Table 2 or Table 11G, or the scFv or scFv-Fcs encoded by the nucleotide sequence of Table 11D, 11E, or 11F, or having an amino acid sequence of Table 11B, 11C, or 11F. In other embodiments for expressing an intact or substantially intact mAb, the constructs described herein comprise the following components: (1) AAV2 inverted terminal repeats that flank the expression cassette; (2) regulatory control elements, a) promoter/enhancers, including but not limited to any one of the promoters as in Table 8, b) a poly A signal, and c) optionally an intron; and (3) nucleic acid sequences coding for the light chain and heavy chain of an anti-Tau mAb, wherein the heavy chain (Fab and, optionally Fc region) and the light chain are separated by a self-cleaving furin (F)/F2A or furin (F)/T2A or flexible linker, ensuring expression of adequate amounts of the heavy and the light chain polypeptides.

In other embodiments for expressing a Fab mAb, the constructs described herein comprise the following components: (1) AAV2 inverted terminal repeats that flank the expression cassette; (2) regulatory control elements, a) promoter/enhancers, including but not limited to any one of the promoters as in Table 8, b) a poly A signal, and c) optionally an intron; and (3) nucleic acid sequences for the light chain and heavy chain of an anti-Tau mAb, wherein the heavy chain (Fab region only; VH—C_{H}1, or VH and C_{H}1) and the light chain (VL and CL) are separated by a self-cleaving furin (F)/F2A or furin (F)/T2a or flexible linker, ensuring expression of adequate amounts of the heavy and the light chain polypeptides.

In other embodiments for expressing a Fab′ or F(ab′)₂ mAb, the constructs described herein comprise the following components: (1) AAV2 inverted terminal repeats that flank the expression cassette; (2) regulatory control elements, a) promoter/enhancers, including but not limited to any one of the promoters as in Table 8, b) a poly A signal, and c) optionally an intron; and (3) nucleic acid sequences for the light chain and heavy chain of an anti-Tau mAb, wherein the heavy chain (Fab region only; VH—C_{H}1, or VH, C_{H}1 and all or part of the hinge region) and the light chain (VL and CL) are separated by a self-cleaving furin (F)/F2A or furin (F)/T2a or flexible linker, ensuring expression of adequate amounts of the heavy and the light chain polypeptides, wherein the F(ab′)₂ fragment has two antigen-binding Fab portions linked together by disulfide bonds. In other embodiments, the nucleic acid transgene encodes a polypeptide of Table 2 or 5 or Table 11B, 11C, 11F, or 11G.

In embodiments related to expression of an ScFv or ScFv-Fc, the constructs described herein comprise the following components: (1) AAV2 inverted terminal repeats that flank the expression cassette; (2) regulatory control elements, a) promoter/enhancers, including but not limited to any one of the promoters as in Table 8, b) a poly A signal, and c) optionally an intron; and (3) nucleic acid sequences encoding for an anti-Tau mAb, wherein the heavy chain variable domain and the light chain variable domain as in Table 1, or a codon-optimized and/or CpG-depleted variant thereof, are separated by a flexible linker (scFvs, for example, as encoded by nucleotide sequences of Table 11C; or scFv-Fcs, for example, as encoded by nucleotide sequences of Table 11E or 11F); and optionally 4) operably linked to a nucleic acid sequence encoding an Fc domain, containing at least a C_{H}2 and C_{H}3 domain. FIGS. 1A and 1B. In some aspects, the ITR to ITR expression cassette comprises the nucleotides sequence of SEQ ID NO: 369 or 372.

In other embodiments for expressing a diabody, the constructs described herein comprise the following components: (1) AAV2 inverted terminal repeats that flank the expression cassette; (2) regulatory control elements, a) promoter/enhancers, including but not limited to any one of the promoters as in Table 8, b) a poly A signal, and c) optionally an intron; and (3) nucleic acid sequences for the heavy chain variable region (VH) and light chain variable region (VL) of an anti-Tau mAb, wherein the VH and the VL are separated by a flexible linker, ensuring expression of adequate amounts of the VH-VL or VL-VH polypeptides such that each single-chain polypeptide dimerizes with its complement. FIGS. 1A and 1C.

In the various embodiments, the target tissue may be neural tissue, or endothelial tissue, such as the cellular matrix of the blood brain barrier, or a particular receptor, and the regulatory agent is derived from a heterologous protein or domain that specifically recognizes and/or binds that tissue, particularly in the CNS. The transgenes may also be expressed in liver, or muscle and liver, if administered systemically allowing for systemic (or serum) expression, since circulating antibody proteins are known to cross the blood brain barrier to the CNS thus delivering the Tau therapeutics to the CNS.

The provided nucleic acids and methods are suitable for use in the production of any isolated recombinant AAV particles, in the production of a composition comprising any isolated recombinant AAV particles, or in the method for treating a Tau-related disease or disorder in a subject in need thereof comprising the administration of any isolated recombinant AAV particles. As such, the rAAV may be of any serotype, modification, or derivative, known in the art, or any combination thereof (e.g., a population of rAAV particles that comprises two or more serotypes, e.g., comprising two or more of rAAV2, rAAV8, and rAAV9 particles) known in the art. In some embodiments, the rAAV particles are AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or other rAAV particles, or combinations of two or more thereof.

In some embodiments, rAAV particles have a capsid protein from an AAV serotype selected from AAV1, AAV1, AAV2, rAAV3, AAV4, AAV5, AAV6, AAV7, AAV8, AAV9, AAV10, AAV11, AAV-12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, rAAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise a capsid protein from an AAV capsid serotype selected from AAV1, AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74.v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16, or a derivative, modification, or pseudotype thereof. In some embodiments, rAAV particles comprise a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to e.g., VP1, VP2 and/or VP3 sequence of an AAV capsid serotype selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In some embodiments, rAAV particles comprise the capsid of Anc80 or Anc80L65, as described in Zinn et al., 2015, Cell Rep. 12(6): 1056-1068, which is incorporated by reference in its entirety. In certain embodiments, the rAAV particles comprise the capsid with one of the following amino acid insertions: LGETTRP or LALGETTRPA, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise the capsid of AAV.7m8, as described in U.S. Pat. Nos. 9,193,956; 9,458,517; and 9,587,282 and US patent application publication no. 2016/0376323, each of which is incorporated herein by reference in its entirety. In some embodiments, rAAV particles comprise any AAV capsid disclosed in U.S. Pat. No. 9,585,971, such as AAV-PHP.B. In some embodiments, rAAV particles comprise any AAV capsid disclosed in U.S. Pat. No. 9,840,719 and WO 2015/013313, such as AAV.Rh74 and RHM4-1, each of which is incorporated herein by reference in its entirety. In some embodiments, rAAV particles comprise any AAV capsid disclosed in WO 2014/172669, such as AAV rh.74, which is incorporated herein by reference in its entirety. In some embodiments, rAAV particles comprise the capsid of AAV2/5, as described in Georgiadis et al., 2016, Gene Therapy 23: 857-862 and Georgiadis et al., 2018, Gene Therapy 25: 450, each of which is incorporated by reference in its entirety. In some embodiments, rAAV particles comprise any AAV capsid disclosed in WO 2017/070491, such as AAV2tYF, which is incorporated herein by reference in its entirety. In some embodiments, rAAV particles comprise the capsids of AAVLK03 or AAV3B, as described in Puzzo et al., 2017, Sci. Transl. Med. 29(9): 418, which is incorporated by reference in its entirety. In some embodiments, rAAV particles comprise any AAV capsid disclosed in U.S. Pat. Nos. 8,628,966; 8,927,514; 9,923,120 and WO 2016/049230, such as HSC1, HSC2, HSC3, HSC4, HSC5, HSC6, HSC7, HSC8, HSC9, HSC10, HSC11, HSC12, HSC13, HSC14, HSC15, or HSC16, each of which is incorporated by reference in its entirety.

In some embodiments, rAAV particles comprise an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in any of the following patents and patent applications, each of which is incorporated herein by reference in its entirety: U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; and International Patent Application Nos. PCT/US2015/034799; PCT/EP2015/053335.

In some embodiments, rAAV particles have a capsid protein disclosed in Intl. Appl. Publ. No. WO 2003/052051 (see, e.g., SEQ ID NO: 2 in ′051), PCT/US2019/032387 and PCT/US2019/055756, such as VOY101 (SEQ ID NO: 221), VOY201 (SEQ ID NO: 222), VOY701 (SEQ ID NO: 223), VOY801 (SEQ ID NO: 224), VOY1101 (SEQ ID NO: 225) (see Table 10 above), WO 2005/033321 (see, e.g., SEQ ID NOs: 123 and 88 in ′321), WO 03/042397 (see, e.g., SEQ ID NOs: 2, 81, 85, and 97 in ′397), WO 2006/068888 (see, e.g., SEQ ID NOs: 1 and 3-6 in ′888), WO 2006/110689, (see, e.g., SEQ ID NOs: 5-38 in ′689) WO2009/104964 (see, e.g., SEQ ID NOs: 1-5, 7, 9, 20, 22, 24 and 31 in ′964), WO 2010/127097 (see, e.g., SEQ ID NOs: 5-38 in ′097), and WO 2015/191508 (see, e.g., SEQ ID NOs: 80-294 in ′508), and U. S. Appl. Publ. No. 20150023924 (see, e.g., SEQ ID NOs: 1, 5-10 in ′924), the contents of each of which is herein incorporated by reference in its entirety. In some embodiments, rAAV particles have a capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of an AAV capsid disclosed in Intl. Appl. Publ. No. WO 2003/052051 (see, e.g., SEQ ID NO: 2 in ′051), WO 2005/033321 (see, e.g., SEQ ID NOs: 123 and 88 in ′321), WO 03/042397 (see, e.g., SEQ ID NOs: 2, 81, 85, and 97 in ′397), WO 2006/068888 (see, e.g., SEQ ID NOs: 1 and 3-6 in ′888), WO 2006/110689 (see, e.g., SEQ ID NOs: 5-38 in ′689) WO2009/104964 (see, e.g., SEQ ID NOs: 1-5, 7, 9, 20, 22, 24 and 31 in ′964), WO 2010/127097 (see, e.g., SEQ ID NOs: 5-38 in ′097), and WO 2015/191508 (see, e.g., SEQ ID NOs: 80-294 of in ′508), and U.S. Appl. Publ. No. 20150023924 (see, e.g., SEQ ID NOs: 1, 5-10 in ′924).

Nucleic acid sequences of AAV based viral vectors and methods of making recombinant AAV and AAV capsids are taught, for example, in U.S. Pat. Nos. 7,282,199; 7,906,111; 8,524,446; 8,999,678; 8,628,966; 8,927,514; 8,734,809; 9,284,357; 9,409,953; 9,169,299; 9,193,956; 9,458,517; and 9,587,282; US patent application publication nos. 2015/0374803; 2015/0126588; 2017/0067908; 2013/0224836; 2016/0215024; 2017/0051257; International Patent Application Nos. PCT/US2015/034799; PCT/EP2015/053335; WO 2003/052051, WO 2005/033321, WO 03/042397, WO 2006/068888, WO 2006/110689, WO2009/104964, WO 2010/127097, and WO 2015/191508, and U.S. Appl. Publ. No. 20150023924.

The provided methods are suitable for used in the production of recombinant AAV encoding an antibody transgene. In some embodiments, provided herein are rAAV viral vectors encoding an anti-Tau Fab. In some embodiments, provided herein are rAAV9-based viral vectors encoding a Tau-specific mAb. In some embodiments, provided herein are rAAV viral vectors encoding a full-length Tau-specific (anti-Tau) mAb. In some embodiments, provided herein are rAAV9-based or rAAVrh10-based viral vectors encoding Fab or full-length Tau-specific mAb.

In additional embodiments, rAAV particles comprise a pseudotyped AAV capsid. In some embodiments, the pseudotyped AAV capsids are rAAV2/9 or rAAV2/rh10 pseudotyped AAV capsids. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles comprise a capsid containing a capsid protein chimeric of two or more AAV capsid serotypes. In some embodiments, the capsid protein is a chimeric of 2 or more AAV capsid proteins from AAV serotypes selected from AAV1, AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16.

In certain embodiments, a single-stranded AAV (ssAAV) can be used. In certain embodiments, a self-complementary vector, e.g., scAAV, can be used (see, e.g., Wu, 2007, Human Gene Therapy, 18(2):171-82, McCarty et al, 2001, Gene Therapy, Vol. 8, Number 16, Pages 1248-1254; and U.S. Pat. Nos. 6,596,535; 7,125,717; and 7,456,683, each of which is incorporated herein by reference in its entirety).

In some embodiments, rAAV particles in the clarified feed comprise a capsid protein from an AAV capsid serotype selected from AAV9 (SEQ ID NO: 132) or AAVrh10 (SEQ ID NO: 133), AAV.PHP.eB (SEQ ID NO: 219), or AAV.PHP.B (SEQ ID NO: 220). In some embodiments, the rAAV particles have an AAV capsid serotype of AAV1 or a derivative, modification, or pseudotype thereof. In some embodiments, the rAAV particles have an AAV capsid serotype of AAV4 or a derivative, modification, or pseudotype thereof. In some embodiments, the rAAV particles have an AAV capsid serotype of AAV5 or a derivative, modification, or pseudotype thereof. In some embodiments, the rAAV particles have an AAV capsid serotype of AAV9 or a derivative, modification, or pseudotype thereof. In some embodiments, the rAAV particles have an AAV capsid serotype of AAVrh10 or a derivative, modification, or pseudotype thereof.

In some embodiments, rAAV particles in the clarified feed comprise a capsid protein that is a derivative, modification, or pseudotype of AAV9 (e.g. AAV.PHP.eB or AAV.PHP.B) or AAVrh10 capsid protein. In some embodiments, rAAV particles in the clarified feed comprise a capsid protein that has an AAV9 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV9 capsid protein. In some embodiments, rAAV particles in the clarified feed comprise a capsid protein that has an AAV.PHP.eB capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV.PHP.eB capsid protein. In some embodiments, rAAV particles in the clarified feed comprise a capsid protein that has an AAV.PHP.B capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAV.PHP.B capsid protein.

In some embodiments, rAAV particles in the clarified feed comprise a capsid protein that is a derivative, modification, or pseudotype of AAVrh10 capsid protein. In some embodiments, rAAV particles in the clarified feed comprise a capsid protein that has an AAVrh10 capsid protein at least 80% or more identical, e.g., 85%, 85%, 87%, 88%, 89%, 90%, 91%, 92%, 93%, 94%, 95%, 96%, 97%, 98%, 99%, 99.5%, etc., i.e. up to 100% identical, to the VP1, VP2 and/or VP3 sequence of AAVrh10 capsid protein.

In additional embodiments, rAAV particles in the clarified feed comprise a mosaic capsid. Mosaic AAV particles are composed of a mixture of viral capsid proteins from different serotypes of AAV. In some embodiments, rAAV particles in the clarified feed comprise a mosaic capsid containing capsid proteins of a serotype selected from AAV1, AAV2, AAV3, AAV4, AAV5, AAV6, AAV7, AAV8, AAV9, AAV10, AAV11, AAV12, AAV13, AAV14, AAV15, AAV-16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, rAAV particles in the clarified feed comprise a mosaic capsid containing capsid proteins of a serotype selected from AAV1, AAV2, AAV5, AAV6, AAV7, AAV8, AAV9, AAV.PHP.eB, AAV.PHP.B, AAV10, AAVrh.8, and AAVrh10. In additional embodiments, rAAV particles in the clarified feed comprise a pseudotyped rAAV particle. In some embodiments, the pseudotyped rAAV particle comprises (a) a nucleic acid vector comprising AAV ITRs and (b) a capsid comprised of capsid proteins derived from AAVx (e.g., AAV1, AAV3, AAV4, AAV5, AAV6, AAV7, AAV8, AAV9, AAV10 AAV11, AAV12, AAV13, AAV14, AAV15 and AAV16). In additional embodiments, rAAV particles in the clarified feed comprise a pseudotyped rAAV particle comprised of a capsid protein of an AAV serotype selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In additional embodiments, rAAV particles in the clarified feed comprise a pseudotyped rAAV particle containing AAV9 capsid protein. In additional embodiments, rAAV particles in the clarified feed comprise a pseudotyped rAAV particle containing AAV.PHP.B capsid protein. In additional embodiments, rAAV particles in the clarified feed comprise a pseudotyped rAAV particle containing AAV.PHP.eB capsid protein. In additional embodiments, rAAV particles in the clarified feed comprise a pseudotyped rAAV particle is comprised of AAVrh10 capsid protein. In some embodiments, the pseudotyped rAAV9 or rAAVrh10 particles are rAAV2/9 or rAAV2/rh10 pseudotyped particles. Methods for producing and using pseudotyped rAAV particles are known in the art (see, e.g., Duan et al., J. Virol., 75:7662-7671 (2001); Halbert et al., J. Virol., 74:1524-1532 (2000); Zolotukhin et al., Methods 28:158-167 (2002); and Auricchio et al., Hum. Molec. Genet. 10:3075-3081, (2001).

In additional embodiments, rAAV particles in the clarified feed comprise a capsid containing a capsid protein chimeric of two or more AAV capsid serotypes. In further embodiments, the capsid protein is a chimeric of 2 or more AAV capsid proteins from AAV serotypes selected from AAV1, AAV2, rAAV3, AAV4, AAV5, AAV6, AAV7, AAV8, AAV9, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, rAAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In further embodiments, the capsid protein is a chimeric of 2 or more AAV capsid proteins from AAV serotypes selected from AAV1, AAV2, AAV5, AAV6, AAV7, AAV8, AAV9, AAV10, AAVrh.8, and AAVrh.10.

In some embodiments, the rAAV particles comprise an AAV capsid protein chimeric of AAV9 capsid protein and one or more AAV capsid proteins from an AAV serotype selected from AAV1, AAV2, AAV3, AAV4, AAV5, AAV6, AAV7, AAV8, AAV10, AAV11, AAV12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16. In some embodiments, the rAAV particles comprise an AAV capsid protein chimeric of AAV9 capsid protein and one or more AAV capsid proteins from an AAV serotype selected from AAV1, AAV2, AAV5, AAV6, AAV7, AAV8, AAV.PHP.eB, AAV.PHP.B, AAV10, AAVrh.8, and AAVrh.10.

In some embodiments, the rAAV particles comprise an AAV capsid protein chimeric of AAVrh10 capsid protein the capsid protein of one or more AAV capsid serotypes selected from AAV1, AAV2, AAV3, AAV4, AAV5, AAV6, AAV7, AAV8, AAV9, AAV10, AAV11, AAV12, AAV13, AAV14, AAV15, AAV16, AAV.rh8, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, and AAV.HSC16.

In some embodiments, the rAAV particles comprise an AAV capsid protein chimeric of AAV.rh10, capsid protein the capsid protein of one or more AAV capsid serotypes selected from AAV1, AAV2, AAV3, AAV4, AAV5, AAV6, AAV7, AAV8, AAV9, AAV.PHP.eB, AAV.PHP.B, and AAVrh.8.

### 5.8. Methods of Making rAAV Virions Packaging Antibody Transgenes

Another aspect of the present invention involves making molecules disclosed herein. In some embodiments, a molecule according to the invention is made by providing a polynucleotide comprising the nucleic acid sequence encoding an AAV capsid protein; and using a packaging cell system to prepare corresponding rAAV particles with capsid coats made up of the capsid protein. In some embodiments, the nucleic acid sequence encodes a sequence having at least 60%, 70%, 80%, 85%, 90%, or 95%, preferably 96%, 97%, 98%, 99% or 99.9%, identity to the sequence of a capsid protein molecule described herein, and retains (or substantially retains) biological function of the capsid protein and the inserted peptide from a heterologous protein or domain thereof. In some embodiments, the nucleic acid encodes a sequence having at least 60%, 70%, 80%, 85%, 90%, or 95%, preferably 96%, 97%, 98%, 99% or 99.9%, identity to a particular sequence of the AAV capsid protein, while retaining (or substantially retaining) biological function of the AAV capsid protein.

The capsid protein, coat, and rAAV particles may be produced by techniques known in the art. In some embodiments, the viral genome comprises at least one inverted terminal repeat to allow packaging into a vector. In some embodiments, the viral genome further comprises a cap gene and/or a rep gene for expression and splicing of the cap gene. In certain embodiments, the cap and rep genes are provided by a packaging cell and not present in the viral genome.

In some embodiments, the nucleic acid encoding the capsid protein is cloned into an AAV Rep-Cap helper plasmid in place of the existing capsid gene. When introduced together into host cells, this plasmid helps package an rAAV genome into the capsid protein as the capsid coat. Packaging cells can be any cell type possessing the genes necessary to promote AAV genome replication, capsid assembly, and packaging. Nonlimiting examples include 293 cells or derivatives thereof, HELA cells, or insect cells.

Standard techniques can be used for recombinant DNA, oligonucleotide synthesis, and tissue culture and transformation (e.g., electroporation, lipofection). Enzymatic reactions and purification techniques can be performed according to manufacturer's specifications or as commonly accomplished in the art or as described herein. The foregoing techniques and procedures can be generally performed according to conventional methods well known in the art and as described in various general and more specific references that are cited and discussed throughout the present specification. See, e.g., Sambrook et al., Molecular Cloning: A Laboratory Manual (2d ed., Cold Spring Harbor Laboratory Press, Cold Spring Harbor, N.Y. (1989)), which is incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well-known and commonly used in the art. Standard techniques can be used for chemical syntheses, chemical analyses, pharmaceutical preparation, formulation, and delivery, and treatment of patients. Nucleic acid sequences of AAV-based viral vectors, and methods of making recombinant AAV and AAV capsids, are taught, e.g., in U.S. Pat. Nos. 7,282,199; 7,790,449; 8,318,480; 8,962,332; and PCT/EP2014/076466, each of which is incorporated herein by reference in its entirety.

In preferred embodiments, the rAAVs provide transgene delivery vectors that can be used in therapeutic and prophylactic applications, as discussed in more detail below. The rAAV vector also includes the regulatory control elements discussed supra to influence the expression of the RNA and/or protein products encoded by nucleic acids (transgenes) within target cells of the subject.

Provided in particular embodiments are AAV vectors comprising a viral genome comprising an expression cassette for expression of the transgene, under the control of regulatory elements, and flanked by ITRs and an engineered viral capsid as described herein or is at least 95%, 96%, 97%, 98%, 99% or 99.9% identical to the amino acid sequence of the AAV capsid protein.

The recombinant adenovirus can be a first generation vector, with an E1 deletion, with or without an E3 deletion, and with the expression cassette inserted into either deleted region. The recombinant adenovirus can be a second generation vector, which contains full or partial deletions of the E2 and E4 regions. A helper-dependent adenovirus retains only the adenovirus inverted terminal repeats and the packaging signal (phi). The transgene generally is inserted between the packaging signal and the 3′ITR, with or without stuffer sequences to keep the genome close to wild-type size of approximately 36 kb. An exemplary protocol for production of adenoviral vectors may be found in Alba et al., 2005, “Gutless adenovirus: last generation adenovirus for gene therapy,” Gene Therapy 12:S18-S27, which is incorporated by reference herein in its entirety.

The rAAV vector for delivering the transgene to target tissues, cells, or organs, may also have a tropism for that particular target tissue, cell, or organ, e.g. liver and/or muscle, in conjunction with the use of tissue-specific promoters as described herein. The construct can further include additional expression control elements such as introns that enhance expression of the transgene (e.g., introns such as the chicken j-actin intron, minute virus of mice (MVM) intron, human factor IX intron (e.g., FIX truncated intron 1), β-globin splice donor/immunoglobulin heavy chain splice acceptor intron, adenovirus splice donor/immunoglobulin splice acceptor intron, SV40 late splice donor/splice acceptor (19S/16S) intron, and hybrid adenovirus splice donor/IgG splice acceptor intron and polyA signals such as the rabbit β-globin polyA signal, human growth hormone (hGH) polyA signal, SV40 late polyA signal, synthetic polyA (SPA) signal, and bovine growth hormone (bGH) polyA signal. See, e.g., Powell and Rivera-Soto, 2015, Discov. Med., 19(102):49-57.

In certain embodiments, nucleic acids sequences disclosed herein may be codon-optimized, for example, via any codon-optimization technique known to one of skill in the art (see, e.g., review by Quax et al., 2015, Mol Cell 59:149-161). In addition or alternatively, the antibody coding sequences and/or the transgene sequences may be depleted of CpG to reduce immunogenicity.

In one aspect the nucleic acid sequence encoding the transgene cassette is modified by codon optimization and CpG island depletion. Immune response against an anti-Tau antibody transgene is reduced for a codon-optimized and CpG deleted transgene sequence.

AAV-directed immune responses can be inhibited by reducing the number of CpG di-nucleotides in the AAV genome [Faust, S. M., et al., CpG-depleted adeno-associated virus vectors evade immune detection. J Clin Invest, 2013. 123(7): p. 2994-3001]. Depleting the transgene sequence of CpG motifs may diminish the role of TLR9 in activation of innate immunity upon recognition of the transgene as non-self, and thus provide stable and prolonged transgene expression. [See also Wang, D., P. W. L. Tai, and G. Gao, Adeno-associated virus vector as a platform for gene therapy delivery. Nat Rev Drug Discov, 2019. 18(5): p. 358-378.; and Rabinowitz, J., Y. K. Chan, and R. J. Samulski, Adeno-associated Virus (AAV) versus Immune Response. Viruses, 2019. 11(2)]. In embodiments, the antibody-encoding cassette is human codon-optimized with CpG depletion. Codon-optimized and CpG depleted nucleic acid sequences may be designed by any method known in the art, including for example, by Thermo Fisher Scientific GeneArt Gene Synthesis tools utilizing GeneOptimizer (Waltham, MA USA)).

In one embodiment, the constructs described herein comprise the following components: (1) AAV2 inverted terminal repeats that flank the expression cassette; (2) control elements, which include a) a CNS-specific or a ubiquitous promoter, d) a rabbit β-globin poly A signal and e) optionally a chimeric intron derived from human β-globin and Ig heavy chain, or other intron; and (3) transgene providing (e.g., coding for) at least one heavy chain and at least one light chain of the Tau-specific mAbs of interest.

In another embodiment, the constructs described herein comprise the following components: (1) AAV2 inverted terminal repeats that flank the expression cassette; (2) regulatory control elements, which include a) a CNS-specific or a ubiquitous promoter, d) a rabbit β-globin poly A signal and e) optionally a chimeric intron derived from human 3-globin and Ig heavy chain, or other intron; and (3) transgene encoding for an anti-Tau mAb, wherein the heavy chain variable domain and the light chain variable domain as in Table 1, or a codon-optimized and/or CpG-depleted variant thereof, are separated by a flexible linker (scFvs or scFv-Fcs, for example, having the amino acid sequences in Table 11C or 11E or 11F or as encoded by nucleotide sequences of Table 11D or 11E or 11F, which encoded the components of the scFvs); and optionally 4) operably linked to a nucleic acid sequence encoding an Fc domain, containing at least a C_{H}2 and C_{H}3 domain. FIGS. 1A and 1B. In another embodiment, the constructs described herein comprise the following components: (1) AAV2 inverted terminal repeats that flank the expression cassette; (2) regulatory control elements, which include a) a CNS-specific or a ubiquitous promoter, d) a rabbit β-globin poly A signal and e) optionally a chimeric intron derived from human 3-globin and Ig heavy chain, or other intron; and (3) transgene encoding for the light chain and heavy chain of an anti-Tau mAb, wherein the heavy chain (Fab region only; VH and C_{H}1) and the light chain (VL and CL) are separated by a self-cleaving furin (F)/F2A or furin (F)/T2a or flexible linker, ensuring expression of adequate amounts of the heavy and the light chain polypeptides.

The viral vectors provided herein may be manufactured using host cells, e.g., mammalian host cells, including host cells from humans, monkeys, mice, rats, rabbits, or hamsters. Nonlimiting examples include: A549, WEHI, 10T1/2, BHK, MDCK, COS1, COS7, BSC 1, BSC 40, BMT 10, VERO, W138, HeLa, 293, Saos, C2C12, L, HT1080, HepG2, primary fibroblast, hepatocyte, and myoblast cells. Typically, the host cells are stably transformed with the sequences encoding the transgene and associated elements (i.e., the vector genome), and genetic components for producing viruses in the host cells, such as the replication and capsid genes (e.g., the rep and cap genes of AAV). For a method of producing recombinant AAV vectors with AAV8 capsids, see Section IV of the Detailed Description of U.S. Pat. No. 7,282,199 B2, which is incorporated herein by reference in its entirety. Genome copy titers of said vectors may be determined, for example, by TAQMAN® analysis. Virions may be recovered, for example, by CsCl₂ sedimentation. Alternatively, baculovirus expression systems in insect cells may be used to produce AAV vectors. For a review, see Aponte-Ubillus et al., 2018, Appl. Microbiol. Biotechnol. 102:1045-1054, which is incorporated by reference herein in its entirety for manufacturing techniques.

In vitro assays, e.g., cell culture assays, can be used to measure transgene expression from a vector described herein, thus indicating, e.g., potency of the vector. For example, the PER.C6® Cell Line (Lonza), a cell line derived from human embryonic retinal cells, or retinal pigment epithelial cells, e.g., the retinal pigment epithelial cell line hTERT RPE-1 (available from ATCC®), can be used to assess transgene expression. Alternatively, cell lines derived from liver or muscle or other cell types may be used, for example, but not limited, to HuH-7, HEK293, fibrosarcoma HT-1080, HKB-11, C2C12 myoblasts, and CAP cells. Once expressed, characteristics of the expressed product (transgene product) can also be determined, including serum half-life, functional activity of the protein (e.g. enzymatic activity or binding to a target), determination of the glycosylation and tyrosine sulfation patterns, and other assays known in the art for determining protein characteristics.

In vitro relative potency assays may be performed to relate the vector genome concentration (vector GC or VGC, or titer) to gene expression using ddPCR. The in vitro bioassay may be performed by transducing HEK293 cells and assaying the cell culture supernatant for anti-Tau mAb (e.g. Fab or IgG or ScFv) protein levels. HEK293 cells are plated onto three poly-D-lysine-coated 96-well tissue culture plates overnight. The cells are then pre-infected with wild-type human Ad5 virus followed by transduction with three independently prepared serial dilutions of AAV vector reference standard and test article, with each preparation plated onto separate plates at different positions. On the third day following transduction, the cell culture media is collected from the plates and measured for Tau-binding protein levels via ELISA. For the ELISA, 96-well ELISA plates coated with Tau are blocked and then incubated with the collected cell culture media to capture anti-Tau mAb produced by HEK293 cells. mAb-specific anti-human IgG antibody is used to detect the Tau-captured mAb protein. After washing, horseradish peroxidase (HRP) substrate solution is added, allowed to develop, stopped with stop buffer, and the plates are read in a plate reader. The absorbance or OD of the HRP product is plotted versus log dilution, and the relative potency of each test article is calculated relative to the reference standard on the same plate fitted with a four-parameter logistic regression model after passing the parallelism similarity test, using the formula: EC50 reference÷EC50 test article. The potency of the test article is reported as a percentage of the reference standard potency, calculated from the weighted average of the three plates.

To relate the ddPCR GC titer to functional gene expression, an in vitro bioassay may be performed by transducing HEK293 cells and assaying for transgene (e.g. enzyme) activity. HEK293 cells are plated onto three 96-well tissue culture plates overnight. The cells are then pre-infected with wild-type human adenovirus serotype 5 virus followed by transduction with three independently prepared serial dilutions of enzyme reference standard and test article, with each preparation plated onto separate plates at different positions. On the second day following transduction, the cells are lysed, treated with low pH to activate the enzyme, and assayed for enzyme activity using a peptide substrate that yields increased fluorescence signal upon cleavage by transgene (enzyme). The fluorescence or RFU is plotted versus log dilution, and the relative potency of each test article is calculated relative to the reference standard on the same plate fitted with a four-parameter logistic regression model after passing the parallelism similarity test, using the formula: EC50 reference+EC50 test article. The potency of the test article is reported as a percentage of the reference standard potency, calculated from the weighted average of the three plates. Vector genome concentration GC can also be evaluated using ddPCR

### 5.9. Therapeutic and Prophylactic Uses

Another aspect relates to therapies which involve administering a transgene via a rAAV vector according to the invention to a subject in need thereof, for delaying, preventing, treating, and/or managing a Tau-related disease or disorder or tauopathy, and/or ameliorating one or more symptoms associated therewith. A subject in need thereof includes a subject suffering from the Tau-related disease or disorder or tauopathy, or a subject pre-disposed thereto, e.g., a subject at risk of developing or having a recurrence of the Tau-related disease or disorder or tauopathy.

Tables 1, 5, 11D, 11E, 11F, and 11G hereinabove provide transgenes and nucleic acid sequences for generating transgenes that may be used in any of the rAAV vectors described herein, in particular, to treat or ameliorate various tauopathies and amino acid sequences of the anti-Tau mAbs, heavy and light chain variable domains thereof, constant domains thereof and recombinant forms that may be encoded by the transgenes are provided in Tables 2, 5, 11B, and 11C and described herein. Neurodegenerative tauopathies are a diverse group of neurodegenerative disorders that share a common pathologic lesion consisting of intracellular aggregates of abnormal filaments that are mainly composed of pathologically hyperphosphorylated Tau in neurons and/or glial cells. Clinical features of the tauopathies are heterogeneous and characterized by dementia and/or motor syndromes. The progressive accumulation of filamentous Tau inclusions may cause neuronal and glial degeneration in combination with other deposits as, e.g., beta-amyloid in AD or as a sole pathogenic entity as illustrated by mutations in the tau gene that are associated with familial forms of FTDP-17. Because of the heterogeneity of their clinical manifestations a potentially non-exhaustive list of tauopathic diseases may be provided including AD, ALS-PDC, AGD, British type amyloid angiopathy, cerebral amyloid angiopathy, DBD, CID, dementia pugilistica, diffuse neurofibrillary tangles with calcification, Down's syndrome, FTD, FTDP-17, frontotemporal lobar degeneration, Gerstmann-Straussler-Scheinker disease, Hallervorden-Spatz disease, inclusion body myositis, multiple system atrophy, myotonic dystrophy, NP—C, non-Guamanian motor neuron disease with neurofibrillary tangles, PiD, postencephalitic parkinsonism, prion protein cerebral amyloid angiopathy, progressive subcoitical gliosis, PSP, subacute sclerosing panencephalitis, tangle only dementia, multi-infarct dementia and ischemic stroke; see for a review, e.g., Lee et al. (2001) Annu. Rev. Neurosci. 24, 1121-1159 in which Table 1 catalogs the unique members of tauopathies or Sergeant et al. (2005), Bioch. Biophy. Acta 1739, 179-97, with a list in FIG. 2 therein.

As described herein, the AAV vector may be engineered as described herein to target the appropriate tissue (CNS) for delivery of the transgene to effect the therapeutic or prophylactic use. The appropriate AAV serotype may be chosen to engineer to optimize the tissue tropism and transduction of the vector.

Generally, the rAAV vector is administered to the CNS, intracerebroventricular (ICV), intracisternal (IC), including direct injection into the cisterna magna, lumbar intrathecal (IT) or intraparenchymal administration, and following transduction, the vector's production of the protein product is enhanced by an expression cassette employing engineered CNS-specific nucleic acid regulatory elements. In other examples, the rAAV vector may cross the blood brain barrier if provided systemically by intravenous, intramuscular, and/or intra-peritoneal administration. Alternatively, the rAAV is delivered, for example, intravenously, intramuscularly or other parenteral administration such that the rAAV is delivered to the liver where the rAAV transduces liver cells, generating a depot for production of the anti-Tau binding protein and deliver of that protein to the circulation.

Following transduction of target cells, the expression of the protein product is enhanced by employing such CNS-specific vector delivery methods. Such enhancement may be measured by the following non-limiting list of determinations such as 1) protein titer by assays known to the skilled person, not limited to sandwich ELISA, Western Blot, histological staining, and liquid chromatography tandem mass spectrometry (LC-MS/MS); 2) protein activity, by assays such as binding assays, functional assays, enzymatic assays and/or substrate detection assays; and/or 3) serum half-life or long-term expression. Enhancement of transgene expression may be determined as efficacious and suitable for human treatment (Hintze, J. P. et al, Biomarker Insights 2011:6 69-78). Assessment of the quantitative and functional properties of a Tau-specific mAb transgene using such in vitro and in vivo cellular, blood and tissue studies have been shown to correlate to the efficacy of certain therapies (Hintze, J. P. et al, 2011, supra), and are utilized to evaluate response to gene therapy treatment of the transgene with the vectors described herein.

The agents may be provided as pharmaceutically acceptable compositions as known in the art and/or as described herein. In some embodiments, the rAAV molecule may be administered alone or in combination with other prophylactic and/or therapeutic agents, such as with prophylactic immunosuppressants.

The dosage amounts and frequencies of administration provided herein are encompassed by the terms therapeutically effective and prophylactically effective. The dosage and frequency will typically vary according to factors specific for each patient depending on the specific therapeutic or prophylactic agents administered, the severity and type of disease, the route of administration, as well as age, body weight, response, and the past medical history of the patient, and should be decided according to the judgment of the practitioner and each patient's circumstances. Suitable regimens can be selected by one skilled in the art by considering such factors and by following, for example, dosages reported in the literature and recommended in the Physician's Desk Reference (56^{th} ed., 2002). Prophylactic and/or therapeutic agents can be administered repeatedly. Several aspects of the procedure may vary such as the temporal regimen of administering the prophylactic or therapeutic agents, and whether such agents are administered separately or as an admixture.

The amount of an agent of the invention that will be effective can be determined by standard clinical techniques. Effective doses may be extrapolated from dose-response curves derived from in vitro or animal model test systems. For any agent used in the method of the invention, the therapeutically effective dose can be estimated initially from cell culture assays. A dose may be formulated in animal models to achieve a circulating plasma concentration range that includes the IC₅₀ (i.e., the concentration of the test compound that achieves a half-maximal inhibition of symptoms) as determined in cell culture. Such information can be used to more accurately determine useful doses in humans. Levels in brain tissue or plasma may be measured, for example, by ELISA or high performance liquid chromatography (HPLC).

Prophylactic and/or therapeutic agents, as well as combinations thereof, can be tested in suitable animal model systems prior to use in humans. Such animal model systems include, but are not limited to, rats, mice, chicken, cows, monkeys, pigs, dogs, rabbits, etc. Any animal system well-known in the art may be used. Such model systems are widely used and well known to the skilled artisan. In some preferred embodiments, animal model systems for a CNS condition are used that are based on rats, mice, or other small mammal other than a primate.

Once the prophylactic and/or therapeutic agents of the invention have been tested in an animal model, they can be tested in clinical trials to establish their efficacy. Establishing clinical trials will be done in accordance with common methodologies known to one skilled in the art, and the optimal dosages and routes of administration as well as toxicity profiles of agents of the invention can be established. For example, a clinical trial can be designed to test a rAAV molecule of the invention for efficacy and toxicity in human patients.

A rAAV molecule of the invention generally will be administered for a time and in an amount effective for obtain a desired therapeutic and/or prophylactic benefit. The data obtained from the cell culture assays and animal studies can be used in formulating a range and/or schedule for dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized.

A therapeutically effective dosage of an rAAV vector for patients is generally from about 0.1 ml to about 100 ml of solution containing concentrations of from about 1×10⁹ to about 1×10¹⁶ genomes rAAV vector, or about 1×10¹⁰ to about 1×10¹⁵, about 1×10¹² to about 1×10¹⁶, or about 1×10¹⁴ to about 1×10¹⁶ AAV genomes. Levels of expression of the transgene can be monitored to determine/adjust dosage amounts, frequency, scheduling, and the like.

Treatment of a subject with a therapeutically or prophylactically effective amount of the agents of the invention can include a single treatment or can include a series of treatments. For example, pharmaceutical compositions comprising an agent of the invention may be administered once a day, twice a day, or three times a day. In some embodiments, the agent may be administered once a day, every other day, once a week, twice a week, once every two weeks, once a month, once every six weeks, once every two months, twice a year, or once per year. It will also be appreciated that the effective dosage of certain agents, e.g., the effective dosage of agents comprising a dual antigen-binding molecule of the invention, may increase or decrease over the course of treatment.

Methods of administering agents of the invention include, but are not limited to, intracerebroventricular (ICV), intracisternal (IC), including direct injection into the cisterna magna, lumbar intrathecal (IT) or intraparenchymal administration.

In certain embodiments, the agents of the invention are administered intravenously and may be administered together with other biologically active agents. In certain embodiments, the transgene is administered intravenously even if intended to be expressed in the CNS.

### 5.10. Pharmaceutical Compositions and Kits

The invention further provides a pharmaceutical composition comprising a pharmaceutically acceptable carrier and an agent of the invention, said agent comprising a rAAV molecule of the invention comprising a transgene cassette wherein the transgene expression is driven by the chimeric regulatory elements described herein. In preferred embodiments, the pharmaceutical composition comprises rAAV combined with a pharmaceutically acceptable carrier for administration to a subject. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's complete and incomplete adjuvant), excipient, or vehicle with which the agent is administered. Such pharmaceutical carriers can be sterile liquids, such as water and oils, including those of petroleum, animal, vegetable, or synthetic origin, including, e.g., peanut oil, soybean oil, mineral oil, sesame oil and the like. Water is a common carrier when the pharmaceutical composition is administered intravenously. Saline solutions and aqueous dextrose and glycerol solutions can also be employed as liquid carriers, particularly for injectable solutions. Suitable pharmaceutically acceptable carriers, excipients, and stabilizers are employed. These compositions can take the form of solutions, suspensions, emulsion, tablets, pills, capsules, powders, sustained-release formulations and the like.

In certain embodiments of the invention, pharmaceutical compositions are provided for use in accordance with the methods of the invention, said pharmaceutical compositions comprising a therapeutically and/or prophylactically effective amount of an agent of the invention along with a pharmaceutically acceptable carrier.

In preferred embodiments, the agent of the invention is substantially purified (i.e., substantially free from substances that limit its effect or produce undesired side-effects). In a specific embodiment, the host or subject is an animal, preferably a mammal such as non-primate (e.g., cows, pigs, horses, cats, dogs, rats etc.) and a primate (e.g., monkey such as, a cynomolgus monkey and a human). In a preferred embodiment, the host is a human.

The invention provides further kits that can be used in the above methods. In one embodiment, a kit comprises one or more agents of the invention, e.g., in one or more containers. In another embodiment, a kit further comprises one or more other prophylactic or therapeutic agents useful for the treatment of a condition, in one or more containers.

The invention also provides agents of the invention packaged in a hermetically sealed container such as an ampoule or sachette indicating the quantity of the agent or active agent. In one embodiment, the agent is supplied as a dry sterilized lyophilized powder or water free concentrate in a hermetically sealed container and can be reconstituted, e.g., with water or saline, to the appropriate concentration for administration to a subject. Typically, the agent is supplied as a dry sterile lyophilized powder in a hermetically sealed container at a unit dosage of at least 5 mg, more often at least 10 mg, at least 15 mg, at least 25 mg, at least 35 mg, at least 45 mg, at least 50 mg, or at least 75 mg. The lyophilized agent should be stored at between 2 and 8° C. in its original container and the agent should be administered within 12 hours, usually within 6 hours, within 5 hours, within 3 hours, or within 1 hour after being reconstituted. In an alternative embodiment, an agent of the invention is supplied in liquid form in a hermetically sealed container indicating the quantity and concentration of agent or active agent. Typically, the liquid form of the agent is supplied in a hermetically sealed container at least 1 mg/ml, at least 2.5 mg/ml, at least 5 mg/ml, at least 8 mg/ml, at least 10 mg/ml, at least 15 mg/kg, or at least 25 mg/ml.

The invention further provides a pharmaceutical pack or kit comprising one or more containers filled with one or more of the agents of the invention. Additionally, one or more other prophylactic or therapeutic agents useful for the treatment of the target disease or disorder can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use, or sale for human administration.

Generally, the ingredients of compositions of the invention are supplied either separately or mixed together in unit dosage form, for example, as a dry lyophilized powder or water-free concentrate in a hermetically sealed container such as an ampoule or sachette indicating the quantity of agent or active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

### 6. EXAMPLES

### 6.1. Example 1—Construction of Expression Cassettes

### 6.1.1. Tau-Specific Full-Length mAb Transgenes

FIGS. 1B and 1C depict various arrangements of expression cassettes for use with any mAb or mAb fragment transgene described herein. Full-length vectorized IgG antibody gene cassettes (nucleotide sequences encoding the antibodies listed in Table 5) were cloned into AAV proviral plasmids using standard restriction enzyme and T4 ligation based cloning methods. Specifically, proviral plasmids containing the AAV cassette were digested with EcoRI and XhoI restriction enzymes. Coding sequences for each vectorized antibody were digested with these same restriction enzymes to produce sticky ends for ligation into the AAV cassette using T4 DNA ligase. The cassettes carry both a heavy chain and light chain gene that following expression of the heavy chain and light chain polypeptides will heterodimerize to form a full antibody quadroma. The following is a description of each construct:

43A11 IgG2a (plasmid pAAV.CAG.chim.tau.43a11):

This cassette encodes an isotype control full-length antibody (43a11) used in studies as a comparator. The variable regions of control antibody 43a11 do not recognize a specific antigen. This construct contains conserved regions including the mouse IgG2a constant region and kappa light chain constant region. The transgene is driven by a CAG promoter.

4P3 IgG2a (plasmid pAAV.CAG.chim.tau.4P3):

This cassette encodes a chimeric human/mouse full-length antibody 4.P3. The variable regions recognize human tau at 217-TPPTREPKKVA-227 (SEQ ID NO: 120) and 249-PMPDLKN-255 (SEQ ID NO: 121). Conserved regions include the mouse IgG2a and kappa light chain. The transgene is driven by a CAG promoter.

4P3 IgG2a LALAPG (plasmid pAAV.CAG.chim.tau.4P3.LALAPGmut):

This cassette encodes a chimeric human/mouse full-length antibody 4.P3. The variable regions recognize human tau at 217-TPPTREPKKVA-227 and 249-PMPDLKN-255. Conserved regions include the mouse IgG2a and kappa light chain. The IgG2a sequence contains amino acid substitutions at positions 234, 235 and 329 that ablate antibody effector functions. The transgene is driven by a CAG promoter.

31B6 IgG2a (plasmid pAAV.CAG.chim.tau.31.B6):

This cassette encodes a chimeric human/mouse full-length antibody 31.B6. The variable regions recognize human tau at phosphorylated residues pS202/pT205. Conserved regions include the mouse IgG2a and lambda light chain. The transgene is driven by a CAG promoter.

31B6 IgG2a LALAPG (plasmid pAAV.CAG.chim.tau.31B6.LALAPGmut):

This cassette encodes a chimeric human/mouse full-length antibody 31.B6. The variable regions recognize human tau at phosphorylated residues pS202/pT205. Conserved regions include the mouse IgG2a and lambda light chain. The IgG2a sequence contains amino acid substitutions at positions 234, 235 and 329 that ablate antibody effector functions. The transgene is driven by a CAG promoter.

8H1 IgG2a (plasmid pAAV.CAG.chim.tau.8H1):

This cassette encodes a chimeric human/mouse full-length antibody 8.H1. The variable regions recognize phosphorylated human tau at positions pT212/pS214. Conserved regions include the mouse IgG2a and kappa light chain. The transgene is driven by a CAG promoter.

8H1 IgG2a LALAPG (plasmid pAAV.CAG.chim.tau.8H1.LALAPGmut):

This cassette encodes a chimeric human/mouse full-length antibody 8.H1. The variable regions recognize phosphorylated human tau at positions pT212/pS214. Conserved regions include the mouse IgG2a and kappa light chain. The IgG2a sequence contains amino acid substitutions at positions 234, 235 and 329 that ablate antibody effector functions. The transgene is driven by a CAG promoter.

4P3 IgG1 (plasmid pAAV.CAG.hu.tau.4p3):

This cassette encodes a fully human full-length antibody 4.P3. The variable regions recognize human tau at 217-TPPTREPKKVA-227 and 249-PMPDLKN-255. Conserved regions include the human IgG1 and kappa light chain. The transgene is driven by a CAG promoter.

31B6 IgG1 (plasmid pAAV.CAG.hu.tau.31b6):

This cassette encodes a fully human full-length antibody 31.B6. The variable regions recognize human tau at phosphorylated residues pS202/pT205. Conserved regions include the human IgG1 and lambda light chain. The transgene is driven by a CAG promoter.

8H1 IgG1 (plasmid pAAV.CAG.hu.tau.8h1):

This cassette encodes a fully human full-length antibody 8.H1. The variable regions recognize phosphorylated human tau at positions pT212/p S214. Conserved regions include the human IgG1 and kappa light chain. The transgene is driven by a CAG promoter.

### 6.1.2. Tau-Specific Single Chain mAb Transgenes

Nucleic acid sequences encoding ScFv and ScFv-Fc constructs (single-chain Fv polypeptides contiguous with an Fc domain) were prepared analogously and expressed from Cis plasmids.

TABLE 11A
ScFv and ScFv-Fc transgene Component Amino Acid Sequences

| | SEQ | |
|---|---|---|
| Name | ID NO: | Amino Acid Sequence |
| 4P3 VH | 97 | EVQLVQSGAEVKKPGESLKISCKVSGYDFSKYWIGWVRQMPGKGLEWMGLI |
| | | YPGDSDTRYSPSLEGQVSISADKSISAAYLQFSSLKASDSAMYYCVRKGYS |
| | | AYLGGWFDPWGQGTLVTVSS |
| 4P3 VL | 98 | DIQLTQSPDSLAASLGERVTINCRSSQNIFYNSNNKNFLAWYQQKAGQSPK |
| | | LLIYWASTRESGVPDRFSGSGSGTDFTLTISGLQAGDVAVYFCQQYFTDAP |
| | | TFGGGTKVEIK |
| 31B6 VH | 99 | EVQLLQSGGGVVQPGGSLRLSCEASGFTFSSYSMHWVRQAPGKGLEWLTLI |
| | | SYNGVNKYYAKSVKGRFAISRDDSKSTLFLQMNSLTTDDTAVYYCARCPIP |
| | | RAASYRNGDNYFEFGQGTLVTVSS |
| 31B6 VL | 100 | QAVVTQEPSLSVSPGGTVTLTCALSSASVSTSSYPSWYQQTPGQPPRMLIH |
| | | NTDTRSSGVPDRFSGSILGKKAALTITGAQADDESDYYCVVYLGNGFWVFG |
| | | GGTKLTVL |
| 8H1 VH | 101 | EVQLVESGGGLVQPGGSLRLSCAASGFTFSIYTMSWVRQAPGKGLEWVSAI |
| | | LSDGSTYYADSVEGRFTISRDNSKTTLYLQMNSLTAEDTAVYYCTKRVEGS |
| | | FRGWGQGTLVTVSS |
| 8H1 VL | 102 | EIVMTQSPGTLSLSPGESATLSCRASQSVSTTYLAWYQQKPGQAPRLLIYG |
| | | ASSRATGIPDRFSGSGSGTDFTLTISRLEPEDFAVYYCQQYGGSRLITFGQ |
| | | GTRLEIK |
| 4GS2 | 226 | GGGGSGGGGS |
| 4GS3 | 227 | GGGGSGGGGSGGGGS |
| 4GS4 | 228 | GGGGSGGGGSGGGGSGGGGS |
| IgG2a Hinge | 229 | EPRGPTIKPCPPCKCP |
| 5G Modified | 230 | GGGGGEPRGPTIKPCPPCKCP |
| IgG2a Hinge | | |
| 9G Modified | 231 | GGGGGGGGGEPRGPTIKPCPPCKCP |
| IgG2a Hinge | | |
| IgG2a | 232 | APNLLGGPSVFIFPPKIKDVLMISLSPIVTCVVVDVSEDDPDVQISWFVNN |
| CH2/CH3 | | VEVHTAQTQTHREDYNSTLRVVSALPIQHQDWMSGKEFKCKVNNKDLPAPI |
| (IgG2a Fc) | | ERTISKPKGSVRAPQVYVLPPPEEEMTKKQVTLTCMVTDEMPEDIYVEWTN |
| | | NGKTELNYKNTEPVLDSDGSYFMYSKLRVEKKNWVERNSYSCSVVHEGLHN |
| | | HHTTKSFSRTPGK |
| | | Lower hinge is shown in italic. |
| IgG2a | 233 | APNAAGGPSVFIFPPKIKDVLMISLSPIVTCVVVDVSEDDPDVQISWFVNN |
| CH2/CH3- | | VEVHTAQTQTHREDYNSTLRVVSALPIQHQDWMSGKEFKCKVNNKDLGAPI |
| LALAPG | | ERTISKPKGSVRAPQVYVLPPPEEEMTKKQVTLTCMVTDEMPEDIYVEWTN |
| (IgG2a Fc- | | NGKTELNYKNTEPVLDSDGSYFMYSKLRVEKKNWVERNSYSCSVVHEGLHN |
| LALAPG) | | HHTTKSFSRTPGK |
| | | Lower hinge is shown in italic. LALA-PG is shown in bold. |

Component parts listed in Table 6 and Table 11A were arranged to construct various ScFv-Fc polypeptides, the amino acid sequences of which are provided in Table 11B and exemplary nucleotide sequences encoding certain components are provided in Table 11D. Nucleic acid sequences encoding the ScFv-Fc polypeptides of Table 11E were cloned into Cis plasmids for testing and for making rAAV. Exemplary nucleotide sequences encoding the ScFv-Fc polypeptides are provided below in Table 11E.

TABLE 11B
ScFv-Fc Amino Acid Sequences

| | ScFv-Fc | | | | IgG hinge or | |
|---|---|---|---|---|---|---|
| ScFv-Fc | SEQ ID | | Linker | | Glycine Modified | IgG |
| Name | NO: | VH or VL | (4GS)ₓ | VL or VH | IgG hinge | CH2/CH3 |
| 4P3- | 234 | 4P3 VH | 4GS2 | 4P3 VL | IgG2a Hinge | IgG2a |
| VH2VL- | | | | | | |
| IgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDFMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 4P3- | 235 | 4P3 VH | 4GS2 | 4P3 VL | IgG4 Hinge | IgG4 |
| VH2VL- | EVQLVQSGAEVKKPGESLKISCKVSGYDFSKYWIGWVRQMPGKGLEWMGLIYPGDSDTRYSP | | | | | |
| IgG4 | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKESK | | | | | |
| | YGPPCPSCPAPEFLGGPSVFLFPPKPKDTLMISRTPEVTCVVVDVSQEDPEVQFNWYVDGVE | | | | | |
| | VHNAKTKPREEQFNSTYRVVSVLTVLHQDWLNGKEYKCKVSNKGLPSSIEKTISKAKGQPRE | | | | | |
| | PQVYTLPPSQEEMTKNQVSLTCLVKGFYPSDIAVEWESNGQPENNYKTTPPVLDSDGSFFLY | | | | | |
| | SRLTVDKSRWQEGNVFSCSVMHEALHNHYTQKSLSLSLGK | | | | | |
| 4P3- | 236 | 4P3 VH | 4GS2 | 4P3 VL | 5G Modified IgG2a | IgG2a |
| VH2VL- | | | | | Hinge | |
| 5GIgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKGGG | | | | | |
| | GGEPRGPTIKPCPPCKCPAPNLLGGPSVFIFPPKIKDVLMISLSPIVTCVVVDVSEDDPDVQ | | | | | |
| | ISWFVNNVEVHTAQTQTHREDYNSTLRVVSALPIQHQDWMSGKEFKCKVNNKDLPAPIERTI | | | | | |
| | SKPKGSVRAPQVYVLPPPEEEMTKKQVTLTCMVTDEMPEDIYVEWTNNGKTELNYKNTEPVL | | | | | |
| | DSDGSYFMYSKLRVEKKNWVERNSYSCSVVHEGLHNHHTTKSFSRTPGK | | | | | |
| 4P3- | 237 | 4P3 VH | 4GS2 | 4P3 VL | 5G Modified IgG4 | IgG4 |
| VH2VL- | | | | | Hinge | |
| IgG4 | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKGGG | | | | | |
| | GGESKYGPPCPSCPAPEFLGGPSVFLFPPKPKDTLMISRTPEVTCVVVDVSQEDPEVQFNWY | | | | | |
| | VDGVEVHNAKTKPREEQFNSTYRVVSVLTVLHQDWLNGKEYKCKVSNKGLPSSIEKTISKAK | | | | | |
| | GQPREPQVYTLPPSQEEMTKNQVSLTCLVKGFYPSDIAVEWESNGQPENNYKTTPPVLDSDG | | | | | |
| | SFFLYSRLTVDKSRWQEGNVFSCSVMHEALHNHYTQKSLSLSLGK | | | | | |
| 4P3- | 238 | 4P3 VH | 4GS2 | 4P3 VL | 9G Modified IgG2a | IgG2a |
| VH2VL- | | | | | Hinge | |
| 9GIgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKGGG | | | | | |
| | GGGGGGEPRGPTIKPCPPCKCPAPNLLGGPSVFIFPPKIKDVLMISLSPIVTCVVVDVSEDD | | | | | |
| | PDVQISWFVNNVEVHTAQTQTHREDYNSTLRVVSALPIQHQDWMSGKEFKCKVNNKDLPAPI | | | | | |
| | ERTISKPKGSVRAPQVYVLPPPEEEMTKKQVTLTCMVTDFMPEDIYVEWTNNGKTELNYKNT | | | | | |
| | EPVLDSDGSYFMYSKLRVEKKNWVERNSYSCSVVHEGLHNHHTTKSFSRTPGK | | | | | |
| 4P3- | 239 | 4P3 VH | 4GS2 | 4P3 VL | 9G Modified IgG4 | IgG4 |
| VH2VL- | | | | | Hinge | |
| 9GIgG4 | EVQLVQSGAEVKKPGESLKISCKVSGYDESKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSDIQLTQSPDSLAASLGERVTINCRSSQNIFYNSNNKNFLAWYQQKAGQSPKLLI | | | | | |
| | YWASTRESGVPDRFSGSGSGTDFTLTISGLQAGDVAVYFCQQYFTDAPTFGGGTKVEIKGGG | | | | | |
| | GGGGGGESKYGPPCPSCPAPEFLGGPSVFLFPPKPKDTLMISRTPEVTCVVVDVSQEDPEVQ | | | | | |
| | FNWYVDGVEVHNAKTKPREEQFNSTYRVVSVLTVLHQDWLNGKEYKCKVSNKGLPSSIEKTI | | | | | |
| | SKAKGQPREPQVYTLPPSQEEMTKNQVSLTCLVKGFYPSDIAVEWESNGQPENNYKTTPPVL | | | | | |
| | DSDGSFFLYSRLTVDKSRWQEGNVFSCSVMHEALHNHYTQKSLSLSLGK | | | | | |
| 4P3- | 240 | 4P3 VH | 4GS3 | 4P3 VL | IgG2a Hinge | IgG2a |
| VH3VL- | EVQLVQSGAEVKKPGESLKISCKVSGYDFSKYWIGWVRQMPGKGLEWMGLIYPGDSDTRYSP | | | | | |
| IgG2a | SLEGQVSISADKSISAAYLQFSSLKASDSAMYYCVRKGYSAYLGGWFDPWGQGTLVTVSSGG | | | | | |
| | GGSGGGGSGGGGSDIQLTQSPDSLAASLGERVTINCRSSQNIFYNSNNKNFLAWYQQKAGQS | | | | | |
| | PKLLIYWASTRESGVPDRFSGSGSGTDFTLTISGLQAGDVAVYFCQQYFTDAPTFGGGTKVE | | | | | |
| | IKEPRGPTIKPCPPCKCPAPNLLGGPSVFIFPPKIKDVLMISLSPIVTCVVVDVSEDDPDVQ | | | | | |
| | ISWFVNNVEVHTAQTQTHREDYNSTLRVVSALPIQHQDWMSGKEFKCKVNNKDLPAPIERTI | | | | | |
| | SKPKGSVRAPQVYVLPPPEEEMTKKQVTLTCMVTDEMPEDIYVEWTNNGKTELNYKNTEPVL | | | | | |
| | DSDGSYFMYSKLRVEKKNWVERNSYSCSVVHEGLHNHHTTKSFSRTPGK | | | | | |
| 4P3- | 241 | 4P3 VH | 4GS3 | 4P3 VL | 5G Modified IgG2a | IgG2a |
| VH3VL- | | | | | Hinge | |
| 5GIgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSGGGGSDIQLTQSPDSLAASLGERVTINCRSSQNIFYNSNNKNFLAWYQQKAGQS | | | | | |
| | PKLLIYWASTRESGVPDRFSGSGSGTDFTLTISGLQAGDVAVYFCQQYFTDAPTFGGGTKVE | | | | | |
| | IKGGGGGEPRGPTIKPCPPCKCPAPNLLGGPSVFIFPPKIKDVLMISLSPIVTCVVVDVSED | | | | | |
| | DPDVQISWFVNNVEVHTAQTQTHREDYNSTLRVVSALPIQHQDWMSGKEFKCKVNNKDLPAP | | | | | |
| | IERTISKPKGSVRAPQVYVLPPPEEEMTKKQVTLTCMVTDEMPEDIYVEWTNNGKTELNYKN | | | | | |
| | TEPVLDSDGSYFMYSKLRVEKKNWVERNSYSCSVVHEGLHNHHTTKSFSRTPGK | | | | | |
| 4P3- | 242 | 4P3 VH | 4GS3 | 4P3 VL | 9G Modified IgG2a | IgG2a |
| VH3VL- | | | | | Hinge | |
| 9GIgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSGGGGSDIQLTQSPDSLAASLGERVTINCRSSQNIFYNSNNKNFLAWYQQKAGQS | | | | | |
| | PKLLIYWASTRESGVPDRFSGSGSGTDFTLTISGLQAGDVAVYFCQQYFTDAPTFGGGTKVE | | | | | |
| | IKGGGGGGGGGEPRGPTIKPCPPCKCPAPNLLGGPSVFIFPPKIKDVLMISLSPIVTCVVVD | | | | | |
| | VSEDDPDVQISWFVNNVEVHTAQTQTHREDYNSTLRVVSALPIQHQDWMSGKEFKCKVNNKD | | | | | |
| | LPAPIERTISKPKGSVRAPQVYVLPPPEEEMTKKQVTLTCMVTDFMPEDIYVEWTNNGKTEL | | | | | |
| | NYKNTEPVLDSDGSYFMYSKLRVEKKNWVERNSYSCSVVHEGLHNHHTTKSFSRTPGK | | | | | |
| 4P3- | 243 | 4P3 VH | 4GS4 | 4P3 VL | IgG2a Hinge | IgG2a |
| VH4VL- | EVQLVQSGAEVKKPGESLKISCKVSGYDFSKYWIGWVRQMPGKGLEWMGLIYPGDSDTRYSP | | | | | |
| IgG2a | SLEGQVSISADKSISAAYLQFSSLKASDSAMYYCVRKGYSAYLGGWFDPWGQGTLVTVSSGG | | | | | |
| | GGSGGGGSGGGGSGGGGSDIQLTQSPDSLAASLGERVTINCRSSQNIFYNSNNKNFLAWYQQ | | | | | |
| | KAGQSPKLLIYWASTRESGVPDRFSGSGSGTDFTLTISGLQAGDVAVYFCQQYFTDAPTFGG | | | | | |
| | GTKVEIKEPRGPTIKPCPPCKCPAPNLLGGPSVFIFPPKIKDVLMISLSPIVTCVVVDVSED | | | | | |
| | DPDVQISWFVNNVEVHTAQTQTHREDYNSTLRVVSALPIQHQDWMSGKEFKCKVNNKDLPAP | | | | | |
| | IERTISKPKGSVRAPQVYVLPPPEEEMTKKQVTLTCMVTDEMPEDIYVEWTNNGKTELNYKN | | | | | |
| | TEPVLDSDGSYFMYSKLRVEKKNWVERNSYSCSVVHEGLHNHHTTKSFSRTPGK | | | | | |
| 4P3- | 244 | 4P3 VH | 4GS4 | 4P3 VL | 5G Modified IgG2a | IgG2a |
| VH4VL- | | | | | Hinge | |
| 5GIgG2a | EVQLVQSGAEVKKPGESLKISCKVSGYDESKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSGGGGSGGGGSDIQLTQSPDSLAASLGERVTINCRSSQNIFYNSNNKNFLAWYQQ | | | | | |
| | KAGQSPKLLIYWASTRESGVPDRFSGSGSGTDFTLTISGLQAGDVAVYFCQQYFTDAPTFGG | | | | | |
| | GTKVEIKGGGGGEPRGPTIKPCPPCKCPAPNLLGGPSVFIFPPKIKDVLMISLSPIVTCVVV | | | | | |
| | DVSEDDPDVQISWFVNNVEVHTAQTQTHREDYNSTLRVVSALPIQHQDWMSGKEFKCKVNNK | | | | | |
| | DLPAPIERTISKPKGSVRAPQVYVLPPPEEEMTKKQVTLTCMVTDEMPEDIYVEWTNNGKTE | | | | | |
| | LNYKNTEPVLDSDGSYFMYSKLRVEKKNWVERNSYSCSVVHEGLHNHHTTKSFSRTPGK | | | | | |
| 4P3- | 245 | 4P3 VH | 4GS4 | 4P3 VL | 9G Modified IgG2a | IgG2a |
| VH4VL- | | | | | Hinge | |
| 9GIgG2a | EVQLVQSGAEVKKPGESLKISCKVSGYDFSKYWIGWVRQMPGKGLEWMGLIYPGDSDTRYSP | | | | | |
| | SLEGQVSISADKSISAAYLQFSSLKASDSAMYYCVRKGYSAYLGGWFDPWGQGTLVTVSSGG | | | | | |
| | GGSGGGGSGGGGSGGGGSDIQLTQSPDSLAASLGERVTINCRSSQNIFYNSNNKNFLAWYQQ | | | | | |
| | KAGQSPKLLIYWASTRESGVPDRFSGSGSGTDFTLTISGLQAGDVAVYFCQQYFTDAPTFGG | | | | | |
| | GTKVEIKGGGGGGGGGEPRGPTIKPCPPCKCPAPNLLGGPSVFIFPPKIKDVLMISLSPIVT | | | | | |
| | CVVVDVSEDDPDVQISWFVNNVEVHTAQTQTHREDYNSTLRVVSALPIQHQDWMSGKEFKCK | | | | | |
| | VNNKDLPAPIERTISKPKGSVRAPQVYVLPPPEEEMTKKQVTLTCMVTDEMPEDIYVEWINN | | | | | |
| | GKTELNYKNTEPVLDSDGSYFMYSKLRVEKKNWVERNSYSCSVVHEGLHNHHTTKSFSRTPG | | | | | |
| | K | | | | | |
| 4P3- | 246 | 4P3 VL | 4GS2 | 4P3 VH | IgG2a Hinge | IgG2a |
| VL2VH- | DIQLTQSPDSLAASLGERVTINCRSSQNIFYNSNNKNFLAWYQQKAGQSPKLLIYWASTRES | | | | | |
| IgG2a | GVPDRFSGSGSGTDFTLTISGLQAGDVAVYFCQQYFTDAPTFGGGTKVEIKGGGGSGGGGSD | | | | | |
| | IQLTQSPDSLAASLGERVTINCRSSQNIFYNSNNKNFLAWYQQKAGQSPKLLIYWASTRESG | | | | | |
| | VPDRFSGSGSGTDFTLTISGLQAGDVAVYFCQQYFTDAPTFGGGTKVEIKEPRGPTIKPCPP | | | | | |
| | CKCPAPNLLGGPSVFIFPPKIKDVLMISLSPIVTCVVVDVSEDDPDVQISWFVNNVEVHTAQ | | | | | |
| | TQTHREDYNSTLRVVSALPIQHQDWMSGKEFKCKVNNKDLPAPIERTISKPKGSVRAPQVYV | | | | | |
| | LPPPEEEMTKKQVTLTCMVTDEMPEDIYVEWTNNGKTELNYKNTEPVLDSDGSYFMYSKLRV | | | | | |
| | EKKNWVERNSYSCSVVHEGLHNHHTTKSFSRTPGK | | | | | |
| 4P3- | 247 | 4P3 VL | 4GS2 | 4P3 VH | 5G Modified IgG2a | IgG2a |
| VL2VH- | | | | | Hinge | |
| 5GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSD | | | | | |
| | IQLTQSPDSLAASLGERVTINCRSSQNIFYNSNNKNFLAWYQQKAGQSPKLLIYWASTRESG | | | | | |
| | VPDRFSGSGSGTDFTLTISGLQAGDVAVYFCQQYFTDAPTFGGGTKVEIKGGGGGEPRGPTI | | | | | |
| | KPCPPCKCPAPNLLGGPSVFIFPPKIKDVLMISLSPIVTCVVVDVSEDDPDVQISWFVNNVE | | | | | |
| | VHTAQTQTHREDYNSTLRVVSALPIQHQDWMSGKEFKCKVNNKDLPAPIERTISKPKGSVRA | | | | | |
| | PQVYVLPPPEEEMTKKQVTLTCMVTDFMPEDIYVEWTNNGKTELNYKNTEPVLDSDGSYFMY | | | | | |
| | SKLRVEKKNWVERNSYSCSVVHEGLHNHHTTKSFSRTPGK | | | | | |
| 4P3- | 248 | 4P3 VL | 4GS2 | 4P3 VH | 9G Modified IgG2a | IgG2a |
| VL2VH- | | | | | Hinge | |
| 9GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSD | | | | | |
| | IQLTQSPDSLAASLGERVTINCRSSQNIFYNSNNKNFLAWYQQKAGQSPKLLIYWASTRESG | | | | | |
| | VPDRFSGSGSGTDFTLTISGLQAGDVAVYFCQQYFTDAPTFGGGTKVEIKGGGGGGGGGEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDEMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 4P3- | 249 | 4P3 VL | 4GS3 | 4P3 VH | IgG2a Hinge | IgG2a |
| VL3VH- | DIQLTQSPDSLAASLGERVTINCRSSQNIFYNSNNKNFLAWYQQKAGQSPKLLIYWASTRES | | | | | |
| IgG2a | GVPDRFSGSGSGTDFTLTISGLQAGDVAVYFCQQYFTDAPTFGGGTKVEIKGGGGSGGGGSG | | | | | |
| | GGGSDIQLTQSPDSLAASLGERVTINCRSSQNIFYNSNNKNFLAWYQQKAGQSPKLLIYWAS | | | | | |
| | TRESGVPDRFSGSGSGTDFTLTISGLQAGDVAVYFCQQYFTDAPTFGGGTKVEIKEPRGPTI | | | | | |
| | KPCPPCKCPAPNLLGGPSVFIFPPKIKDVLMISLSPIVTCVVVDVSEDDPDVQISWFVNNVE | | | | | |
| | VHTAQTQTHREDYNSTLRVVSALPIQHQDWMSGKEFKCKVNNKDLPAPIERTISKPKGSVRA | | | | | |
| | PQVYVLPPPEEEMTKKQVTLTCMVTDFMPEDIYVEWTNNGKTELNYKNTEPVLDSDGSYFMY | | | | | |
| | SKLRVEKKNWVERNSYSCSVVHEGLHNHHTTKSFSRTPGK | | | | | |
| 4P3- | 250 | 4P3 VL | 4GS3 | 4P3 VH | 5G Modified IgG2a | IgG2a |
| VL3 VH- | | | | | Hinge | |
| 5GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSG | | | | | |
| | GGGSDIQLTQSPDSLAASLGERVTINCRSSQNIFYNSNNKNFLAWYQQKAGQSPKLLIYWAS | | | | | |
| | TRESGVPDRFSGSGSGTDFTLTISGLQAGDVAVYFCQQYFTDAPTFGGGTKVEIKGGGGGEP | | | | | |
| | RGPTIKPCPPCKCPAPNLLGGPSVFIFPPKIKDVLMISLSPIVTCVVVDVSEDDPDVQISWE | | | | | |
| | VNNVEVHTAQTQTHREDYNSTLRVVSALPIQHQDWMSGKEFKCKVNNKDLPAPIERTISKPK | | | | | |
| | GSVRAPQVYVLPPPEEEMTKKQVTLTCMVTDFMPEDIYVEWTNNGKTELNYKNTEPVLDSDG | | | | | |
| | SYFMYSKLRVEKKNWVERNSYSCSVVHEGLHNHHTTKSFSRTPGK | | | | | |
| 4P3- | 251 | 4P3 VL | 4GS3 | 4P3 VH | 9G Modified IgG2a | IgG2a |
| VL3 VH- | | | | | Hinge | |
| 9GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSG | | | | | |
| | GGGSDIQLTQSPDSLAASLGERVTINCRSSQNIFYNSNNKNFLAWYQQKAGQSPKLLIYWAS | | | | | |
| | TRESGVPDRFSGSGSGTDFTLTISGLQAGDVAVYFCQQYFTDAPTFGGGTKVEIKGGGGGGG | | | | | |
| | GGEPRGPTIKPCPPCKCPAPNLLGGPSVFIFPPKIKDVLMISLSPIVTCVVVDVSEDDPDVQ | | | | | |
| | ISWFVNNVEVHTAQTQTHREDYNSTLRVVSALPIQHQDWMSGKEFKCKVNNKDLPAPIERTI | | | | | |
| | SKPKGSVRAPQVYVLPPPEEEMTKKQVTLTCMVTDFMPEDIYVEWTNNGKTELNYKNTEPVL | | | | | |
| | DSDGSYFMYSKLRVEKKNWVERNSYSCSVVHEGLHNHHTTKSFSRTPGK | | | | | |
| 4P3- | 252 | 4P3 VL | 4GS4 | 4P3 VH | IgG2a Hinge | IgG2a |
| VL4VH- | DIQLTQSPDSLAASLGERVTINCRSSQNIFYNSNNKNFLAWYQQKAGQSPKLLIYWASTRES | | | | | |
| IgG2a | GVPDRFSGSGSGTDFTLTISGLQAGDVAVYFCQQYFTDAPTFGGGTKVEIKGGGGSGGGGSG | | | | | |
| | GGGSGGGGSDIQLTQSPDSLAASLGERVTINCRSSQNIFYNSNNKNFLAWYQQKAGQSPKLL | | | | | |
| | IYWASTRESGVPDRFSGSGSGTDFTLTISGLQAGDVAVYFCQQYFTDAPTFGGGTKVEIKEP | | | | | |
| | RGPTIKPCPPCKCPAPNLLGGPSVFIFPPKIKDVLMISLSPIVTCVVVDVSEDDPDVQISWF | | | | | |
| | VNNVEVHTAQTQTHREDYNSTLRVVSALPIQHQDWMSGKEFKCKVNNKDLPAPIERTISKPK | | | | | |
| | GSVRAPQVYVLPPPEEEMTKKQVTLTCMVTDEMPEDIYVEWTNNGKTELNYKNTEPVLDSDG | | | | | |
| | SYFMYSKLRVEKKNWVERNSYSCSVVHEGLHNHHTTKSFSRTPGK | | | | | |
| 4P3- | 253 | 4P3 VL | 4GS4 | 4P3 VH | 5G Modified IgG2a | IgG2a |
| VL4VH- | | | | | Hinge | |
| 5GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSG | | | | | |
| | GGGSGGGGSDIQLTQSPDSLAASLGERVTINCRSSQNIFYNSNNKNFLAWYQQKAGQSPKLL | | | | | |
| | IYWASTRESGVPDRFSGSGSGTDFTLTISGLQAGDVAVYFCQQYFTDAPTFGGGTKVEIKGG | | | | | |
| | GGGEPRGPTIKPCPPCKCPAPNLLGGPSVFIFPPKIKDVLMISLSPIVTCVVVDVSEDDPDV | | | | | |
| | QISWFVNNVEVHTAQTQTHREDYNSTLRVVSALPIQHQDWMSGKEFKCKVNNKDLPAPIERT | | | | | |
| | ISKPKGSVRAPQVYVLPPPEEEMTKKQVTLTCMVTDFMPEDIYVEWTNNGKTELNYKNTEPV | | | | | |
| | LDSDGSYFMYSKLRVEKKNWVERNSYSCSVVHEGLHNHHTTKSFSRTPGK | | | | | |
| 4P3- | 254 | 4P3 VL | 4GS4 | 4P3 VH | 9G Modified IgG2a | IgG2a |
| VL4VH- | | | | | Hinge | |
| 9GIgG2a | DIQLTQSPDSLAASLGERVTINCRSSQNIFYNSNNKNFLAWYQQKAGQSPKLLIYWASTRES | | | | | |
| | GVPDRFSGSGSGTDFTLTISGLQAGDVAVYFCQQYFTDAPTFGGGTKVEIKGGGGSGGGGSG | | | | | |
| | GGGSGGGGSDIQLTQSPDSLAASLGERVTINCRSSQNIFYNSNNKNFLAWYQQKAGQSPKLL | | | | | |
| | IYWASTRESGVPDRFSGSGSGTDFTLTISGLQAGDVAVYFCQQYFTDAPTFGGGTKVEIKGG | | | | | |
| | GGGGGGGEPRGPTIKPCPPCKCPAPNLLGGPSVFIFPPKIKDVLMISLSPIVTCVVVDVSED | | | | | |
| | DPDVQISWFVNNVEVHTAQTQTHREDYNSTLRVVSALPIQHQDWMSGKEFKCKVNNKDLPAP | | | | | |
| | IERTISKPKGSVRAPQVYVLPPPEEEMTKKQVTLTCMVTDEMPEDIYVEWTNNGKTELNYKN | | | | | |
| | TEPVLDSDGSYFMYSKLRVEKKNWVERNSYSCSVVHEGLHNHHTTKSFSRTPGK | | | | | |
| 31B6- | 255 | 31B6 VH | 4GS2 | 31B6 VL | IgG2a Hinge | IgG2a |
| VH2VL- | EVQLLQSGGGVVQPGGSLRLSCEASGFTESSYSMHWVRQAPGKGLEWLTLISYNGVNKYYAK | | | | | |
| IgG2a | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLEP | | | | | |
| | RGPTIKPCPPCKCPAPNLLGGPSVFIFPPKIKDVLMISLSPIVTCVVVDVSEDDPDVQISWF | | | | | |
| | VNNVEVHTAQTQTHREDYNSTLRVVSALPIQHQDWMSGKEFKCKVNNKDLPAPIERTISKPK | | | | | |
| | GSVRAPQVYVLPPPEEEMTKKQVTLTCMVTDFMPEDIYVEWTNNGKTELNYKNTEPVLDSDG | | | | | |
| | SYFMYSKLRVEKKNWVERNSYSCSVVHEGLHNHHTTKSFSRTPGK | | | | | |
| 31B6- | 256 | 31B6 VH | 4GS2 | 31B6 VL | IgG4 Hinge | IgG4 |
| VH2VL- | EVQLLQSGGGVVQPGGSLRLSCEASGFTFSSYSMHWVRQAPGKGLEWLTLISYNGVNKYYAK | | | | | |
| IgG4 | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLES | | | | | |
| | KYGPPCPSCPAPEFLGGPSVFLFPPKPKDTLMISRTPEVTCVVVDVSQEDPEVQFNWYVDGV | | | | | |
| | EVHNAKTKPREEQFNSTYRVVSVLTVLHQDWLNGKEYKCKVSNKGLPSSIEKTISKAKGQPR | | | | | |
| | EPQVYTLPPSQEEMTKNQVSLTCLVKGFYPSDIAVEWESNGQPENNYKTTPPVLDSDGSFFL | | | | | |
| | YSRLTVDKSRWQEGNVFSCSVMHEALHNHYTQKSLSLSLGK | | | | | |
| 31B6- | 257 | 31B6 VH | 4GS2 | 31B6 VL | 5G Modified IgG2a | IgG2a |
| VH2VL- | | | | | Hinge | |
| 5GIgG2a | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLGG | | | | | |
| | GGGEPRGPTIKPCPPCKCPAPNLLGGPSVFIFPPKIKDVLMISLSPIVTCVVVDVSEDDPDV | | | | | |
| | QISWFVNNVEVHTAQTQTHREDYNSTLRVVSALPIQHQDWMSGKEFKCKVNNKDLPAPIERT | | | | | |
| | ISKPKGSVRAPQVYVLPPPEEEMTKKQVTLTCMVTDEMPEDIYVEWTNNGKTELNYKNTEPV | | | | | |
| | LDSDGSYFMYSKLRVEKKNWVERNSYSCSVVHEGLHNHHTTKSFSRTPGK | | | | | |
| 31B6- | 258 | 31B6 VH | 4GS2 | 31B6 VL | 5G Modified IgG4 | IgG4 |
| VH2VL- | | | | | Hinge | |
| 5GIgG4 | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLGG | | | | | |
| | GGGESKYGPPCPSCPAPEFLGGPSVFLFPPKPKDTLMISRTPEVTCVVVDVSQEDPEVQFNW | | | | | |
| | YVDGVEVHNAKTKPREEQFNSTYRVVSVLTVLHQDWLNGKEYKCKVSNKGLPSSIEKTISKA | | | | | |
| | KGQPREPQVYTLPPSQEEMTKNQVSLTCLVKGFYPSDIAVEWESNGQPENNYKTTPPVLDSD | | | | | |
| | GSFFLYSRLTVDKSRWQEGNVFSCSVMHEALHNHYTQKSLSLSLGK | | | | | |
| 31B6- | 259 | 31B6 VH | 4GS2 | 31B6 VL | 9G Modified IgG2a | IgG2a |
| VH2VL- | | | | | Hinge | |
| 9GIgG2a | EVQLLQSGGGVVQPGGSLRLSCEASGFTESSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLGG | | | | | |
| | GGGGGGGEPRGPTIKPCPPCKCPAPNLLGGPSVFIFPPKIKDVLMISLSPIVTCVVVDVSED | | | | | |
| | DPDVQISWFVNNVEVHTAQTQTHREDYNSTLRVVSALPIQHQDWMSGKEFKCKVNNKDLPAP | | | | | |
| | IERTISKPKGSVRAPQVYVLPPPEEEMTKKQVTLTCMVTDFMPEDIYVEWTNNGKTELNYKN | | | | | |
| | TEPVLDSDGSYFMYSKLRVEKKNWVERNSYSCSVVHEGLHNHHTTKSFSRTPGK | | | | | |
| 31B6- | 260 | 31B6 VH | 4GS2 | 31B6 VL | 9G Modified IgG4 | IgG4 |
| VH2VL- | | | | | Hinge | |
| 9GIgG4 | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSQAVVTQEPSLSVSPGGTVTLTCALSSASVSTSSYPSWYQQTPGQPPRMLI | | | | | |
| | HNTDTRSSGVPDRFSGSILGKKAALTITGAQADDESDYYCVVYLGNGFWVFGGGTKLTVLGG | | | | | |
| | GGGGGGGESKYGPPCPSCPAPEFLGGPSVFLFPPKPKDTLMISRTPEVTCVVVDVSQEDPEV | | | | | |
| | QFNWYVDGVEVHNAKTKPREEQFNSTYRVVSVLTVLHQDWLNGKEYKCKVSNKGLPSSIEKT | | | | | |
| | ISKAKGQPREPQVYTLPPSQEEMTKNQVSLTCLVKGFYPSDIAVEWESNGQPENNYKTTPPV | | | | | |
| | LDSDGSFFLYSRLTVDKSRWQEGNVFSCSVMHEALHNHYTQKSLSLSLGK | | | | | |
| 31B6- | 261 | 31B6 VH | 4GS3 | 31B6 VL | IgG2a Hinge | IgG2a |
| VH3VL- | EVQLLQSGGGVVQPGGSLRLSCEASGFTFSSYSMHWVRQAPGKGLEWLTLISYNGVNKYYAK | | | | | |
| IgG2a | SVKGRFAISRDDSKSTLFLQMNSLTTDDTAVYYCARCPIPRAASYRNGDNYFEFGQGTLVTV | | | | | |
| | SSGGGGSGGGGSGGGGSQAVVTQEPSLSVSPGGTVTLTCALSSASVSTSSYPSWYQQTPGQP | | | | | |
| | PRMLIHNTDTRSSGVPDRFSGSILGKKAALTITGAQADDESDYYCVVYLGNGFWVFGGGTKL | | | | | |
| | TVLEPRGPTIKPCPPCKCPAPNLLGGPSVFIFPPKIKDVLMISLSPIVTCVVVDVSEDDPDV | | | | | |
| | QISWFVNNVEVHTAQTQTHREDYNSTLRVVSALPIQHQDWMSGKEFKCKVNNKDLPAPIERT | | | | | |
| | ISKPKGSVRAPQVYVLPPPEEEMTKKQVTLTCMVTDFMPEDIYVEWTNNGKTELNYKNTEPV | | | | | |
| | LDSDGSYFMYSKLRVEKKNWVERNSYSCSVVHEGLHNHHTTKSFSRTPGK | | | | | |
| 31B6- | 262 | 31B6 VH | 4GS3 | 31B6 VL | 5G Modified IgG2a | IgG2a |
| VH3VL- | | | | | Hinge | |
| 5GIgG2a | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSGGGGSQAVVTQEPSLSVSPGGTVTLTCALSSASVSTSSYPSWYQQTPGQP | | | | | |
| | PRMLIHNTDTRSSGVPDRFSGSILGKKAALTITGAQADDESDYYCVVYLGNGFWVFGGGTKL | | | | | |
| | TVLGGGGGEPRGPTIKPCPPCKCPAPNLLGGPSVFIFPPKIKDVLMISLSPIVTCVVVDVSE | | | | | |
| | DDPDVQISWFVNNVEVHTAQTQTHREDYNSTLRVVSALPIQHQDWMSGKEFKCKVNNKDLPA | | | | | |
| | PIERTISKPKGSVRAPQVYVLPPPEEEMTKKQVTLTCMVTDFMPEDIYVEWTNNGKTELNYK | | | | | |
| | NTEPVLDSDGSYFMYSKLRVEKKNWVERNSYSCSVVHEGLHNHHTTKSFSRTPGK | | | | | |
| 31B6- | 263 | 31B6 VH | 4GS3 | 31B6 VL | 9G Modified IgG2a | IgG2a |
| VH3VL- | | | | | Hinge | |
| 9GIgG2a | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSGGGGSGGGGSQAVVTQEPSLSVSPGGTVTLTCALSSASVSTSSYPSWYQQ | | | | | |
| | TPGQPPRMLIHNTDTRSSGVPDRFSGSILGKKAALTITGAQADDESDYYCVVYLGNGFWVFG | | | | | |
| | GGTKLTVLEPRGPTIKPCPPCKCPAPNLLGGPSVFIFPPKIKDVLMISLSPIVTCVVVDVSE | | | | | |
| | DDPDVQISWFVNNVEVHTAQTQTHREDYNSTLRVVSALPIQHQDWMSGKEFKCKVNNKDLPA | | | | | |
| | PIERTISKPKGSVRAPQVYVLPPPEEEMTKKQVTLTCMVTDEMPEDIYVEWTNNGKTELNYK | | | | | |
| | NTEPVLDSDGSYFMYSKLRVEKKNWVERNSYSCSVVHEGLHNHHTTKSFSRTPGK | | | | | |
| 31B6- | 264 | 31B6 VH | 4GS4 | 31B6 VL | IgG2a Hinge | IgG2a |
| VH4VL- | EVQLLQSGGGVVQPGGSLRLSCEASGFTFSSYSMHWVRQAPGKGLEWLTLISYNGVNKYYAK | | | | | |
| IgG2a | SVKGRFAISRDDSKSTLFLQMNSLTTDDTAVYYCARCPIPRAASYRNGDNYFEFGQGTLVTV | | | | | |
| | SSGGGGSGGGGSGGGGSGGGGSQAVVTQEPSLSVSPGGTVTLTCALSSASVSTSSYPSWYQQ | | | | | |
| | TPGQPPRMLIHNTDTRSSGVPDRFSGSILGKKAALTITGAQADDESDYYCVVYLGNGFWVFG | | | | | |
| | GGTKLTVLEPRGPTIKPCPPCKCPAPNLLGGPSVFIFPPKIKDVLMISLSPIVTCVVVDVSE | | | | | |
| | DDPDVQISWFVNNVEVHTAQTQTHREDYNSTLRVVSALPIQHQDWMSGKEFKCKVNNKDLPA | | | | | |
| | PIERTISKPKGSVRAPQVYVLPPPEEEMTKKQVTLTCMVTDEMPEDIYVEWTNNGKTELNYK | | | | | |
| | NTEPVLDSDGSYFMYSKLRVEKKNWVERNSYSCSVVHEGLHNHHTTKSFSRTPGK | | | | | |
| 31B6- | 265 | 31B6 VH | 4GS4 | 31B6 VL | 5G Modified IgG2a | IgG2a |
| VH4VL- | | | | | Hinge | |
| 5GIgG2a | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSGGGGSGGGGSQAVVTQEPSLSVSPGGTVTLTCALSSASVSTSSYPSWYQQ | | | | | |
| | TPGQPPRMLIHNTDTRSSGVPDRFSGSILGKKAALTITGAQADDESDYYCVVYLGNGFWVFG | | | | | |
| | GGTKLTVLGGGGGEPRGPTIKPCPPCKCPAPNLLGGPSVFIFPPKIKDVLMISLSPIVTCVV | | | | | |
| | VDVSEDDPDVQISWFVNNVEVHTAQTQTHREDYNSTLRVVSALPIQHQDWMSGKEFKCKVNN | | | | | |
| | KDLPAPIERTISKPKGSVRAPQVYVLPPPEEEMTKKQVTLTCMVTDFMPEDIYVEWTNNGKT | | | | | |
| | ELNYKNTEPVLDSDGSYFMYSKLRVEKKNWVERNSYSCSVVHEGLHNHHTTKSFSRTPGK | | | | | |
| 31B6- | 266 | 31B6 VH | 4GS4 | 31B6 VL | 9G Modified IgG2a | IgG2a |
| VH4VL- | | | | | Hinge | |
| 9GIgG2a | EVQLLQSGGGVVQPGGSLRLSCEASGFTFSSYSMHWVRQAPGKGLEWLTLISYNGVNKYYAK | | | | | |
| | SVKGRFAISRDDSKSTLFLQMNSLTTDDTAVYYCARCPIPRAASYRNGDNYFEFGQGTLVTV | | | | | |
| | SSGGGGSGGGGSGGGGSGGGGSQAVVTQEPSLSVSPGGTVTLTCALSSASVSTSSYPSWYQQ | | | | | |
| | TPGQPPRMLIHNTDTRSSGVPDRFSGSILGKKAALTITGAQADDESDYYCVVYLGNGFWVFG | | | | | |
| | GGTKLTVLGGGGGGGGGEPRGPTIKPCPPCKCPAPNLLGGPSVFIFPPKIKDVLMISLSPIV | | | | | |
| | TCVVVDVSEDDPDVQISWFVNNVEVHTAQTQTHREDYNSTLRVVSALPIQHQDWMSGKEFKC | | | | | |
| | KVNNKDLPAPIERTISKPKGSVRAPQVYVLPPPEEEMTKKQVTLTCMVTDEMPEDIYVEWTN | | | | | |
| | NGKTELNYKNTEPVLDSDGSYFMYSKLRVEKKNWVERNSYSCSVVHEGLHNHHTTKSFSRTP | | | | | |
| | GK | | | | | |
| 31B6- | 267 | 31B6 VL | 4GS2 | 31B6 VH | IgG2a Hinge | IgG2a |
| VL2VH- | QAVVTQEPSLSVSPGGTVTLTCALSSASVSTSSYPSWYQQTPGQPPRMLIHNTDTRSSGVPD | | | | | |
| IgG2a | RFSGSILGKKAALTITGAQADDESDYYCVVYLGNGFWVFGGGTKLTVLGGGGSGGGGSEVQL | | | | | |
| | LQSGGGVVQPGGSLRLSCEASGFTFSSYSMHWVRQAPGKGLEWLTLISYNGVNKYYAKSVKG | | | | | |
| | RFAISRDDSKSTLFLQMNSLTTDDTAVYYCARCPIPRAASYRNGDNYFEFGQGTLVTVSSEP | | | | | |
| | RGPTIKPCPPCKCPAPNLLGGPSVFIFPPKIKDVLMISLSPIVTCVVVDVSEDDPDVQISWF | | | | | |
| | VNNVEVHTAQTQTHREDYNSTLRVVSALPIQHQDWMSGKEFKCKVNNKDLPAPIERTISKPK | | | | | |
| | GSVRAPQVYVLPPPEEEMTKKQVTLTCMVTDEMPEDIYVEWTNNGKTELNYKNTEPVLDSDG | | | | | |
| | SYFMYSKLRVEKKNWVERNSYSCSVVHEGLHNHHTTKSFSRTPGK | | | | | |
| 31B6- | 268 | 31B6 VL | 4GS2 | 31B6 VH | 5G Modified IgG2a | IgG2a |
| VL2VH- | | | | | Hinge | |
| 5GIgG2a | QAVVTQEPSLSVSPGGTVTLTCALSSASVSTSSYPSWYQQTPGQPPRMLIHNTDTRSSGVPD | | | | | |
| | RFSGSILGKKAALTITGAQADDESDYYCVVYLGNGFWVFGGGTKLTVLGGGGSGGGGSEVQL | | | | | |
| | LQSGGGVVQPGGSLRLSCEASGFTFSSYSMHWVRQAPGKGLEWLTLISYNGVNKYYAKSVKG | | | | | |
| | RFAISRDDSKSTLFLQMNSLTTDDTAVYYCARCPIPRAASYRNGDNYFEFGQGTLVTVSSGG | | | | | |
| | GGGEPRGPTIKPCPPCKCPAPNLLGGPSVFIFPPKIKDVLMISLSPIVTCVVVDVSEDDPDV | | | | | |
| | QISWFVNNVEVHTAQTQTHREDYNSTLRVVSALPIQHQDWMSGKEFKCKVNNKDLPAPIERT | | | | | |
| | ISKPKGSVRAPQVYVLPPPEEEMTKKQVTLTCMVTDFMPEDIYVEWTNNGKTELNYKNTEPV | | | | | |
| | LDSDGSYFMYSKLRVEKKNWVERNSYSCSVVHEGLHNHHTTKSFSRTPGK | | | | | |
| 31B6- | 269 | 31B6 VL | 4GS2 | 31B6 VH | 9G Modified IgG2a | IgG2a |
| VL2VH- | | | | | Hinge | |
| 9GIgG2a | QAVVTQEPSLSVSPGGTVTLTCALSSASVSTSSYPSWYQQTPGQPPRMLIHNTDTRSSGVPD | | | | | |
| | RFSGSILGKKAALTITGAQADDESDYYCVVYLGNGFWVFGGGTKLTVLGGGGSGGGGSEVQL | | | | | |
| | LQSGGGVVQPGGSLRLSCEASGFTFSSYSMHWVRQAPGKGLEWLTLISYNGVNKYYAKSVKG | | | | | |
| | RFAISRDDSKSTLFLQMNSLTTDDTAVYYCARCPIPRAASYRNGDNYFEFGQGTLVTVSSGG | | | | | |
| | GGGGGGGEPRGPTIKPCPPCKCPAPNLLGGPSVFIFPPKIKDVLMISLSPIVTCVVVDVSED | | | | | |
| | DPDVQISWFVNNVEVHTAQTQTHREDYNSTLRVVSALPIQHQDWMSGKEFKCKVNNKDLPAP | | | | | |
| | IERTISKPKGSVRAPQVYVLPPPEEEMTKKQVTLTCMVTDEMPEDIYVEWTNNGKTELNYKN | | | | | |
| | TEPVLDSDGSYFMYSKLRVEKKNWVERNSYSCSVVHEGLHNHHTTKSFSRTPGK | | | | | |
| 31B6- | 270 | 31B6 VL | 4GS3 | 31B6 VH | IgG2a Hinge | IgG2a |
| VL3VH- | QAVVTQEPSLSVSPGGTVTLTCALSSASVSTSSYPSWYQQTPGQPPRMLIHNTDTRSSGVPD | | | | | |
| IgG2a | RFSGSILGKKAALTITGAQADDESDYYCVVYLGNGFWVFGGGTKLTVLGGGGSGGGGSGGGG | | | | | |
| | SEVQLLQSGGGVVQPGGSLRLSCEASGFTFSSYSMHWVRQAPGKGLEWLTLISYNGVNKYYA | | | | | |
| | KSVKGRFAISRDDSKSTLFLQMNSLTTDDTAVYYCARCPIPRAASYRNGDNYFEFGQGTLVT | | | | | |
| | VSSEPRGPTIKPCPPCKCPAPNLLGGPSVFIFPPKIKDVLMISLSPIVTCVVVDVSEDDPDV | | | | | |
| | QISWFVNNVEVHTAQTQTHREDYNSTLRVVSALPIQHQDWMSGKEFKCKVNNKDLPAPIERT | | | | | |
| | ISKPKGSVRAPQVYVLPPPEEEMTKKQVTLTCMVTDFMPEDIYVEWTNNGKTELNYKNTEPV | | | | | |
| | LDSDGSYFMYSKLRVEKKNWVERNSYSCSVVHEGLHNHHTTKSFSRTPGK | | | | | |
| 31B6- | 271 | 31B6 VL | 4GS3 | 31B6 VH | 5G Modified IgG2a | IgG2a |
| VL3 VH- | | | | | Hinge | |

| | |
|---|---|
| 5GIgG2a | QAVVTQEPSLSVSPGGTVTLTCALSSASVSTSSYPSWYQQTPGQPPRMLIHNTDTRSSGVPD |
| | RFSGSILGKKAALTITGAQADDESDYYCVVYLGNGFWVFGGGTKLTVLGGGGSGGGGSGGGG |
| | SEVQLLQSGGGVVQPGGSLRLSCEASGFTFSSYSMHWVRQAPGKGLEWLTLISYNGVNKYYA |
| | KSVKGRFAISRDDSKSTLFLQMNSLTTDDTAVYYCARCPIPRAASYRNGDNYFEFGQGTLVT |
| | VSSGGGGGEPRGPTIKPCPPCKCPAPNLLGGPSVFIFPPKIKDVLMISLSPIVTCVVVDVSE |
| | DDPDVQISWFVNNVEVHTAQTQTHREDYNSTLRVVSALPIQHQDWMSGKEFKCKVNNKDLPA |
| | PIERTISKPKGSVRAPQVYVLPPPEEEMTKKQVTLTCMVTDEMPEDIYVEWTNNGKTELNYK |
| | NTEPVLDSDGSYFMYSKLRVEKKNWVERNSYSCSVVHEGLHNHHTTKSFSRTPGK |

| 31B6- | 272 | 31B6 VL | 4GS3 | 31B6 VH | 9G Modified IgG2a | IgG2a |
|---|---|---|---|---|---|---|
| VL3VH- | | | | | Hinge | |
| 9GIgG2a | QAVVTQEPSLSVSPGGTVTLTCALSSASVSTSSYPSWYQQTPGQPPRMLIHNTDTRSSGVPD | | | | | |
| | RFSGSILGKKAALTITGAQADDESDYYCVVYLGNGFWVFGGGTKLTVLGGGGGGGGSGGGG | | | | | |
| | SEVQLLQSGGGVVQPGGSLRLSCEASGFTFSSYSMHWVRQAPGKGLEWLTLISYNGVNKYYA | | | | | |
| | KSVKGRFAISRDDSKSTLFLQMNSLTTDDTAVYYCARCPIPRAASYRNGDNYFEFGQGTLVT | | | | | |
| | VSSGGGGGGGGGEPRGPTIKPCPPCKCPAPNLLGGPSVFIFPPKIKDVLMISLSPIVTCVVV | | | | | |
| | DVSEDDPDVQISWFVNNVEVHTAQTQTHREDYNSTLRVVSALPIQHQDWMSGKEFKCKVNNK | | | | | |
| | DLPAPIERTISKPKGSVRAPQVYVLPPPEEEMTKKQVTLTCMVTDFMPEDIYVEWTNNGKTE | | | | | |
| | LNYKNTEPVLDSDGSYFMYSKLRVEKKNWVERNSYSCSVVHEGLHNHHTTKSFSRTPGK | | | | | |
| 31B6- | 273 | 31B6 VL | 4GS4 | 31B6 VH | IgG2a Hinge | IgG2a |
| VL4VH- | QAVVTQEPSLSVSPGGTVTLTCALSSASVSTSSYPSWYQQTPGQPPRMLIHNTDTRSSGVPD | | | | | |
| IgG2a | RFSGSILGKKAALTITGAQADDESDYYCVVYLGNGFWVFGGGTKLTVLGGGGSGGGGSGGGG | | | | | |
| | SGGGGSEVQLLQSGGGVVQPGGSLRLSCEASGFTESSYSMHWVRQAPGKGLEWLTLISYNGV | | | | | |
| | NKYYAKSVKGRFAISRDDSKSTLFLQMNSLTTDDTAVYYCARCPIPRAASYRNGDNYFEFGQ | | | | | |
| | GTLVTVSSEPRGPTIKPCPPCKCPAPNLLGGPSVFIFPPKIKDVLMISLSPIVTCVVVDVSE | | | | | |
| | DDPDVQISWFVNNVEVHTAQTQTHREDYNSTLRVVSALPIQHQDWMSGKEFKCKVNNKDLPA | | | | | |
| | PIERTISKPKGSVRAPQVYVLPPPEEEMTKKQVTLTCMVTDEMPEDIYVEWTNNGKTELNYK | | | | | |
| | NTEPVLDSDGSYFMYSKLRVEKKNWVERNSYSCSVVHEGLHNHHTTKSFSRTPGK | | | | | |
| 31B6- | 274 | 31B6 VL | 4GS4 | 31B6 VH | 5G Modified IgG2a | IgG2a |
| VL4VH- | | | | | Hinge | |
| 5GIgG2a | QAVVTQEPSLSVSPGGTVTLTCALSSASVSTSSYPSWYQQTPGQPPRMLIHNTDTRSSGVPD | | | | | |
| | RFSGSILGKKAALTITGAQADDESDYYCVVYLGNGFWVFGGGTKLTVLGGGGSGGGGSGGGG | | | | | |
| | SGGGGSEVQLLQSGGGVVQPGGSLRLSCEASGFTFSSYSMHWVRQAPGKGLEWLTLISYNGV | | | | | |
| | NKYYAKSVKGRFAISRDDSKSTLFLQMNSLTTDDTAVYYCARCPIPRAASYRNGDNYFEFGQ | | | | | |
| | GTLVTVSSGGGGGEPRGPTIKPCPPCKCPAPNLLGGPSVFIFPPKIKDVLMISLSPIVTCVV | | | | | |
| | VDVSEDDPDVQISWFVNNVEVHTAQTQTHREDYNSTLRVVSALPIQHQDWMSGKEFKCKVNN | | | | | |
| | KDLPAPIERTISKPKGSVRAPQVYVLPPPEEEMTKKQVTLTCMVTDFMPEDIYVEWTNNGKT | | | | | |
| | ELNYKNTEPVLDSDGSYFMYSKLRVEKKNWVERNSYSCSVVHEGLHNHHTTKSFSRTPGK | | | | | |
| 31B6- | 275 | 31B6 VL | 4GS4 | 31B6 VH | 9G Modified IgG2a | IgG2a |
| VL4VH- | | | | | Hinge | |
| 9GIgG2a | QAVVTQEPSLSVSPGGTVTLTCALSSASVSTSSYPSWYQQTPGQPPRMLIHNTDTRSSGVPD | | | | | |
| | RFSGSILGKKAALTITGAQADDESDYYCVVYLGNGFWVFGGGTKLTVLGGGGSGGGGSGGGG | | | | | |
| | SGGGGSEVQLLQSGGGVVQPGGSLRLSCEASGFTFSSYSMHWVRQAPGKGLEWLTLISYNGV | | | | | |
| | NKYYAKSVKGRFAISRDDSKSTLFLQMNSLTTDDTAVYYCARCPIPRAASYRNGDNYFEFGQ | | | | | |
| | GTLVTVSSGGGGGGGGGEPRGPTIKPCPPCKCPAPNLLGGPSVFIFPPKIKDVLMISLSPIV | | | | | |
| | TCVVVDVSEDDPDVQISWFVNNVEVHTAQTQTHREDYNSTLRVVSALPIQHQDWMSGKEFKC | | | | | |
| | KVNNKDLPAPIERTISKPKGSVRAPQVYVLPPPEEEMTKKQVTLTCMVTDFMPEDIYVEWTN | | | | | |
| | NGKTELNYKNTEPVLDSDGSYFMYSKLRVEKKNWVERNSYSCSVVHEGLHNHHTTKSFSRTP | | | | | |
| | GK | | | | | |
| 8H1- | 276 | 8H1 VH | 4GS2 | 8H1 VL | IgG2a Hinge | IgG2a |
| VH2VL- | EVQLVESGGGLVQPGGSLRLSCAASGFTFSIYTMSWVRQAPGKGLEWVSAILSDGSTYYADS | | | | | |
| IgG2a | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRFSGSGSGTDFTLTISRLEPEDFAVYYCQQYGGSRLITFGQGTRLEIKEPRGPTIKPCPPC | | | | | |
| | KCPAPNLLGGPSVFIFPPKIKDVLMISLSPIVTCVVVDVSEDDPDVQISWFVNNVEVHTAQT | | | | | |
| | QTHREDYNSTLRVVSALPIQHQDWMSGKEFKCKVNNKDLPAPIERTISKPKGSVRAPQVYVL | | | | | |
| | PPPEEEMTKKQVTLTCMVTDFMPEDIYVEWTNNGKTELNYKNTEPVLDSDGSYFMYSKLRVE | | | | | |
| | KKNWVERNSYSCSVVHEGLHNHHTTKSFSRTPGK | | | | | |
| 8H1- | 277 | 8H1 VH | 4GS2 | 8H1 VL | IgG4 Hinge | IgG4 |
| VH2VL- | EVQLVESGGGLVQPGGSLRLSCAASGFTFSIYTMSWVRQAPGKGLEWVSAILSDGSTYYADS | | | | | |
| IgG4 | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRFSGSGSGTDFTLTISRLEPEDFAVYYCQQYGGSRLITFGQGTRLEIKESKYGPPCPSCPA | | | | | |
| | PEFLGGPSVFLFPPKPKDTLMISRTPEVTCVVVDVSQEDPEVQFNWYVDGVEVHNAKTKPRE | | | | | |
| | EQFNSTYRVVSVLTVLHQDWLNGKEYKCKVSNKGLPSSIEKTISKAKGQPREPQVYTLPPSQ | | | | | |
| | EEMTKNQVSLTCLVKGFYPSDIAVEWESNGQPENNYKTTPPVLDSDGSFFLYSRLTVDKSRW | | | | | |
| | QEGNVFSCSVMHEALHNHYTQKSLSLSLGK | | | | | |
| 8H1- | 278 | 8H1 VH | 4GS2 | 8H1 VL | 5G Modified IgG2a | IgG2a |
| VH2VL- | | | | | Hinge | |
| 5GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRFSGSGSGTDFTLTISRLEPEDFAVYYCQQYGGSRLITFGQGTRLEIKGGGGGEPRGPTIK | | | | | |
| | PCPPCKCPAPNLLGGPSVFIFPPKIKDVLMISLSPIVTCVVVDVSEDDPDVQISWFVNNVEV | | | | | |
| | HTAQTQTHREDYNSTLRVVSALPIQHQDWMSGKEFKCKVNNKDLPAPIERTISKPKGSVRAP | | | | | |
| | QVYVLPPPEEEMTKKQVTLTCMVTDFMPEDIYVEWTNNGKTELNYKNTEPVLDSDGSYFMYS | | | | | |
| | KLRVEKKNWVERNSYSCSVVHEGLHNHHTTKSFSRTPGK | | | | | |
| 8H1- | 279 | 8H1 VH | 4GS2 | 8H1 VL | 5G Modified IgG4 | IgG4 |
| VH2VL- | | | | | Hinge | |
| 5GIgG4 | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRFSGSGSGTDFTLTISRLEPEDFAVYYCQQYGGSRLITFGQGTRLEIKGGGGGESKYGPPC | | | | | |
| | PSCPAPEFLGGPSVFLFPPKPKDTLMISRTPEVTCVVVDVSQEDPEVQFNWYVDGVEVHNAK | | | | | |
| | TKPREEQFNSTYRVVSVLTVLHQDWLNGKEYKCKVSNKGLPSSIEKTISKAKGQPREPQVYT | | | | | |
| | LPPSQEEMTKNQVSLTCLVKGFYPSDIAVEWESNGQPENNYKTTPPVLDSDGSFFLYSRLTV | | | | | |
| | DKSRWQEGNVFSCSVMHEALHNHYTQKSLSLSLGK | | | | | |
| 8H1- | 280 | 8H1 VH | 4GS2 | 8H1 VL | 9G Modified IgG2a | IgG2a |
| VH2VL- | | | | | Hinge | |
| 9GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRFSGSGSGTDFTLTISRLEPEDFAVYYCQQYGGSRLITFGQGTRLEIKGGGGGGGGGEPRG | | | | | |
| | PTIKPCPPCKCPAPNLLGGPSVFIFPPKIKDVLMISLSPIVTCVVVDVSEDDPDVQISWFVN | | | | | |
| | NVEVHTAQTQTHREDYNSTLRVVSALPIQHQDWMSGKEFKCKVNNKDLPAPIERTISKPKGS | | | | | |
| | VRAPQVYVLPPPEEEMTKKQVTLTCMVTDFMPEDIYVEWTNNGKTELNYKNTEPVLDSDGSY | | | | | |
| | FMYSKLRVEKKNWVERNSYSCSVVHEGLHNHHTTKSFSRTPGK | | | | | |
| 8H1- | 281 | 8H1 VH | 4GS2 | 8H1 VL | 9G Modified IgG4 | IgG4 |
| VH2VL- | | | | | Hinge | |
| 9GIgG4 | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSEIVMTQSPGTLSLSPGESATLSCRASQSVSTTYLAWYQQKPGQAPRLLIYGASSRATGIP | | | | | |
| | DRESGSGSGTDFTLTISRLEPEDFAVYYCQQYGGSRLITFGQGTRLEIKGGGGGGGGGESKY | | | | | |
| | GPPCPSCPAPEFLGGPSVFLFPPKPKDTLMISRTPEVTCVVVDVSQEDPEVQFNWYVDGVEV | | | | | |
| | HNAKTKPREEQFNSTYRVVSVLTVLHQDWLNGKEYKCKVSNKGLPSSIEKTISKAKGQPREP | | | | | |
| | QVYTLPPSQEEMTKNQVSLTCLVKGFYPSDIAVEWESNGQPENNYKTTPPVLDSDGSFFLYS | | | | | |
| | RLTVDKSRWQEGNVFSCSVMHEALHNHYTQKSLSLSLGK | | | | | |
| 8H1- | 282 | 8H1 VH | 4GS3 | 8H1 VL | IgG2a Hinge | IgG2a |
| VH3VL- | EVQLVESGGGLVQPGGSLRLSCAASGFTFSIYTMSWVRQAPGKGLEWVSAILSDGSTYYADS | | | | | |
| IgG2a | VEGRFTISRDNSKTTLYLQMNSLTAEDTAVYYCTKRVEGSFRGWGQGTLVTVSSGGGGSGGG | | | | | |
| | GSGGGGSEIVMTQSPGTLSLSPGESATLSCRASQSVSTTYLAWYQQKPGQAPRLLIYGASSR | | | | | |
| | ATGIPDRFSGSGSGTDFTLTISRLEPEDFAVYYCQQYGGSRLITFGQGTRLEIKEPRGPTIK | | | | | |
| | PCPPCKCPAPNLLGGPSVFIFPPKIKDVLMISLSPIVTCVVVDVSEDDPDVQISWFVNNVEV | | | | | |
| | HTAQTQTHREDYNSTLRVVSALPIQHQDWMSGKEFKCKVNNKDLPAPIERTISKPKGSVRAP | | | | | |
| | QVYVLPPPEEEMTKKQVTLTCMVTDFMPEDIYVEWTNNGKTELNYKNTEPVLDSDGSYFMYS | | | | | |
| | KLRVEKKNWVERNSYSCSVVHEGLHNHHTTKSFSRTPGK | | | | | |
| 8H1- | 283 | 8H1 VH | 4GS3 | 8H1 VL | 5G Modified IgG2a | IgG2a |
| VH3VL- | | | | | Hinge | |
| 5GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSGGGGSEIVMTQSPGTLSLSPGESATLSCRASQSVSTTYLAWYQQKPGQAPRLLIYGASSR | | | | | |
| | ATGIPDRFSGSGSGTDFTLTISRLEPEDFAVYYCQQYGGSRLITFGQGTRLEIKGGGGGEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDFMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 8H1- | 284 | 8H1 VH | 4GS3 | 8H1 VL | 9G Modified IgG2a | IgG2a |
| VH3VL- | | | | | Hinge | |
| 9GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSGGGGSEIVMTQSPGTLSLSPGESATLSCRASQSVSTTYLAWYQQKPGQAPRLLIYGASSR | | | | | |
| | ATGIPDRFSGSGSGTDFTLTISRLEPEDFAVYYCQQYGGSRLITFGQGTRLEIKGGGGGGGG | | | | | |
| | GEPRGPTIKPCPPCKCPAPNLLGGPSVFIFPPKIKDVLMISLSPIVTCVVVDVSEDDPDVQI | | | | | |
| | SWFVNNVEVHTAQTQTHREDYNSTLRVVSALPIQHQDWMSGKEFKCKVNNKDLPAPIERTIS | | | | | |
| | KPKGSVRAPQVYVLPPPEEEMTKKQVTLTCMVTDFMPEDIYVEWTNNGKTELNYKNTEPVLD | | | | | |
| | SDGSYFMYSKLRVEKKNWVERNSYSCSVVHEGLHNHHTTKSFSRTPGK | | | | | |
| 8H1- | 285 | 8H1 VH | 4GS4 | 8H1 VL | IgG2a Hinge | IgG2a |
| VH4VL- | EVQLVESGGGLVQPGGSLRLSCAASGFTFSIYTMSWVRQAPGKGLEWVSAILSDGSTYYADS | | | | | |
| IgG2a | VEGRFTISRDNSKTTLYLQMNSLTAEDTAVYYCTKRVEGSFRGWGQGTLVTVSSGGGGSGGG | | | | | |
| | GSGGGGSGGGGSEIVMTQSPGTLSLSPGESATLSCRASQSVSTTYLAWYQQKPGQAPRLLIY | | | | | |
| | GASSRATGIPDRFSGSGSGTDFTLTISRLEPEDFAVYYCQQYGGSRLITFGQGTRLEIKEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDEMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 8H1- | 286 | 8H1 VH | 4GS4 | 8H1 VL | 5G Modified IgG2a | IgG2a |
| VH4VL- | | | | | Hinge | |
| 5GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSGGGGSGGGGSEIVMTQSPGTLSLSPGESATLSCRASQSVSTTYLAWYQQKPGQAPRLLIY | | | | | |
| | GASSRATGIPDRFSGSGSGTDFTLTISRLEPEDFAVYYCQQYGGSRLITFGQGTRLEIKGGG | | | | | |
| | GGEPRGPTIKPCPPCKCPAPNLLGGPSVFIFPPKIKDVLMISLSPIVTCVVVDVSEDDPDVQ | | | | | |
| | ISWFVNNVEVHTAQTQTHREDYNSTLRVVSALPIQHQDWMSGKEFKCKVNNKDLPAPIERTI | | | | | |
| | SKPKGSVRAPQVYVLPPPEEEMTKKQVTLTCMVTDEMPEDIYVEWTNNGKTELNYKNTEPVL | | | | | |
| | DSDGSYFMYSKLRVEKKNWVERNSYSCSVVHEGLHNHHTTKSFSRTPGK | | | | | |
| 8H1- | 287 | 8H1 VH | 4GS4 | 8H1 VL | 9G Modified IgG2a | IgG2a |
| VH4VL- | | | | | Hinge | |
| 9GIgG2a | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGSGGG | | | | | |
| | GSGGGGSGGGGSEIVMTQSPGTLSLSPGESATLSCRASQSVSTTYLAWYQQKPGQAPRLLIY | | | | | |
| | GASSRATGIPDRFSGSGSGTDFTLTISRLEPEDFAVYYCQQYGGSRLITFGQGTRLEIKGGG | | | | | |
| | GGGGGGEPRGPTIKPCPPCKCPAPNLLGGPSVFIFPPKIKDVLMISLSPIVTCVVVDVSEDD | | | | | |
| | PDVQISWFVNNVEVHTAQTQTHREDYNSTLRVVSALPIQHQDWMSGKEFKCKVNNKDLPAPI | | | | | |
| | ERTISKPKGSVRAPQVYVLPPPEEEMTKKQVTLTCMVTDFMPEDIYVEWTNNGKTELNYKNT | | | | | |
| | EPVLDSDGSYFMYSKLRVEKKNWVERNSYSCSVVHEGLHNHHTTKSFSRTPGK | | | | | |
| 8H1- | 288 | 8H1 VL | 4GS2 | 8H1 VH | IgG2a Hinge | IgG2a |
| VL2VH- | | | | | | |
| IgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSEVQLV | | | | | |
| | ESGGGLVQPGGSLRLSCAASGFTFSIYTMSWVRQAPGKGLEWVSAILSDGSTYYADSVEGRF | | | | | |
| | TISRDNSKTTLYLQMNSLTAEDTAVYYCTKRVEGSFRGWGQGTLVTVSSEPRGPTIKPCPPC | | | | | |
| | KCPAPNLLGGPSVFIFPPKIKDVLMISLSPIVTCVVVDVSEDDPDVQISWFVNNVEVHTAQT | | | | | |
| | QTHREDYNSTLRVVSALPIQHQDWMSGKEFKCKVNNKDLPAPIERTISKPKGSVRAPQVYVL | | | | | |
| | PPPEEEMTKKQVTLTCMVTDEMPEDIYVEWTNNGKTELNYKNTEPVLDSDGSYFMYSKLRVE | | | | | |
| | KKNWVERNSYSCSVVHEGLHNHHTTKSFSRTPGK | | | | | |
| 8H1- | 289 | 8H1 VL | 4GS2 | 8H1 VH | 5G Modified IgG2a | IgG2a |
| VL2VH- | | | | | Hinge | |
| 5GIgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSEVQLV | | | | | |
| | ESGGGLVQPGGSLRLSCAASGFTFSIYTMSWVRQAPGKGLEWVSAILSDGSTYYADSVEGRF | | | | | |
| | TISRDNSKTTLYLQMNSLTAEDTAVYYCTKRVEGSFRGWGQGTLVTVSSGGGGGEPRGPTIK | | | | | |
| | PCPPCKCPAPNLLGGPSVFIFPPKIKDVLMISLSPIVTCVVVDVSEDDPDVQISWFVNNVEV | | | | | |
| | HTAQTQTHREDYNSTLRVVSALPIQHQDWMSGKEFKCKVNNKDLPAPIERTISKPKGSVRAP | | | | | |
| | QVYVLPPPEEEMTKKQVTLTCMVTDFMPEDIYVEWTNNGKTELNYKNTEPVLDSDGSYFMYS | | | | | |
| | KLRVEKKNWVERNSYSCSVVHEGLHNHHTTKSFSRTPGK | | | | | |
| 8H1- | 290 | 8H1 VL | 4GS2 | 8H1 VH | 9G Modified IgG2a | IgG2a |
| VL2VH- | | | | | Hinge | |
| 9GIgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSEVQLV | | | | | |
| | ESGGGLVQPGGSLRLSCAASGFTFSIYTMSWVRQAPGKGLEWVSAILSDGSTYYADSVEGRE | | | | | |
| | TISRDNSKTTLYLQMNSLTAEDTAVYYCTKRVEGSFRGWGQGTLVTVSSGGGGGGGGGEPRG | | | | | |
| | PTIKPCPPCKCPAPNLLGGPSVFIFPPKIKDVLMISLSPIVTCVVVDVSEDDPDVQISWFVN | | | | | |
| | NVEVHTAQTQTHREDYNSTLRVVSALPIQHQDWMSGKEFKCKVNNKDLPAPIERTISKPKGS | | | | | |
| | VRAPQVYVLPPPEEEMTKKQVTLTCMVTDFMPEDIYVEWTNNGKTELNYKNTEPVLDSDGSY | | | | | |
| | FMYSKLRVEKKNWVERNSYSCSVVHEGLHNHHTTKSFSRTPGK | | | | | |
| 8H1- | 291 | 8H1 VL | 4GS3 | 8H1 VH | IgG2a Hinge | IgG2a |
| VL3 VH- | EIVMTQSPGTLSLSPGESATLSCRASQSVSTTYLAWYQQKPGQAPRLLIYGASSRATGIPDR | | | | | |
| IgG2a | FSGSGSGTDFTLTISRLEPEDFAVYYCQQYGGSRLITFGQGTRLEIKGGGGSGGGGSGGGGS | | | | | |
| | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSEPRGPTIK | | | | | |
| | PCPPCKCPAPNLLGGPSVFIFPPKIKDVLMISLSPIVTCVVVDVSEDDPDVQISWFVNNVEV | | | | | |
| | HTAQTQTHREDYNSTLRVVSALPIQHQDWMSGKEFKCKVNNKDLPAPIERTISKPKGSVRAP | | | | | |
| | QVYVLPPPEEEMTKKQVTLTCMVTDEMPEDIYVEWTNNGKTELNYKNTEPVLDSDGSYFMYS | | | | | |
| | KLRVEKKNWVERNSYSCSVVHEGLHNHHTTKSFSRTPGK | | | | | |
| 8H1- | 292 | 8H1 VL | 4GS3 | 8H1 VH | 5G Modified IgG2a | IgG2a |
| VL3 VH- | | | | | Hinge | |
| 5GIgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSGGGGS | | | | | |
| | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGGEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDFMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 8H1- | 293 | 8H1 VL | 4GS3 | 8H1 VH | 9G Modified IgG2a | IgG2a |
| VL3 VH- | | | | | Hinge | |
| 9GIgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSGGGGS | | | | | |
| | EVQLVESGGGLVQPGGSLRLSCAASGFTFSIYTMSWVRQAPGKGLEWVSAILSDGSTYYADS | | | | | |
| | VEGRFTISRDNSKTTLYLQMNSLTAEDTAVYYCTKRVEGSFRGWGQGTLVTVSSGGGGGGGG | | | | | |
| | GEPRGPTIKPCPPCKCPAPNLLGGPSVFIFPPKIKDVLMISLSPIVTCVVVDVSEDDPDVQI | | | | | |
| | SWFVNNVEVHTAQTQTHREDYNSTLRVVSALPIQHQDWMSGKEFKCKVNNKDLPAPIERTIS | | | | | |
| | KPKGSVRAPQVYVLPPPEEEMTKKQVTLTCMVTDFMPEDIYVEWTNNGKTELNYKNTEPVLD | | | | | |
| | SDGSYFMYSKLRVEKKNWVERNSYSCSVVHEGLHNHHTTKSFSRTPGK | | | | | |
| 8H1- | 294 | 8H1 VL | 4GS4 | 8H1 VH | IgG2a Hinge | IgG2a |
| VL4VH- | | | | | | |
| IgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSGGGGS | | | | | |
| | GGGGSEVQLVESGGGLVQPGGSLRLSCAASGFTFSIYTMSWVRQAPGKGLEWVSAILSDGST | | | | | |
| | YYADSVEGRFTISRDNSKTTLYLQMNSLTAEDTAVYYCTKRVEGSFRGWGQGTLVTVSSEPR | | | | | |
| | GPTIKPCPPCKCPAPNLLGGPSVFIFPPKIKDVLMISLSPIVTCVVVDVSEDDPDVQISWFV | | | | | |
| | NNVEVHTAQTQTHREDYNSTLRVVSALPIQHQDWMSGKEFKCKVNNKDLPAPIERTISKPKG | | | | | |
| | SVRAPQVYVLPPPEEEMTKKQVTLTCMVTDFMPEDIYVEWTNNGKTELNYKNTEPVLDSDGS | | | | | |
| | YFMYSKLRVEKKNWVERNSYSCSVVHEGLHNHHTTKSFSRTPGK | | | | | |
| 8H1- | 295 | 8H1 VL | 4GS4 | 8H1 VH | 5G Modified IgG2a | IgG2a |
| VL4VH- | | | | | Hinge | |
| 5GIgG2a | EIVMTQSPGTLSLSPGESATLSCRASQSVSTTYLAWYQQKPGQAPRLLIYGASSRATGIPDR | | | | | |
| | FSGSGSGTDFTLTISRLEPEDFAVYYCQQYGGSRLITFGQGTRLEIKGGGGSGGGGSGGGGS | | | | | |
| | GGGGSEVQLVESGGGLVQPGGSLRLSCAASGFTFSIYTMSWVRQAPGKGLEWVSAILSDGST | | | | | |
| | YYADSVEGRFTISRDNSKTTLYLQMNSLTAEDTAVYYCTKRVEGSFRGWGQGTLVTVSSGGG | | | | | |
| | GGEPRGPTIKPCPPCKCPAPNLLGGPSVFIFPPKIKDVLMISLSPIVTCVVVDVSEDDPDVQ | | | | | |
| | ISWFVNNVEVHTAQTQTHREDYNSTLRVVSALPIQHQDWMSGKEFKCKVNNKDLPAPIERTI | | | | | |
| | SKPKGSVRAPQVYVLPPPEEEMTKKQVTLTCMVTDEMPEDIYVEWTNNGKTELNYKNTEPVL | | | | | |
| | DSDGSYFMYSKLRVEKKNWVERNSYSCSVVHEGLHNHHTTKSFSRTPGK | | | | | |
| 8H1- | 296 | 8H1 VL | 4GS4 | 8H1 VH | 9G Modified IgG2a | IgG2a |
| VL4VH- | | | | | Hinge | |

| | |
|---|---|
| 9GIgG2a | EIVMTQSPGTLSLSPGESATLSCRASQSVSTTYLAWYQQKPGQAPRLLIYGASSRATGIPDR |
| | FSGSGSGTDFTLTISRLEPEDFAVYYCQQYGGSRLITFGQGTRLEIKGGGGSGGGGSGGGGS |
| | GGGGSEVQLVESGGGLVQPGGSLRLSCAASGFTFSIYTMSWVRQAPGKGLEWVSAILSDGST |
| | YYADSVEGRFTISRDNSKTTLYLQMNSLTAEDTAVYYCTKRVEGSFRGWGQGTLVTVSSGGG |
| | GGGGGGEPRGPTIKPCPPCKCPAPNLLGGPSVFIFPPKIKDVLMISLSPIVTCVVVDVSEDD |
| | PDVQISWFVNNVEVHTAQTQTHREDYNSTLRVVSALPIQHQDWMSGKEFKCKVNNKDLPAPI |
| | ERTISKPKGSVRAPQVYVLPPPEEEMTKKQVTLTCMVTDEMPEDIYVEWTNNGKTELNYKNT |
| | EPVLDSDGSYFMYSKLRVEKKNWVERNSYSCSVVHEGLHNHHTTKSFSRTPGK |

Component parts, with the amino acid sequences provided in Table 11A were arranged to construct encoding various exemplary ScFv polypeptides, the amino acid sequences of which are provided in Table 11C. Nucleic acid sequences (see Table 11D for components) encoding the ScFv polypeptides and Fc domains were cloned into Cis plasmids for testing and for making rAAV (see Table 11E for ScFv-Fc transgenes).

TABLE 11C
Exemplary scFv Amino Acid Sequences

| | ScFv | | | |
|---|---|---|---|---|
| ScFv | SEQ ID | | Linker | |
| Name | NO: | VH or VL | (4GS)ₓ | VL or VH |
| 8H1- | 297 | 8H1 VH | 4GS2 | 8H1 VL |
| VH2VL | EVQLVESGGGLVQPGGSLRLSCAASGFTFSIYTMSWVRQAPGKGLEWVSAILSDGSTYYADSV | | | |
| | EGRFTISRDNSKTTLYLQMNSLTAEDTAVYYCTKRVEGSFRGWGQGTLVTVSSGGGGSGGGGS | | | |
| | EIVMTQSPGTLSLSPGESATLSCRASQSVSTTYLAWYQQKPGQAPRLLIYGASSRATGIPDRF | | | |
| | SGSGSGTDFTLTISRLEPEDFAVYYCQQYGGSRLITFGQGTRLEIK | | | |
| 8H1- | 298 | 8H1 VH | 4GS3 | 8H1 VL |
| VH3VL | EVQLVESGGGLVQPGGSLRLSCAASGFTFSIYTMSWVRQAPGKGLEWVSAILSDGSTYYADSV | | | |
| | EGRFTISRDNSKTTLYLQMNSLTAEDTAVYYCTKRVEGSFRGWGQGTLVTVSSGGGGSGGGGS | | | |
| | GGGGSEIVMTQSPGTLSLSPGESATLSCRASQSVSTTYLAWYQQKPGQAPRLLIYGASSRATG | | | |
| | IPDRFSGSGSGTDFTLTISRLEPEDFAVYYCQQYGGSRLITFGQGTRLEIK | | | |
| 8H1- | 299 | 8H1 VH | 4GS4 | 8H1 VL |
| VH4VL | EVQLVESGGGLVQPGGSLRLSCAASGFTFSIYTMSWVRQAPGKGLEWVSAILSDGSTYYADSV | | | |
| | EGRFTISRDNSKTTLYLQMNSLTAEDTAVYYCTKRVEGSFRGWGQGTLVTVSSGGGGSGGGGS | | | |
| | GGGGSGGGGSEIVMTQSPGTLSLSPGESATLSCRASQSVSTTYLAWYQQKPGQAPRLLIYGAS | | | |
| | SRATGIPDRFSGSGSGTDFTLTISRLEPEDFAVYYCQQYGGSRLITFGQGTRLEIK | | | |
| 31B6- | 300 | 31B6 VH | 4GS2 | 31B6 VL |
| VH2VL | EVQLLQSGGGVVQPGGSLRLSCEASGFTFSSYSMHWVRQAPGKGLEWLTLISYNGVNKYYAKS | | | |
| | VKGRFAISRDDSKSTLFLQMNSLTTDDTAVYYCARCPIPRAASYRNGDNYFEFGQGTLVTVSS | | | |
| | GGGGSGGGGSQAVVTQEPSLSVSPGGTVTLTCALSSASVSTSSYPSWYQQTPGQPPRMLIHNT | | | |
| | DTRSSGVPDRFSGSILGKKAALTITGAQADDESDYYCVVYLGNGFWVFGGGTKLTVL | | | |
| 31B6- | 301 | 31B6 VH | 4GS3 | 31B6 VL |
| VH3VL | EVQLLQSGGGVVQPGGSLRLSCEASGFTFSSYSMHWVRQAPGKGLEWLTLISYNGVNKYYAKS | | | |
| | VKGRFAISRDDSKSTLFLQMNSLTTDDTAVYYCARCPIPRAASYRNGDNYFEFGQGTLVTVSS | | | |
| | GGGGGGGGSGGGGSQAVVTQEPSLSVSPGGTVTLTCALSSASVSTSSYPSWYQQTPGQPPRM | | | |
| | LIHNTDTRSSGVPDRFSGSILGKKAALTITGAQADDESDYYCVVYLGNGFWVFGGGTKLTVL | | | |
| 31B6- | 302 | 31B6 VH | 4GS4 | 31B6 VL |
| VH4VL | EVQLLQSGGGVVQPGGSLRLSCEASGFTFSSYSMHWVRQAPGKGLEWLTLISYNGVNKYYAKS | | | |
| | VKGRFAISRDDSKSTLFLQMNSLTTDDTAVYYCARCPIPRAASYRNGDNYFEFGQGTLVTVSS | | | |
| | GGGGSGGGGSGGGGSGGGGSQAVVTQEPSLSVSPGGTVTLTCALSSASVSTSSYPSWYQQTPG | | | |
| | QPPRMLIHNTDTRSSGVPDRFSGSILGKKAALTITGAQADDESDYYCVVYLGNGFWVFGGGTK | | | |
| | LTVL | | | |
| 4P3- | 303 | 4P3 VH | 4GS2 | 4P3 VL |
| VH2VL | EVQLVQSGAEVKKPGESLKISCKVSGYDFSKYWIGWVRQMPGKGLEWMGLIYPGDSDTRYSPS | | | |
| | LEGQVSISADKSISAAYLQFSSLKASDSAMYYCVRKGYSAYLGGWFDPWGQGTLVTVSSGGGG | | | |
| | SGGGGSDIQLTQSPDSLAASLGERVTINCRSSQNIFYNSNNKNFLAWYQQKAGQSPKLLIYWA | | | |
| | STRESGVPDRFSGSGSGTDFTLTISGLQAGDVAVYFCQQYFTDAPTFGGGTKVEIK | | | |
| 4P3- | 304 | 4P3 VH | 4GS3 | 4P3 VL |
| VH3VL | EVQLVQSGAEVKKPGESLKISCKVSGYDFSKYWIGWVRQMPGKGLEWMGLIYPGDSDTRYSPS | | | |
| | LEGQVSISADKSISAAYLQFSSLKASDSAMYYCVRKGYSAYLGGWFDPWGQGTLVTVSSGGGG | | | |
| | SGGGGSGGGGSDIQLTQSPDSLAASLGERVTINCRSSQNIFYNSNNKNFLAWYQQKAGQSPKL | | | |
| | LIYWASTRESGVPDRFSGSGSGTDFTLTISGLQAGDVAVYFCQQYFTDAPTFGGGTKVEIK | | | |
| 4P3- | 305 | 4P3 VH | 4GS4 | 4P3 VL |

| | |
|---|---|
| VH4VL | EVQLVQSGAEVKKPGESLKISCKVSGYDESKYWIGWVRQMPGKGLEWMGLIYPGDSDTRYSPS |
| | LEGQVSISADKSISAAYLQFSSLKASDSAMYYCVRKGYSAYLGGWFDPWGQGTLVTVSSGGGG |
| | SGGGGSGGGGSGGGGSDIQLTQSPDSLAASLGERVTINCRSSQNIFYNSNNKNFLAWYQQKAG |
| | QSPKLLIYWASTRESGVPDRFSGSGSGTDFTLTISGLQAGDVAVYFCQQYFTDAPTFGGGTKV |
| | EIK |

Nucleic acid sequences encoding the ScFv-Fc polypeptides were cloned into Cis plasmids for testing and for making rAAV. Component parts, exemplary nucleotide sequence of which are listed in Table 11D were arranged to construct various ScFv-Fc polypeptides listed in Table 11B.

TABLE 11D
ScFv and ScFv-Fc Component Nucleic Acid Sequences

| | SEQ | |
|---|---|---|
| Name | ID NO: | Nucleotide Sequence |
| 4P3 VH | 306 | GAGGTGCAGCTGGTGCAGTCTGGGGCTGAAGTGAAGAAGCCTGGGGAGAGCCTGA |
| | | AGATCAGCTGCAAAGTGTCTGGCTATGACTTCAGCAAGTACTGGATTGGCTGGGT |
| | | CAGACAGATGCCTGGCAAAGGCCTGGAATGGATGGGACTGATCTACCCTGGGGAC |
| | | TCTGACACCAGATACAGCCCTAGCCTGGAAGGCCAGGTGTCCATCTCTGCTGACA |
| | | AGAGCATCTCTGCAGCCTACCTGCAGTTCAGCAGCCTGAAGGCCTCTGACTCTGC |
| | | CATGTACTACTGTGTCAGAAAGGGCTACTCTGCCTACCTTGGAGGATGGTTTGAT |
| | | CCTTGGGGCCAGGGCACACTGGTCACAGTGTCCTCT |
| 4P3 VL | 307 | GACATCCAGCTGACACAGAGCCCTGACAGCCTGGCTGCTTCTCTGGGAGAGAGAG |
| | | TGACCATCAACTGCAGAAGCTCCCAGAACATCTTCTACAACAGCAACAACAAGAA |
| | | CTTCCTGGCCTGGTATCAGCAGAAGGCTGGCCAGTCTCCTAAGCTGCTGATCTAC |
| | | TGGGCCAGCACCAGAGAATCTGGGGTGCCAGATAGATTTTCTGGCAGTGGCTCTG |
| | | GCACAGACTTCACCCTGACAATCAGTGGACTGCAGGCTGGGGATGTTGCTGTGTA |
| | | CTTCTGCCAGCAGTACTTCACAGATGCCCCTACCTTTGGTGGTGGCACCAAGGTG |
| | | GAAATCAAG |
| 31B6 VH | 308 | GAGGTGCAGCTGCTGCAGAGTGGTGGTGGTGTTGTGCAGCCTGGTGGCTCTCTGA |
| | | GACTGTCTTGTGAAGCCTCTGGCTTCACCTTCAGCAGCTACAGCATGCACTGGGT |
| | | CAGACAGGCCCCTGGCAAAGGACTGGAATGGCTGACCCTGATCAGCTACAATGGG |
| | | GTCAACAAGTACTATGCCAAGTCTGTGAAGGGCAGATTTGCCATCAGCAGAGATG |
| | | ACAGCAAGTCTACCCTGTTCCTGCAGATGAACAGCCTGACCACAGATGACACTGC |
| | | TGTGTACTACTGTGCCAGATGTCCCATTCCTAGAGCTGCCAGCTACAGAAATGGG |
| | | GACAACTACTTTGAGTTTGGCCAGGGCACCCTGGTCACAGTGTCCTCT |
| 31B6 VL | 309 | CAGGCTGTGGTCACCCAAGAGCCTAGCCTGTCTGTGTCTCCTGGTGGCACAGTGA |
| | | CACTGACATGTGCCCTGTCCTCTGCCTCTGTGTCTACCAGCAGCTACCCCAGCTG |
| | | GTATCAGCAGACACCTGGCCAGCCTCCAAGAATGCTGATCCACAACACAGACACC |
| | | AGATCCTCTGGGGTGCCAGACAGATTCTCTGGCAGCATCCTGGGCAAGAAGGCTG |
| | | CCCTGACAATCACTGGTGCCCAGGCTGATGATGAGTCTGACTACTACTGTGTGGT |
| | | GTACCTTGGCAATGGCTTCTGGGTGTTTGGTGGTGGCACCAAGCTGACAGTGCTG |
| 8H1 VH | 310 | GAGGTGCAGCTGGTGGAAAGTGGTGGTGGACTGGTTCAGCCTGGTGGCAGCCTGA |
| | | GACTGTCTTGTGCTGCCTCTGGCTTCACCTTCAGCATCTACACCATGAGCTGGGT |
| | | CAGACAGGCTCCTGGCAAAGGCCTGGAATGGGTGTCAGCTATCCTGTCTGATGGC |
| | | AGCACCTACTATGCTGACTCTGTGGAAGGCAGATTCACCATCAGCAGAGACAACA |
| | | GCAAGACCACACTGTACCTGCAGATGAACAGCCTGACAGCTGAGGACACTGCTGT |
| | | GTACTACTGCACCAAGAGAGTGGAAGGCTCCTTCAGAGGCTGGGGACAGGGAACA |
| | | CTGGTCACTGTGTCCTCT |
| 8H1 VL | 311 | GAGATTGTGATGACACAGAGCCCTGGCACACTGTCTCTGAGCCCTGGGGAATCTG |
| | | CCACACTGAGCTGTAGAGCCAGCCAGTCTGTGTCTACCACCTACCTGGCCTGGTA |
| | | TCAGCAGAAGCCTGGACAGGCTCCCAGACTGCTGATCTATGGGGCCTCTTCTAGA |
| | | GCCACAGGCATCCCTGACAGATTTTCTGGCAGTGGCTCTGGCACAGACTTCACCC |
| | | TGACCATCAGCAGACTGGAACCTGAGGACTTTGCTGTGTACTACTGCCAGCAGTA |
| | | TGGTGGCAGCAGGCTGATCACATTTGGCCAGGGCACAAGACTGGAAATCAAG |
| 4GS2 | 312 | GGCGGAGGTGGCAGCGGAGGTGGCGGATCA |
| 4GS3 | 313 | GGTGGAGGCGGTTCAGGCGGAGGTGGCAGCGGCGGTGGAGGGTCG |
| 4GS4 | 314 | GGTGGAGGCGGTTCAGGCGGAGGTGGCAGCGGCGGTGGAGGGTCAGGTGGAGGTG |
| | | GATCA |
| 9G | 315 | GGCGGAGGAGGCGGAGGTGGAGGAGGA |
| IgG2a | 316 | GAGCCTAGAGGCCCCACCATCAAGCCCTGTCCTCCATGCAAGTGTCCTGCTCCTA |
| Hinge/CH2/ | | ACCTGCTTGGAGGCCCCTCTGTGTTCATCTTCCCACCTAAGATCAAGGATGTGCT |
| CH3 | | GATGATCTCTCTGAGCCCCATTGTGACCTGTGTGGTGGTGGATGTGTCTGAGGAT |
| (IgG2a | | GACCCTGATGTGCAGATCTCTTGGTTTGTGAACAATGTGGAAGTGCACACAGCCC |
| Fc) | | AGACACAGACCCACAGAGAGGACTACAACAGCACCCTGAGAGTGGTGTCTGCCCT |
| | | GCCTATCCAGCACCAGGACTGGATGAGTGGCAAAGAGTTCAAGTGCAAAGTGAAC |
| | | AACAAGGACCTGCCTGCTCCAATTGAGAGAACCATCAGCAAGCCCAAGGGCTCTG |
| | | TTAGAGCCCCTCAAGTGTATGTGCTGCCTCCACCTGAGGAAGAGATGACCAAGAA |
| | | ACAAGTGACCCTGACATGCATGGTCACAGACTTCATGCCTGAGGACATCTATGTG |
| | | GAATGGACCAACAATGGCAAGACAGAGCTGAACTACAAGAACACTGAGCCTGTGC |
| | | TGGACTCTGATGGCAGCTACTTCATGTACAGCAAGCTGAGGGTTGAGAAGAAGAA |
| | | CTGGGTTGAGAGAAACAGCTACAGCTGCTCTGTGGTGCATGAGGGCCTGCACAAT |
| | | CACCACACCACCAAGAGCTTCAGCAGAACCCCTGGCAAG |
| | | Hinge is in italics. |
| Human | 344 | GAACCCAAGAGCTGTGACAAGACCCACACCTGTCCTCCATGTCCTGCTCCAGAAC |
| IgG1 Fc | | TGCTTGGAGGCCCTTCTGTGTTCCTGTTTCCTCCAAAGCCTAAGGACACCCTGAT |
| domain | | GATCAGCAGAACCCCTGAAGTGACCTGTGTGGTGGTTGATGTGTCCCATGAGGAC |
| exemplary | | CCAGAAGTGAAGTTCAATTGGTATGTGGATGGGGTTGAAGTGCACAATGCCAAGA |
| nucleotide | | CCAAGCCTAGAGAGGAACAGTACAACTCCACCTACAGAGTGGTGTCTGTGCTGAC |
| sequence | | AGTGCTGCACCAGGACTGGCTGAATGGCAAAGAGTACAAGTGCAAGGTGTCCAAC |
| (CH2-CH3) | | AAGGCCCTGCCTGCTCCTATTGAGAAAACCATCTCCAAGGCCAAGGGCCAGCCAA |
| | | GAGAACCCCAGGTTTACACCCTGCCACCTAGCAGAGAAGAGATGACCAAGAACCA |
| | | GGTGTCCCTGACCTGCCTGGTTAAGGGCTTCTACCCCTCTGACATTGCTGTGGAA |
| | | TGGGAGAGCAATGGCCAGCCTGAAAACAACTACAAGACAACCCCTCCTGTGCTGG |
| | | ACTCAGATGGCTCATTCTTCCTGTACAGCAAGCTGACTGTGGACAAGTCCAGGTG |
| | | GCAGCAGGGAAATGTGTTCAGCTGCTCTGTGATGCATGAGGCCCTGCACAACCAC |
| | | TACACCCAGAAAAGTCTGAGTCTGAGCCCTGGCAAG |
| | | Hinge is in italics. |

TABLE 11E
Nucleotide sequences encoding ScFv-Fcs

| | ScFv-Fc | | | | | |
|---|---|---|---|---|---|---|
| | Transgene | | | | IgG hinge or | |
| ScFv-Fc | SEQ ID | | Linker | | Glycine Modified | IgG |
| Name | NO: | VH or VL | (4GS)ₓ | VL or VH | IgG hinge | CH2/CH3 |
| 4P3- | 317 | 4P3 VH | 4GS2 | 4P3 VL | 9G Modified IgG2a | IgG2a |
| VH2VL- | | | | | Hinge | |
| 9GIgG2a | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGCGGAGGTGGCAGCGGA | | | | | |
| | GGTGGCGGATCAGACATCCAGCTGACACAGAGCCCTGACAGCCTGGCTGCTTCTCTGGGAGAGA | | | | | |
| | GAGTGACCATCAACTGCAGAAGCTCCCAGAACATCTTCTACAACAGCAACAACAAGAACTTCCT | | | | | |
| | GGCCTGGTATCAGCAGAAGGCTGGCCAGTCTCCTAAGCTGCTGATCTACTGGGCCAGCACCAGA | | | | | |
| | GAATCTGGGGTGCCAGATAGATTTTCTGGCAGTGGCTCTGGCACAGACTTCACCCTGACAATCA | | | | | |
| | GTGGACTGCAGGCTGGGGATGTTGCTGTGTACTTCTGCCAGCAGTACTTCACAGATGCCCCTAC | | | | | |
| | CTTTGGTGGTGGCACCAAGGTGGAAATCAAGGGCGGAGGAGGCGGAGGTGGAGGAGGAGAGCCT | | | | | |
| | AGAGGCCCCACCATCAAGCCCTGTCCTCCATGCAAGTGTCCTGCTCCTAACCTGCTTGGAGGCC | | | | | |
| | CCTCTGTGTTCATCTTCCCACCTAAGATCAAGGATGTGCTGATGATCTCTCTGAGCCCCATTGT | | | | | |
| | GACCTGTGTGGTGGTGGATGTGTCTGAGGATGACCCTGATGTGCAGATCTCTTGGTTTGTGAAC | | | | | |
| | AATGTGGAAGTGCACACAGCCCAGACACAGACCCACAGAGAGGACTACAACAGCACCCTGAGAG | | | | | |
| | TGGTGTCTGCCCTGCCTATCCAGCACCAGGACTGGATGAGTGGCAAAGAGTTCAAGTGCAAAGT | | | | | |
| | GAACAACAAGGACCTGCCTGCTCCAATTGAGAGAACCATCAGCAAGCCCAAGGGCTCTGTTAGA | | | | | |
| | GCCCCTCAAGTGTATGTGCTGCCTCCACCTGAGGAAGAGATGACCAAGAAACAAGTGACCCTGA | | | | | |
| | CATGCATGGTCACAGACTTCATGCCTGAGGACATCTATGTGGAATGGACCAACAATGGCAAGAC | | | | | |
| | AGAGCTGAACTACAAGAACACTGAGCCTGTGCTGGACTCTGATGGCAGCTACTTCATGTACAGC | | | | | |
| | AAGCTGAGGGTTGAGAAGAAGAACTGGGTTGAGAGAAACAGCTACAGCTGCTCTGTGGTGCATG | | | | | |
| | AGGGCCTGCACAATCACCACACCACCAAGAGCTTCAGCAGAACCCCTGGCAAG | | | | | |
| 4P3- | 345 | 4P3 VH | 4GS2 | 4P3 VL | 9G Modified IgG1 | IgG1 |
| VH2VL- | | | | | Hinge | |
| 9GIgG1 | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGCGGAGGTGGCAGCGGA | | | | | |
| | GGTGGCGGATCAGACATCCAGCTGACACAGAGCCCTGACAGCCTGGCTGCTTCTCTGGGAGAGA | | | | | |
| | GAGTGACCATCAACTGCAGAAGCTCCCAGAACATCTTCTACAACAGCAACAACAAGAACTTCCT | | | | | |
| | GGCCTGGTATCAGCAGAAGGCTGGCCAGTCTCCTAAGCTGCTGATCTACTGGGCCAGCACCAGA | | | | | |
| | GAATCTGGGGTGCCAGATAGATTTTCTGGCAGTGGCTCTGGCACAGACTTCACCCTGACAATCA | | | | | |
| | GTGGACTGCAGGCTGGGGATGTTGCTGTGTACTTCTGCCAGCAGTACTTCACAGATGCCCCTAC | | | | | |
| | CTTTGGTGGTGGCACCAAGGTGGAAATCAAGGGCGGAGGAGGCGGAGGTGGAGGAGGAGAACCC | | | | | |
| | AAGAGCTGTGACAAGACCCACACCTGTCCTCCATGTCCTGCTCCAGAACTGCTTGGAGGCCCTT | | | | | |
| | CTGTGTTCCTGTTTCCTCCAAAGCCTAAGGACACCCTGATGATCAGCAGAACCCCTGAAGTGAC | | | | | |
| | CTGTGTGGTGGTTGATGTGTCCCATGAGGACCCAGAAGTGAAGTTCAATTGGTATGTGGATGGG | | | | | |
| | GTTGAAGTGCACAATGCCAAGACCAAGCCTAGAGAGGAACAGTACAACTCCACCTACAGAGTGG | | | | | |
| | TGTCTGTGCTGACAGTGCTGCACCAGGACTGGCTGAATGGCAAAGAGTACAAGTGCAAGGTGTC | | | | | |
| | CAACAAGGCCCTGCCTGCTCCTATTGAGAAAACCATCTCCAAGGCCAAGGGCCAGCCAAGAGAA | | | | | |
| | CCCCAGGTTTACACCCTGCCACCTAGCAGAGAAGAGATGACCAAGAACCAGGTGTCCCTGACCT | | | | | |
| | GCCTGGTTAAGGGCTTCTACCCCTCTGACATTGCTGTGGAATGGGAGAGCAATGGCCAGCCTGA | | | | | |
| | AAACAACTACAAGACAACCCCTCCTGTGCTGGACTCAGATGGCTCATTCTTCCTGTACAGCAAG | | | | | |
| | CTGACTGTGGACAAGTCCAGGTGGCAGCAGGGAAATGTGTTCAGCTGCTCTGTGATGCATGAGG | | | | | |
| | CCCTGCACAACCACTACACCCAGAAAAGTCTGAGTCTGAGCCCTGGCAAG | | | | | |
| 4P3- | 318 | 4P3 VH | 4GS3 | 4P3 VL | 9G Modified IgG2a | IgG2a |
| VH3VL- | | | | | Hinge | |
| 9GIgG2a | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGTGGAGGCGGTTCAGGC | | | | | |
| | GGAGGTGGCAGCGGCGGTGGAGGGTCGGACATCCAGCTGACACAGAGCCCTGACAGCCTGGCTG | | | | | |
| | CTTCTCTGGGAGAGAGAGTGACCATCAACTGCAGAAGCTCCCAGAACATCTTCTACAACAGCAA | | | | | |
| | CAACAAGAACTTCCTGGCCTGGTATCAGCAGAAGGCTGGCCAGTCTCCTAAGCTGCTGATCTAC | | | | | |
| | TGGGCCAGCACCAGAGAATCTGGGGTGCCAGATAGATTTTCTGGCAGTGGCTCTGGCACAGACT | | | | | |
| | TCACCCTGACAATCAGTGGACTGCAGGCTGGGGATGTTGCTGTGTACTTCTGCCAGCAGTACTT | | | | | |
| | CACAGATGCCCCTACCTTTGGTGGTGGCACCAAGGTGGAAATCAAGGGCGGAGGAGGCGGAGGT | | | | | |
| | GGAGGAGGAGAGCCTAGAGGCCCCACCATCAAGCCCTGTCCTCCATGCAAGTGTCCTGCTCCTA | | | | | |
| | ACCTGCTTGGAGGCCCCTCTGTGTTCATCTTCCCACCTAAGATCAAGGATGTGCTGATGATCTC | | | | | |
| | TCTGAGCCCCATTGTGACCTGTGTGGTGGTGGATGTGTCTGAGGATGACCCTGATGTGCAGATC | | | | | |
| | TCTTGGTTTGTGAACAATGTGGAAGTGCACACAGCCCAGACACAGACCCACAGAGAGGACTACA | | | | | |
| | ACAGCACCCTGAGAGTGGTGTCTGCCCTGCCTATCCAGCACCAGGACTGGATGAGTGGCAAAGA | | | | | |
| | GTTCAAGTGCAAAGTGAACAACAAGGACCTGCCTGCTCCAATTGAGAGAACCATCAGCAAGCCC | | | | | |
| | AAGGGCTCTGTTAGAGCCCCTCAAGTGTATGTGCTGCCTCCACCTGAGGAAGAGATGACCAAGA | | | | | |
| | AACAAGTGACCCTGACATGCATGGTCACAGACTTCATGCCTGAGGACATCTATGTGGAATGGAC | | | | | |
| | CAACAATGGCAAGACAGAGCTGAACTACAAGAACACTGAGCCTGTGCTGGACTCTGATGGCAGC | | | | | |
| | TACTTCATGTACAGCAAGCTGAGGGTTGAGAAGAAGAACTGGGTTGAGAGAAACAGCTACAGCT | | | | | |
| | GCTCTGTGGTGCATGAGGGCCTGCACAATCACCACACCACCAAGAGCTTCAGCAGAACCCCTGG | | | | | |
| | CAAG | | | | | |
| 4P3- | 319 | 4P3 VH | 4GS4 | 4P3 VL | 9G Modified IgG2a | IgG2a |
| VH4VL- | | | | | Hinge | |
| 9GIgG2a | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGTGGAGGCGGTTCAGGC | | | | | |
| | GGAGGTGGCAGCGGCGGTGGAGGGTCAGGTGGAGGTGGATCAGACATCCAGCTGACACAGAGCC | | | | | |
| | CTGACAGCCTGGCTGCTTCTCTGGGAGAGAGAGTGACCATCAACTGCAGAAGCTCCCAGAACAT | | | | | |
| | CTTCTACAACAGCAACAACAAGAACTTCCTGGCCTGGTATCAGCAGAAGGCTGGCCAGTCTCCT | | | | | |
| | AAGCTGCTGATCTACTGGGCCAGCACCAGAGAATCTGGGGTGCCAGATAGATTTTCTGGCAGTG | | | | | |
| | GCTCTGGCACAGACTTCACCCTGACAATCAGTGGACTGCAGGCTGGGGATGTTGCTGTGTACTT | | | | | |
| | CTGCCAGCAGTACTTCACAGATGCCCCTACCTTTGGTGGTGGCACCAAGGTGGAAATCAAGGGC | | | | | |
| | GGAGGAGGCGGAGGTGGAGGAGGAGAGCCTAGAGGCCCCACCATCAAGCCCTGTCCTCCATGCA | | | | | |
| | AGTGTCCTGCTCCTAACCTGCTTGGAGGCCCCTCTGTGTTCATCTTCCCACCTAAGATCAAGGA | | | | | |
| | TGTGCTGATGATCTCTCTGAGCCCCATTGTGACCTGTGTGGTGGTGGATGTGTCTGAGGATGAC | | | | | |
| | CCTGATGTGCAGATCTCTTGGTTTGTGAACAATGTGGAAGTGCACACAGCCCAGACACAGACCC | | | | | |
| | ACAGAGAGGACTACAACAGCACCCTGAGAGTGGTGTCTGCCCTGCCTATCCAGCACCAGGACTG | | | | | |
| | GATGAGTGGCAAAGAGTTCAAGTGCAAAGTGAACAACAAGGACCTGCCTGCTCCAATTGAGAGA | | | | | |
| | ACCATCAGCAAGCCCAAGGGCTCTGTTAGAGCCCCTCAAGTGTATGTGCTGCCTCCACCTGAGG | | | | | |
| | AAGAGATGACCAAGAAACAAGTGACCCTGACATGCATGGTCACAGACTTCATGCCTGAGGACAT | | | | | |
| | CTATGTGGAATGGACCAACAATGGCAAGACAGAGCTGAACTACAAGAACACTGAGCCTGTGCTG | | | | | |
| | GACTCTGATGGCAGCTACTTCATGTACAGCAAGCTGAGGGTTGAGAAGAAGAACTGGGTTGAGA | | | | | |
| | GAAACAGCTACAGCTGCTCTGTGGTGCATGAGGGCCTGCACAATCACCACACCACCAAGAGCTT | | | | | |
| | CAGCAGAACCCCTGGCAAG | | | | | |
| 4P3- | 346 | 4P3 VH | 4GS4 | 4P3 VL | 9G Modified IgG1 | IgG1 |
| VH4VL- | | | | | Hinge | |
| 9GIgG1 | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGTGGAGGCGGTTCAGGC | | | | | |
| | GGAGGTGGCAGCGGCGGTGGAGGGTCAGGTGGAGGTGGATCAGACATCCAGCTGACACAGAGCC | | | | | |
| | CTGACAGCCTGGCTGCTTCTCTGGGAGAGAGAGTGACCATCAACTGCAGAAGCTCCCAGAACAT | | | | | |
| | CTTCTACAACAGCAACAACAAGAACTTCCTGGCCTGGTATCAGCAGAAGGCTGGCCAGTCTCCT | | | | | |
| | AAGCTGCTGATCTACTGGGCCAGCACCAGAGAATCTGGGGTGCCAGATAGATTTTCTGGCAGTG | | | | | |
| | GCTCTGGCACAGACTTCACCCTGACAATCAGTGGACTGCAGGCTGGGGATGTTGCTGTGTACTT | | | | | |
| | CTGCCAGCAGTACTTCACAGATGCCCCTACCTTTGGTGGTGGCACCAAGGTGGAAATCAAGGGC | | | | | |
| | GGAGGAGGCGGAGGTGGAGGAGGAGAACCCAAGAGCTGTGACAAGACCCACACCTGTCCTCCAT | | | | | |
| | GTCCTGCTCCAGAACTGCTTGGAGGCCCTTCTGTGTTCCTGTTTCCTCCAAAGCCTAAGGACAC | | | | | |
| | CCTGATGATCAGCAGAACCCCTGAAGTGACCTGTGTGGTGGTTGATGTGTCCCATGAGGACCCA | | | | | |
| | GAAGTGAAGTTCAATTGGTATGTGGATGGGGTTGAAGTGCACAATGCCAAGACCAAGCCTAGAG | | | | | |
| | AGGAACAGTACAACTCCACCTACAGAGTGGTGTCTGTGCTGACAGTGCTGCACCAGGACTGGCT | | | | | |
| | GAATGGCAAAGAGTACAAGTGCAAGGTGTCCAACAAGGCCCTGCCTGCTCCTATTGAGAAAACC | | | | | |
| | ATCTCCAAGGCCAAGGGCCAGCCAAGAGAACCCCAGGTTTACACCCTGCCACCTAGCAGAGAAG | | | | | |
| | AGATGACCAAGAACCAGGTGTCCCTGACCTGCCTGGTTAAGGGCTTCTACCCCTCTGACATTGC | | | | | |
| | TGTGGAATGGGAGAGCAATGGCCAGCCTGAAAACAACTACAAGACAACCCCTCCTGTGCTGGAC | | | | | |
| | TCAGATGGCTCATTCTTCCTGTACAGCAAGCTGACTGTGGACAAGTCCAGGTGGCAGCAGGGAA | | | | | |
| | ATGTGTTCAGCTGCTCTGTGATGCATGAGGCCCTGCACAACCACTACACCCAGAAAAGTCTGAG | | | | | |
| | TCTGAGCCCTGGCAAG | | | | | |
| 4P3- | 320 | 4P3 VL | 4GS2 | 4P3 VH | 9G Modified IgG2a | IgG2a |
| VL2VH- | | | | | Hinge | |
| 9GIgG2a | GACATCCAGCTGACACAGAGCCCTGACAGCCTGGCTGCTTCTCTGGGAGAGAGAGTGACCATCA | | | | | |
| | ACTGCAGAAGCTCCCAGAACATCTTCTACAACAGCAACAACAAGAACTTCCTGGCCTGGTATCA | | | | | |
| | GCAGAAGGCTGGCCAGTCTCCTAAGCTGCTGATCTACTGGGCCAGCACCAGAGAATCTGGGGTG | | | | | |
| | CCAGATAGATTTTCTGGCAGTGGCTCTGGCACAGACTTCACCCTGACAATCAGTGGACTGCAGG | | | | | |
| | CTGGGGATGTTGCTGTGTACTTCTGCCAGCAGTACTTCACAGATGCCCCTACCTTTGGTGGTGG | | | | | |
| | CACCAAGGTGGAAATCAAGGGCGGAGGTGGCAGCGGAGGTGGCGGATCAGAGGTGCAGCTGGTG | | | | | |
| | CAGTCTGGGGCTGAAGTGAAGAAGCCTGGGGAGAGCCTGAAGATCAGCTGCAAAGTGTCTGGCT | | | | | |
| | ATGACTTCAGCAAGTACTGGATTGGCTGGGTCAGACAGATGCCTGGCAAAGGCCTGGAATGGAT | | | | | |
| | GGGACTGATCTACCCTGGGGACTCTGACACCAGATACAGCCCTAGCCTGGAAGGCCAGGTGTCC | | | | | |
| | ATCTCTGCTGACAAGAGCATCTCTGCAGCCTACCTGCAGTTCAGCAGCCTGAAGGCCTCTGACT | | | | | |
| | CTGCCATGTACTACTGTGTCAGAAAGGGCTACTCTGCCTACCTTGGAGGATGGTTTGATCCTTG | | | | | |
| | GGGCCAGGGCACACTGGTCACAGTGTCCTCTGGCGGAGGAGGCGGAGGTGGAGGAGGAGAGCCT | | | | | |
| | AGAGGCCCCACCATCAAGCCCTGTCCTCCATGCAAGTGTCCTGCTCCTAACCTGCTTGGAGGCC | | | | | |
| | CCTCTGTGTTCATCTTCCCACCTAAGATCAAGGATGTGCTGATGATCTCTCTGAGCCCCATTGT | | | | | |
| | GACCTGTGTGGTGGTGGATGTGTCTGAGGATGACCCTGATGTGCAGATCTCTTGGTTTGTGAAC | | | | | |
| | AATGTGGAAGTGCACACAGCCCAGACACAGACCCACAGAGAGGACTACAACAGCACCCTGAGAG | | | | | |
| | TGGTGTCTGCCCTGCCTATCCAGCACCAGGACTGGATGAGTGGCAAAGAGTTCAAGTGCAAAGT | | | | | |
| | GAACAACAAGGACCTGCCTGCTCCAATTGAGAGAACCATCAGCAAGCCCAAGGGCTCTGTTAGA | | | | | |
| | GCCCCTCAAGTGTATGTGCTGCCTCCACCTGAGGAAGAGATGACCAAGAAACAAGTGACCCTGA | | | | | |
| | CATGCATGGTCACAGACTTCATGCCTGAGGACATCTATGTGGAATGGACCAACAATGGCAAGAC | | | | | |
| | AGAGCTGAACTACAAGAACACTGAGCCTGTGCTGGACTCTGATGGCAGCTACTTCATGTACAGC | | | | | |
| | AAGCTGAGGGTTGAGAAGAAGAACTGGGTTGAGAGAAACAGCTACAGCTGCTCTGTGGTGCATG | | | | | |
| | AGGGCCTGCACAATCACCACACCACCAAGAGCTTCAGCAGAACCCCTGGCAAG | | | | | |
| 4P3- | 321 | 4P3 VL | 4GS3 | 4P3 VH | 9G Modified IgG2a | IgG2a |
| VL3 VH- | | | | | Hinge | |
| 9GIgG2a | GACATCCAGCTGACACAGAGCCCTGACAGCCTGGCTGCTTCTCTGGGAGAGAGAGTGACCATCA | | | | | |
| | ACTGCAGAAGCTCCCAGAACATCTTCTACAACAGCAACAACAAGAACTTCCTGGCCTGGTATCA | | | | | |
| | GCAGAAGGCTGGCCAGTCTCCTAAGCTGCTGATCTACTGGGCCAGCACCAGAGAATCTGGGGTG | | | | | |
| | CCAGATAGATTTTCTGGCAGTGGCTCTGGCACAGACTTCACCCTGACAATCAGTGGACTGCAGG | | | | | |
| | CTGGGGATGTTGCTGTGTACTTCTGCCAGCAGTACTTCACAGATGCCCCTACCTTTGGTGGTGG | | | | | |
| | CACCAAGGTGGAAATCAAGGGTGGAGGCGGTTCAGGCGGAGGTGGCAGCGGCGGTGGAGGGTCG | | | | | |
| | GAGGTGCAGCTGGTGCAGTCTGGGGCTGAAGTGAAGAAGCCTGGGGAGAGCCTGAAGATCAGCT | | | | | |
| | GCAAAGTGTCTGGCTATGACTTCAGCAAGTACTGGATTGGCTGGGTCAGACAGATGCCTGGCAA | | | | | |
| | AGGCCTGGAATGGATGGGACTGATCTACCCTGGGGACTCTGACACCAGATACAGCCCTAGCCTG | | | | | |
| | GAAGGCCAGGTGTCCATCTCTGCTGACAAGAGCATCTCTGCAGCCTACCTGCAGTTCAGCAGCC | | | | | |
| | TGAAGGCCTCTGACTCTGCCATGTACTACTGTGTCAGAAAGGGCTACTCTGCCTACCTTGGAGG | | | | | |
| | ATGGTTTGATCCTTGGGGCCAGGGCACACTGGTCACAGTGTCCTCTGGCGGAGGAGGCGGAGGT | | | | | |
| | GGAGGAGGAGAGCCTAGAGGCCCCACCATCAAGCCCTGTCCTCCATGCAAGTGTCCTGCTCCTA | | | | | |
| | ACCTGCTTGGAGGCCCCTCTGTGTTCATCTTCCCACCTAAGATCAAGGATGTGCTGATGATCTC | | | | | |
| | TCTGAGCCCCATTGTGACCTGTGTGGTGGTGGATGTGTCTGAGGATGACCCTGATGTGCAGATC | | | | | |
| | TCTTGGTTTGTGAACAATGTGGAAGTGCACACAGCCCAGACACAGACCCACAGAGAGGACTACA | | | | | |
| | ACAGCACCCTGAGAGTGGTGTCTGCCCTGCCTATCCAGCACCAGGACTGGATGAGTGGCAAAGA | | | | | |
| | GTTCAAGTGCAAAGTGAACAACAAGGACCTGCCTGCTCCAATTGAGAGAACCATCAGCAAGCCC | | | | | |
| | AAGGGCTCTGTTAGAGCCCCTCAAGTGTATGTGCTGCCTCCACCTGAGGAAGAGATGACCAAGA | | | | | |
| | AACAAGTGACCCTGACATGCATGGTCACAGACTTCATGCCTGAGGACATCTATGTGGAATGGAC | | | | | |
| | CAACAATGGCAAGACAGAGCTGAACTACAAGAACACTGAGCCTGTGCTGGACTCTGATGGCAGC | | | | | |
| | TACTTCATGTACAGCAAGCTGAGGGTTGAGAAGAAGAACTGGGTTGAGAGAAACAGCTACAGCT | | | | | |
| | GCTCTGTGGTGCATGAGGGCCTGCACAATCACCACACCACCAAGAGCTTCAGCAGAACCCCTGG | | | | | |
| | CAAG | | | | | |
| 4P3- | 322 | 4P3 VL | 4GS4 | 4P3 VH | 9G Modified IgG2a | IgG2a |
| VL4VH- | | | | | Hinge | |
| 9GIgG2a | GACATCCAGCTGACACAGAGCCCTGACAGCCTGGCTGCTTCTCTGGGAGAGAGAGTGACCATCA | | | | | |
| | ACTGCAGAAGCTCCCAGAACATCTTCTACAACAGCAACAACAAGAACTTCCTGGCCTGGTATCA | | | | | |
| | GCAGAAGGCTGGCCAGTCTCCTAAGCTGCTGATCTACTGGGCCAGCACCAGAGAATCTGGGGTG | | | | | |
| | CCAGATAGATTTTCTGGCAGTGGCTCTGGCACAGACTTCACCCTGACAATCAGTGGACTGCAGG | | | | | |
| | CTGGGGATGTTGCTGTGTACTTCTGCCAGCAGTACTTCACAGATGCCCCTACCTTTGGTGGTGG | | | | | |
| | CACCAAGGTGGAAATCAAGGGTGGAGGCGGTTCAGGCGGAGGTGGCAGCGGCGGTGGAGGGTCA | | | | | |
| | GGTGGAGGTGGATCAGAGGTGCAGCTGGTGCAGTCTGGGGCTGAAGTGAAGAAGCCTGGGGAGA | | | | | |
| | GCCTGAAGATCAGCTGCAAAGTGTCTGGCTATGACTTCAGCAAGTACTGGATTGGCTGGGTCAG | | | | | |
| | ACAGATGCCTGGCAAAGGCCTGGAATGGATGGGACTGATCTACCCTGGGGACTCTGACACCAGA | | | | | |
| | TACAGCCCTAGCCTGGAAGGCCAGGTGTCCATCTCTGCTGACAAGAGCATCTCTGCAGCCTACC | | | | | |
| | TGCAGTTCAGCAGCCTGAAGGCCTCTGACTCTGCCATGTACTACTGTGTCAGAAAGGGCTACTC | | | | | |
| | TGCCTACCTTGGAGGATGGTTTGATCCTTGGGGCCAGGGCACACTGGTCACAGTGTCCTCTGGC | | | | | |
| | GGAGGAGGCGGAGGTGGAGGAGGAGAGCCTAGAGGCCCCACCATCAAGCCCTGTCCTCCATGCA | | | | | |
| | AGTGTCCTGCTCCTAACCTGCTTGGAGGCCCCTCTGTGTTCATCTTCCCACCTAAGATCAAGGA | | | | | |
| | TGTGCTGATGATCTCTCTGAGCCCCATTGTGACCTGTGTGGTGGTGGATGTGTCTGAGGATGAC | | | | | |
| | CCTGATGTGCAGATCTCTTGGTTTGTGAACAATGTGGAAGTGCACACAGCCCAGACACAGACCC | | | | | |
| | ACAGAGAGGACTACAACAGCACCCTGAGAGTGGTGTCTGCCCTGCCTATCCAGCACCAGGACTG | | | | | |
| | GATGAGTGGCAAAGAGTTCAAGTGCAAAGTGAACAACAAGGACCTGCCTGCTCCAATTGAGAGA | | | | | |
| | ACCATCAGCAAGCCCAAGGGCTCTGTTAGAGCCCCTCAAGTGTATGTGCTGCCTCCACCTGAGG | | | | | |
| | AAGAGATGACCAAGAAACAAGTGACCCTGACATGCATGGTCACAGACTTCATGCCTGAGGACAT | | | | | |
| | CTATGTGGAATGGACCAACAATGGCAAGACAGAGCTGAACTACAAGAACACTGAGCCTGTGCTG | | | | | |
| | GACTCTGATGGCAGCTACTTCATGTACAGCAAGCTGAGGGTTGAGAAGAAGAACTGGGTTGAGA | | | | | |
| | GAAACAGCTACAGCTGCTCTGTGGTGCATGAGGGCCTGCACAATCACCACACCACCAAGAGCTT | | | | | |
| | CAGCAGAACCCCTGGCAAG | | | | | |
| 31B6- | 323 | 31B6 VH | 4GS2 | 31B6 VL | 9G Modified IgG2a | IgG2a |
| VH2VL- | | | | | Hinge | |
| 9GIgG2a | GAGGTGCAGCTGCTGCAGAGTGGTGGTGGTGTTGTGCAGCCTGGTGGCTCTCTGAGACTGTCTT | | | | | |
| | GTGAAGCCTCTGGCTTCACCTTCAGCAGCTACAGCATGCACTGGGTCAGACAGGCCCCTGGCAA | | | | | |
| | AGGACTGGAATGGCTGACCCTGATCAGCTACAATGGGGTCAACAAGTACTATGCCAAGTCTGTG | | | | | |
| | AAGGGCAGATTTGCCATCAGCAGAGATGACAGCAAGTCTACCCTGTTCCTGCAGATGAACAGCC | | | | | |
| | TGACCACAGATGACACTGCTGTGTACTACTGTGCCAGATGTCCCATTCCTAGAGCTGCCAGCTA | | | | | |
| | CAGAAATGGGGACAACTACTTTGAGTTTGGCCAGGGCACCCTGGTCACAGTGTCCTCTGGCGGA | | | | | |
| | GGTGGCAGCGGAGGTGGCGGATCACAGGCTGTGGTCACCCAAGAGCCTAGCCTGTCTGTGTCTC | | | | | |
| | CTGGTGGCACAGTGACACTGACATGTGCCCTGTCCTCTGCCTCTGTGTCTACCAGCAGCTACCC | | | | | |
| | CAGCTGGTATCAGCAGACACCTGGCCAGCCTCCAAGAATGCTGATCCACAACACAGACACCAGA | | | | | |
| | TCCTCTGGGGTGCCAGACAGATTCTCTGGCAGCATCCTGGGCAAGAAGGCTGCCCTGACAATCA | | | | | |
| | CTGGTGCCCAGGCTGATGATGAGTCTGACTACTACTGTGTGGTGTACCTTGGCAATGGCTTCTG | | | | | |
| | GGTGTTTGGTGGTGGCACCAAGCTGACAGTGCTGGGCGGAGGAGGCGGAGGTGGAGGAGGAGAG | | | | | |
| | CCTAGAGGCCCCACCATCAAGCCCTGTCCTCCATGCAAGTGTCCTGCTCCTAACCTGCTTGGAG | | | | | |
| | GCCCCTCTGTGTTCATCTTCCCACCTAAGATCAAGGATGTGCTGATGATCTCTCTGAGCCCCAT | | | | | |
| | TGTGACCTGTGTGGTGGTGGATGTGTCTGAGGATGACCCTGATGTGCAGATCTCTTGGTTTGTG | | | | | |
| | AACAATGTGGAAGTGCACACAGCCCAGACACAGACCCACAGAGAGGACTACAACAGCACCCTGA | | | | | |
| | GAGTGGTGTCTGCCCTGCCTATCCAGCACCAGGACTGGATGAGTGGCAAAGAGTTCAAGTGCAA | | | | | |
| | AGTGAACAACAAGGACCTGCCTGCTCCAATTGAGAGAACCATCAGCAAGCCCAAGGGCTCTGTT | | | | | |
| | AGAGCCCCTCAAGTGTATGTGCTGCCTCCACCTGAGGAAGAGATGACCAAGAAACAAGTGACCC | | | | | |
| | TGACATGCATGGTCACAGACTTCATGCCTGAGGACATCTATGTGGAATGGACCAACAATGGCAA | | | | | |
| | GACAGAGCTGAACTACAAGAACACTGAGCCTGTGCTGGACTCTGATGGCAGCTACTTCATGTAC | | | | | |
| | AGCAAGCTGAGGGTTGAGAAGAAGAACTGGGTTGAGAGAAACAGCTACAGCTGCTCTGTGGTGC | | | | | |
| | ATGAGGGCCTGCACAATCACCACACCACCAAGAGCTTCAGCAGAACCCCTGGCAAG | | | | | |
| 31B6- | 347 | 31B6 VH | 4GS2 | 31B6 VL | 9G Modified IgG1 | IgG1 |
| VH2VL- | | | | | Hinge | |
| 9GIgG1 | GAGGTGCAGCTGCTGCAGAGTGGTGGTGGTGTTGTGCAGCCTGGTGGCTCTCTGAGACTGTCTT | | | | | |
| | GTGAAGCCTCTGGCTTCACCTTCAGCAGCTACAGCATGCACTGGGTCAGACAGGCCCCTGGCAA | | | | | |
| | AGGACTGGAATGGCTGACCCTGATCAGCTACAATGGGGTCAACAAGTACTATGCCAAGTCTGTG | | | | | |
| | AAGGGCAGATTTGCCATCAGCAGAGATGACAGCAAGTCTACCCTGTTCCTGCAGATGAACAGCC | | | | | |
| | TGACCACAGATGACACTGCTGTGTACTACTGTGCCAGATGTCCCATTCCTAGAGCTGCCAGCTA | | | | | |
| | CAGAAATGGGGACAACTACTTTGAGTTTGGCCAGGGCACCCTGGTCACAGTGTCCTCTGGCGGA | | | | | |
| | GGTGGCAGCGGAGGTGGCGGATCACAGGCTGTGGTCACCCAAGAGCCTAGCCTGTCTGTGTCTC | | | | | |
| | CTGGTGGCACAGTGACACTGACATGTGCCCTGTCCTCTGCCTCTGTGTCTACCAGCAGCTACCC | | | | | |
| | CAGCTGGTATCAGCAGACACCTGGCCAGCCTCCAAGAATGCTGATCCACAACACAGACACCAGA | | | | | |
| | TCCTCTGGGGTGCCAGACAGATTCTCTGGCAGCATCCTGGGCAAGAAGGCTGCCCTGACAATCA | | | | | |
| | CTGGTGCCCAGGCTGATGATGAGTCTGACTACTACTGTGTGGTGTACCTTGGCAATGGCTTCTG | | | | | |
| | GGTGTTTGGTGGTGGCACCAAGCTGACAGTGCTGGGCGGAGGAGGCGGAGGTGGAGGAGGAGAA | | | | | |
| | CCCAAGAGCTGTGACAAGACCCACACCTGTCCTCCATGTCCTGCTCCAGAACTGCTTGGAGGCC | | | | | |
| | CTTCTGTGTTCCTGTTTCCTCCAAAGCCTAAGGACACCCTGATGATCAGCAGAACCCCTGAAGT | | | | | |
| | GACCTGTGTGGTGGTTGATGTGTCCCATGAGGACCCAGAAGTGAAGTTCAATTGGTATGTGGAT | | | | | |
| | GGGGTTGAAGTGCACAATGCCAAGACCAAGCCTAGAGAGGAACAGTACAACTCCACCTACAGAG | | | | | |
| | TGGTGTCTGTGCTGACAGTGCTGCACCAGGACTGGCTGAATGGCAAAGAGTACAAGTGCAAGGT | | | | | |
| | GTCCAACAAGGCCCTGCCTGCTCCTATTGAGAAAACCATCTCCAAGGCCAAGGGCCAGCCAAGA | | | | | |
| | GAACCCCAGGTTTACACCCTGCCACCTAGCAGAGAAGAGATGACCAAGAACCAGGTGTCCCTGA | | | | | |
| | CCTGCCTGGTTAAGGGCTTCTACCCCTCTGACATTGCTGTGGAATGGGAGAGCAATGGCCAGCC | | | | | |
| | TGAAAACAACTACAAGACAACCCCTCCTGTGCTGGACTCAGATGGCTCATTCTTCCTGTACAGC | | | | | |
| | AAGCTGACTGTGGACAAGTCCAGGTGGCAGCAGGGAAATGTGTTCAGCTGCTCTGTGATGCATG | | | | | |
| | AGGCCCTGCACAACCACTACACCCAGAAAAGTCTGAGTCTGAGCCCTGGCAAG | | | | | |
| 31B6- | 324 | 31B6 VH | 4GS3 | 31B6 VL | 9G Modified IgG2a | IgG2a |
| VH3VL- | | | | | Hinge | |
| 9GIgG2a | GAGGTGCAGCTGCTGCAGAGTGGTGGTGGTGTTGTGCAGCCTGGTGGCTCTCTGAGACTGTCTT | | | | | |
| | GTGAAGCCTCTGGCTTCACCTTCAGCAGCTACAGCATGCACTGGGTCAGACAGGCCCCTGGCAA | | | | | |
| | AGGACTGGAATGGCTGACCCTGATCAGCTACAATGGGGTCAACAAGTACTATGCCAAGTCTGTG | | | | | |
| | AAGGGCAGATTTGCCATCAGCAGAGATGACAGCAAGTCTACCCTGTTCCTGCAGATGAACAGCC | | | | | |
| | TGACCACAGATGACACTGCTGTGTACTACTGTGCCAGATGTCCCATTCCTAGAGCTGCCAGCTA | | | | | |
| | CAGAAATGGGGACAACTACTTTGAGTTTGGCCAGGGCACCCTGGTCACAGTGTCCTCTGGTGGA | | | | | |
| | GGCGGTTCAGGCGGAGGTGGCAGCGGCGGTGGAGGGTCGCAGGCTGTGGTCACCCAAGAGCCTA | | | | | |
| | GCCTGTCTGTGTCTCCTGGTGGCACAGTGACACTGACATGTGCCCTGTCCTCTGCCTCTGTGTC | | | | | |
| | TACCAGCAGCTACCCCAGCTGGTATCAGCAGACACCTGGCCAGCCTCCAAGAATGCTGATCCAC | | | | | |
| | AACACAGACACCAGATCCTCTGGGGTGCCAGACAGATTCTCTGGCAGCATCCTGGGCAAGAAGG | | | | | |
| | CTGCCCTGACAATCACTGGTGCCCAGGCTGATGATGAGTCTGACTACTACTGTGTGGTGTACCT | | | | | |
| | TGGCAATGGCTTCTGGGTGTTTGGTGGTGGCACCAAGCTGACAGTGCTGGGCGGAGGAGGCGGA | | | | | |
| | GGTGGAGGAGGAGAGCCTAGAGGCCCCACCATCAAGCCCTGTCCTCCATGCAAGTGTCCTGCTC | | | | | |
| | CTAACCTGCTTGGAGGCCCCTCTGTGTTCATCTTCCCACCTAAGATCAAGGATGTGCTGATGAT | | | | | |
| | CTCTCTGAGCCCCATTGTGACCTGTGTGGTGGTGGATGTGTCTGAGGATGACCCTGATGTGCAG | | | | | |
| | ATCTCTTGGTTTGTGAACAATGTGGAAGTGCACACAGCCCAGACACAGACCCACAGAGAGGACT | | | | | |
| | ACAACAGCACCCTGAGAGTGGTGTCTGCCCTGCCTATCCAGCACCAGGACTGGATGAGTGGCAA | | | | | |
| | AGAGTTCAAGTGCAAAGTGAACAACAAGGACCTGCCTGCTCCAATTGAGAGAACCATCAGCAAG | | | | | |
| | CCCAAGGGCTCTGTTAGAGCCCCTCAAGTGTATGTGCTGCCTCCACCTGAGGAAGAGATGACCA | | | | | |
| | AGAAACAAGTGACCCTGACATGCATGGTCACAGACTTCATGCCTGAGGACATCTATGTGGAATG | | | | | |
| | GACCAACAATGGCAAGACAGAGCTGAACTACAAGAACACTGAGCCTGTGCTGGACTCTGATGGC | | | | | |
| | AGCTACTTCATGTACAGCAAGCTGAGGGTTGAGAAGAAGAACTGGGTTGAGAGAAACAGCTACA | | | | | |
| | GCTGCTCTGTGGTGCATGAGGGCCTGCACAATCACCACACCACCAAGAGCTTCAGCAGAACCCC | | | | | |
| | TGGCAAG | | | | | |
| 31B6- | 325 | 31B6 VH | 4GS4 | 31B6 VL | 9G Modified IgG2a | IgG2a |
| VH4VL- | | | | | Hinge | |
| 9GIgG2a | GAGGTGCAGCTGCTGCAGAGTGGTGGTGGTGTTGTGCAGCCTGGTGGCTCTCTGAGACTGTCTT | | | | | |
| | GTGAAGCCTCTGGCTTCACCTTCAGCAGCTACAGCATGCACTGGGTCAGACAGGCCCCTGGCAA | | | | | |
| | AGGACTGGAATGGCTGACCCTGATCAGCTACAATGGGGTCAACAAGTACTATGCCAAGTCTGTG | | | | | |
| | AAGGGCAGATTTGCCATCAGCAGAGATGACAGCAAGTCTACCCTGTTCCTGCAGATGAACAGCC | | | | | |
| | TGACCACAGATGACACTGCTGTGTACTACTGTGCCAGATGTCCCATTCCTAGAGCTGCCAGCTA | | | | | |
| | CAGAAATGGGGACAACTACTTTGAGTTTGGCCAGGGCACCCTGGTCACAGTGTCCTCTGGTGGA | | | | | |
| | GGCGGTTCAGGCGGAGGTGGCAGCGGCGGTGGAGGGTCAGGTGGAGGTGGATCACAGGCTGTGG | | | | | |
| | TCACCCAAGAGCCTAGCCTGTCTGTGTCTCCTGGTGGCACAGTGACACTGACATGTGCCCTGTC | | | | | |
| | CTCTGCCTCTGTGTCTACCAGCAGCTACCCCAGCTGGTATCAGCAGACACCTGGCCAGCCTCCA | | | | | |
| | AGAATGCTGATCCACAACACAGACACCAGATCCTCTGGGGTGCCAGACAGATTCTCTGGCAGCA | | | | | |
| | TCCTGGGCAAGAAGGCTGCCCTGACAATCACTGGTGCCCAGGCTGATGATGAGTCTGACTACTA | | | | | |
| | CTGTGTGGTGTACCTTGGCAATGGCTTCTGGGTGTTTGGTGGTGGCACCAAGCTGACAGTGCTG | | | | | |
| | GGCGGAGGAGGCGGAGGTGGAGGAGGAGAGCCTAGAGGCCCCACCATCAAGCCCTGTCCTCCAT | | | | | |
| | GCAAGTGTCCTGCTCCTAACCTGCTTGGAGGCCCCTCTGTGTTCATCTTCCCACCTAAGATCAA | | | | | |
| | GGATGTGCTGATGATCTCTCTGAGCCCCATTGTGACCTGTGTGGTGGTGGATGTGTCTGAGGAT | | | | | |
| | GACCCTGATGTGCAGATCTCTTGGTTTGTGAACAATGTGGAAGTGCACACAGCCCAGACACAGA | | | | | |
| | CCCACAGAGAGGACTACAACAGCACCCTGAGAGTGGTGTCTGCCCTGCCTATCCAGCACCAGGA | | | | | |
| | CTGGATGAGTGGCAAAGAGTTCAAGTGCAAAGTGAACAACAAGGACCTGCCTGCTCCAATTGAG | | | | | |
| | AGAACCATCAGCAAGCCCAAGGGCTCTGTTAGAGCCCCTCAAGTGTATGTGCTGCCTCCACCTG | | | | | |
| | AGGAAGAGATGACCAAGAAACAAGTGACCCTGACATGCATGGTCACAGACTTCATGCCTGAGGA | | | | | |
| | CATCTATGTGGAATGGACCAACAATGGCAAGACAGAGCTGAACTACAAGAACACTGAGCCTGTG | | | | | |
| | CTGGACTCTGATGGCAGCTACTTCATGTACAGCAAGCTGAGGGTTGAGAAGAAGAACTGGGTTG | | | | | |
| | AGAGAAACAGCTACAGCTGCTCTGTGGTGCATGAGGGCCTGCACAATCACCACACCACCAAGAG | | | | | |
| | CTTCAGCAGAACCCCTGGCAAG | | | | | |
| 31B6- | 348 | 31B6 VH | 4GS4 | 31B6 VL | 9G Modified IgG1 | IgG1 |
| VH4VL- | | | | | Hinge | |
| 9GIgG1 | GAGGTGCAGCTGCTGCAGAGTGGTGGTGGTGTTGTGCAGCCTGGTGGCTCTCTGAGACTGTCTT | | | | | |
| | GTGAAGCCTCTGGCTTCACCTTCAGCAGCTACAGCATGCACTGGGTCAGACAGGCCCCTGGCAA | | | | | |
| | AGGACTGGAATGGCTGACCCTGATCAGCTACAATGGGGTCAACAAGTACTATGCCAAGTCTGTG | | | | | |
| | AAGGGCAGATTTGCCATCAGCAGAGATGACAGCAAGTCTACCCTGTTCCTGCAGATGAACAGCC | | | | | |
| | TGACCACAGATGACACTGCTGTGTACTACTGTGCCAGATGTCCCATTCCTAGAGCTGCCAGCTA | | | | | |
| | CAGAAATGGGGACAACTACTTTGAGTTTGGCCAGGGCACCCTGGTCACAGTGTCCTCTGGTGGA | | | | | |
| | GGCGGTTCAGGCGGAGGTGGCAGCGGCGGTGGAGGGTCAGGTGGAGGTGGATCACAGGCTGTGG | | | | | |
| | TCACCCAAGAGCCTAGCCTGTCTGTGTCTCCTGGTGGCACAGTGACACTGACATGTGCCCTGTC | | | | | |
| | CTCTGCCTCTGTGTCTACCAGCAGCTACCCCAGCTGGTATCAGCAGACACCTGGCCAGCCTCCA | | | | | |
| | AGAATGCTGATCCACAACACAGACACCAGATCCTCTGGGGTGCCAGACAGATTCTCTGGCAGCA | | | | | |
| | TCCTGGGCAAGAAGGCTGCCCTGACAATCACTGGTGCCCAGGCTGATGATGAGTCTGACTACTA | | | | | |
| | CTGTGTGGTGTACCTTGGCAATGGCTTCTGGGTGTTTGGTGGTGGCACCAAGCTGACAGTGCTG | | | | | |
| | GGCGGAGGAGGCGGAGGTGGAGGAGGAGAACCCAAGAGCTGTGACAAGACCCACACCTGTCCTC | | | | | |
| | CATGTCCTGCTCCAGAACTGCTTGGAGGCCCTTCTGTGTTCCTGTTTCCTCCAAAGCCTAAGGA | | | | | |
| | CACCCTGATGATCAGCAGAACCCCTGAAGTGACCTGTGTGGTGGTTGATGTGTCCCATGAGGAC | | | | | |
| | CCAGAAGTGAAGTTCAATTGGTATGTGGATGGGGTTGAAGTGCACAATGCCAAGACCAAGCCTA | | | | | |
| | GAGAGGAACAGTACAACTCCACCTACAGAGTGGTGTCTGTGCTGACAGTGCTGCACCAGGACTG | | | | | |
| | GCTGAATGGCAAAGAGTACAAGTGCAAGGTGTCCAACAAGGCCCTGCCTGCTCCTATTGAGAAA | | | | | |
| | ACCATCTCCAAGGCCAAGGGCCAGCCAAGAGAACCCCAGGTTTACACCCTGCCACCTAGCAGAG | | | | | |
| | AAGAGATGACCAAGAACCAGGTGTCCCTGACCTGCCTGGTTAAGGGCTTCTACCCCTCTGACAT | | | | | |
| | TGCTGTGGAATGGGAGAGCAATGGCCAGCCTGAAAACAACTACAAGACAACCCCTCCTGTGCTG | | | | | |
| | GACTCAGATGGCTCATTCTTCCTGTACAGCAAGCTGACTGTGGACAAGTCCAGGTGGCAGCAGG | | | | | |
| | GAAATGTGTTCAGCTGCTCTGTGATGCATGAGGCCCTGCACAACCACTACACCCAGAAAAGTCT | | | | | |
| | GAGTCTGAGCCCTGGCAAG | | | | | |
| 31B6- | 326 | 31B6 VL | 4GS2 | 31B6 VH | 9G Modified IgG2a | IgG2a |
| VL2VH- | | | | | Hinge | |
| 9GIgG2a | CAGGCTGTGGTCACCCAAGAGCCTAGCCTGTCTGTGTCTCCTGGTGGCACAGTGACACTGACAT | | | | | |
| | GTGCCCTGTCCTCTGCCTCTGTGTCTACCAGCAGCTACCCCAGCTGGTATCAGCAGACACCTGG | | | | | |
| | CCAGCCTCCAAGAATGCTGATCCACAACACAGACACCAGATCCTCTGGGGTGCCAGACAGATTC | | | | | |
| | TCTGGCAGCATCCTGGGCAAGAAGGCTGCCCTGACAATCACTGGTGCCCAGGCTGATGATGAGT | | | | | |
| | CTGACTACTACTGTGTGGTGTACCTTGGCAATGGCTTCTGGGTGTTTGGTGGTGGCACCAAGCT | | | | | |
| | GACAGTGCTGGGCGGAGGTGGCAGCGGAGGTGGCGGATCAGAGGTGCAGCTGCTGCAGAGTGGT | | | | | |
| | GGTGGTGTTGTGCAGCCTGGTGGCTCTCTGAGACTGTCTTGTGAAGCCTCTGGCTTCACCTTCA | | | | | |
| | GCAGCTACAGCATGCACTGGGTCAGACAGGCCCCTGGCAAAGGACTGGAATGGCTGACCCTGAT | | | | | |
| | CAGCTACAATGGGGTCAACAAGTACTATGCCAAGTCTGTGAAGGGCAGATTTGCCATCAGCAGA | | | | | |
| | GATGACAGCAAGTCTACCCTGTTCCTGCAGATGAACAGCCTGACCACAGATGACACTGCTGTGT | | | | | |
| | ACTACTGTGCCAGATGTCCCATTCCTAGAGCTGCCAGCTACAGAAATGGGGACAACTACTTTGA | | | | | |
| | GTTTGGCCAGGGCACCCTGGTCACAGTGTCCTCTGGCGGAGGAGGCGGAGGTGGAGGAGGAGAG | | | | | |
| | CCTAGAGGCCCCACCATCAAGCCCTGTCCTCCATGCAAGTGTCCTGCTCCTAACCTGCTTGGAG | | | | | |
| | GCCCCTCTGTGTTCATCTTCCCACCTAAGATCAAGGATGTGCTGATGATCTCTCTGAGCCCCAT | | | | | |
| | TGTGACCTGTGTGGTGGTGGATGTGTCTGAGGATGACCCTGATGTGCAGATCTCTTGGTTTGTG | | | | | |
| | AACAATGTGGAAGTGCACACAGCCCAGACACAGACCCACAGAGAGGACTACAACAGCACCCTGA | | | | | |
| | GAGTGGTGTCTGCCCTGCCTATCCAGCACCAGGACTGGATGAGTGGCAAAGAGTTCAAGTGCAA | | | | | |
| | AGTGAACAACAAGGACCTGCCTGCTCCAATTGAGAGAACCATCAGCAAGCCCAAGGGCTCTGTT | | | | | |
| | AGAGCCCCTCAAGTGTATGTGCTGCCTCCACCTGAGGAAGAGATGACCAAGAAACAAGTGACCC | | | | | |
| | TGACATGCATGGTCACAGACTTCATGCCTGAGGACATCTATGTGGAATGGACCAACAATGGCAA | | | | | |
| | GACAGAGCTGAACTACAAGAACACTGAGCCTGTGCTGGACTCTGATGGCAGCTACTTCATGTAC | | | | | |
| | AGCAAGCTGAGGGTTGAGAAGAAGAACTGGGTTGAGAGAAACAGCTACAGCTGCTCTGTGGTGC | | | | | |
| | ATGAGGGCCTGCACAATCACCACACCACCAAGAGCTTCAGCAGAACCCCTGGCAAG | | | | | |
| 31B6- | 327 | 31B6 VL | 4GS3 | 31B6 VH | 9G Modified IgG2a | IgG2a |
| VL3 VH- | | | | | Hinge | |
| 9GIgG2a | CAGGCTGTGGTCACCCAAGAGCCTAGCCTGTCTGTGTCTCCTGGTGGCACAGTGACACTGACAT | | | | | |
| | GTGCCCTGTCCTCTGCCTCTGTGTCTACCAGCAGCTACCCCAGCTGGTATCAGCAGACACCTGG | | | | | |
| | CCAGCCTCCAAGAATGCTGATCCACAACACAGACACCAGATCCTCTGGGGTGCCAGACAGATTC | | | | | |
| | TCTGGCAGCATCCTGGGCAAGAAGGCTGCCCTGACAATCACTGGTGCCCAGGCTGATGATGAGT | | | | | |
| | CTGACTACTACTGTGTGGTGTACCTTGGCAATGGCTTCTGGGTGTTTGGTGGTGGCACCAAGCT | | | | | |
| | GACAGTGCTGGGTGGAGGCGGTTCAGGCGGAGGTGGCAGCGGCGGTGGAGGGTCGGAGGTGCAG | | | | | |
| | CTGCTGCAGAGTGGTGGTGGTGTTGTGCAGCCTGGTGGCTCTCTGAGACTGTCTTGTGAAGCCT | | | | | |
| | CTGGCTTCACCTTCAGCAGCTACAGCATGCACTGGGTCAGACAGGCCCCTGGCAAAGGACTGGA | | | | | |
| | ATGGCTGACCCTGATCAGCTACAATGGGGTCAACAAGTACTATGCCAAGTCTGTGAAGGGCAGA | | | | | |
| | TTTGCCATCAGCAGAGATGACAGCAAGTCTACCCTGTTCCTGCAGATGAACAGCCTGACCACAG | | | | | |
| | ATGACACTGCTGTGTACTACTGTGCCAGATGTCCCATTCCTAGAGCTGCCAGCTACAGAAATGG | | | | | |
| | GGACAACTACTTTGAGTTTGGCCAGGGCACCCTGGTCACAGTGTCCTCTGGCGGAGGAGGCGGA | | | | | |
| | GGTGGAGGAGGAGAGCCTAGAGGCCCCACCATCAAGCCCTGTCCTCCATGCAAGTGTCCTGCTC | | | | | |
| | CTAACCTGCTTGGAGGCCCCTCTGTGTTCATCTTCCCACCTAAGATCAAGGATGTGCTGATGAT | | | | | |
| | CTCTCTGAGCCCCATTGTGACCTGTGTGGTGGTGGATGTGTCTGAGGATGACCCTGATGTGCAG | | | | | |
| | ATCTCTTGGTTTGTGAACAATGTGGAAGTGCACACAGCCCAGACACAGACCCACAGAGAGGACT | | | | | |
| | ACAACAGCACCCTGAGAGTGGTGTCTGCCCTGCCTATCCAGCACCAGGACTGGATGAGTGGCAA | | | | | |
| | AGAGTTCAAGTGCAAAGTGAACAACAAGGACCTGCCTGCTCCAATTGAGAGAACCATCAGCAAG | | | | | |
| | CCCAAGGGCTCTGTTAGAGCCCCTCAAGTGTATGTGCTGCCTCCACCTGAGGAAGAGATGACCA | | | | | |
| | AGAAACAAGTGACCCTGACATGCATGGTCACAGACTTCATGCCTGAGGACATCTATGTGGAATG | | | | | |
| | GACCAACAATGGCAAGACAGAGCTGAACTACAAGAACACTGAGCCTGTGCTGGACTCTGATGGC | | | | | |
| | AGCTACTTCATGTACAGCAAGCTGAGGGTTGAGAAGAAGAACTGGGTTGAGAGAAACAGCTACA | | | | | |
| | GCTGCTCTGTGGTGCATGAGGGCCTGCACAATCACCACACCACCAAGAGCTTCAGCAGAACCCC | | | | | |
| | TGGCAAG | | | | | |
| 31B6- | 328 | 31B6 VL | 4GS4 | 31B6 VH | 9G Modified IgG2a | IgG2a |
| VL4VH- | | | | | Hinge | |
| 9GIgG2a | CAGGCTGTGGTCACCCAAGAGCCTAGCCTGTCTGTGTCTCCTGGTGGCACAGTGACACTGACAT | | | | | |
| | GTGCCCTGTCCTCTGCCTCTGTGTCTACCAGCAGCTACCCCAGCTGGTATCAGCAGACACCTGG | | | | | |
| | CCAGCCTCCAAGAATGCTGATCCACAACACAGACACCAGATCCTCTGGGGTGCCAGACAGATTC | | | | | |
| | TCTGGCAGCATCCTGGGCAAGAAGGCTGCCCTGACAATCACTGGTGCCCAGGCTGATGATGAGT | | | | | |
| | CTGACTACTACTGTGTGGTGTACCTTGGCAATGGCTTCTGGGTGTTTGGTGGTGGCACCAAGCT | | | | | |
| | GACAGTGCTGGGTGGAGGCGGTTCAGGCGGAGGTGGCAGCGGCGGTGGAGGGTCAGGTGGAGGT | | | | | |
| | GGATCAGAGGTGCAGCTGCTGCAGAGTGGTGGTGGTGTTGTGCAGCCTGGTGGCTCTCTGAGAC | | | | | |
| | TGTCTTGTGAAGCCTCTGGCTTCACCTTCAGCAGCTACAGCATGCACTGGGTCAGACAGGCCCC | | | | | |
| | TGGCAAAGGACTGGAATGGCTGACCCTGATCAGCTACAATGGGGTCAACAAGTACTATGCCAAG | | | | | |
| | TCTGTGAAGGGCAGATTTGCCATCAGCAGAGATGACAGCAAGTCTACCCTGTTCCTGCAGATGA | | | | | |
| | ACAGCCTGACCACAGATGACACTGCTGTGTACTACTGTGCCAGATGTCCCATTCCTAGAGCTGC | | | | | |
| | CAGCTACAGAAATGGGGACAACTACTTTGAGTTTGGCCAGGGCACCCTGGTCACAGTGTCCTCT | | | | | |
| | GGCGGAGGAGGCGGAGGTGGAGGAGGAGAGCCTAGAGGCCCCACCATCAAGCCCTGTCCTCCAT | | | | | |
| | GCAAGTGTCCTGCTCCTAACCTGCTTGGAGGCCCCTCTGTGTTCATCTTCCCACCTAAGATCAA | | | | | |
| | GGATGTGCTGATGATCTCTCTGAGCCCCATTGTGACCTGTGTGGTGGTGGATGTGTCTGAGGAT | | | | | |
| | GACCCTGATGTGCAGATCTCTTGGTTTGTGAACAATGTGGAAGTGCACACAGCCCAGACACAGA | | | | | |
| | CCCACAGAGAGGACTACAACAGCACCCTGAGAGTGGTGTCTGCCCTGCCTATCCAGCACCAGGA | | | | | |
| | CTGGATGAGTGGCAAAGAGTTCAAGTGCAAAGTGAACAACAAGGACCTGCCTGCTCCAATTGAG | | | | | |
| | AGAACCATCAGCAAGCCCAAGGGCTCTGTTAGAGCCCCTCAAGTGTATGTGCTGCCTCCACCTG | | | | | |
| | AGGAAGAGATGACCAAGAAACAAGTGACCCTGACATGCATGGTCACAGACTTCATGCCTGAGGA | | | | | |
| | CATCTATGTGGAATGGACCAACAATGGCAAGACAGAGCTGAACTACAAGAACACTGAGCCTGTG | | | | | |
| | CTGGACTCTGATGGCAGCTACTTCATGTACAGCAAGCTGAGGGTTGAGAAGAAGAACTGGGTTG | | | | | |
| | AGAGAAACAGCTACAGCTGCTCTGTGGTGCATGAGGGCCTGCACAATCACCACACCACCAAGAG | | | | | |
| | CTTCAGCAGAACCCCTGGCAAG | | | | | |
| 8H1- | 329 | 8H1 VH | 4GS2 | 8H1 VL | 9G Modified IgG2a | IgG2a |
| VH2VL- | | | | | Hinge | |
| 9GIgG2a | GAGGTGCAGCTGGTGGAAAGTGGTGGTGGACTGGTTCAGCCTGGTGGCAGCCTGAGACTGTCTT | | | | | |
| | GTGCTGCCTCTGGCTTCACCTTCAGCATCTACACCATGAGCTGGGTCAGACAGGCTCCTGGCAA | | | | | |
| | AGGCCTGGAATGGGTGTCAGCTATCCTGTCTGATGGCAGCACCTACTATGCTGACTCTGTGGAA | | | | | |
| | GGCAGATTCACCATCAGCAGAGACAACAGCAAGACCACACTGTACCTGCAGATGAACAGCCTGA | | | | | |
| | CAGCTGAGGACACTGCTGTGTACTACTGCACCAAGAGAGTGGAAGGCTCCTTCAGAGGCTGGGG | | | | | |
| | ACAGGGAACACTGGTCACTGTGTCCTCTGGCGGAGGTGGCAGCGGAGGTGGCGGATCAGAGATT | | | | | |
| | GTGATGACACAGAGCCCTGGCACACTGTCTCTGAGCCCTGGGGAATCTGCCACACTGAGCTGTA | | | | | |
| | GAGCCAGCCAGTCTGTGTCTACCACCTACCTGGCCTGGTATCAGCAGAAGCCTGGACAGGCTCC | | | | | |
| | CAGACTGCTGATCTATGGGGCCTCTTCTAGAGCCACAGGCATCCCTGACAGATTTTCTGGCAGT | | | | | |
| | GGCTCTGGCACAGACTTCACCCTGACCATCAGCAGACTGGAACCTGAGGACTTTGCTGTGTACT | | | | | |
| | ACTGCCAGCAGTATGGTGGCAGCAGGCTGATCACATTTGGCCAGGGCACAAGACTGGAAATCAA | | | | | |
| | GGGCGGAGGAGGCGGAGGTGGAGGAGGAGAGCCTAGAGGCCCCACCATCAAGCCCTGTCCTCCA | | | | | |
| | TGCAAGTGTCCTGCTCCTAACCTGCTTGGAGGCCCCTCTGTGTTCATCTTCCCACCTAAGATCA | | | | | |
| | AGGATGTGCTGATGATCTCTCTGAGCCCCATTGTGACCTGTGTGGTGGTGGATGTGTCTGAGGA | | | | | |
| | TGACCCTGATGTGCAGATCTCTTGGTTTGTGAACAATGTGGAAGTGCACACAGCCCAGACACAG | | | | | |
| | ACCCACAGAGAGGACTACAACAGCACCCTGAGAGTGGTGTCTGCCCTGCCTATCCAGCACCAGG | | | | | |
| | ACTGGATGAGTGGCAAAGAGTTCAAGTGCAAAGTGAACAACAAGGACCTGCCTGCTCCAATTGA | | | | | |
| | GAGAACCATCAGCAAGCCCAAGGGCTCTGTTAGAGCCCCTCAAGTGTATGTGCTGCCTCCACCT | | | | | |
| | GAGGAAGAGATGACCAAGAAACAAGTGACCCTGACATGCATGGTCACAGACTTCATGCCTGAGG | | | | | |
| | ACATCTATGTGGAATGGACCAACAATGGCAAGACAGAGCTGAACTACAAGAACACTGAGCCTGT | | | | | |
| | GCTGGACTCTGATGGCAGCTACTTCATGTACAGCAAGCTGAGGGTTGAGAAGAAGAACTGGGTT | | | | | |
| | GAGAGAAACAGCTACAGCTGCTCTGTGGTGCATGAGGGCCTGCACAATCACCACACCACCAAGA | | | | | |
| | GCTTCAGCAGAACCCCTGGCAAG | | | | | |
| 8H1- | 349 | 8H1 VH | 4GS2 | 8H1 VL | 9G Modified IgG1 | IgG1 |
| VH2VL- | | | | | Hinge | |
| 9GIgG1 | GAGGTGCAGCTGGTGGAAAGTGGTGGTGGACTGGTTCAGCCTGGTGGCAGCCTGAGACTGTCTT | | | | | |
| | GTGCTGCCTCTGGCTTCACCTTCAGCATCTACACCATGAGCTGGGTCAGACAGGCTCCTGGCAA | | | | | |
| | AGGCCTGGAATGGGTGTCAGCTATCCTGTCTGATGGCAGCACCTACTATGCTGACTCTGTGGAA | | | | | |
| | GGCAGATTCACCATCAGCAGAGACAACAGCAAGACCACACTGTACCTGCAGATGAACAGCCTGA | | | | | |
| | CAGCTGAGGACACTGCTGTGTACTACTGCACCAAGAGAGTGGAAGGCTCCTTCAGAGGCTGGGG | | | | | |
| | ACAGGGAACACTGGTCACTGTGTCCTCTGGCGGAGGTGGCAGCGGAGGTGGCGGATCAGAGATT | | | | | |
| | GTGATGACACAGAGCCCTGGCACACTGTCTCTGAGCCCTGGGGAATCTGCCACACTGAGCTGTA | | | | | |
| | GAGCCAGCCAGTCTGTGTCTACCACCTACCTGGCCTGGTATCAGCAGAAGCCTGGACAGGCTCC | | | | | |
| | CAGACTGCTGATCTATGGGGCCTCTTCTAGAGCCACAGGCATCCCTGACAGATTTTCTGGCAGT | | | | | |
| | GGCTCTGGCACAGACTTCACCCTGACCATCAGCAGACTGGAACCTGAGGACTTTGCTGTGTACT | | | | | |
| | ACTGCCAGCAGTATGGTGGCAGCAGGCTGATCACATTTGGCCAGGGCACAAGACTGGAAATCAA | | | | | |
| | GGGCGGAGGAGGCGGAGGTGGAGGAGGAGAACCCAAGAGCTGTGACAAGACCCACACCTGTCCT | | | | | |
| | CCATGTCCTGCTCCAGAACTGCTTGGAGGCCCTTCTGTGTTCCTGTTTCCTCCAAAGCCTAAGG | | | | | |
| | ACACCCTGATGATCAGCAGAACCCCTGAAGTGACCTGTGTGGTGGTTGATGTGTCCCATGAGGA | | | | | |
| | CCCAGAAGTGAAGTTCAATTGGTATGTGGATGGGGTTGAAGTGCACAATGCCAAGACCAAGCCT | | | | | |
| | AGAGAGGAACAGTACAACTCCACCTACAGAGTGGTGTCTGTGCTGACAGTGCTGCACCAGGACT | | | | | |
| | GGCTGAATGGCAAAGAGTACAAGTGCAAGGTGTCCAACAAGGCCCTGCCTGCTCCTATTGAGAA | | | | | |
| | AACCATCTCCAAGGCCAAGGGCCAGCCAAGAGAACCCCAGGTTTACACCCTGCCACCTAGCAGA | | | | | |
| | GAAGAGATGACCAAGAACCAGGTGTCCCTGACCTGCCTGGTTAAGGGCTTCTACCCCTCTGACA | | | | | |
| | TTGCTGTGGAATGGGAGAGCAATGGCCAGCCTGAAAACAACTACAAGACAACCCCTCCTGTGCT | | | | | |
| | GGACTCAGATGGCTCATTCTTCCTGTACAGCAAGCTGACTGTGGACAAGTCCAGGTGGCAGCAG | | | | | |
| | GGAAATGTGTTCAGCTGCTCTGTGATGCATGAGGCCCTGCACAACCACTACACCCAGAAAAGTC | | | | | |
| | TGAGTCTGAGCCCTGGCAAG | | | | | |
| 8H1- | 330 | 8H1 VH | 4GS3 | 8H1 VL | 9G Modified IgG2a | IgG2a |
| VH3VL- | | | | | Hinge | |
| 9GIgG2a | GAGGTGCAGCTGGTGGAAAGTGGTGGTGGACTGGTTCAGCCTGGTGGCAGCCTGAGACTGTCTT | | | | | |
| | GTGCTGCCTCTGGCTTCACCTTCAGCATCTACACCATGAGCTGGGTCAGACAGGCTCCTGGCAA | | | | | |
| | AGGCCTGGAATGGGTGTCAGCTATCCTGTCTGATGGCAGCACCTACTATGCTGACTCTGTGGAA | | | | | |
| | GGCAGATTCACCATCAGCAGAGACAACAGCAAGACCACACTGTACCTGCAGATGAACAGCCTGA | | | | | |
| | CAGCTGAGGACACTGCTGTGTACTACTGCACCAAGAGAGTGGAAGGCTCCTTCAGAGGCTGGGG | | | | | |
| | ACAGGGAACACTGGTCACTGTGTCCTCTGGTGGAGGCGGTTCAGGCGGAGGTGGCAGCGGCGGT | | | | | |
| | GGAGGGTCGGAGATTGTGATGACACAGAGCCCTGGCACACTGTCTCTGAGCCCTGGGGAATCTG | | | | | |
| | CCACACTGAGCTGTAGAGCCAGCCAGTCTGTGTCTACCACCTACCTGGCCTGGTATCAGCAGAA | | | | | |
| | GCCTGGACAGGCTCCCAGACTGCTGATCTATGGGGCCTCTTCTAGAGCCACAGGCATCCCTGAC | | | | | |
| | AGATTTTCTGGCAGTGGCTCTGGCACAGACTTCACCCTGACCATCAGCAGACTGGAACCTGAGG | | | | | |
| | ACTTTGCTGTGTACTACTGCCAGCAGTATGGTGGCAGCAGGCTGATCACATTTGGCCAGGGCAC | | | | | |
| | AAGACTGGAAATCAAGGGCGGAGGAGGCGGAGGTGGAGGAGGAGAGCCCAGGGGCCCCACCATC | | | | | |
| | AAGCCCTGCCCCCCCTGTAAGTGTCCAGCTCCCAACCTGCTGGGCGGACCCAGCGTGTTCATCT | | | | | |
| | TCCCCCCCAAGATCAAGGACGTGCTGATGATCAGCCTGAGCCCCATCGTGACCTGCGTGGTGGT | | | | | |
| | GGACGTGAGCGAGGACGACCCCGACGTGCAGATCAGCTGGTTCGTGAACAACGTGGAGGTGCAC | | | | | |
| | ACCGCCCAGACCCAGACCCACAGAGAGGACTACAACTCTACTCTGAGGGTCGTGTCCGCCCTGC | | | | | |
| | CCATCCAGCACCAGGACTGGATGAGCGGCAAAGAGTTCAAGTGCAAGGTCAACAACAAGGACCT | | | | | |
| | GCCAGCCCCCATCGAGAGGACCATCAGCAAGCCCAAGGGCAGCGTGAGAGCCCCACAGGTCTAC | | | | | |
| | GTGCTGCCCCCACCCGAGGAAGAGATGACCAAGAAGCAGGTCACACTGACCTGCATGGTGACCG | | | | | |
| | ACTTCATGCCCGAGGACATCTACGTGGAGTGGACCAACAACGGCAAGACCGAGCTGAACTACAA | | | | | |
| | GAACACCGAGCCAGTGCTGGACAGCGACGGCAGCTACTTCATGTACAGCAAGCTGAGGGTGGAG | | | | | |
| | AAGAAGAACTGGGTCGAGAGGAACAGCTACAGCTGCAGCGTGGTGCACGAGGGCCTGCACAACC | | | | | |
| | ACCACACCACCAAGAGCTTCAGCAGGACCCCCGGCAAG | | | | | |
| 8H1- | 331 | 8H1 VH | 4GS4 | 8H1 VL | 9G Modified IgG2a | IgG2a |
| VH4VL- | | | | | Hinge | |
| 9GIgG2a | GAGGTGCAGCTGGTGGAAAGTGGTGGTGGACTGGTTCAGCCTGGTGGCAGCCTGAGACTGTCTT | | | | | |
| | GTGCTGCCTCTGGCTTCACCTTCAGCATCTACACCATGAGCTGGGTCAGACAGGCTCCTGGCAA | | | | | |
| | AGGCCTGGAATGGGTGTCAGCTATCCTGTCTGATGGCAGCACCTACTATGCTGACTCTGTGGAA | | | | | |
| | GGCAGATTCACCATCAGCAGAGACAACAGCAAGACCACACTGTACCTGCAGATGAACAGCCTGA | | | | | |
| | CAGCTGAGGACACTGCTGTGTACTACTGCACCAAGAGAGTGGAAGGCTCCTTCAGAGGCTGGGG | | | | | |
| | ACAGGGAACACTGGTCACTGTGTCCTCTGGTGGAGGCGGTTCAGGCGGAGGTGGCAGCGGCGGT | | | | | |
| | GGAGGGTCAGGTGGAGGTGGATCAGAGATTGTGATGACACAGAGCCCTGGCACACTGTCTCTGA | | | | | |
| | GCCCTGGGGAATCTGCCACACTGAGCTGTAGAGCCAGCCAGTCTGTGTCTACCACCTACCTGGC | | | | | |
| | CTGGTATCAGCAGAAGCCTGGACAGGCTCCCAGACTGCTGATCTATGGGGCCTCTTCTAGAGCC | | | | | |
| | ACAGGCATCCCTGACAGATTTTCTGGCAGTGGCTCTGGCACAGACTTCACCCTGACCATCAGCA | | | | | |
| | GACTGGAACCTGAGGACTTTGCTGTGTACTACTGCCAGCAGTATGGTGGCAGCAGGCTGATCAC | | | | | |
| | ATTTGGCCAGGGCACAAGACTGGAAATCAAGGGCGGAGGAGGCGGAGGTGGAGGAGGAGAGCCT | | | | | |
| | AGAGGCCCCACCATCAAGCCCTGTCCTCCATGCAAGTGTCCTGCTCCTAACCTGCTTGGAGGCC | | | | | |
| | CCTCTGTGTTCATCTTCCCACCTAAGATCAAGGATGTGCTGATGATCTCTCTGAGCCCCATTGT | | | | | |
| | GACCTGTGTGGTGGTGGATGTGTCTGAGGATGACCCTGATGTGCAGATCTCTTGGTTTGTGAAC | | | | | |
| | AATGTGGAAGTGCACACAGCCCAGACACAGACCCACAGAGAGGACTACAACAGCACCCTGAGAG | | | | | |
| | TGGTGTCTGCCCTGCCTATCCAGCACCAGGACTGGATGAGTGGCAAAGAGTTCAAGTGCAAAGT | | | | | |
| | GAACAACAAGGACCTGCCTGCTCCAATTGAGAGAACCATCAGCAAGCCCAAGGGCTCTGTTAGA | | | | | |
| | GCCCCTCAAGTGTATGTGCTGCCTCCACCTGAGGAAGAGATGACCAAGAAACAAGTGACCCTGA | | | | | |
| | CATGCATGGTCACAGACTTCATGCCTGAGGACATCTATGTGGAATGGACCAACAATGGCAAGAC | | | | | |
| | AGAGCTGAACTACAAGAACACTGAGCCTGTGCTGGACTCTGATGGCAGCTACTTCATGTACAGC | | | | | |
| | AAGCTGAGGGTTGAGAAGAAGAACTGGGTTGAGAGAAACAGCTACAGCTGCTCTGTGGTGCATG | | | | | |
| | AGGGCCTGCACAATCACCACACCACCAAGAGCTTCAGCAGAACCCCTGGCAAG | | | | | |
| 8H1- | 350 | 8H1 VH | 4GS4 | 8H1 VL | 9G Modified IgG1 | IgG1 |
| VH4VL- | | | | | Hinge | |
| 9GIgG1 | GAGGTGCAGCTGGTGGAAAGTGGTGGTGGACTGGTTCAGCCTGGTGGCAGCCTGAGACTGTCTT | | | | | |
| | GTGCTGCCTCTGGCTTCACCTTCAGCATCTACACCATGAGCTGGGTCAGACAGGCTCCTGGCAA | | | | | |
| | AGGCCTGGAATGGGTGTCAGCTATCCTGTCTGATGGCAGCACCTACTATGCTGACTCTGTGGAA | | | | | |
| | GGCAGATTCACCATCAGCAGAGACAACAGCAAGACCACACTGTACCTGCAGATGAACAGCCTGA | | | | | |
| | CAGCTGAGGACACTGCTGTGTACTACTGCACCAAGAGAGTGGAAGGCTCCTTCAGAGGCTGGGG | | | | | |
| | ACAGGGAACACTGGTCACTGTGTCCTCTGGTGGAGGCGGTTCAGGCGGAGGTGGCAGCGGCGGT | | | | | |
| | GGAGGGTCAGGTGGAGGTGGATCAGAGATTGTGATGACACAGAGCCCTGGCACACTGTCTCTGA | | | | | |
| | GCCCTGGGGAATCTGCCACACTGAGCTGTAGAGCCAGCCAGTCTGTGTCTACCACCTACCTGGC | | | | | |
| | CTGGTATCAGCAGAAGCCTGGACAGGCTCCCAGACTGCTGATCTATGGGGCCTCTTCTAGAGCC | | | | | |
| | ACAGGCATCCCTGACAGATTTTCTGGCAGTGGCTCTGGCACAGACTTCACCCTGACCATCAGCA | | | | | |
| | GACTGGAACCTGAGGACTTTGCTGTGTACTACTGCCAGCAGTATGGTGGCAGCAGGCTGATCAC | | | | | |
| | ATTTGGCCAGGGCACAAGACTGGAAATCAAGGGCGGAGGAGGCGGAGGTGGAGGAGGAGAACCC | | | | | |
| | AAGAGCTGTGACAAGACCCACACCTGTCCTCCATGTCCTGCTCCAGAACTGCTTGGAGGCCCTT | | | | | |
| | CTGTGTTCCTGTTTCCTCCAAAGCCTAAGGACACCCTGATGATCAGCAGAACCCCTGAAGTGAC | | | | | |
| | CTGTGTGGTGGTTGATGTGTCCCATGAGGACCCAGAAGTGAAGTTCAATTGGTATGTGGATGGG | | | | | |
| | GTTGAAGTGCACAATGCCAAGACCAAGCCTAGAGAGGAACAGTACAACTCCACCTACAGAGTGG | | | | | |
| | TGTCTGTGCTGACAGTGCTGCACCAGGACTGGCTGAATGGCAAAGAGTACAAGTGCAAGGTGTC | | | | | |
| | CAACAAGGCCCTGCCTGCTCCTATTGAGAAAACCATCTCCAAGGCCAAGGGCCAGCCAAGAGAA | | | | | |
| | CCCCAGGTTTACACCCTGCCACCTAGCAGAGAAGAGATGACCAAGAACCAGGTGTCCCTGACCT | | | | | |
| | GCCTGGTTAAGGGCTTCTACCCCTCTGACATTGCTGTGGAATGGGAGAGCAATGGCCAGCCTGA | | | | | |
| | AAACAACTACAAGACAACCCCTCCTGTGCTGGACTCAGATGGCTCATTCTTCCTGTACAGCAAG | | | | | |
| | CTGACTGTGGACAAGTCCAGGTGGCAGCAGGGAAATGTGTTCAGCTGCTCTGTGATGCATGAGG | | | | | |
| | CCCTGCACAACCACTACACCCAGAAAAGTCTGAGTCTGAGCCCTGGCAAG | | | | | |
| 8H1- | 332 | 8H1 VL | 4GS2 | 8H1 VH | 9G Modified IgG2a | IgG2a |
| VL2VH- | | | | | Hinge | |
| 9GIgG2a | GAGATTGTGATGACACAGAGCCCTGGCACACTGTCTCTGAGCCCTGGGGAATCTGCCACACTGA | | | | | |
| | GCTGTAGAGCCAGCCAGTCTGTGTCTACCACCTACCTGGCCTGGTATCAGCAGAAGCCTGGACA | | | | | |
| | GGCTCCCAGACTGCTGATCTATGGGGCCTCTTCTAGAGCCACAGGCATCCCTGACAGATTTTCT | | | | | |
| | GGCAGTGGCTCTGGCACAGACTTCACCCTGACCATCAGCAGACTGGAACCTGAGGACTTTGCTG | | | | | |
| | TGTACTACTGCCAGCAGTATGGTGGCAGCAGGCTGATCACATTTGGCCAGGGCACAAGACTGGA | | | | | |
| | AATCAAGGGCGGAGGTGGCAGCGGAGGTGGCGGATCAGAGGTGCAGCTGGTGGAAAGTGGTGGT | | | | | |
| | GGACTGGTTCAGCCTGGTGGCAGCCTGAGACTGTCTTGTGCTGCCTCTGGCTTCACCTTCAGCA | | | | | |
| | TCTACACCATGAGCTGGGTCAGACAGGCTCCTGGCAAAGGCCTGGAATGGGTGTCAGCTATCCT | | | | | |
| | GTCTGATGGCAGCACCTACTATGCTGACTCTGTGGAAGGCAGATTCACCATCAGCAGAGACAAC | | | | | |
| | AGCAAGACCACACTGTACCTGCAGATGAACAGCCTGACAGCTGAGGACACTGCTGTGTACTACT | | | | | |
| | GCACCAAGAGAGTGGAAGGCTCCTTCAGAGGCTGGGGACAGGGAACACTGGTCACTGTGTCCTC | | | | | |
| | TGGCGGAGGAGGCGGAGGTGGAGGAGGAGAGCCTAGAGGCCCCACCATCAAGCCCTGTCCTCCA | | | | | |
| | TGCAAGTGTCCTGCTCCTAACCTGCTTGGAGGCCCCTCTGTGTTCATCTTCCCACCTAAGATCA | | | | | |
| | AGGATGTGCTGATGATCTCTCTGAGCCCCATTGTGACCTGTGTGGTGGTGGATGTGTCTGAGGA | | | | | |
| | TGACCCTGATGTGCAGATCTCTTGGTTTGTGAACAATGTGGAAGTGCACACAGCCCAGACACAG | | | | | |
| | ACCCACAGAGAGGACTACAACAGCACCCTGAGAGTGGTGTCTGCCCTGCCTATCCAGCACCAGG | | | | | |
| | ACTGGATGAGTGGCAAAGAGTTCAAGTGCAAAGTGAACAACAAGGACCTGCCTGCTCCAATTGA | | | | | |
| | GAGAACCATCAGCAAGCCCAAGGGCTCTGTTAGAGCCCCTCAAGTGTATGTGCTGCCTCCACCT | | | | | |
| | GAGGAAGAGATGACCAAGAAACAAGTGACCCTGACATGCATGGTCACAGACTTCATGCCTGAGG | | | | | |
| | ACATCTATGTGGAATGGACCAACAATGGCAAGACAGAGCTGAACTACAAGAACACTGAGCCTGT | | | | | |
| | GCTGGACTCTGATGGCAGCTACTTCATGTACAGCAAGCTGAGGGTTGAGAAGAAGAACTGGGTT | | | | | |
| | GAGAGAAACAGCTACAGCTGCTCTGTGGTGCATGAGGGCCTGCACAATCACCACACCACCAAGA | | | | | |
| | GCTTCAGCAGAACCCCTGGCAAG | | | | | |
| 8H1- | 333 | 8H1 VL | 4GS3 | 8H1 VH | 9G Modified IgG2a | IgG2a |
| VL3VH- | | | | | Hinge | |
| 9GIgG2a | GAGATTGTGATGACACAGAGCCCTGGCACACTGTCTCTGAGCCCTGGGGAATCTGCCACACTGA | | | | | |
| | GCTGTAGAGCCAGCCAGTCTGTGTCTACCACCTACCTGGCCTGGTATCAGCAGAAGCCTGGACA | | | | | |
| | GGCTCCCAGACTGCTGATCTATGGGGCCTCTTCTAGAGCCACAGGCATCCCTGACAGATTTTCT | | | | | |
| | GGCAGTGGCTCTGGCACAGACTTCACCCTGACCATCAGCAGACTGGAACCTGAGGACTTTGCTG | | | | | |
| | TGTACTACTGCCAGCAGTATGGTGGCAGCAGGCTGATCACATTTGGCCAGGGCACAAGACTGGA | | | | | |
| | AATCAAGGGTGGAGGCGGTTCAGGCGGAGGTGGCAGCGGCGGTGGAGGGTCGGAGGTGCAGCTG | | | | | |
| | GTGGAAAGTGGTGGTGGACTGGTTCAGCCTGGTGGCAGCCTGAGACTGTCTTGTGCTGCCTCTG | | | | | |
| | GCTTCACCTTCAGCATCTACACCATGAGCTGGGTCAGACAGGCTCCTGGCAAAGGCCTGGAATG | | | | | |
| | GGTGTCAGCTATCCTGTCTGATGGCAGCACCTACTATGCTGACTCTGTGGAAGGCAGATTCACC | | | | | |
| | ATCAGCAGAGACAACAGCAAGACCACACTGTACCTGCAGATGAACAGCCTGACAGCTGAGGACA | | | | | |
| | CTGCTGTGTACTACTGCACCAAGAGAGTGGAAGGCTCCTTCAGAGGCTGGGGACAGGGAACACT | | | | | |
| | GGTCACTGTGTCCTCTGGCGGAGGAGGCGGAGGTGGAGGAGGAGAGCCTAGAGGCCCCACCATC | | | | | |
| | AAGCCCTGTCCTCCATGCAAGTGTCCTGCTCCTAACCTGCTTGGAGGCCCCTCTGTGTTCATCT | | | | | |
| | TCCCACCTAAGATCAAGGATGTGCTGATGATCTCTCTGAGCCCCATTGTGACCTGTGTGGTGGT | | | | | |
| | GGATGTGTCTGAGGATGACCCTGATGTGCAGATCTCTTGGTTTGTGAACAATGTGGAAGTGCAC | | | | | |
| | ACAGCCCAGACACAGACCCACAGAGAGGACTACAACAGCACCCTGAGAGTGGTGTCTGCCCTGC | | | | | |
| | CTATCCAGCACCAGGACTGGATGAGTGGCAAAGAGTTCAAGTGCAAAGTGAACAACAAGGACCT | | | | | |
| | GCCTGCTCCAATTGAGAGAACCATCAGCAAGCCCAAGGGCTCTGTTAGAGCCCCTCAAGTGTAT | | | | | |
| | GTGCTGCCTCCACCTGAGGAAGAGATGACCAAGAAACAAGTGACCCTGACATGCATGGTCACAG | | | | | |
| | ACTTCATGCCTGAGGACATCTATGTGGAATGGACCAACAATGGCAAGACAGAGCTGAACTACAA | | | | | |
| | GAACACTGAGCCTGTGCTGGACTCTGATGGCAGCTACTTCATGTACAGCAAGCTGAGGGTTGAG | | | | | |
| | AAGAAGAACTGGGTTGAGAGAAACAGCTACAGCTGCTCTGTGGTGCATGAGGGCCTGCACAATC | | | | | |
| | ACCACACCACCAAGAGCTTCAGCAGAACCCCTGGCAAG | | | | | |
| 8H1- | 334 | 8H1 VL | 4GS4 | 8H1 VH | 9G Modified IgG2a | IgG2a |
| VL4VH- | | | | | Hinge | |
| 9GIgG2a | GAGATTGTGATGACACAGAGCCCTGGCACACTGTCTCTGAGCCCTGGGGAATCTGCCACACTGA | | | | | |
| | GCTGTAGAGCCAGCCAGTCTGTGTCTACCACCTACCTGGCCTGGTATCAGCAGAAGCCTGGACA | | | | | |
| | GGCTCCCAGACTGCTGATCTATGGGGCCTCTTCTAGAGCCACAGGCATCCCTGACAGATTTTCT | | | | | |
| | GGCAGTGGCTCTGGCACAGACTTCACCCTGACCATCAGCAGACTGGAACCTGAGGACTTTGCTG | | | | | |
| | TGTACTACTGCCAGCAGTATGGTGGCAGCAGGCTGATCACATTTGGCCAGGGCACAAGACTGGA | | | | | |
| | AATCAAGGGTGGAGGCGGTTCAGGCGGAGGTGGCAGCGGCGGTGGAGGGTCAGGTGGAGGTGGA | | | | | |
| | TCAGAGGTGCAGCTGGTGGAAAGTGGTGGTGGACTGGTTCAGCCTGGTGGCAGCCTGAGACTGT | | | | | |
| | CTTGTGCTGCCTCTGGCTTCACCTTCAGCATCTACACCATGAGCTGGGTCAGACAGGCTCCTGG | | | | | |
| | CAAAGGCCTGGAATGGGTGTCAGCTATCCTGTCTGATGGCAGCACCTACTATGCTGACTCTGTG | | | | | |
| | GAAGGCAGATTCACCATCAGCAGAGACAACAGCAAGACCACACTGTACCTGCAGATGAACAGCC | | | | | |
| | TGACAGCTGAGGACACTGCTGTGTACTACTGCACCAAGAGAGTGGAAGGCTCCTTCAGAGGCTG | | | | | |
| | GGGACAGGGAACACTGGTCACTGTGTCCTCTGGCGGAGGAGGCGGAGGTGGAGGAGGAGAGCCT | | | | | |
| | AGAGGCCCCACCATCAAGCCCTGTCCTCCATGCAAGTGTCCTGCTCCTAACCTGCTTGGAGGCC | | | | | |
| | CCTCTGTGTTCATCTTCCCACCTAAGATCAAGGATGTGCTGATGATCTCTCTGAGCCCCATTGT | | | | | |
| | GACCTGTGTGGTGGTGGATGTGTCTGAGGATGACCCTGATGTGCAGATCTCTTGGTTTGTGAAC | | | | | |
| | AATGTGGAAGTGCACACAGCCCAGACACAGACCCACAGAGAGGACTACAACAGCACCCTGAGAG | | | | | |
| | TGGTGTCTGCCCTGCCTATCCAGCACCAGGACTGGATGAGTGGCAAAGAGTTCAAGTGCAAAGT | | | | | |
| | GAACAACAAGGACCTGCCTGCTCCAATTGAGAGAACCATCAGCAAGCCCAAGGGCTCTGTTAGA | | | | | |
| | GCCCCTCAAGTGTATGTGCTGCCTCCACCTGAGGAAGAGATGACCAAGAAACAAGTGACCCTGA | | | | | |
| | CATGCATGGTCACAGACTTCATGCCTGAGGACATCTATGTGGAATGGACCAACAATGGCAAGAC | | | | | |
| | AGAGCTGAACTACAAGAACACTGAGCCTGTGCTGGACTCTGATGGCAGCTACTTCATGTACAGC | | | | | |
| | AAGCTGAGGGTTGAGAAGAAGAACTGGGTTGAGAGAAACAGCTACAGCTGCTCTGTGGTGCATG | | | | | |
| | AGGGCCTGCACAATCACCACACCACCAAGAGCTTCAGCAGAACCCCTGGCAAG | | | | | |
| 43A11- | 335 | 843A11 VH | 4GS2 | 43A11 VL | 9G Modified IgG2a | IgG2a |
| VH2VL- | | | | | Hinge | |
| 9GIgG2a | CAGGTCCAGCTGCAAGAGTCTGGCCCTGGACTGGTCAAGCCTTCTGGCACACTGAGCCTGACCT | | | | | |
| | GTGCTGTGACTGGTGGCAGCATCAGCAGCAGCAATTGGTGGTCTTGGGTCAGACAGAGCCCTGG | | | | | |
| | CAAAGGCCTGGAATGGATTGGAGAGATCCACCATGATGGCAACCTGAACTACAACCCTCTGCTG | | | | | |
| | AAGTCCAGAGTGTCCATGAGCCTGGACAGAAGCAAGAACCAGTTCAGCCTGAAGCTGACCTCTG | | | | | |
| | TGACAGCTGCTGACACTGCTGTGTACTACTGTGCCAGATGGGACTTCTTCTTTGACTCCTCCTA | | | | | |
| | CTACATCAGGGGCTTTGACCTGTGGGGCCAGGGCACAATGGTTACAGTGTCCTCTGGTGGAGGC | | | | | |
| | GGTTCAGGCGGAGGTGGCAGCGGCGGTGGAGGGTCAGGTGGAGGTGGATCAGAGACAACCCTGA | | | | | |
| | CACAGAGCCCTGGCACATTGTCTCTGAGCCCTGGGGAGAGAGTGACCCTGTCTTGTAGAGCCAG | | | | | |
| | CCAGTCTGTGGACAGAAACTACCTGGCCTGGTATCAGCAGAAGCCTGGCCAGTCTCCTAGACTG | | | | | |
| | CTGATCTACTCTGCCTCTAGGAGAGCCACAGGCATCCCTGACAGATTTTCTGGCAGTGGCTCTG | | | | | |
| | GCACAGACTTCACCCTGACCATCAGCAGACTGGAACCTGAGGACTTTGTGGTGTACTACTGCCA | | | | | |
| | GCAGTATGGTGGCAGCCCTCCTATCACATTTGGCCAGGGCACAAGACTGGAAATCAAGGGCGGA | | | | | |
| | GGAGGCGGAGGTGGAGGAGGAGAGCCTAGAGGCCCCACCATCAAGCCCTGTCCTCCATGCAAGT | | | | | |
| | GTCCTGCTCCTAACCTGCTTGGAGGCCCCTCTGTGTTCATCTTCCCACCTAAGATCAAGGATGT | | | | | |
| | GCTGATGATCTCTCTGAGCCCCATTGTGACCTGTGTGGTGGTGGATGTGTCTGAGGATGACCCT | | | | | |
| | GATGTGCAGATCTCTTGGTTTGTGAACAATGTGGAAGTGCACACAGCCCAGACACAGACCCACA | | | | | |
| | GAGAGGACTACAACAGCACCCTGAGAGTGGTGTCTGCCCTGCCTATCCAGCACCAGGACTGGAT | | | | | |
| | GAGTGGCAAAGAGTTCAAGTGCAAAGTGAACAACAAGGACCTGCCTGCTCCAATTGAGAGAACC | | | | | |
| | ATCAGCAAGCCCAAGGGCTCTGTTAGAGCCCCTCAAGTGTATGTGCTGCCTCCACCTGAGGAAG | | | | | |
| | AGATGACCAAGAAACAAGTGACCCTGACATGCATGGTCACAGACTTCATGCCTGAGGACATCTA | | | | | |
| | TGTGGAATGGACCAACAATGGCAAGACAGAGCTGAACTACAAGAACACTGAGCCTGTGCTGGAC | | | | | |
| | TCTGATGGCAGCTACTTCATGTACAGCAAGCTGAGGGTTGAGAAGAAGAACTGGGTTGAGAGAA | | | | | |
| | ACAGCTACAGCTGCTCTGTGGTGCATGAGGGCCTGCACAATCACCACACCACCAAGAGCTTCAG | | | | | |
| | CAGAACCCCTGGCAAG | | | | | |
| 43A11- | 336 | 43A11 VH | 4GS3 | 43A11 VL | 9G Modified IgG2a | IgG2a |
| VH3VL- | | | | | Hinge | |
| 9GIgG2a | CAGGTCCAGCTGCAAGAGTCTGGCCCTGGACTGGTCAAGCCTTCTGGCACACTGAGCCTGACCT | | | | | |
| | GTGCTGTGACTGGTGGCAGCATCAGCAGCAGCAATTGGTGGTCTTGGGTCAGACAGAGCCCTGG | | | | | |
| | CAAAGGCCTGGAATGGATTGGAGAGATCCACCATGATGGCAACCTGAACTACAACCCTCTGCTG | | | | | |
| | AAGTCCAGAGTGTCCATGAGCCTGGACAGAAGCAAGAACCAGTTCAGCCTGAAGCTGACCTCTG | | | | | |
| | TGACAGCTGCTGACACTGCTGTGTACTACTGTGCCAGATGGGACTTCTTCTTTGACTCCTCCTA | | | | | |
| | CTACATCAGGGGCTTTGACCTGTGGGGCCAGGGCACAATGGTTACAGTGTCCTCTGGTGGAGGC | | | | | |
| | GGTTCAGGCGGAGGTGGCAGCGGCGGTGGAGGGTCGGAGACAACCCTGACACAGAGCCCTGGCA | | | | | |
| | CATTGTCTCTGAGCCCTGGGGAGAGAGTGACCCTGTCTTGTAGAGCCAGCCAGTCTGTGGACAG | | | | | |
| | AAACTACCTGGCCTGGTATCAGCAGAAGCCTGGCCAGTCTCCTAGACTGCTGATCTACTCTGCC | | | | | |
| | TCTAGGAGAGCCACAGGCATCCCTGACAGATTTTCTGGCAGTGGCTCTGGCACAGACTTCACCC | | | | | |
| | TGACCATCAGCAGACTGGAACCTGAGGACTTTGTGGTGTACTACTGCCAGCAGTATGGTGGCAG | | | | | |
| | CCCTCCTATCACATTTGGCCAGGGCACAAGACTGGAAATCAAGGGCGGAGGAGGCGGAGGTGGA | | | | | |
| | GGAGGAGAGCCTAGAGGCCCCACCATCAAGCCCTGTCCTCCATGCAAGTGTCCTGCTCCTAACC | | | | | |
| | TGCTTGGAGGCCCCTCTGTGTTCATCTTCCCACCTAAGATCAAGGATGTGCTGATGATCTCTCT | | | | | |
| | GAGCCCCATTGTGACCTGTGTGGTGGTGGATGTGTCTGAGGATGACCCTGATGTGCAGATCTCT | | | | | |
| | TGGTTTGTGAACAATGTGGAAGTGCACACAGCCCAGACACAGACCCACAGAGAGGACTACAACA | | | | | |
| | GCACCCTGAGAGTGGTGTCTGCCCTGCCTATCCAGCACCAGGACTGGATGAGTGGCAAAGAGTT | | | | | |
| | CAAGTGCAAAGTGAACAACAAGGACCTGCCTGCTCCAATTGAGAGAACCATCAGCAAGCCCAAG | | | | | |
| | GGCTCTGTTAGAGCCCCTCAAGTGTATGTGCTGCCTCCACCTGAGGAAGAGATGACCAAGAAAC | | | | | |
| | AAGTGACCCTGACATGCATGGTCACAGACTTCATGCCTGAGGACATCTATGTGGAATGGACCAA | | | | | |
| | CAATGGCAAGACAGAGCTGAACTACAAGAACACTGAGCCTGTGCTGGACTCTGATGGCAGCTAC | | | | | |
| | TTCATGTACAGCAAGCTGAGGGTTGAGAAGAAGAACTGGGTTGAGAGAAACAGCTACAGCTGCT | | | | | |
| | CTGTGGTGCATGAGGGCCTGCACAATCACCACACCACCAAGAGCTTCAGCAGAACCCCTGGCAA | | | | | |
| | G | | | | | |
| 43A11- | 337 | 8H1 VH | 4GS4 | 43 A11VL | 9G Modified IgG2a | IgG2a |
| VH4VL- | | | | | Hinge | |
| 9GIgG2a | CAGGTCCAGCTGCAAGAGTCTGGCCCTGGACTGGTCAAGCCTTCTGGCACACTGAGCCTGACCT | | | | | |
| | GTGCTGTGACTGGTGGCAGCATCAGCAGCAGCAATTGGTGGTCTTGGGTCAGACAGAGCCCTGG | | | | | |
| | CAAAGGCCTGGAATGGATTGGAGAGATCCACCATGATGGCAACCTGAACTACAACCCTCTGCTG | | | | | |
| | AAGTCCAGAGTGTCCATGAGCCTGGACAGAAGCAAGAACCAGTTCAGCCTGAAGCTGACCTCTG | | | | | |
| | TGACAGCTGCTGACACTGCTGTGTACTACTGTGCCAGATGGGACTTCTTCTTTGACTCCTCCTA | | | | | |
| | CTACATCAGGGGCTTTGACCTGTGGGGCCAGGGCACAATGGTTACAGTGTCCTCTGGCGGAGGT | | | | | |
| | GGCAGCGGAGGTGGCGGATCAGAGACAACCCTGACACAGAGCCCTGGCACATTGTCTCTGAGCC | | | | | |
| | CTGGGGAGAGAGTGACCCTGTCTTGTAGAGCCAGCCAGTCTGTGGACAGAAACTACCTGGCCTG | | | | | |
| | GTATCAGCAGAAGCCTGGCCAGTCTCCTAGACTGCTGATCTACTCTGCCTCTAGGAGAGCCACA | | | | | |
| | GGCATCCCTGACAGATTTTCTGGCAGTGGCTCTGGCACAGACTTCACCCTGACCATCAGCAGAC | | | | | |
| | TGGAACCTGAGGACTTTGTGGTGTACTACTGCCAGCAGTATGGTGGCAGCCCTCCTATCACATT | | | | | |
| | TGGCCAGGGCACAAGACTGGAAATCAAGGGCGGAGGAGGCGGAGGTGGAGGAGGAGAGCCTAGA | | | | | |
| | GGCCCCACCATCAAGCCCTGTCCTCCATGCAAGTGTCCTGCTCCTAACCTGCTTGGAGGCCCCT | | | | | |
| | CTGTGTTCATCTTCCCACCTAAGATCAAGGATGTGCTGATGATCTCTCTGAGCCCCATTGTGAC | | | | | |
| | CTGTGTGGTGGTGGATGTGTCTGAGGATGACCCTGATGTGCAGATCTCTTGGTTTGTGAACAAT | | | | | |
| | GTGGAAGTGCACACAGCCCAGACACAGACCCACAGAGAGGACTACAACAGCACCCTGAGAGTGG | | | | | |
| | TGTCTGCCCTGCCTATCCAGCACCAGGACTGGATGAGTGGCAAAGAGTTCAAGTGCAAAGTGAA | | | | | |
| | CAACAAGGACCTGCCTGCTCCAATTGAGAGAACCATCAGCAAGCCCAAGGGCTCTGTTAGAGCC | | | | | |
| | CCTCAAGTGTATGTGCTGCCTCCACCTGAGGAAGAGATGACCAAGAAACAAGTGACCCTGACAT | | | | | |
| | GCATGGTCACAGACTTCATGCCTGAGGACATCTATGTGGAATGGACCAACAATGGCAAGACAGA | | | | | |
| | GCTGAACTACAAGAACACTGAGCCTGTGCTGGACTCTGATGGCAGCTACTTCATGTACAGCAAG | | | | | |
| | CTGAGGGTTGAGAAGAAGAACTGGGTTGAGAGAAACAGCTACAGCTGCTCTGTGGTGCATGAGG | | | | | |
| | GCCTGCACAATCACCACACCACCAAGAGCTTCAGCAGAACCCCTGGCAAG | | | | | |
| 43A11- | 338 | 43A11 VL | 4GS2 | 43A11 | 9G Modified IgG2a | IgG2a |
| VL2VH- | | | | VH | Hinge | |
| 9GIgG2a | GAGACAACCCTGACACAGAGCCCTGGCACATTGTCTCTGAGCCCTGGGGAGAGAGTGACCCTGT | | | | | |
| | CTTGTAGAGCCAGCCAGTCTGTGGACAGAAACTACCTGGCCTGGTATCAGCAGAAGCCTGGCCA | | | | | |
| | GTCTCCTAGACTGCTGATCTACTCTGCCTCTAGGAGAGCCACAGGCATCCCTGACAGATTTTCT | | | | | |
| | GGCAGTGGCTCTGGCACAGACTTCACCCTGACCATCAGCAGACTGGAACCTGAGGACTTTGTGG | | | | | |
| | TGTACTACTGCCAGCAGTATGGTGGCAGCCCTCCTATCACATTTGGCCAGGGCACAAGACTGGA | | | | | |
| | AATCAAGGGTGGAGGCGGTTCAGGCGGAGGTGGCAGCGGCGGTGGAGGGTCAGGTGGAGGTGGA | | | | | |
| | TCACAGGTCCAGCTGCAAGAGTCTGGCCCTGGACTGGTCAAGCCTTCTGGCACACTGAGCCTGA | | | | | |
| | CCTGTGCTGTGACTGGTGGCAGCATCAGCAGCAGCAATTGGTGGTCTTGGGTCAGACAGAGCCC | | | | | |
| | TGGCAAAGGCCTGGAATGGATTGGAGAGATCCACCATGATGGCAACCTGAACTACAACCCTCTG | | | | | |
| | CTGAAGTCCAGAGTGTCCATGAGCCTGGACAGAAGCAAGAACCAGTTCAGCCTGAAGCTGACCT | | | | | |
| | CTGTGACAGCTGCTGACACTGCTGTGTACTACTGTGCCAGATGGGACTTCTTCTTTGACTCCTC | | | | | |
| | CTACTACATCAGGGGCTTTGACCTGTGGGGCCAGGGCACAATGGTTACAGTGTCCTCTGGCGGA | | | | | |
| | GGAGGCGGAGGTGGAGGAGGAGAGCCTAGAGGCCCCACCATCAAGCCCTGTCCTCCATGCAAGT | | | | | |
| | GTCCTGCTCCTAACCTGCTTGGAGGCCCCTCTGTGTTCATCTTCCCACCTAAGATCAAGGATGT | | | | | |
| | GCTGATGATCTCTCTGAGCCCCATTGTGACCTGTGTGGTGGTGGATGTGTCTGAGGATGACCCT | | | | | |
| | GATGTGCAGATCTCTTGGTTTGTGAACAATGTGGAAGTGCACACAGCCCAGACACAGACCCACA | | | | | |
| | GAGAGGACTACAACAGCACCCTGAGAGTGGTGTCTGCCCTGCCTATCCAGCACCAGGACTGGAT | | | | | |
| | GAGTGGCAAAGAGTTCAAGTGCAAAGTGAACAACAAGGACCTGCCTGCTCCAATTGAGAGAACC | | | | | |
| | ATCAGCAAGCCCAAGGGCTCTGTTAGAGCCCCTCAAGTGTATGTGCTGCCTCCACCTGAGGAAG | | | | | |
| | AGATGACCAAGAAACAAGTGACCCTGACATGCATGGTCACAGACTTCATGCCTGAGGACATCTA | | | | | |
| | TGTGGAATGGACCAACAATGGCAAGACAGAGCTGAACTACAAGAACACTGAGCCTGTGCTGGAC | | | | | |
| | TCTGATGGCAGCTACTTCATGTACAGCAAGCTGAGGGTTGAGAAGAAGAACTGGGTTGAGAGAA | | | | | |
| | ACAGCTACAGCTGCTCTGTGGTGCATGAGGGCCTGCACAATCACCACACCACCAAGAGCTTCAG | | | | | |
| | CAGAACCCCTGGCAAG | | | | | |
| 43A11- | 339 | 43A11 VL | 4GS3 | 43A11 VH | 9G Modified IgG2a | IgG2a |
| VL3VH- | | | | | Hinge | |
| 9GIgG2a | GAGACAACCCTGACACAGAGCCCTGGCACATTGTCTCTGAGCCCTGGGGAGAGAGTGACCCTGT | | | | | |
| | CTTGTAGAGCCAGCCAGTCTGTGGACAGAAACTACCTGGCCTGGTATCAGCAGAAGCCTGGCCA | | | | | |
| | GTCTCCTAGACTGCTGATCTACTCTGCCTCTAGGAGAGCCACAGGCATCCCTGACAGATTTTCT | | | | | |
| | GGCAGTGGCTCTGGCACAGACTTCACCCTGACCATCAGCAGACTGGAACCTGAGGACTTTGTGG | | | | | |
| | TGTACTACTGCCAGCAGTATGGTGGCAGCCCTCCTATCACATTTGGCCAGGGCACAAGACTGGA | | | | | |
| | AATCAAGGGTGGAGGCGGTTCAGGCGGAGGTGGCAGCGGCGGTGGAGGGTCGCAGGTCCAGCTG | | | | | |
| | CAAGAGTCTGGCCCTGGACTGGTCAAGCCTTCTGGCACACTGAGCCTGACCTGTGCTGTGACTG | | | | | |
| | GTGGCAGCATCAGCAGCAGCAATTGGTGGTCTTGGGTCAGACAGAGCCCTGGCAAAGGCCTGGA | | | | | |
| | ATGGATTGGAGAGATCCACCATGATGGCAACCTGAACTACAACCCTCTGCTGAAGTCCAGAGTG | | | | | |
| | TCCATGAGCCTGGACAGAAGCAAGAACCAGTTCAGCCTGAAGCTGACCTCTGTGACAGCTGCTG | | | | | |
| | ACACTGCTGTGTACTACTGTGCCAGATGGGACTTCTTCTTTGACTCCTCCTACTACATCAGGGG | | | | | |
| | CTTTGACCTGTGGGGCCAGGGCACAATGGTTACAGTGTCCTCTGGCGGAGGAGGCGGAGGTGGA | | | | | |
| | GGAGGAGAGCCTAGAGGCCCCACCATCAAGCCCTGTCCTCCATGCAAGTGTCCTGCTCCTAACC | | | | | |
| | TGCTTGGAGGCCCCTCTGTGTTCATCTTCCCACCTAAGATCAAGGATGTGCTGATGATCTCTCT | | | | | |
| | GAGCCCCATTGTGACCTGTGTGGTGGTGGATGTGTCTGAGGATGACCCTGATGTGCAGATCTCT | | | | | |
| | TGGTTTGTGAACAATGTGGAAGTGCACACAGCCCAGACACAGACCCACAGAGAGGACTACAACA | | | | | |
| | GCACCCTGAGAGTGGTGTCTGCCCTGCCTATCCAGCACCAGGACTGGATGAGTGGCAAAGAGTT | | | | | |
| | CAAGTGCAAAGTGAACAACAAGGACCTGCCTGCTCCAATTGAGAGAACCATCAGCAAGCCCAAG | | | | | |
| | GGCTCTGTTAGAGCCCCTCAAGTGTATGTGCTGCCTCCACCTGAGGAAGAGATGACCAAGAAAC | | | | | |
| | AAGTGACCCTGACATGCATGGTCACAGACTTCATGCCTGAGGACATCTATGTGGAATGGACCAA | | | | | |
| | CAATGGCAAGACAGAGCTGAACTACAAGAACACTGAGCCTGTGCTGGACTCTGATGGCAGCTAC | | | | | |
| | TTCATGTACAGCAAGCTGAGGGTTGAGAAGAAGAACTGGGTTGAGAGAAACAGCTACAGCTGCT | | | | | |
| | CTGTGGTGCATGAGGGCCTGCACAATCACCACACCACCAAGAGCTTCAGCAGAACCCCTGGCAA | | | | | |
| | G | | | | | |
| 43A11- | 340 | 43A11 VL | 4GS4 | 43A11 VH | 9G Modified IgG2a | IgG2a |
| VL4VH- | | | | | Hinge | |

| | |
|---|---|
| 9GIgG2a | GAGACAACCCTGACACAGAGCCCTGGCACATTGTCTCTGAGCCCTGGGGAGAGAGTGACCCTGT |
| | CTTGTAGAGCCAGCCAGTCTGTGGACAGAAACTACCTGGCCTGGTATCAGCAGAAGCCTGGCCA |
| | GTCTCCTAGACTGCTGATCTACTCTGCCTCTAGGAGAGCCACAGGCATCCCTGACAGATTTTCT |
| | GGCAGTGGCTCTGGCACAGACTTCACCCTGACCATCAGCAGACTGGAACCTGAGGACTTTGTGG |
| | TGTACTACTGCCAGCAGTATGGTGGCAGCCCTCCTATCACATTTGGCCAGGGCACAAGACTGGA |
| | AATCAAGGGCGGAGGTGGCAGCGGAGGTGGCGGATCACAGGTCCAGCTGCAAGAGTCTGGCCCT |
| | GGACTGGTCAAGCCTTCTGGCACACTGAGCCTGACCTGTGCTGTGACTGGTGGCAGCATCAGCA |
| | GCAGCAATTGGTGGTCTTGGGTCAGACAGAGCCCTGGCAAAGGCCTGGAATGGATTGGAGAGAT |
| | CCACCATGATGGCAACCTGAACTACAACCCTCTGCTGAAGTCCAGAGTGTCCATGAGCCTGGAC |
| | AGAAGCAAGAACCAGTTCAGCCTGAAGCTGACCTCTGTGACAGCTGCTGACACTGCTGTGTACT |
| | ACTGTGCCAGATGGGACTTCTTCTTTGACTCCTCCTACTACATCAGGGGCTTTGACCTGTGGGG |
| | CCAGGGCACAATGGTTACAGTGTCCTCTGGCGGAGGAGGCGGAGGTGGAGGAGGAGAGCCTAGA |
| | GGCCCCACCATCAAGCCCTGTCCTCCATGCAAGTGTCCTGCTCCTAACCTGCTTGGAGGCCCCT |
| | CTGTGTTCATCTTCCCACCTAAGATCAAGGATGTGCTGATGATCTCTCTGAGCCCCATTGTGAC |
| | CTGTGTGGTGGTGGATGTGTCTGAGGATGACCCTGATGTGCAGATCTCTTGGTTTGTGAACAAT |
| | GTGGAAGTGCACACAGCCCAGACACAGACCCACAGAGAGGACTACAACAGCACCCTGAGAGTGG |
| | TGTCTGCCCTGCCTATCCAGCACCAGGACTGGATGAGTGGCAAAGAGTTCAAGTGCAAAGTGAA |
| | CAACAAGGACCTGCCTGCTCCAATTGAGAGAACCATCAGCAAGCCCAAGGGCTCTGTTAGAGCC |
| | CCTCAAGTGTATGTGCTGCCTCCACCTGAGGAAGAGATGACCAAGAAACAAGTGACCCTGACAT |
| | GCATGGTCACAGACTTCATGCCTGAGGACATCTATGTGGAATGGACCAACAATGGCAAGACAGA |
| | GCTGAACTACAAGAACACTGAGCCTGTGCTGGACTCTGATGGCAGCTACTTCATGTACAGCAAG |
| | CTGAGGGTTGAGAAGAAGAACTGGGTTGAGAGAAACAGCTACAGCTGCTCTGTGGTGCATGAGG |
| | GCCTGCACAATCACCACACCACCAAGAGCTTCAGCAGAACCCCTGGCAAG |

Table F shows exemplary anti-Tau ScFv-Fc constructs. In these constructs the VH of the anti-Tau mAb is upstream of the VL, with a 3xG4S linker between the variable regions (“VH3VL”). A 9 glycine (9G) linker is placed upstream of the Fc domain so itis between the scFv and the Fc domain. The sequences provided include the nucleotide sequence encoding the scFv-Fc, regulatory elements, such as the CAG promoter and polyA signal, flanked by ITR sequences, the nucleotide sequence encoding the scFv-Fc construct (including the signal sequence), and the amino acid sequence.

TABLE 11F
ScFv-Fc Constructs and Components

| Construct | |
|---|---|
| or | |
| Componentt | Sequence |
| 4P3-HL-L3 | CTGCGCGCTCGCTCGCTCACTGAGGCCGCCCGGGCAAAGCCCGGGCGTCGGGCG |
| scFv-Fc (ITR to | ACCTTTGGTCGCCCGGCCTCAGTGAGCGAGCGAGCGCGCAGAGAGGGAGTGGCC |
| ITR) | AACTCCATCACTAGGGGTTCCTTGTAGTTAATGATTAACCCGCCATGCTACTTA |
| | TCTACCAGGGTAATGGGGATCCTCTAGAACTATAGCTAGTCGACATTGATTATT |
| | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGCGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGCGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCGCCACCATGTA |
| | CAGAATGCAGCTGCTGCTGCTCATTGCCCTGTCTCTGGCCCTGGTCACCAATTC |
| | TGAGGTGCAGCTGGTGCAGTCTGGGGCTGAAGTGAAGAAGCCTGGGGAGAGCCT |
| | GAAGATCAGCTGCAAAGTGTCTGGCTATGACTTCAGCAAGTACTGGATTGGCTG |
| | GGTCAGACAGATGCCTGGCAAAGGCCTGGAATGGATGGGACTGATCTACCCTGG |
| | GGACTCTGACACCAGATACAGCCCTAGCCTGGAAGGCCAGGTGTCCATCTCTGC |
| | TGACAAGAGCATCTCTGCAGCCTACCTGCAGTTCAGCAGCCTGAAGGCCTCTGA |
| | CTCTGCCATGTACTACTGTGTCAGAAAGGGCTACTCTGCCTACCTTGGAGGATG |
| | GTTTGATCCTTGGGGCCAGGGCACACTGGTCACAGTGTCCTCTGGTGGAGGCGG |
| | TTCAGGCGGAGGTGGCAGCGGCGGTGGAGGGTCGGACATCCAGCTGACACAGAG |
| | CCCTGACAGCCTGGCTGCTTCTCTGGGAGAGAGAGTGACCATCAACTGCAGAAG |
| | CTCCCAGAACATCTTCTACAACAGCAACAACAAGAACTTCCTGGCCTGGTATCA |
| | GCAGAAGGCTGGCCAGTCTCCTAAGCTGCTGATCTACTGGGCCAGCACCAGAGA |
| | ATCTGGGGTGCCAGATAGATTTTCTGGCAGTGGCTCTGGCACAGACTTCACCCT |
| | GACAATCAGTGGACTGCAGGCTGGGGATGTTGCTGTGTACTTCTGCCAGCAGTA |
| | CTTCACAGATGCCCCTACCTTTGGTGGTGGCACCAAGGTGGAAATCAAGGGCGG |
| | AGGAGGCGGAGGTGGAGGAGGAGAGCCTAGAGGCCCCACCATCAAGCCCTGTCC |
| | TCCATGCAAGTGTCCTGCTCCTAACCTGCTTGGAGGCCCCTCTGTGTTCATCTT |
| | CCCACCTAAGATCAAGGATGTGCTGATGATCTCTCTGAGCCCCATTGTGACCTG |
| | TGTGGTGGTGGATGTGTCTGAGGATGACCCTGATGTGCAGATCTCTTGGTTTGT |
| | GAACAATGTGGAAGTGCACACAGCCCAGACACAGACCCACAGAGAGGACTACAA |
| | CAGCACCCTGAGAGTGGTGTCTGCCCTGCCTATCCAGCACCAGGACTGGATGAG |
| | TGGCAAAGAGTTCAAGTGCAAAGTGAACAACAAGGACCTGCCTGCTCCAATTGA |
| | GAGAACCATCAGCAAGCCCAAGGGCTCTGTTAGAGCCCCTCAAGTGTATGTGCT |
| | GCCTCCACCTGAGGAAGAGATGACCAAGAAACAAGTGACCCTGACATGCATGGT |
| | CACAGACTTCATGCCTGAGGACATCTATGTGGAATGGACCAACAATGGCAAGAC |
| | AGAGCTGAACTACAAGAACACTGAGCCTGTGCTGGACTCTGATGGCAGCTACTT |
| | CATGTACAGCAAGCTGAGGGTTGAGAAGAAGAACTGGGTTGAGAGAAACAGCTA |
| | CAGCTGCTCTGTGGTGCATGAGGGCCTGCACAATCACCACACCACCAAGAGCTT |
| | CAGCAGAACCCCTGGCAAGTAATGACTCGAGGACGGGGTGAACTACGCCTGAGG |
| | ATCCGATCTTTTTCCCTCTGCCAAAAATTATGGGGACATCATGAAGCCCCTTGA |
| | GCATCTGACTTCTGGCTAATAAAGGAAATTTATTTTCATTGCAATAGTGTGTTG |
| | GAATTTTTTGTGTCTCTCACTCGGAAGCAATTCGTTGATCTGAATTTCGACCAC |
| | CCATAATACCCATTACCCTGGTAGATAAGTAGCATGGCGGGTTAATCATTAACT |
| | ACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGG |
| | CCTCAGTGAGCGAGCGAGCGCGCAG (SEQ ID NO: 369) |
| 4p3 ScFv-Fc | ATGTACAGAATGCAGCTGCTGCTGCTCATTGCCCTGTCTCTGGCCCTGGTCACC |
| polynucleotide | AATTCTGAGGTGCAGCTGGTGCAGTCTGGGGCTGAAGTGAAGAAGCCTGGGGAG |
| (includes signal | AGCCTGAAGATCAGCTGCAAAGTGTCTGGCTATGACTTCAGCAAGTACTGGATT |
| peptide): | GGCTGGGTCAGACAGATGCCTGGCAAAGGCCTGGAATGGATGGGACTGATCTAC |
| (mIL2-4P3- | CCTGGGGACTCTGACACCAGATACAGCCCTAGCCTGGAAGGCCAGGTGTCCATC |
| VH3VL-9IgG2a) | TCTGCTGACAAGAGCATCTCTGCAGCCTACCTGCAGTTCAGCAGCCTGAAGGCC |
| | TCTGACTCTGCCATGTACTACTGTGTCAGAAAGGGCTACTCTGCCTACCTTGGA |
| | GGATGGTTTGATCCTTGGGGCCAGGGCACACTGGTCACAGTGTCCTCTGGTGGA |
| | GGCGGTTCAGGCGGAGGTGGCAGCGGCGGTGGAGGGTCGGACATCCAGCTGACA |
| | CAGAGCCCTGACAGCCTGGCTGCTTCTCTGGGAGAGAGAGTGACCATCAACTGC |
| | AGAAGCTCCCAGAACATCTTCTACAACAGCAACAACAAGAACTTCCTGGCCTGG |
| | TATCAGCAGAAGGCTGGCCAGTCTCCTAAGCTGCTGATCTACTGGGCCAGCACC |
| | AGAGAATCTGGGGTGCCAGATAGATTTTCTGGCAGTGGCTCTGGCACAGACTTC |
| | ACCCTGACAATCAGTGGACTGCAGGCTGGGGATGTTGCTGTGTACTTCTGCCAG |
| | CAGTACTTCACAGATGCCCCTACCTTTGGTGGTGGCACCAAGGTGGAAATCAAG |
| | GGCGGAGGAGGCGGAGGTGGAGGAGGAGAGCCTAGAGGCCCCACCATCAAGCCC |
| | TGTCCTCCATGCAAGTGTCCTGCTCCTAACCTGCTTGGAGGCCCCTCTGTGTTC |
| | ATCTTCCCACCTAAGATCAAGGATGTGCTGATGATCTCTCTGAGCCCCATTGTG |
| | ACCTGTGTGGTGGTGGATGTGTCTGAGGATGACCCTGATGTGCAGATCTCTTGG |
| | TTTGTGAACAATGTGGAAGTGCACACAGCCCAGACACAGACCCACAGAGAGGAC |
| | TACAACAGCACCCTGAGAGTGGTGTCTGCCCTGCCTATCCAGCACCAGGACTGG |
| | ATGAGTGGCAAAGAGTTCAAGTGCAAAGTGAACAACAAGGACCTGCCTGCTCCA |
| | ATTGAGAGAACCATCAGCAAGCCCAAGGGCTCTGTTAGAGCCCCTCAAGTGTAT |
| | GTGCTGCCTCCACCTGAGGAAGAGATGACCAAGAAACAAGTGACCCTGACATGC |
| | ATGGTCACAGACTTCATGCCTGAGGACATCTATGTGGAATGGACCAACAATGGC |
| | AAGACAGAGCTGAACTACAAGAACACTGAGCCTGTGCTGGACTCTGATGGCAGC |
| | TACTTCATGTACAGCAAGCTGAGGGTTGAGAAGAAGAACTGGGTTGAGAGAAAC |
| | AGCTACAGCTGCTCTGTGGTGCATGAGGGCCTGCACAATCACCACACCACCAAG |
| | AGCTTCAGCAGAACCCCTGGCAAG (SEQ ID NO: 370) |
| Full 4p3 ScFv-Fc | MYRMQLLLLIALSLALVINSEVQLVQSGAEVKKPGESLKISCKVSGYDESKYWI |
| polypeptide | GWVRQMPGKGLEWMGLIYPGDSDTRYSPSLEGQVSISADKSISAAYLQFSSLKA |
| (includes signal | SDSAMYYCVRKGYSAYLGGWFDPWGQGTLVTVSSGGGGSGGGGSGGGGSDIQLT |
| peptide) | QSPDSLAASLGERVTINCRSSQNIFYNSNNKNFLAWYQQKAGQSPKLLIYWAST |
| | RESGVPDRFSGSGSGTDFTLTISGLQAGDVAVYFCQQYFTDAPTFGGGTKVEIK |
| | GGGGGGGGGEPRGPTIKPCPPCKCPAPNLLGGPSVFIFPPKIKDVLMISLSPIV |
| | TCVVVDVSEDDPDVQISWFVNNVEVHTAQTQTHREDYNSTLRVVSALPIQHQDW |
| | MSGKEFKCKVNNKDLPAPIERTISKPKGSVRAPQVYVLPPPEEEMTKKQVTLTC |
| | MVTDFMPEDIYVEWTNNGKTELNYKNTEPVLDSDGSYFMYSKLRVEKKNWVERN |
| | SYSCSVVHEGLHNHHTTKSFSRTPGK (SEQ ID NO: 371) |
| | Bold/underline = 3xG4S linker |
| | Underline = 9G linker |
| Full 4p3 ScFv-Fc | CTGCGCGCTCGCTCGCTCACTGAGGCCGCCCGGGCAAAGCCCGGGCGTCGGGCG |
| polypeptide | ACCTTTGGTCGCCCGGCCTCAGTGAGCGAGCGAGCGCGCAGAGAGGGAGTGGCC |
| (includes signal | AACTCCATCACTAGGGGTTCCTTGTAGTTAATGATTAACCCGCCATGCTACTTA |
| peptide) | TCTACCAGGGTAATGGGGATCCTCTAGAACTATAGCTAGTCGACATTGATTATT |
| | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGCGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGGGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCGCCACCATGTA |
| | CAGAATGCAGCTGCTGCTGCTCATTGCCCTGTCTCTGGCCCTGGTCACCAATTC |
| | TGAGGTGCAGCTGCTGCAGAGTGGTGGTGGTGTTGTGCAGCCTGGTGGCTCTCT |
| | GAGACTGTCTTGTGAAGCCTCTGGCTTCACCTTCAGCAGCTACAGCATGCACTG |
| | GGTCAGACAGGCCCCTGGCAAAGGACTGGAATGGCTGACCCTGATCAGCTACAA |
| | TGGGGTCAACAAGTACTATGCCAAGTCTGTGAAGGGCAGATTTGCCATCAGCAG |
| | AGATGACAGCAAGTCTACCCTGTTCCTGCAGATGAACAGCCTGACCACAGATGA |
| | CACTGCTGTGTACTACTGTGCCAGATGTCCCATTCCTAGAGCTGCCAGCTACAG |
| | AAATGGGGACAACTACTTTGAGTTTGGCCAGGGCACCCTGGTCACAGTGTCCTC |
| | TGGTGGAGGCGGTTCAGGCGGAGGTGGCAGCGGCGGTGGAGGGTCGCAGGCTGT |
| | GGTCACCCAAGAGCCTAGCCTGTCTGTGTCTCCTGGTGGCACAGTGACACTGAC |
| | ATGTGCCCTGTCCTCTGCCTCTGTGTCTACCAGCAGCTACCCCAGCTGGTATCA |
| | GCAGACACCTGGCCAGCCTCCAAGAATGCTGATCCACAACACAGACACCAGATC |
| | CTCTGGGGTGCCAGACAGATTCTCTGGCAGCATCCTGGGCAAGAAGGCTGCCCT |
| | GACAATCACTGGTGCCCAGGCTGATGATGAGTCTGACTACTACTGTGTGGTGTA |
| | CCTTGGCAATGGCTTCTGGGTGTTTGGTGGTGGCACCAAGCTGACAGTGCTGGG |
| | CGGAGGAGGCGGAGGTGGAGGAGGAGAGCCTAGAGGCCCCACCATCAAGCCCTG |
| | TCCTCCATGCAAGTGTCCTGCTCCTAACCTGCTTGGAGGCCCCTCTGTGTTCAT |
| | CTTCCCACCTAAGATCAAGGATGTGCTGATGATCTCTCTGAGCCCCATTGTGAC |
| | CTGTGTGGTGGTGGATGTGTCTGAGGATGACCCTGATGTGCAGATCTCTTGGTT |
| | TGTGAACAATGTGGAAGTGCACACAGCCCAGACACAGACCCACAGAGAGGACTA |
| | CAACAGCACCCTGAGAGTGGTGTCTGCCCTGCCTATCCAGCACCAGGACTGGAT |
| | GAGTGGCAAAGAGTTCAAGTGCAAAGTGAACAACAAGGACCTGCCTGCTCCAAT |
| | TGAGAGAACCATCAGCAAGCCCAAGGGCTCTGTTAGAGCCCCTCAAGTGTATGT |
| | GCTGCCTCCACCTGAGGAAGAGATGACCAAGAAACAAGTGACCCTGACATGCAT |
| | GGTCACAGACTTCATGCCTGAGGACATCTATGTGGAATGGACCAACAATGGCAA |
| | GACAGAGCTGAACTACAAGAACACTGAGCCTGTGCTGGACTCTGATGGCAGCTA |
| | CTTCATGTACAGCAAGCTGAGGGTTGAGAAGAAGAACTGGGTTGAGAGAAACAG |
| | CTACAGCTGCTCTGTGGTGCATGAGGGCCTGCACAATCACCACACCACCAAGAG |
| | CTTCAGCAGAACCCCTGGCAAGTAATGACTCGAGGACGGGGTGAACTACGCCTG |
| | AGGATCCGATCTTTTTCCCTCTGCCAAAAATTATGGGGACATCATGAAGCCCCT |
| | TGAGCATCTGACTTCTGGCTAATAAAGGAAATTTATTTTCATTGCAATAGTGTG |
| | TTGGAATTTTTTGTGTCTCTCACTCGGAAGCAATTCGTTGATCTGAATTTCGAC |
| | CACCCATAATACCCATTACCCTGGTAGATAAGTAGCATGGCGGGTTAATCATTA |
| | ACTACAAGGAACCCCTAGTGATGGAGTTGGCCACTCCCTCTCTGCGCGCTCGCT |
| | CGCTCACTGAGGCCGGGCGACCAAAGGTCGCCCGACGCCCGGGCTTTGCCCGGG |
| | CGGCCTCAGTGAGCGAGCGAGCGCGCAG (SEQ ID NO: 372) |
| 31B6 ScFv-Fc | ATGTACAGAATGCAGCTGCTGCTGCTCATTGCCCTGTCTCTGGCCCTGGTCACC |
| polynucleotide | AATTCTGAGGTGCAGCTGCTGCAGAGTGGTGGTGGTGTTGTGCAGCCTGGTGGC |
| (includes signal | TCTCTGAGACTGTCTTGTGAAGCCTCTGGCTTCACCTTCAGCAGCTACAGCATG |
| peptide) | CACTGGGTCAGACAGGCCCCTGGCAAAGGACTGGAATGGCTGACCCTGATCAGC |
| (mIL2-31B6- | TACAATGGGGTCAACAAGTACTATGCCAAGTCTGTGAAGGGCAGATTTGCCATC |
| VH3VL- | AGCAGAGATGACAGCAAGTCTACCCTGTTCCTGCAGATGAACAGCCTGACCACA |
| 9GIgG2a) | GATGACACTGCTGTGTACTACTGTGCCAGATGTCCCATTCCTAGAGCTGCCAGC |
| | TACAGAAATGGGGACAACTACTTTGAGTTTGGCCAGGGCACCCTGGTCACAGTG |
| | TCCTCTggtggaggcggttcaggcggaggtggcagcggcggtggagggtcgCAG |
| | GCTGTGGTCACCCAAGAGCCTAGCCTGTCTGTGTCTCCTGGTGGCACAGTGACA |
| | CTGACATGTGCCCTGTCCTCTGCCTCTGTGTCTACCAGCAGCTACCCCAGCTGG |
| | TATCAGCAGACACCTGGCCAGCCTCCAAGAATGCTGATCCACAACACAGACACC |
| | AGATCCTCTGGGGTGCCAGACAGATTCTCTGGCAGCATCCTGGGCAAGAAGGCT |
| | GCCCTGACAATCACTGGTGCCCAGGCTGATGATGAGTCTGACTACTACTGTGTG |
| | GTGTACCTTGGCAATGGCTTCTGGGTGTTTGGTGGTGGCACCAAGCTGACAGTG |
| | CTGGGCGGAGGAGGCGGAGGTGGAGGAGGAGAGCCTAGAGGCCCCACCATCAAG |
| | CCCTGTCCTCCATGCAAGTGTCCTGCTCCTAACCTGCTTGGAGGCCCCTCTGTG |
| | TTCATCTTCCCACCTAAGATCAAGGATGTGCTGATGATCTCTCTGAGCCCCATT |
| | GTGACCTGTGTGGTGGTGGATGTGTCTGAGGATGACCCTGATGTGCAGATCTCT |
| | TGGTTTGTGAACAATGTGGAAGTGCACACAGCCCAGACACAGACCCACAGAGAG |
| | GACTACAACAGCACCCTGAGAGTGGTGTCTGCCCTGCCTATCCAGCACCAGGAC |
| | TGGATGAGTGGCAAAGAGTTCAAGTGCAAAGTGAACAACAAGGACCTGCCTGCT |
| | CCAATTGAGAGAACCATCAGCAAGCCCAAGGGCTCTGTTAGAGCCCCTCAAGTG |
| | TATGTGCTGCCTCCACCTGAGGAAGAGATGACCAAGAAACAAGTGACCCTGACA |
| | TGCATGGTCACAGACTTCATGCCTGAGGACATCTATGTGGAATGGACCAACAAT |
| | GGCAAGACAGAGCTGAACTACAAGAACACTGAGCCTGTGCTGGACTCTGATGGC |
| | AGCTACTTCATGTACAGCAAGCTGAGGGTTGAGAAGAAGAACTGGGTTGAGAGA |
| | AACAGCTACAGCTGCTCTGTGGTGCATGAGGGCCTGCACAATCACCACACCACC |
| | AAGAGCTTCAGCAGAACCCCTGGCAAG (SEQ ID NO: 373) |
| Full 31B6 ScFv- | MYRMQLLLLIALSLALVINSEVQLLQSGGGVVQPGGSLRLSCEASGFTFSSYSM |
| Fc polypeptide | HWVRQAPGKGLEWLTLISYNGVNKYYAKSVKGRFAISRDDSKSTLFLQMNSLTT |
| (includes signal | DDTAVYYCARCPIPRAASYRNGDNYFEFGQGTLVTVSSGGGGSGGGGSGGGGSQ |
| peptide): | AVVTQEPSLSVSPGGTVTLTCALSSASVSTSSYPSWYQQTPGQPPRMLIHNTDT |
| | RSSGVPDRESGSILGKKAALTITGAQADDESDYYCVVYLGNGFWVFGGGTKLTV |
| | LGGGGGGGGGEPRGPTIKPCPPCKCPAPNLLGGPSVFIFPPKIKDVLMISLSPI |
| | VTCVVVDVSEDDPDVQISWFVNNVEVHTAQTQTHREDYNSTLRVVSALPIQHQD |
| | WMSGKEFKCKVNNKDLPAPIERTISKPKGSVRAPQVYVLPPPEEEMTKKQVTLT |
| | CMVTDFMPEDIYVEWTNNGKTELNYKNTEPVLDSDGSYFMYSKLRVEKKNWVER |
| | NSYSCSVVHEGLHNHHTTKSFSRTPGK (SEQ ID NO: 374) |
| | Bold/underline = 3xG4S linker |
| | Underline = 9G linker |
| 43A11-HL-L3 | CTGCGCGCTCGCTCGCTCACTGAGGCCGCCCGGGCAAAGCCCGGGCGTCGGGCG |
| scFv-Fc | ACCTTTGGTCGCCCGGCCTCAGTGAGCGAGCGAGCGCGCAGAGAGGGAGTGGCC |
| [43A11 = control | AACTCCATCACTAGGGGTTCCTTGTAGTTAATGATTAACCCGCCATGCTACTTA |
| mAb)] | TCTACCAGGGTAATGGGGATCCTCTAGAACTATAGCTAGTCGACATTGATTATT |
| ITR-ITR: | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGCGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGCGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCGCCACCATGTA |
| | CAGAATGCAGCTGCTGCTGCTCATTGCCCTGTCTCTGGCCCTGGTCACCAATTC |
| | TCAGGTCCAGCTGCAAGAGTCTGGCCCTGGACTGGTCAAGCCTTCTGGCACACT |
| | GAGCCTGACCTGTGCTGTGACTGGTGGCAGCATCAGCAGCAGCAATTGGTGGTC |
| | TTGGGTCAGACAGAGCCCTGGCAAAGGCCTGGAATGGATTGGAGAGATCCACCA |
| | TGATGGCAACCTGAACTACAACCCTCTGCTGAAGTCCAGAGTGTCCATGAGCCT |
| | GGACAGAAGCAAGAACCAGTTCAGCCTGAAGCTGACCTCTGTGACAGCTGCTGA |
| | CACTGCTGTGTACTACTGTGCCAGATGGGACTTCTTCTTTGACTCCTCCTACTA |
| | CATCAGGGGCTTTGACCTGTGGGGCCAGGGCACAATGGTTACAGTGTCCTCTGG |
| | TGGAGGCGGTTCAGGCGGAGGTGGCAGCGGCGGTGGAGGGTCGGAGACAACCCT |
| | GACACAGAGCCCTGGCACATTGTCTCTGAGCCCTGGGGAGAGAGTGACCCTGTC |
| | TTGTAGAGCCAGCCAGTCTGTGGACAGAAACTACCTGGCCTGGTATCAGCAGAA |
| | GCCTGGCCAGTCTCCTAGACTGCTGATCTACTCTGCCTCTAGGAGAGCCACAGG |
| | CATCCCTGACAGATTTTCTGGCAGTGGCTCTGGCACAGACTTCACCCTGACCAT |
| | CAGCAGACTGGAACCTGAGGACTTTGTGGTGTACTACTGCCAGCAGTATGGTGG |
| | CAGCCCTCCTATCACATTTGGCCAGGGCACAAGACTGGAAATCAAGGGCGGAGG |
| | AGGCGGAGGTGGAGGAGGAGAGCCTAGAGGCCCCACCATCAAGCCCTGTCCTCC |
| | ATGCAAGTGTCCTGCTCCTAACCTGCTTGGAGGCCCCTCTGTGTTCATCTTCCC |
| | ACCTAAGATCAAGGATGTGCTGATGATCTCTCTGAGCCCCATTGTGACCTGTGT |
| | GGTGGTGGATGTGTCTGAGGATGACCCTGATGTGCAGATCTCTTGGTTTGTGAA |
| | CAATGTGGAAGTGCACACAGCCCAGACACAGACCCACAGAGAGGACTACAACAG |
| | CACCCTGAGAGTGGTGTCTGCCCTGCCTATCCAGCACCAGGACTGGATGAGTGG |
| | CAAAGAGTTCAAGTGCAAAGTGAACAACAAGGACCTGCCTGCTCCAATTGAGAG |
| | AACCATCAGCAAGCCCAAGGGCTCTGTTAGAGCCCCTCAAGTGTATGTGCTGCC |
| | TCCACCTGAGGAAGAGATGACCAAGAAACAAGTGACCCTGACATGCATGGTCAC |
| | AGACTTCATGCCTGAGGACATCTATGTGGAATGGACCAACAATGGCAAGACAGA |
| | GCTGAACTACAAGAACACTGAGCCTGTGCTGGACTCTGATGGCAGCTACTTCAT |
| | GTACAGCAAGCTGAGGGTTGAGAAGAAGAACTGGGTTGAGAGAAACAGCTACAG |
| | CTGCTCTGTGGTGCATGAGGGCCTGCACAATCACCACACCACCAAGAGCTTCAG |
| | CAGAACCCCTGGCAAGTAATGACTCGAGGACGGGGTGAACTACGCCTGAGGATC |
| | CGATCTTTTTCCCTCTGCCAAAAATTATGGGGACATCATGAAGCCCCTTGAGCA |
| | TCTGACTTCTGGCTAATAAAGGAAATTTATTTTCATTGCAATAGTGTGTTGGAA |
| | TTTTTTGTGTCTCTCACTCGGAAGCAATTCGTTGATCTGAATTTCGACCACCCA |
| | TAATACCCATTACCCTGGTAGATAAGTAGCATGGCGGGTTAATCATTAACTACA |
| | AGGAACCCCTAGTGATGGAGTTGGCCACTCCCTCTCTGCGCGCTCGCTCGCTCA |
| | CTGAGGCCGGGCGACCAAAGGTCGCCCGACGCCCGGGCTTTGCCCGGGCGGCCT |
| | CAGTGAGCGAGCGAGCGCGCAG (SEQ ID NO: 375) |
| 43A11-ScFv-Fc | ATGTACAGAATGCAGCTGCTGCTGCTCATTGCCCTGTCTCTGGCCCTGGTCACC |
| polynucleotide | AATTCTCAGGTCCAGCTGCAAGAGTCTGGCCCTGGACTGGTCAAGCCTTCTGGC |
| (includes signal | ACACTGAGCCTGACCTGTGCTGTGACTGGTGGCAGCATCAGCAGCAGCAATTGG |
| peptide): | TGGTCTTGGGTCAGACAGAGCCCTGGCAAAGGCCTGGAATGGATTGGAGAGATC |
| (mIL2-43A11- | CACCATGATGGCAACCTGAACTACAACCCTCTGCTGAAGTCCAGAGTGTCCATG |
| VH3VL- | AGCCTGGACAGAAGCAAGAACCAGTTCAGCCTGAAGCTGACCTCTGTGACAGCT |
| 9GIgG2) | GCTGACACTGCTGTGTACTACTGTGCCAGATGGGACTTCTTCTTTGACTCCTCC |
| | TACTACATCAGGGGCTTTGACCTGTGGGGCCAGGGCACAATGGTTACAGTGTCC |
| | TCTGGTGGAGGCGGTTCAGGCGGAGGTGGCAGCGGCGGTGGAGGGTCGGAGACA |
| | ACCCTGACACAGAGCCCTGGCACATTGTCTCTGAGCCCTGGGGAGAGAGTGACC |
| | CTGTCTTGTAGAGCCAGCCAGTCTGTGGACAGAAACTACCTGGCCTGGTATCAG |
| | CAGAAGCCTGGCCAGTCTCCTAGACTGCTGATCTACTCTGCCTCTAGGAGAGCC |
| | ACAGGCATCCCTGACAGATTTTCTGGCAGTGGCTCTGGCACAGACTTCACCCTG |
| | ACCATCAGCAGACTGGAACCTGAGGACTTTGTGGTGTACTACTGCCAGCAGTAT |
| | GGTGGCAGCCCTCCTATCACATTTGGCCAGGGCACAAGACTGGAAATCAAGGGC |
| | GGAGGAGGCGGAGGTGGAGGAGGAGAGCCTAGAGGCCCCACCATCAAGCCCTGT |
| | CCTCCATGCAAGTGTCCTGCTCCTAACCTGCTTGGAGGCCCCTCTGTGTTCATC |
| | TTCCCACCTAAGATCAAGGATGTGCTGATGATCTCTCTGAGCCCCATTGTGACC |
| | TGTGTGGTGGTGGATGTGTCTGAGGATGACCCTGATGTGCAGATCTCTTGGTTT |
| | GTGAACAATGTGGAAGTGCACACAGCCCAGACACAGACCCACAGAGAGGACTAC |
| | AACAGCACCCTGAGAGTGGTGTCTGCCCTGCCTATCCAGCACCAGGACTGGATG |
| | AGTGGCAAAGAGTTCAAGTGCAAAGTGAACAACAAGGACCTGCCTGCTCCAATT |
| | GAGAGAACCATCAGCAAGCCCAAGGGCTCTGTTAGAGCCCCTCAAGTGTATGTG |
| | CTGCCTCCACCTGAGGAAGAGATGACCAAGAAACAAGTGACCCTGACATGCATG |
| | GTCACAGACTTCATGCCTGAGGACATCTATGTGGAATGGACCAACAATGGCAAG |
| | ACAGAGCTGAACTACAAGAACACTGAGCCTGTGCTGGACTCTGATGGCAGCTAC |
| | TTCATGTACAGCAAGCTGAGGGTTGAGAAGAAGAACTGGGTTGAGAGAAACAGC |
| | TACAGCTGCTCTGTGGTGCATGAGGGCCTGCACAATCACCACACCACCAAGAGC |
| | TTCAGCAGAACCCCTGGCAAG (SEQ ID NO: 376) |
| Full 43A11 | MYRMQLLLLIALSLALVTNSQVQLQESGPGLVKPSGTLSLTCAVTGGSISSSNW |
| ScFv-Fc | WSWVRQSPGKGLEWIGEIHHDGNLNYNPLLKSRVSMSLDRSKNQFSLKLTSVTA |
| polypeptide | ADTAVYYCARWDFFFDSSYYIRGFDLWGQGTMVTVSSGGGGSGGGGSGGGGSET |
| (includes signal | TLTQSPGTLSLSPGERVTLSCRASQSVDRNYLAWYQQKPGQSPRLLIYSASRRA |
| peptide) | TGIPDRFSGSGSGTDFTLTISRLEPEDFVVYYCQQYGGSPPITFGQGTRLEIKG |
| | GGGGGGGGEPRGPTIKPCPPCKCPAPNLLGGPSVFIFPPKIKDVLMISLSPIVT |
| | CVVVDVSEDDPDVQISWFVNNVEVHTAQTQTHREDYNSTLRVVSALPIQHQDWM |
| | SGKEFKCKVNNKDLPAPIERTISKPKGSVRAPQVYVLPPPEEEMTKKQVTLTCM |
| | VTDFMPEDIYVEWTNNGKTELNYKNTEPVLDSDGSYFMYSKLRVEKKNWVERNS |
| | YSCSVVHEGLHNHHTTKSFSRTPGK (SEQ ID NO: 377) |
| | Bold/underline = 3xG4S linker |
| | Underline = 9G linker |

ScFv-Fc mAbs are expressed as single-chain polypeptides which dimerize to provide antibodies capable of bivalent binding to antigen, similarly to full-length quadroma antibodies.

Table 11G shows examples of anti-Tau Fab constructs. Each construct utilizes the Furin (GSG) T2A linker system: RKRR GSG EGRGSLLTCGDVEENPGP between the heavy and light chains. The hinge region at the 3′ end of Fab Heavy Chain (following C_{H}1) is: EPKSCDKTH. Included in Table 11G are expression constructs that include the nucleotide sequence encoding the heavy and light chain liked by a Furin(GSG) T2A linker, regulatory elements, including a CAG promoter and polyadenylation signal sequence, flanked by ITR sequences, the sequence encoding the Fab fragment, and the amino acid sequence of the encoded polypeptide (the heavy and light chain linked via the linker).

TABLE 11G
Anti-Tau Fab Fragment Constructs

| Construct | Sequence |
|---|---|
| pAAV.CAG.hu. | CTGCGCGCTCGCTCGCTCACTGAGGCCGCCCGGGCAAAGCCCGGGCGTCGGGCG |
| Tau.4p3-Fab | ACCTTTGGTCGCCCGGCCTCAGTGAGCGAGCGAGCGCGCAGAGAGGGAGTGGCC |
| (ITR to ITR) | AACTCCATCACTAGGGGTTCCTTGTAGTTAATGATTAACCCGCCATGCTACTTA |
| | TCTACCAGGGTAATGGGGATCCTCTAGAACTATAGCTAGTCGACATTGATTATT |
| | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGGGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGCGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCGCCACCATGTA |
| | CAGAATGCAGCTGCTGCTGCTCATTGCCCTGTCTCTGGCCCTGGTCACCAATTC |
| | TGAAGTGCAGCTGGTGCAGTCTGGGGCTGAAGTGAAGAAGCCTGGGGAGAGCCT |
| | GAAGATCAGCTGCAAAGTGTCTGGCTATGACTTCAGCAAGTACTGGATTGGCTG |
| | GGTCAGACAGATGCCTGGCAAAGGCCTGGAATGGATGGGCCTGATCTACCCTGG |
| | GGACTCTGACACCAGATACAGCCCTAGCCTGGAAGGCCAGGTGTCCATCTCTGC |
| | TGACAAGAGCATCTCTGCAGCCTACCTGCAGTTCAGCAGCCTGAAGGCCTCTGA |
| | CTCTGCCATGTACTACTGTGTCAGAAAGGGCTACTCTGCCTACCTTGGAGGATG |
| | GTTTGATCCTTGGGGCCAGGGCACACTGGTTACAGTGTCCTCTGCCAGCACAAA |
| | GGGCCCCTCTGTTTTTCCACTGGCTCCCAGCAGCAAGTCCACCAGTGGTGGTAC |
| | AGCTGCCCTGGGCTGTCTGGTCAAGGATTACTTCCCTGAGCCTGTGACTGTGTC |
| | CTGGAACTCTGGTGCTCTGACCTCTGGGGTGCACACCTTTCCAGCTGTGCTGCA |
| | GTCCTCTGGCCTGTACTCTCTGTCCTCTGTGGTCACAGTGCCTAGCTCTAGCCT |
| | GGGCACCCAGACCTACATCTGCAATGTGAACCACAAGCCTAGCAACACCAAGGT |
| | GGACAAGAGAGTGGAACCCAAGAGCTGTGACAAGACCCACAGAAAGAGAAGAGG |
| | CTCTGGAGAAGGCAGAGGCTCCCTGCTGACATGTGGGGATGTTGAAGAGAATCC |
| | TGGGCCTATGTATAGGATGCAACTGCTCCTCCTGATTGCTCTGAGCCTGGCTCT |
| | TGTGACCAACTCTGATATCCAGCTGACACAGAGCCCTGACAGCCTGGCTGCTTC |
| | TCTGGGAGAGAGAGTGACCATCAACTGCAGAAGCTCCCAGAACATCTTCTACAA |
| | CTCCAACAACAAGAACTTCCTGGCCTGGTATCAGCAAAAGGCTGGACAGAGCCC |
| | CAAGCTGCTGATCTACTGGGCCTCTACCAGAGAGTCTGGGGTCCCAGACAGATT |
| | CTCTGGCTCTGGATCTGGCACAGACTTCACCCTGACCATCAGTGGACTGCAGGC |
| | TGGGGATGTTGCTGTGTACTTCTGCCAGCAGTACTTCACAGATGCCCCTACCTT |
| | TGGTGGTGGCACAAAGGTGGAAATCAAGAGAACAGTGGCTGCCCCATCTGTGTT |
| | CATCTTCCCACCATCTGATGAACAGCTGAAGTCTGGCACTGCCTCTGTTGTGTG |
| | CCTGCTGAACAACTTCTACCCTAGAGAAGCCAAGGTGCAGTGGAAAGTGGATAA |
| | TGCCCTCCAGTCTGGCAATAGCCAAGAGTCAGTGACAGAGCAGGACTCCAAGGA |
| | TTCCACCTACAGCCTGAGCAGCACCCTGACACTGAGCAAGGCTGACTATGAGAA |
| | GCACAAAGTGTATGCCTGTGAAGTGACACACCAGGGACTGAGCAGCCCAGTGAC |
| | CAAGAGCTTCAACAGGGGAGAGTGCTGATAACTCGAGGACGGGGTGAACTACGC |
| | CTGAGGATCCGATCTTTTTCCCTCTGCCAAAAATTATGGGGACATCATGAAGCC |
| | CCTTGAGCATCTGACTTCTGGCTAATAAAGGAAATTTATTTTCATTGCAATAGT |
| | GTGTTGGAATTTTTTGTGTCTCTCACTCGGAAGCAATTCGTTGATCTGAATTTC |
| | GACCACCCATAATACCCATTACCCTGGTAGATAAGTAGCATGGCGGGTTAATCA |
| | TTAACTACAAGGAACCCCTAGTGATGGAGTTGGCCACTCCCTCTCTGCGCGCTC |
| | GCTCGCTCACTGAGGCCGGGCGACCAAAGGTCGCCCGACGCCCGGGCTTTGCCC |
| | GGGCGGCCTCAGTGAGCGAGCGAGCGCGCAG (SEQ ID NO: 378) |
| 4p3 Fab | ATGTACAGAATGCAGCTGCTGCTGCTCATTGCCCTGTCTCTGGCCCTGGTCACC |
| polynucleotide | AATTCTGAAGTGCAGCTGGTGCAGTCTGGGGCTGAAGTGAAGAAGCCTGGGGAG |
| (mIL2- | AGCCTGAAGATCAGCTGCAAAGTGTCTGGCTATGACTTCAGCAAGTACTGGATT |
| 4p3_HC_Fab- | GGCTGGGTCAGACAGATGCCTGGCAAAGGCCTGGAATGGATGGGCCTGATCTAC |
| FurinT2A-mIL2- | CCTGGGGACTCTGACACCAGATACAGCCCTAGCCTGGAAGGCCAGGTGTCCATC |
| 4p3_LC) | TCTGCTGACAAGAGCATCTCTGCAGCCTACCTGCAGTTCAGCAGCCTGAAGGCC |
| | TCTGACTCTGCCATGTACTACTGTGTCAGAAAGGGCTACTCTGCCTACCTTGGA |
| | GGATGGTTTGATCCTTGGGGCCAGGGCACACTGGTTACAGTGTCCTCTGCCAGC |
| | ACAAAGGGCCCCTCTGTTTTTCCACTGGCTCCCAGCAGCAAGTCCACCAGTGGT |
| | GGTACAGCTGCCCTGGGCTGTCTGGTCAAGGATTACTTCCCTGAGCCTGTGACT |
| | GTGTCCTGGAACTCTGGTGCTCTGACCTCTGGGGTGCACACCTTTCCAGCTGTG |
| | CTGCAGTCCTCTGGCCTGTACTCTCTGTCCTCTGTGGTCACAGTGCCTAGCTCT |
| | AGCCTGGGCACCCAGACCTACATCTGCAATGTGAACCACAAGCCTAGCAACACC |
| | AAGGTGGACAAGAGAGTGGAACCCAAGAGCTGTGACAAGACCCACAGAAAGAGA |
| | AGAGGCTCTGGAGAAGGCAGAGGCTCCCTGCTGACATGTGGGGATGTTGAAGAG |
| | AATCCTGGGCCTATGTATAGGATGCAACTGCTCCTCCTGATTGCTCTGAGCCTG |
| | GCTCTTGTGACCAACTCTGATATCCAGCTGACACAGAGCCCTGACAGCCTGGCT |
| | GCTTCTCTGGGAGAGAGAGTGACCATCAACTGCAGAAGCTCCCAGAACATCTTC |
| | TACAACTCCAACAACAAGAACTTCCTGGCCTGGTATCAGCAAAAGGCTGGACAG |
| | AGCCCCAAGCTGCTGATCTACTGGGCCTCTACCAGAGAGTCTGGGGTCCCAGAC |
| | AGATTCTCTGGCTCTGGATCTGGCACAGACTTCACCCTGACCATCAGTGGACTG |
| | CAGGCTGGGGATGTTGCTGTGTACTTCTGCCAGCAGTACTTCACAGATGCCCCT |
| | ACCTTTGGTGGTGGCACAAAGGTGGAAATCAAGAGAACAGTGGCTGCCCCATCT |
| | GTGTTCATCTTCCCACCATCTGATGAACAGCTGAAGTCTGGCACTGCCTCTGTT |
| | GTGTGCCTGCTGAACAACTTCTACCCTAGAGAAGCCAAGGTGCAGTGGAAAGTG |
| | GATAATGCCCTCCAGTCTGGCAATAGCCAAGAGTCAGTGACAGAGCAGGACTCC |
| | AAGGATTCCACCTACAGCCTGAGCAGCACCCTGACACTGAGCAAGGCTGACTAT |
| | GAGAAGCACAAAGTGTATGCCTGTGAAGTGACACACCAGGGACTGAGCAGCCCA |
| | GTGACCAAGAGCTTCAACAGGGGAGAGTGCTGA (SEQ ID NO: 379) |
| 4p3 Fab | HC (with signal peptide): |
| polypeptides | MYRMQLLLLIALSLALVTNSEVQLVQSGAEVKKPGESLKISCKVSGYDESKYWI |
| | GWVRQMPGKGLEWMGLIYPGDSDTRYSPSLEGQVSISADKSISAAYLQFSSLKA |
| | SDSAMYYCVRKGYSAYLGGWFDPWGQGTLVTVSSASTKGPSVFPLAPSSKSTSG |
| | GTAALGCLVKDYFPEPVTVSWNSGALTSGVHTFPAVLQSSGLYSLSSVVTVPSS |
| | SLGTQTYICNVNHKPSNTKVDKRVEPKSCDKTH (SEQ ID NO: 380) |
| | HC (no signal): |
| | EVQLVQSGAEVKKPGESLKISCKVSGYDFSKYWIGWVRQMPGKGLEWMGLIYPG |
| | DSDTRYSPSLEGQVSISADKSISAAYLQFSSLKASDSAMYYCVRKGYSAYLGGW |
| | FDPWGQGTLVTVSSASTKGPSVFPLAPSSKSTSGGTAALGCLVKDYFPEPVTVS |
| | WNSGALTSGVHTFPAVLQSSGLYSLSSVVTVPSSSLGTQTYICNVNHKPSNTKV |
| | DKRVEPKSCDKTH (SEQ ID NO: 381) |
| | LC (kappa) (with signal peptide): |
| | MYRMQLLLLIALSLALVINSDIQLTQSPDSLAASLGERVTINCRSSQNIFYNSN |
| | NKNFLAWYQQKAGQSPKLLIYWASTRESGVPDRFSGSGSGTDFTLTISGLQAGD |
| | VAVYFCQQYFTDAPTFGGGTKVEIKRTVAAPSVFIFPPSDEQLKSGTASVVCLL |
| | NNFYPREAKVQWKVDNALQSGNSQESVTEQDSKDSTYSLSSTLTLSKADYEKHK |
| | VYACEVTHQGLSSPVTKSENRGEC (SEQ ID NO: 382) |
| | LC (kappa)(no signal) |
| | DIQLTQSPDSLAASLGERVTINCRSSQNIFYNSNNKNFLAWYQQKAGQSPKLLI |
| | YWASTRESGVPDRFSGSGSGTDFTLTISGLQAGDVAVYFCQQYFTDAPTFGGGT |
| | KVEIKRTVAAPSVFIFPPSDEQLKSGTASVVCLLNNFYPREAKVQWKVDNALQS |
| | GNSQESVTEQDSKDSTYSLSSTLTLSKADYEKHKVYACEVTHQGLSSPVTKSFN |
| | RGEC (SEQ ID NO: 383) |
| pAAV.CAG.hu. | CTGCGCGCTCGCTCGCTCACTGAGGCCGCCCGGGCAAAGCCCGGGCGTCGGGCG |
| Tau8H1-Fab | ACCTTTGGTCGCCCGGCCTCAGTGAGCGAGCGAGCGCGCAGAGAGGGAGTGGCC |
| (ITR to ITR) | AACTCCATCACTAGGGGTTCCTTGTAGTTAATGATTAACCCGCCATGCTACTTA |
| | TCTACCAGGGTAATGGGGATCCTCTAGAACTATAGCTAGTCGACATTGATTATT |
| | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGCGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGGGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCGCCACCATGTA |
| | CAGAATGCAGCTGCTGCTGCTCATTGCCCTGTCTCTGGCCCTGGTCACCAATTC |
| | TGAAGTGCAGCTGGTGGAAAGTGGTGGTGGACTGGTTCAGCCTGGTGGCAGCCT |
| | GAGACTGTCTTGTGCTGCCTCTGGCTTCACCTTCAGCATCTACACCATGAGCTG |
| | GGTCAGACAGGCCCCTGGCAAAGGACTGGAATGGGTGTCAGCCATCCTGTCTGA |
| | TGGCAGCACCTACTATGCTGACTCTGTGGAAGGCAGATTCACCATCAGCAGAGA |
| | CAACAGCAAGACCACACTGTACCTGCAGATGAACAGCCTGACAGCTGAGGACAC |
| | AGCAGTGTACTACTGCACCAAGAGAGTGGAAGGCTCCTTCAGAGGCTGGGGCCA |
| | GGGAACACTGGTTACAGTGTCCTCTGCCAGCACAAAGGGCCCCTCTGTTTTTCC |
| | ACTGGCTCCCAGCAGCAAGAGCACCAGTGGTGGAACAGCTGCCCTGGGCTGTCT |
| | GGTCAAGGATTACTTCCCTGAGCCTGTGACTGTGTCCTGGAACTCTGGTGCACT |
| | GACCTCTGGGGTGCACACCTTTCCAGCTGTGCTGCAGTCCTCTGGCCTGTACTC |
| | TCTGTCCTCTGTGGTCACAGTGCCTAGCTCTAGCCTGGGCACCCAGACCTACAT |
| | CTGCAATGTGAACCACAAGCCTAGCAACACCAAGGTGGACAAAAGAGTGGAACC |
| | CAAGAGCTGTGACAAGACCCACAGAAAGAGAAGAGGCTCTGGAGAAGGCAGAGG |
| | CTCCCTGCTGACATGTGGGGATGTTGAAGAGAATCCTGGGCCTATGTATAGGAT |
| | GCAACTGCTCCTCCTGATTGCTCTGAGCCTGGCTCTTGTGACCAACTCTGAGAT |
| | TGTGATGACACAGAGCCCAGGCACACTGTCACTGTCTCCAGGGGAATCTGCCAC |
| | TCTGAGCTGTAGAGCCAGCCAGTCTGTGTCTACCACCTACCTGGCCTGGTATCA |
| | GCAAAAGCCTGGACAGGCTCCCAGACTGCTGATCTATGGGGCCTCTTCTAGAGC |
| | CACAGGCATCCCTGACAGATTTTCTGGCAGTGGCTCTGGCACAGACTTCACCCT |
| | GACCATCTCTAGACTGGAACCTGAGGACTTTGCTGTCTACTACTGCCAGCAGTA |
| | TGGTGGCTCCAGACTGATCACCTTTGGCCAGGGCACCAGACTGGAAATCAAGAG |
| | AACAGTGGCTGCCCCATCTGTGTTCATCTTCCCACCATCTGATGAGCAGCTCAA |
| | GTCTGGCACTGCCTCTGTTGTGTGCCTGCTGAACAACTTCTACCCTAGAGAAGC |
| | CAAGGTGCAGTGGAAGGTTGACAATGCCCTGCAGTCTGGCAATAGCCAAGAATC |
| | TGTGACAGAGCAGGACTCCAAGGATAGCACCTACAGCCTGTCTAGCACCCTGAC |
| | ACTGAGCAAGGCTGACTATGAGAAGCACAAAGTGTATGCCTGTGAAGTGACACA |
| | CCAGGGCCTGAGCAGCCCAGTGACCAAGAGCTTCAATAGAGGGGAGTGCTGATA |
| | ACTCGAGGACGGGGTGAACTACGCCTGAGGATCCGATCTTTTTCCCTCTGCCAA |
| | AAATTATGGGGACATCATGAAGCCCCTTGAGCATCTGACTTCTGGCTAATAAAG |
| | GAAATTTATTTTCATTGCAATAGTGTGTTGGAATTTTTTGTGTCTCTCACTCGG |
| | AAGCAATTCGTTGATCTGAATTTCGACCACCCATAATACCCATTACCCTGGTAG |
| | ATAAGTAGCATGGCGGGTTAATCATTAACTACAAGGAACCCCTAGTGATGGAGT |
| | TGGCCACTCCCTCTCTGCGCGCTCGCTCGCTCACTGAGGCCGGGCGACCAAAGG |
| | TCGCCCGACGCCCGGGCTTTGCCCGGGCGGCCTCAGTGAGCGAGCGAGCGCGCA |
| | G (SEQ ID NO: 384) |
| 8H1 Fab | ATGTACAGAATGCAGCTGCTGCTGCTCATTGCCCTGTCTCTGGCCCTGGTCACC |
| nucleotide: | AATTCTGAAGTGCAGCTGGTGGAAAGTGGTGGTGGACTGGTTCAGCCTGGTGGC |
| (mIL2- | AGCCTGAGACTGTCTTGTGCTGCCTCTGGCTTCACCTTCAGCATCTACACCATG |
| 8H1_HC_Fab- | AGCTGGGTCAGACAGGCCCCTGGCAAAGGACTGGAATGGGTGTCAGCCATCCTG |
| FurinT2A-mIL2- | TCTGATGGCAGCACCTACTATGCTGACTCTGTGGAAGGCAGATTCACCATCAGC |
| 8H1_LC) | AGAGACAACAGCAAGACCACACTGTACCTGCAGATGAACAGCCTGACAGCTGAG |
| | GACACAGCAGTGTACTACTGCACCAAGAGAGTGGAAGGCTCCTTCAGAGGCTGG |
| | GGCCAGGGAACACTGGTTACAGTGTCCTCTGCCAGCACAAAGGGCCCCTCTGTT |
| | TTTCCACTGGCTCCCAGCAGCAAGAGCACCAGTGGTGGAACAGCTGCCCTGGGC |
| | TGTCTGGTCAAGGATTACTTCCCTGAGCCTGTGACTGTGTCCTGGAACTCTGGT |
| | GCACTGACCTCTGGGGTGCACACCTTTCCAGCTGTGCTGCAGTCCTCTGGCCTG |
| | TACTCTCTGTCCTCTGTGGTCACAGTGCCTAGCTCTAGCCTGGGCACCCAGACC |
| | TACATCTGCAATGTGAACCACAAGCCTAGCAACACCAAGGTGGACAAAAGAGTG |
| | GAACCCAAGAGCTGTGACAAGACCCACAGAAAGAGAAGAGGCTCTGGAGAAGGC |
| | AGAGGCTCCCTGCTGACATGTGGGGATGTTGAAGAGAATCCTGGGCCTATGTAT |
| | AGGATGCAACTGCTCCTCCTGATTGCTCTGAGCCTGGCTCTTGTGACCAACTCT |
| | GAGATTGTGATGACACAGAGCCCAGGCACACTGTCACTGTCTCCAGGGGAATCT |
| | GCCACTCTGAGCTGTAGAGCCAGCCAGTCTGTGTCTACCACCTACCTGGCCTGG |
| | TATCAGCAAAAGCCTGGACAGGCTCCCAGACTGCTGATCTATGGGGCCTCTTCT |
| | AGAGCCACAGGCATCCCTGACAGATTTTCTGGCAGTGGCTCTGGCACAGACTTC |
| | ACCCTGACCATCTCTAGACTGGAACCTGAGGACTTTGCTGTCTACTACTGCCAG |
| | CAGTATGGTGGCTCCAGACTGATCACCTTTGGCCAGGGCACCAGACTGGAAATC |
| | AAGAGAACAGTGGCTGCCCCATCTGTGTTCATCTTCCCACCATCTGATGAGCAG |
| | CTCAAGTCTGGCACTGCCTCTGTTGTGTGCCTGCTGAACAACTTCTACCCTAGA |
| | GAAGCCAAGGTGCAGTGGAAGGTTGACAATGCCCTGCAGTCTGGCAATAGCCAA |
| | GAATCTGTGACAGAGCAGGACTCCAAGGATAGCACCTACAGCCTGTCTAGCACC |
| | CTGACACTGAGCAAGGCTGACTATGAGAAGCACAAAGTGTATGCCTGTGAAGTG |
| | ACACACCAGGGCCTGAGCAGCCCAGTGACCAAGAGCTTCAATAGAGGGGAGTGC |
| | (SEQ ID NO: 385) |
| 8H1 Fab | HC (with signal peptide): |
| polypeptides | MYRMQLLLLIALSLALVTNSEVQLVESGGGLVQPGGSLRLSCAASGFTFSIYTM |
| | SWVRQAPGKGLEWVSAILSDGSTYYADSVEGRFTISRDNSKTTLYLQMNSLTAE |
| | DTAVYYCTKRVEGSFRGWGQGTLVTVSSASTKGPSVFPLAPSSKSTSGGTAALG |
| | CLVKDYFPEPVTVSWNSGALTSGVHTFPAVLQSSGLYSLSSVVTVPSSSLGTQT |
| | YICNVNHKPSNTKVDKRVEPKSCDKTH (SEQ ID NO: 386) |
| | HC (no signal): |
| | EVQLVESGGGLVQPGGSLRLSCAASGFTFSIYTMSWVRQAPGKGLEWVSAILSD |
| | GSTYYADSVEGRFTISRDNSKTTLYLQMNSLTAEDTAVYYCTKRVEGSFRGWGQ |
| | GTLVTVSSASTKGPSVFPLAPSSKSTSGGTAALGCLVKDYFPEPVTVSWNSGAL |
| | TSGVHTFPAVLQSSGLYSLSSVVTVPSSSLGTQTYICNVNHKPSNTKVDKRVEP |
| | KSCDKTH (SEQ ID NO: 387) |
| | LC (kappa) (with signal peptide): |
| | MYRMQLLLLIALSLALVTNSEIVMTQSPGTLSLSPGESATLSCRASQSVSTTYL |
| | AWYQQKPGQAPRLLIYGASSRATGIPDRFSGSGSGTDFTLTISRLEPEDFAVYY |
| | CQQYGGSRLITFGQGTRLEIKRTVAAPSVFIFPPSDEQLKSGTASVVCLLNNFY |
| | PREAKVQWKVDNALQSGNSQESVTEQDSKDSTYSLSSTLTLSKADYEKHKVYAC |
| | EVTHQGLSSPVTKSFNRGEC (SEQ ID NO: 388) |
| | LC (kappa) (no signal) |
| | EIVMTQSPGTLSLSPGESATLSCRASQSVSTTYLAWYQQKPGQAPRLLIYGASS |
| | RATGIPDRFSGSGSGTDFTLTISRLEPEDFAVYYCQQYGGSRLITFGQGTRLEI |
| | KRTVAAPSVFIFPPSDEQLKSGTASVVCLLNNFYPREAKVQWKVDNALQSGNSQ |
| | ESVTEQDSKDSTYSLSSTLTLSKADYEKHKVYACEVTHQGLSSPVTKSFNRGEC |
| | (SEQ ID NO: 389) |
| pAAV.CAG.hu. | CTGCGCGCTCGCTCGCTCACTGAGGCCGCCCGGGCAAAGCCCGGGCGTCGGGCG |
| Tau.31B6-Fab | ACCTTTGGTCGCCCGGCCTCAGTGAGCGAGCGAGCGCGCAGAGAGGGAGTGGCC |
| (ITR to ITR) | AACTCCATCACTAGGGGTTCCTTGTAGTTAATGATTAACCCGCCATGCTACTTA |
| | TCTACCAGGGTAATGGGGATCCTCTAGAACTATAGCTAGTCGACATTGATTATT |
| | GACTAGTTATTAATAGTAATCAATTACGGGGTCATTAGTTCATAGCCCATATAT |
| | GGAGTTCCGCGTTACATAACTTACGGTAAATGGCCCGCCTGGCTGACCGCCCAA |
| | CGACCCCCGCCCATTGACGTCAATAATGACGTATGTTCCCATAGTAACGCCAAT |
| | AGGGACTTTCCATTGACGTCAATGGGTGGAGTATTTACGGTAAACTGCCCACTT |
| | GGCAGTACATCAAGTGTATCATATGCCAAGTACGCCCCCTATTGACGTCAATGA |
| | CGGTAAATGGCCCGCCTGGCATTATGCCCAGTACATGACCTTATGGGACTTTCC |
| | TACTTGGCAGTACATCTACGTATTAGTCATCGCTATTACCATGGTCGAGGTGAG |
| | CCCCACGTTCTGCTTCACTCTCCCCATCTCCCCCCCCTCCCCACCCCCAATTTT |
| | GTATTTATTTATTTTTTAATTATTTTGTGCAGCGATGGGGGCGGGGGGGGGGGG |
| | GGGGCGCGCGCCAGGCGGGGCGGGGCGGGGCGAGGGGCGGGGCGGGGCGAGGCG |
| | GAGAGGTGCGGCGGCAGCCAATCAGAGCGGCGCGCTCCGAAAGTTTCCTTTTAT |
| | GGCGAGGCGGCGGCGGCGGCGGCCCTATAAAAAGCGAAGCGCGCGGCGGGCGGG |
| | AGTCGCTGCGCGCTGCCTTCGCCCCGTGCCCCGCTCCGCCGCCGCCTCGCGCCG |
| | CCCGCCCCGGCTCTGACTGACCGCGTTACTCCCACAGGTGAGCGGGCGGGACGG |
| | CCCTTCTCCTCCGGGCTGTAATTAGCGCTTGGTTTAATGACGGCTTGTTTCTTT |
| | TCTGTGGCTGCGTGAAAGCCTTGAGGGGCTCCGGGAGGGCCCTTTGTGCGGGGG |
| | GAGCGGCTCGGGGGGTGCGTGCGTGTGTGTGTGCGTGGGGAGCGCCGCGTGCGG |
| | CTCCGCGCTGCCCGGCGGCTGTGAGCGCTGCGGGCGCGGCGCGGGGCTTTGTGC |
| | GCTCCGCAGTGTGCGCGAGGGGAGCGCGGCCGGGGGCGGTGCCCCGCGGTGCGG |
| | GGGGGGCTGCGAGGGGAACAAAGGCTGCGTGCGGGGTGTGTGCGTGGGGGGGTG |
| | AGCAGGGGGTGTGGGCGCGTCGGTCGGGCTGCAACCCCCCCTGCACCCCCCTCC |
| | CCGAGTTGCTGAGCACGGCCCGGCTTCGGGTGCGGGGCTCCGTACGGGGCGTGG |
| | CGCGGGGCTCGCCGTGCCGGGCGGGGGGTGGCGGCAGGTGGGGGTGCCGGGCGG |
| | GGCGGGGCCGCCTCGGGCCGGGGAGGGCTCGGGGGAGGGGCGCGGCGGCCCCCG |
| | GAGCGCCGGCGGCTGTCGAGGCGCGGCGAGCCGCAGCCATTGCCTTTTATGGTA |
| | ATCGTGCGAGAGGGCGCAGGGACTTCCTTTGTCCCAAATCTGTGCGGAGCCGAA |
| | ATCTGGGAGGCGCCGCCGCACCCCCTCTAGCGGGCGCGGGGCGAAGCGGTGCGG |
| | CGCCGGCAGGAAGGAAATGGGCGGGGAGGGCCTTCGTGCGTCGCCGCGCCGCCG |
| | TCCCCTTCTCCCTCTCCAGCCTCGGGGCTGTCCGCGGGGGGACGGCTGCCTTCG |
| | GGGGGGACGGGGCAGGGCGGGGTTCGGCTTCTGGCGTGTGACCGGCGGCTCTAG |
| | AGCCTCTGCTAACCATGTTCATGCCTTCTTCTTTTTCCTACAGCTCCTGGGCAA |
| | CGTGCTGGTTATTGTGCTGTCTCATCATTTTGGCAAAGAATTCATGTACAGGAT |
| | GCAGCTGCTGCTGCTCATTGCCCTGTCTCTGGCCCTGGTCACCAATTCTGAAGT |
| | GCAGCTTCTGCAGTCTGGTGGTGGTGTTGTGCAGCCTGGTGGCTCTCTGAGACT |
| | GTCTTGTGAAGCCTCTGGCTTCACCTTCAGCAGCTACAGCATGCACTGGGTTAG |
| | ACAGGCCCCTGGCAAAGGCCTGGAATGGCTGACCCTGATCAGCTACAATGGGGT |
| | CAACAAGTACTATGCCAAGTCTGTGAAGGGCAGATTTGCCATCAGCAGAGATGA |
| | CAGCAAGAGCACCCTGTTTCTGCAGATGAACAGCCTGACCACAGATGACACAGC |
| | AGTGTACTACTGTGCCAGATGTCCCATTCCTAGAGCTGCCAGCTACAGAAATGG |
| | GGACAACTACTTTGAGTTTGGCCAGGGCACACTGGTCACAGTGTCCTCTGCCTC |
| | TACAAAGGGCCCCTCTGTGTTTCCTCTGGCTCCTAGCAGCAAGTCCACCAGTGG |
| | TGGTACAGCTGCCCTGGGCTGTCTGGTCAAGGATTACTTCCCTGAGCCTGTGAC |
| | TGTGTCCTGGAATTCTGGGGCTCTGACCTCTGGGGTGCACACCTTTCCAGCTGT |
| | GCTGCAGTCCTCTGGCCTGTACTCTCTGTCCTCTGTGGTTACAGTGCCAAGCAG |
| | CTCTCTGGGCACCCAGACCTACATCTGCAATGTGAACCACAAGCCTAGCAACAC |
| | CAAGGTGGACAAGAGAGTGGAACCCAAGAGCTGTGACAAGACCCACAGAAAGAG |
| | AAGAGGCTCTGGAGAAGGCAGAGGCAGCCTGCTGACATGTGGGGATGTTGAAGA |
| | GAATCCTGGGCCTATGTATAGAATGCAACTGCTCCTCCTGATTGCTCTGAGCCT |
| | GGCTCTGGTTACCAACAGCCAGGCTGTGGTCACACAAGAGCCCAGCCTGTCTGT |
| | GTCTCCTGGTGGAACAGTGACCCTGACTTGTGCCCTCTCTTCTGCCTCTGTGTC |
| | CACAAGCAGCTACCCCAGCTGGTATCAGCAGACACCTGGCCAGCCACCTAGAAT |
| | GCTGATCCACAACACTGACACCAGAAGCTCTGGGGTCCCAGACAGATTCTCTGG |
| | CAGCATCCTGGGAAAGAAGGCTGCCCTGACAATCACTGGTGCCCAGGCTGATGA |
| | TGAGTCTGACTACTATTGTGTGGTGTACCTTGGCAATGGCTTTTGGGTGTTCGG |
| | TGGTGGCACCAAACTGACTGTGCTGGGACAGCCTAAAGCTGCCCCTTCTGTGAC |
| | ACTGTTCCCACCTAGCTCTGAGGAACTGCAGGCCAACAAAGCCACACTTGTGTG |
| | CCTGATCTCTGACTTCTACCCTGGGGCTGTGACAGTGGCTTGGAAGGCTGATAG |
| | CAGCCCTGTGAAGGCTGGGGTTGAGACAACCACACCTAGCAAGCAGAGCAACAA |
| | CAAATATGCTGCCTCCAGCTATCTGTCTCTGACCCCTGAGCAGTGGAAGTCCCA |
| | CAGATCCTACAGCTGTCAAGTGACCCATGAGGGCTCCACAGTGGAAAAGACAGT |
| | GGCCCCAACAGAGTGCAGCTGATGACTCGAGGACGGGGTGAACTACGCCTGAGG |
| | ATCCGATCTTTTTCCCTCTGCCAAAAATTATGGGGACATCATGAAGCCCCTTGA |
| | GCATCTGACTTCTGGCTAATAAAGGAAATTTATTTTCATTGCAATAGTGTGTTG |
| | GAATTTTTTGTGTCTCTCACTCGGAAGCAATTCGTTGATCTGAATTTCGACCAC |
| | CCATAATACCCATTACCCTGGTAGATAAGTAGCATGGCGGGTTAATCATTAACT |
| | ACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGG |
| | CCTCAGTGAGCGAGCGAGCGCGCAG (SEQ ID NO: 390) |
| 31B6 Fab | ATGTACAGGATGCAGCTGCTGCTGCTCATTGCCCTGTCTCTGGCCCTGGTCACC |
| nucleotide | AATTCTGAAGTGCAGCTTCTGCAGTCTGGTGGTGGTGTTGTGCAGCCTGGTGGC |
| (mIL2- | TCTCTGAGACTGTCTTGTGAAGCCTCTGGCTTCACCTTCAGCAGCTACAGCATG |
| 31B6_HC_Fab- | CACTGGGTTAGACAGGCCCCTGGCAAAGGCCTGGAATGGCTGACCCTGATCAGC |
| FurinT2A-mIL2- | TACAATGGGGTCAACAAGTACTATGCCAAGTCTGTGAAGGGCAGATTTGCCATC |
| 31B6_LC) | AGCAGAGATGACAGCAAGAGCACCCTGTTTCTGCAGATGAACAGCCTGACCACA |
| | GATGACACAGCAGTGTACTACTGTGCCAGATGTCCCATTCCTAGAGCTGCCAGC |
| | TACAGAAATGGGGACAACTACTTTGAGTTTGGCCAGGGCACACTGGTCACAGTG |
| | TCCTCTGCCTCTACAAAGGGCCCCTCTGTGTTTCCTCTGGCTCCTAGCAGCAAG |
| | TCCACCAGTGGTGGTACAGCTGCCCTGGGCTGTCTGGTCAAGGATTACTTCCCT |
| | GAGCCTGTGACTGTGTCCTGGAATTCTGGGGCTCTGACCTCTGGGGTGCACACC |
| | TTTCCAGCTGTGCTGCAGTCCTCTGGCCTGTACTCTCTGTCCTCTGTGGTTACA |
| | GTGCCAAGCAGCTCTCTGGGCACCCAGACCTACATCTGCAATGTGAACCACAAG |
| | CCTAGCAACACCAAGGTGGACAAGAGAGTGGAACCCAAGAGCTGTGACAAGACC |
| | CACAGAAAGAGAAGAGGCTCTGGAGAAGGCAGAGGCAGCCTGCTGACATGTGGG |
| | GATGTTGAAGAGAATCCTGGGCCTATGTATAGAATGCAACTGCTCCTCCTGATT |
| | GCTCTGAGCCTGGCTCTGGTTACCAACAGCCAGGCTGTGGTCACACAAGAGCCC |
| | AGCCTGTCTGTGTCTCCTGGTGGAACAGTGACCCTGACTTGTGCCCTCTCTTCT |
| | GCCTCTGTGTCCACAAGCAGCTACCCCAGCTGGTATCAGCAGACACCTGGCCAG |
| | CCACCTAGAATGCTGATCCACAACACTGACACCAGAAGCTCTGGGGTCCCAGAC |
| | AGATTCTCTGGCAGCATCCTGGGAAAGAAGGCTGCCCTGACAATCACTGGTGCC |
| | CAGGCTGATGATGAGTCTGACTACTATTGTGTGGTGTACCTTGGCAATGGCTTT |
| | TGGGTGTTCGGTGGTGGCACCAAACTGACTGTGCTGGGACAGCCTAAAGCTGCC |
| | CCTTCTGTGACACTGTTCCCACCTAGCTCTGAGGAACTGCAGGCCAACAAAGCC |
| | ACACTTGTGTGCCTGATCTCTGACTTCTACCCTGGGGCTGTGACAGTGGCTTGG |
| | AAGGCTGATAGCAGCCCTGTGAAGGCTGGGGTTGAGACAACCACACCTAGCAAG |
| | CAGAGCAACAACAAATATGCTGCCTCCAGCTATCTGTCTCTGACCCCTGAGCAG |
| | TGGAAGTCCCACAGATCCTACAGCTGTCAAGTGACCCATGAGGGCTCCACAGTG |
| | GAAAAGACAGTGGCCCCAACAGAGTGCAGC (SEQ ID NO: 391) |
| 31B6 Fab | HC (with signal peptide): |
| polypeptides | MYRMQLLLLIALSLALVTNSEVQLLQSGGGVVQPGGSLRLSCEASGFTFSSYSM |
| | HWVRQAPGKGLEWLTLISYNGVNKYYAKSVKGRFAISRDDSKSTLFLQMNSLTT |
| | DDTAVYYCARCPIPRAASYRNGDNYFEFGQGTLVTVSSASTKGPSVFPLAPSSK |
| | STSGGTAALGCLVKDYFPEPVTVSWNSGALTSGVHTFPAVLQSSGLYSLSSVVT |
| | VPSSSLGTQTYICNVNHKPSNTKVDKRVEPKSCDKTH (SEQ ID NO: 392) |
| | HC (no signal): |
| | EVQLLQSGGGVVQPGGSLRLSCEASGFTFSSYSMHWVRQAPGKGLEWLTLISYN |
| | GVNKYYAKSVKGRFAISRDDSKSTLFLQMNSLTTDDTAVYYCARCPIPRAASYR |
| | NGDNYFEFGQGTLVTVSSASTKGPSVFPLAPSSKSTSGGTAALGCLVKDYFPEP |
| | VTVSWNSGALTSGVHTFPAVLQSSGLYSLSSVVTVPSSSLGTQTYICNVNHKPS |
| | NTKVDKRVEPKSCDKTH (SEQ ID NO: 393) |
| | LC (lambda)(with signal peptide): |
| | MYRMQLLLLIALSLALVTNSQAVVTQEPSLSVSPGGTVTLTCALSSASVSTSSY |
| | PSWYQQTPGQPPRMLIHNTDTRSSGVPDRFSGSILGKKAALTITGAQADDESDY |
| | YCVVYLGNGFWVFGGGTKLTVLGQPKAAPSVTLFPPSSEELQANKATLVCLISD |
| | FYPGAVTVAWKADSSPVKAGVETTTPSKQSNNKYAASSYLSLTPEQWKSHRSYS |
| | CQVTHEGSTVEKTVAPTECS (SEQ ID NO: 394) |
| | LC (lambda)(no signal) |
| | QAVVTQEPSLSVSPGGTVTLTCALSSASVSTSSYPSWYQQTPGQPPRMLIHNTD |
| | TRSSGVPDRFSGSILGKKAALTITGAQADDESDYYCVVYLGNGFWVFGGGTKLT |
| | VLGQPKAAPSVTLFPPSSEELQANKATLVCLISDFYPGAVTVAWKADSSPVKAG |
| | VETTTPSKQSNNKYAASSYLSLTPEQWKSHRSYSCQVTHEGSTVEKTVAPTECS |
| | (SEQ ID NO: 395) |

Cis plasmids comprising each gene cassette described herein were constructed to express Tau-specific mAbs and controls (transgenes) and the entire cassette was flanked by AAV ITRs. The CAG promoter was utilized in these cassettes and is expected to confer expression of the gene therapy vector in all CNS cell types. AAV proviral (cis) plasmids containing these sequence elements can be packaged into infectious vector particles and purified as products for gene therapy. These plasmids can also be transfected into mammalian cells, such as BTEK293 cells, for direct study of transduction efficiency and gene expression, as described herein.

### 6.2 Example 2—Plasmid Expression of Tau-Specific Antibody Expression Cassettes in 293T Cells

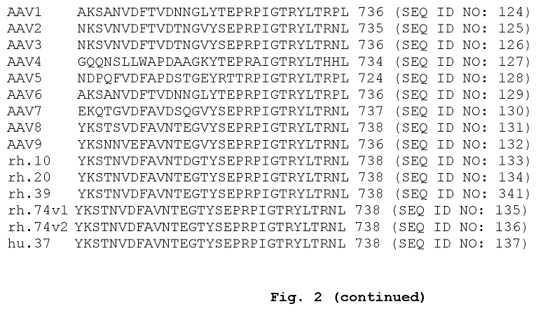

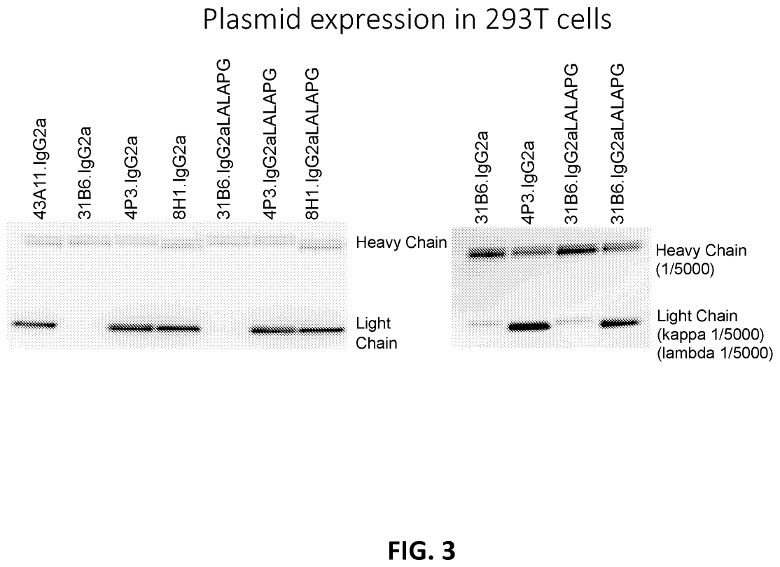

Tau-specific mAb-expressing constructs (AAV cis plasmids) as exemplified in FIG. 1B, including a control construct, all utilizing the universal CAG promoter operably linked to the transgene, were transfected into HEK293T cells. Briefly, 293T cells seeded in 6 well dishes at 800K cells per well, grown in DMEM+10% FBS. 24 hrs later, cells were transfected with 4 ug Tau Plasmid as indicated with 10 ul Lipofectamine 2000 (Invitrogen). Media was replaced with fresh media, without FBS, 8 hrs later. 40 hrs later cell supernatant was collected and subjected to western blotting. Briefly, cell supernatants were spun in a microfuge at 13000 g for 3 minutes to collect cellular debris. Supernatants were prepared to western blotting by mixing with 4× LDS (Invitrogen) and heating to 70° C. for 10 minutes. Equal volumes were loaded onto 4-12% Bis-Tris gels (Invitrogen), electrophoresed then transferred to PVDF. PVDF membrane was immunoblotted with commercially available antibodies to mouse heavy chain and mouse Kappa or Lambda light chains. Antibodies 43A11 IgG2a (43), 31B6 IgG2a (M3), 4P3 IgG2a (M4) and 8H1 IgG2a (M8) are full-length chimeric antibodies having human variable domains and mouse IgG2a Fc. Antibodies 4P3 (M4) and 8H1 (M8) have Kappa light chains, and 31B6 have lambda light chains. All six plasmids express and secrete antibodies that migrate as distinct heavy chain and light chain polypeptides, as anticipated. FIG. 3. Note that 31B6 IgG2a LALAPG, 4P3 IgG2a LALAPG and 8H1 IgG2a LALAPG antibodies have a modified Fc domain (LALAPG) with mutations within the effector domain of the antibodies that reduce effector function.

### 6.3 Example 3—Tau-Specific Binding of Antibodies Expressed from Gene Cassettes in 293T Cells

In analogous cell culture experiments to Example 2, Tau-specific mAb-expressing constructs (AAV cis plasmids) having full-length vectorized antibody expression cassettes as depicted in FIG. 1B, were transfected into HEK293T cells and assessed for gene expression and their binding specificity subject to indirect ELISA.

Indirect ELISA was performed using 96-well half-area microplates (Corning Incorporated, Corning, USA) coated with either recombinant full-length human Tau (rPeptide, Watkinsville, USA) or with BSA (Sigma-Aldrich, Buchs, Switzerland) at a concentration of 3 μg/ml in coating buffer (15 mM Na2CO3, 35 mM NaHCO₃, pH 9.42) overnight at 4° C. or with 96-well half-area microplates (Corning Incorporated, Corning, USA) coated with the synthetic BSA-coupled phosphorylated Tau peptides (Schafer-N, Copenhagen, Denmark) or with BSA (Sigma-Aldrich, Buchs, Switzerland) at a concentration of 5 μg/ml in coating buffer (15 mM Na2CO3, 35 mM NaHCO₃, pH 9.42) overnight at 4° C. Non-specific binding sites were blocked for 1 h at room temperature with PBS/0.1% Tween®-20 containing 2% BSA (Sigma-Aldrich, Buchs, Switzerland).

HEK293T cell supernatants diluted 1:2 or 1:10 in PBS were added and incubated for 2 h at room temperature, followed by incubation with an HRP-conjugated goat anti-mouse IgG (H+L)-specific antibody (Jackson ImmunoResearch Laboratories, Inc, West Grove, USA, 1:4′000). Standard curves were prepared by two-fold serial dilutions of Recombinant 4P3 IgG2a, 31B6 IgG2a and 8H1 IgG2a in PBS with an initial antibody concentration of 100 nM. Binding was determined by measurement of HRP activity in a standard colorimetric assay. Antibody levels in the HEK293T cell supernatants were estimated by linear regression using GraphPad Prism software (San Diego, USA).

Antibody levels in the HEK293T cell supernatants as determined by indirect ELISA, are listed in Table 12—as quantitative measures of binding to the Tau target for each antibody expressed by the AAV cis plasmids.

TABLE 12
AAV Cis plasmid-expressed Antibodies Binding Tau

| Antibody | Target | Concentration | |
|---|---|---|---|
| 31B6 IgG2a | Tau pS202/pT205 | 88 | nM |
| 4P3 IgG2a | Recombinant Tau | 15 | nM |
| 8H1 IgG2a | Tau pT212/pS214 | 39 | nM |
| 31B6 IgG2a LALAPG | Tau pS202/pT205 | 100 | nM |
| 4P3 IgG2a LALAPG | Recombinant Tau | 14 | nM |
| 8H1 IgG2a LALAPG | Tau pT212/pS214 | 60 | nM |

### 6.4 Example 4—Full-Length Tau-Specific Antibodies Expressed in 293T Cell Line Transduced with Viral Vectors

### In Vitro Transduction and Expression of Vectorized Antibody

Cis plasmids expressing vectorized antibody were packaged in AAV9 by well-known benchtop rAAV production methods, starting with “triple” transfection, cells were transfected with polyethylenimine (PEI) and three plasmids encoding 1) transgene (cis plasmid as described herein), 2) AAV2/9 Rep/Cap, and 3) adenovirus helper genes. Transfected cultures were maintained for 5 days following transfection to allow AAV production, AAV was collected and purified from the culture supernatants, then rAAV particles were evaluated for potency of the transduction of the AAV in an HEK cell line expressing AAV receptors. The rAAV transgenes contain antibody light chain and heavy chain IgG transgene which are arranged as multicistrons driven by the same promoter as described in Example 1 and FIG. 1B (full-length antibody light chain and antibody heavy chain genes were separated by a furin T2A linker to ensure separate expression of each antibody chain). The expressed antibodies include the control 43A11, full length 4P3, and 4P3-LALAPG antibody (having a LALAPG mutant Fc), full length 31B6, and 31B6-LALAPG antibody (having a LALAPG mutant Fc), 8H1, and 8H1-LALAPG (having a LALAPG mutant Fc). The entire antibody cassette is flanked by AAV2 ITRs, and the genome is encapsidated in an AAV9 capsid for delivery to HEK293T cells stably expressing an AAV receptor (AAVR) (le10 gc per well).

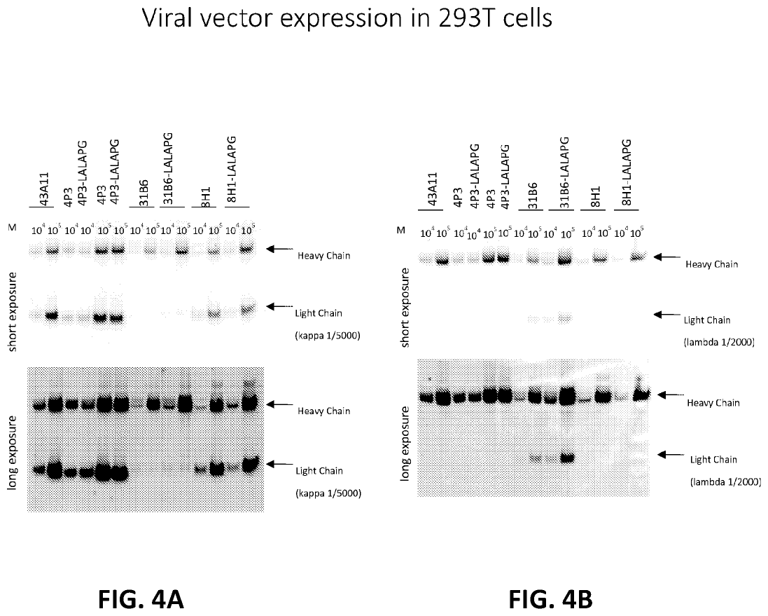

Briefly, HEK293T.AAVR cells were plated and transduced at 1E4 or 1E5 MOI (multiplicity of infection) with AAV2/9-Tau AGT vectors. Media was replaced 8 hours later, and 40 hours later supernatant was collected and immunoblotted to confirm antibody expression. As with plasmid transfection, all vectors produced antibody, including the heavy and light chains, as shown in FIGS. 4A-4B.

### 6.5 Example 5—In Vivo Expression of Vectorized Antibody Injected ICV in Neonatal C57BL/6 Mice

Neonatal (P0/P1) C57BL/6 mice were anaesthetized using isoflurane inhalation. Mice were placed on a heating pad to maintain body temperature during surgery and recovery phase. AAV9 gene therapy vectors encoding 4P3 IgG2a and 4P3 IgG2a LALAPG were administered by bilateral intracerebroventricular (lateral ventricle) injections at three different doses: 2.4e9 gc/side (low dose), 1.2e10 gc/side (mid dose), and 6e10 gc/side (high dose) (n=5 per group, see Table 13). Four weeks post administration, mice were deeply anesthetized (i.p. injection of ketamine 12.5 mg/ml (1.25%) and xylazine 2.5 mg/ml (0.25%) in PBS with 10 l/g body weight) and blood was withdrawn from the vena cava before mice were transcardially perfused with cold phosphate-buffered saline/heparin through the left ventricle. Blood samples were collected into BD Microtainer K2E tubes and processed according to manufacturer's protocol for obtaining blood plasma. Brains were then dissected: left hemibrains were fixed in phosphate-buffered 4% paraformaldehyde solution overnight at 4° C. whereas right hemibrains were further dissected for isolating cortex, hippocampus and striatum that were then fresh frozen on dry ice. Paraformaldehyde-fixed hemibrains were then processed on a Shandon Citadel 2000 Tissue Processor (Thermo Scientific), paraffin-embedded, and cut into 3-μm sagittal sections.

Frozen tissues (cortex, hippocampus, and striatum) were homogenized using DEA buffer (50 mM NaCl (Sigma-Aldrich, Buchs, Switzerland) and 0,2% diethylamine (Sigma-Aldrich, Buchs, Switzerland) in ddH₂0) complemented with PhosphoSTOP and cOmplete tablets (Roche, Basel, Switzerland). Soluble fractions were collected after spinning down the homogenates for 60 min at 18213 rpm. Samples were stored at −80° C. until use.

96-well microplates (Corning Incorporated, Corning, USA) were coated with goat anti-mouse IgG2a antibody (Southern Biotech, Birmingham, USA) at a concentration of 1 μg/ml in PBS overnight at 4° C. Non-specific binding sites were blocked for 1 h at RT with PBS/0.1% Tween®-20 containing 2% BSA (Sigma-Aldrich, Buchs, Switzerland). Plasma samples diluted to either 1:50000 or 1:20000 or 1:1000 in PBS or brain homogenates (5 μl diluted at 1:100) were added and incubated for 2 h at RT, followed by incubation with a goat anti-mouse IgG2a-specific antibody conjugated with HRP (Southern Biotech, Birmingham, USA). Standard curves were prepared by two-fold serial dilutions of recombinant 4P3 IgG2a antibody in PBS with initial antibody concentration at 1 nM or 2 nM. Binding was determined by measurement of HRP activity in a standard colorimetric assay. Drug plasma and brain levels were estimated by linear regression using GraphPad Prism software (San Diego, USA)

Formalin fixed, paraffin-embedded sections (3 μm) were deparaffinized in xylene and rehydrated through graded ethanol. Quenching of endogenous peroxidase activity was achieved by treatment with 3% H₂O₂ in methanol for 10 min at RT. Non-specific binding sites were blocked for 1 h at RT with PBS/5% serum (horse/goat)/4% BSA. After the blocking step, sections were incubated with biotinylated goat anti-mouse IgG2a antibody (1:50 dilution, Southern Biotech, Birmingham, USA) for 3 h at RT. Antibody signal was amplified with the Vectastain Elite ABC kit (Vector Laboratories, Burlingame, USA) and detected with diaminobenzidine (DAB, Thermo Scientific, Rockford, USA). Slides were mounted using Eukitt® mounting medium (O. Kindler GmbH; Freiburg, Germany). Bright-field imaging was performed using a Dotslide VS120 slide scanner (Olympus Schweiz AG, Switzerland).

TABLE 13

| | Mouse | Group | | Dose (gc/ | Application | |
|---|---|---|---|---|---|---|
| Group | line | size | AAV vector | animal) | route | Takedown |
| 1 | C57BL/6 | n = 5 | 4P3 IgG2a | 1.2e11 | icv | 4 weeks p.i. |
| 2 | C57BL/6 | n = 5 | 4P3 IgG2a | 2.4e10 | icv | 4 weeks p.i. |
| 3 | C57BL/6 | n = 5 | 4P3 IgG2a | 4.8e9 | icv | 4 weeks p.i. |
| 4 | C57BL/6 | n = 5 | 4P3 IgG2a LALAPG | 1.2e11 | icv | 4 weeks p.i. |
| 5 | C57BL/6 | n = 5 | 4P3 IgG2a LALAPG | 2.4e10 | icv | 4 weeks p.i. |
| 6 | C57BL/6 | n = 5 | 4P3 IgG2a LALAPG | 4.8e9 | icv | 4 weeks p.i. |

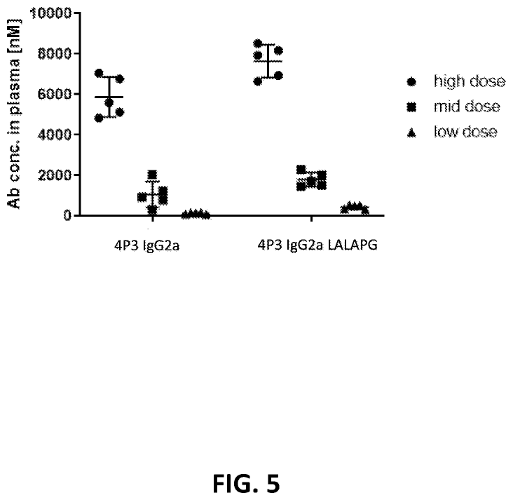

Antibody levels were detected in plasma by sandwich ELISA, in a dose dependent manner, indicating likely peripheral transduction. Antibody levels were detected in the range of 5000 nM to more than 8000 nM in plasma when administered at the highest dose of 1.2e11 gc/animal, as seen in FIG. 5.

Antibody expression was also detected by immunohistochemistry staining (anti-IgG2a antibody) in cells of the hippocampus, and 4P3 IgG2a and 4P3 IgG2a LALAPG expression is considered comparable (data not shown).

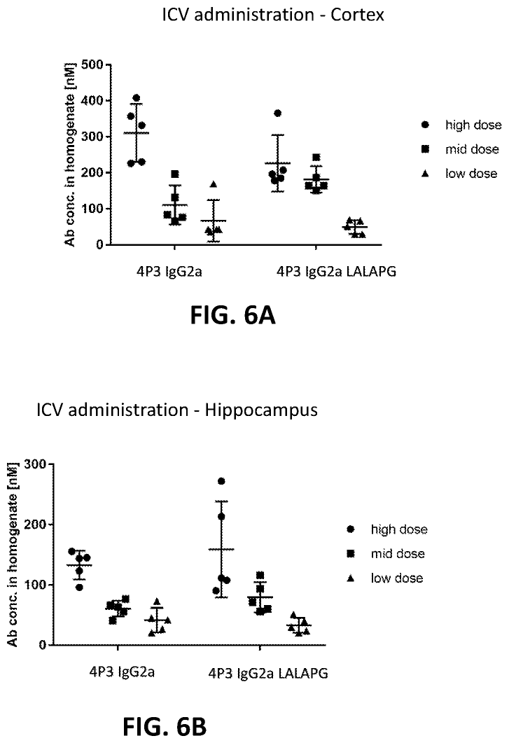

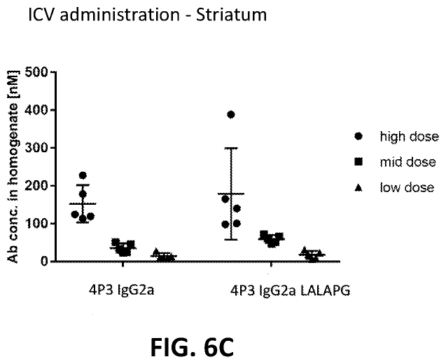

Brain regions hippocampus, striatum and cortex were further collected, homogenized and antibody concentration was determined (sandwich ELISA) showing dose-dependent antibody levels. Antibody concentration (nM) in the homogenates from cortex (FIG. 6A), hippocampus (FIG. 6B) and striatum (FIG. 6C) are shown. ICV administration appears to achieve the highest antibody concentrations in the cortex, followed by hippocampus, followed by striatum, in this experiment.

### 6.6 Example 6—In Vivo Expression of Vectorized Antibody Injected Via Various Routes of CNS Administration in Adult C57BL/6 Mice

Adult C57BL/6 mice were anaesthetized using an intraperitoneal (i.p.) injection of a mixture of fentanyl (0.05 mg/kg), midazolam (5.0 mg/kg), and medetomidin (0.5 mg/kg) in saline. A loss of response to nociceptive stimulation of the tail and between the toes indicated deep sedation. Mice were placed on a heating pad to maintain body temperature during surgery, and eye cream (Viscotears, carbomerum 980, 2.0 mg) was applied onto the eyes to prevent drying out during the surgery. AAV9 gene therapy vector encoding 4P3 IgG2a was administered by bilateral intracerebroventricular (lateral ventricle), intrahippocampal or intrastriatal injections at a dose of 1.2e10 gc/side (mid dose) (n=5 per group, see Table 14). Bilateral stereotaxic injections of 2.0 μl of 4P3 IgG2a were performed by drilling a hole in the skull at the injection site and placing a Hamilton syringe into either the lateral ventricle (A/P, −0.6 mm from bregma; L, ±1.0 mm; D/V, −2.0 mm), or the hippocampus (A/P, −2.0 mm from bregma; L, ±1.6 mm; D/V, −1.7 mm) or the striatum (A/P, +0.2 mm from bregma; L, +2.0 mm; D/V, −3.2 mm). The injection speed was 0.2 μL/minute and the needle was kept in place for an additional 10 minutes before it was slowly withdrawn. The surgical area was cleaned with sterile saline and the incision was sutured. Immediately after the surgery the antidote was administered intraperitoneally, a mixture containing flumazenil (0.5 mg/kg) and atipamezol (2.5 mg/kg) in saline. Mice were monitored until recovery from anesthesia. In addition to the routine monitoring by animal care takers, operated animals were checked daily for the 3 days following surgery for recovery of the wounds, as well as weekly for signs of abnormal behavior until the time of sacrifice for sample collection. Mice were provided with 200 mg/kg paracetamol in the drinking water starting 24 hours prior to the surgery and over a period of the next 7 days. Four weeks post administration, mice were deeply anesthetized (i.p. injection of ketamine 12.5 mg/ml (1.25%) and xylazine 2.5 mg/ml (0.25%) in PBS with 10 μl/g body weight) and blood was withdrawn from the vena cava before mice were transcardially perfused with cold phosphate-buffered saline/heparin through the left ventricle. Blood samples were collected into BD Microtainer K2E tubes and processed according to manufacturer's protocol for obtaining blood plasma. Brains were then dissected: left hemibrains were fixed in phosphate-buffered 4% paraformaldehyde solution overnight at 4° C. whereas right hemibrains were further dissected for isolating cortex, hippocampus and striatum that were then fresh frozen on dry ice. Paraformaldehyde-fixed hemibrains were then processed on a Shandon Citadel 2000 Tissue Processor (Thermo Scientific), paraffin-embedded, and cut into 3-μm sagittal sections.

Frozen tissues (cortex, hippocampus, and striatum) were homogenized using DEA buffer (50 mM NaCl (Sigma-Aldrich, Buchs, Switzerland) and 0,2% diethylamine (Sigma-Aldrich, Buchs, Switzerland) in ddH₂0) complemented with PhosphoSTOP and cOmplete tablets (Roche, Basel, Switzerland). Soluble fractions were collected after spinning down the homogenates for 60 min at 18213 rpm. Samples were stored at −80° C. until use.

96-well microplates (Corning Incorporated, Corning, USA) were coated with goat anti-mouse IgG2a antibody (Southern Biotech, Birmingham, USA) at a concentration of 1 μg/ml in PBS overnight at 4° C. Non-specific binding sites were blocked for 1 h at RT with PBS/0.1% Tween®-20 containing 2% BSA (Sigma-Aldrich, Buchs, Switzerland). Plasma samples diluted to either 1:400 or 1:200 or 1:100 in PBS or brain homogenates (hippocampus, 10 μl diluted at 1:100; cortex, 1 μl undiluted; striatum, 0.5 μl undiluted in PBS) were added and incubated for 2 h at RT, followed by incubation with a goat anti-mouse IgG2a-specific antibody conjugated with HRP (Southern Biotech, Birmingham, USA). Standard curves were prepared by two-fold serial dilutions of recombinant 4P3 IgG2a antibody in PBS with initial antibody concentration at 1 nM or 2 nM. Binding was determined by measurement of HRP activity in a standard colorimetric assay. Drug plasma and brain levels were estimated by linear regression using GraphPad Prism software (San Diego, USA).

Formalin fixed, paraffin-embedded sections (3 μm) were deparaffinized in xylene and rehydrated through graded ethanol. Quenching of endogenous peroxidase activity was achieved by treatment with 3% H₂O₂ in methanol for 10 min at RT. Non-specific binding sites were blocked for 1 h at RT with PBS/5% serum (horse/goat)/4% BSA. After the blocking step, sections were incubated with biotinylated goat anti-mouse IgG2a antibody (1:50 dilution, Southern Biotech, Birmingham, USA) for 3 h at RT. Antibody signal was amplified with the Vectastain Elite ABC kit (Vector Laboratories, Burlingame, USA) and detected with diaminobenzidine (DAB, Thermo Scientific, Rockford, USA). Slides were mounted using Eukitt® mounting medium (O. Kindler GmbH; Freiburg, Germany). Bright-field imaging was performed using a Dotslide VS120 slide scanner (Olympus Schweiz AG, Switzerland).

TABLE 14

| | Mouse | Group | AAV | Dose (gc/ | | |
|---|---|---|---|---|---|---|
| Group | line | size | vector | animal) | Application route | Takedown |
| 1 | C57BL6 | n = 5 | 4P3 IgG2a | 2.4e10 | icv | 4 weeks p.i. |
| 2 | C57BL6 | n = 5 | 4P3 IgG2a | 2.4e10 | intrahippocampal | 4 weeks p.i. |
| 3 | C57BL6 | n = 5 | 4P3 IgG2a | 2.4e10 | intrastriatal | 4 weeks p.i. |

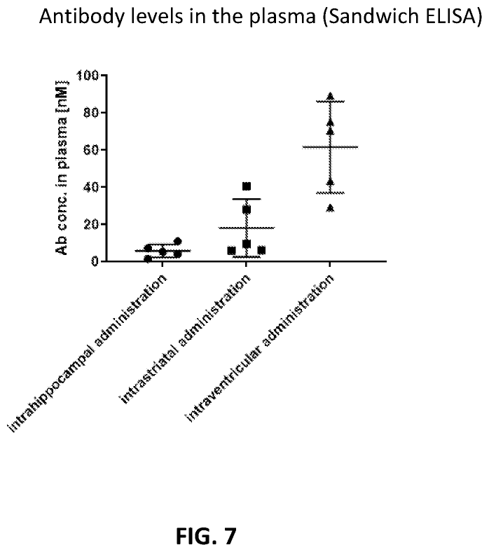

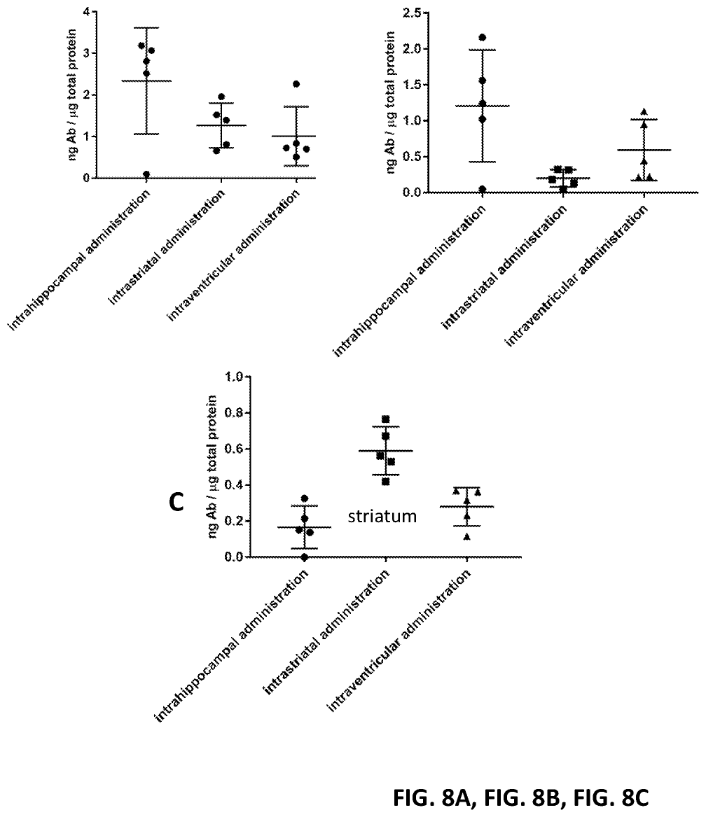

FIG. 7 shows the antibody levels detected in plasma 4 weeks after dosing by intrahippocampal administration, intrastriatal administration or intraventricular administration. The presence of antibody in the plasma indicated likely peripheral transduction, with the highest peripheral concentration coming from vector administered by ICV (FIG. 7). Antibody expression was detected by immunohistochemistry staining (anti-IgG2a antibody) in cells of the hippocampus, and 4P3 IgG2a expression is detectable via all routes of administration tested, intrahippocampal administration, intraventricular administration, and intrastriatal administration (data not shown). Brain regions hippocampus, cortex and striatum, respectively, were collected, homogenized and antibody concentration was determined as depicted in FIGS. 8A-8C.

Intraparenchymal administration showed highest concentration in the brain region where vector was delivered (IP-hippocampus had the highest concentration in the hippocampus, and IP-Striatum gave the highest concentration in the striatum). ICV delivered reasonably consistent antibody levels across all three tissues. See FIGS. 8A-8C.

### 6.7 Example 7—In Vivo Expression of Vectorized scFv-Fc Injected ICV or Intrahippocampal in Adult C57BL/6 Mice

Adult C57BL/6 mice were treated in a similar manner described in Example 6. Table 15 shows the treatment groups, dosage and application route. The sequence identifier for the amino acid sequence of the antibody encoded by the vector administered is indicated. The CAG promoter is the promoter for all constructs in this experiment.

TABLE 15

| Mouse | | Dose | Administration | Group | Time |
|---|---|---|---|---|---|
| line | Treatment group | [vg/side] | route | size | point |
| C57BL/6 | AAV9-null | 1.2 × 10¹⁰ | ICV, bilateral | n = 3 | 4 weeks |
| | 4P3 IgG2a | | | | p.i. |
| | (Encoded by SEQ ID NO: 74) | | | | |
| | 4P3-HL-L3 scFv-Fc | | | | |
| | (SEQ ID NO: 242Construct | | | | |
| | SEQ ID NO: 369) | | | | |
| | 31B6-HL-L3 scFv-Fc | | | | |
| | (SEQ ID NO: 263; | | | | |
| | Construct SEQ ID NO: 372)) | | | | |
| | 43A11-HL-L3 scFv-Fc | | | | |
| | (Construct SEQ ID NO: 375) | | | | |
| | EGFP | | | | |
| | 4P3-HL-L3 scFv-Fc | | Intrahippocamp | | |
| | (SEQ ID NO: 242; | | al, bilateral | | |
| | Construct SEQ ID NO: 369) | | | | |

Adult C57BL/6 mice were anaesthetized using an intraperitoneal (i.p.) injection of a mixture of fentanyl (0.05 mg/kg), midazolam (5.0 mg/kg), and medetomidin (0.5 mg/kg) in saline. A loss of response to nociceptive stimulation of the tail and between the toes indicated deep sedation. Mice were placed on a heating pad to maintain body temperature during surgery, and eye cream (Viscotears, carbomerum 980, 2.0 mg) was applied onto the eyes to prevent drying out during the surgery. Gene therapy vectors were administered by bilateral intracerebroventricular (lateral ventricle) or intrahippocampal injections at a dose of 1.2e10 gc/side (n=3 per group, see Table 15). Bilateral stereotaxic injections of 2.0 μl of AAV viral vectors were performed by drilling a hole in the skull at the injection site and placing a Hamilton syringe into either the lateral ventricle (A/P, −0.6 mm from bregma; L, ±1.0 mm; D/V, −2.0 mm), or the hippocampus (A/P, −2.0 mm from bregma; L, ±1.6 mm; D/V, −1.7 mm). The injection speed was 0.2 μL/minute and the needle was kept in place for an additional 10 minutes before it was slowly withdrawn. The surgical area was cleaned with sterile saline and the incision was sutured. Immediately after the surgery the antidote was administered intraperitoneally, a mixture containing flumazenil (0.5 mg/kg) and atipamezol (2.5 mg/kg) in saline. Mice were monitored until recovery from anesthesia. In addition to the routine monitoring by animal care takers, operated animals were checked daily for the 3 days following surgery for recovery of the wounds, as well as weekly for signs of abnormal behavior until the time of sacrifice for sample collection. Mice were provided with 200 mg/kg paracetamol in the drinking water starting 24 hours prior to the surgery and over a period of the next 7 days. Four weeks post administration, mice were deeply anesthetized (i.p. injection of ketamine 12.5 mg/ml (1.25%) and xylazine 2.5 mg/ml (0.25%) in PBS with 10 μl/g body weight) and blood was withdrawn from the vena cava before mice were transcardially perfused with cold phosphate-buffered saline/heparin through the left ventricle. Blood samples were collected into BD Microtainer K2E tubes and processed according to manufacturer's protocol for obtaining blood plasma. Brains were then dissected: left hemibrains were fixed in phosphate-buffered 4% paraformaldehyde solution overnight at 4° C. whereas right hemibrains were further dissected for isolating cortex, hippocampus and striatum that were then fresh frozen on dry ice. Paraformaldehyde-fixed hemibrains were then processed on a Shandon Citadel 2000 Tissue Processor (Thermo Scientific), paraffin-embedded, and cut into 3-μm sagittal sections.

Frozen tissues (cortex, hippocampus, and striatum) were homogenized using DEA buffer (50 mM NaCl (Sigma-Aldrich, Buchs, Switzerland) and 0,2% diethylamine (Sigma-Aldrich, Buchs, Switzerland) in ddH₂0) complemented with PhosphoSTOP and cOmplete tablets (Roche, Basel, Switzerland). Soluble fractions were collected after spinning down the homogenates for 60 min at 18213 rpm. Samples were stored at −80° C. until use.

96-well microplates (Corning Incorporated, Corning, USA) were coated with goat anti-mouse IgG2a antibody (Southern Biotech, Birmingham, USA) at a concentration of 1 μg/ml in PBS overnight at 4° C. Non-specific binding sites were blocked for 1 h at RT with PBS/0.1% Tween®-20 containing 2% BSA (Sigma-Aldrich, Buchs, Switzerland). Brain homogenates (hippocampus, 30 μl diluted at either 1:200 or 1:500; cortex, 30 μl diluted at either 1:20, 1:40, or 1:200; striatum, 30 μl diluted at either 1:20 or 1:40 in PBS) were added and incubated for 2 h at RT, followed by incubation with a goat anti-mouse IgG2a-specific antibody conjugated with HRP (Southern Biotech, Birmingham, USA). Standard curves were prepared by two-fold serial dilutions of recombinant 4P3 IgG2a antibody in PBS with initial antibody concentration at 1 nM or 2 nM. Binding was determined by measurement of HRP activity in a standard colorimetric assay. Antibody concentrations in brain homogenates were estimated by linear regression using GraphPad Prism software (San Diego, USA).

Formalin fixed, paraffin-embedded sections (3 μm) were deparaffinized in xylene and rehydrated through graded ethanol. Quenching of endogenous peroxidase activity was achieved by treatment with 3% H₂O₂ in methanol for 10 min at RT. Non-specific binding sites were blocked for 1 h at RT with PBS/5% serum (horse/goat)/4% BSA. After the blocking step, sections were incubated with biotinylated goat anti-mouse IgG2a antibody (1:50 dilution, Southern Biotech, Birmingham, USA) for 3 h at RT. Antibody signal was amplified with the Vectastain Elite ABC kit (Vector Laboratories, Burlingame, USA) and detected with diaminobenzidine (DAB, Thermo Scientific, Rockford, USA). Slides were mounted using Eukitt® mounting medium (O. Kindler GmbH; Freiburg, Germany). Bright-field imaging was performed using a Dotslide VS120 slide scanner (Olympus Schweiz AG, Switzerland).

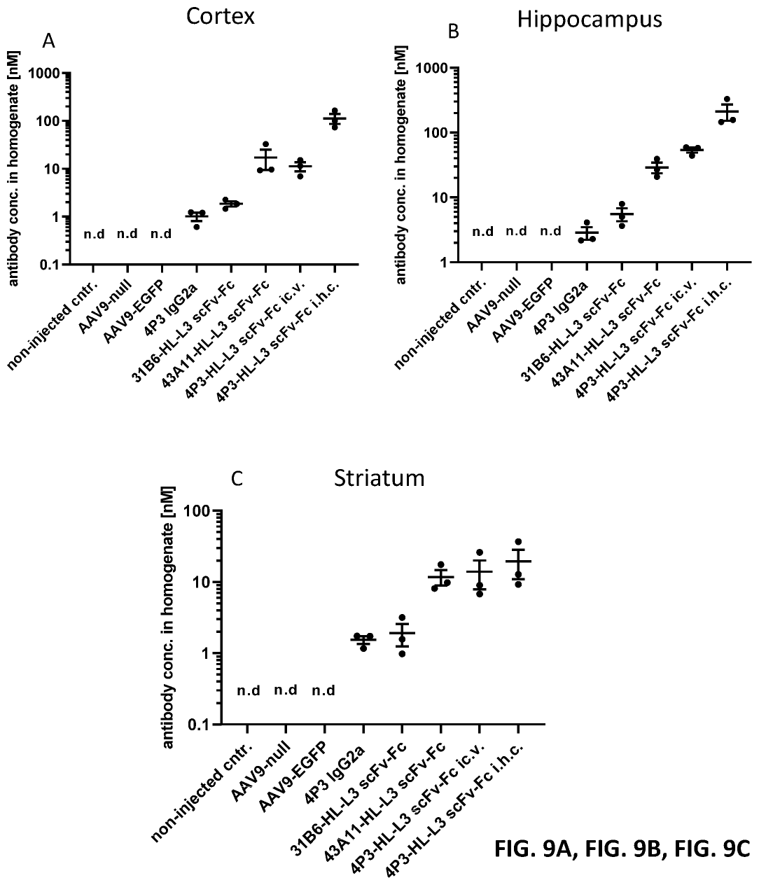

Brain regions hippocampus, striatum and cortex were collected, homogenized and scFv-Fc concentration was determined. scFv-Fc expression was also detected by immunohistochemistry staining (anti-IgG2a antibody) in cells of the hippocampus after administration either intrahippocampal or ICV (data not shown). FIGS. 9A-9C show antibody concentration (nM) in the collected homogenates from the cortex (A), hippocampus (B), and striatum (C). 4P3 IgG2a expression is detectable via all routes of administration tested in all brain regions.

It is noted that the concentration of scFv-Fc derivative is underestimated due to the use of 4P3 IgG2a as standard curve (⅓ MW difference). Nevertheless, the concentrations are semi-quantitative and accounting for the underestimate, the scFv-Fc constructs, delivered either ICV or by intrahippocampal administration, are expressed at higher levels than the 4P3 IgG2a antibody. Both the full length 4P3 IgG2a and the scFv-Fc proteins are expressed in the hippocampus and the cortex by ICB administration.

The eGFP sequences used in the described experiments are:

eGFP coding sequence
(SEQ ID NO: 396)
ATGGTGAGCAAGGGCGAGGAGCTGTTCACCGGGGTGGTGCCCATCCTGGTCGAGCTGGACGGCGACGTAAA
CGGCCACAAGTTCAGCGTGTCCGGCGAGGGCGAGGGCGATGCCACCTACGGCAAGCTGACCCTGAAGTTCA
TCTGCACCACCGGCAAGCTGCCCGTGCCCTGGCCCACCCTCGTGACCACCCTGACCTACGGCGTGCAGTGC
TTCAGCCGCTACCCCGACCACATGAAGCAGCACGACTTCTTCAAGTCCGCCATGCCCGAAGGCTACGTCCA
GGAGCGCACCATCTTCTTCAAGGACGACGGCAACTACAAGACCCGCGCCGAGGTGAAGTTCGAGGGCGACA
CCCTGGTGAACCGCATCGAGCTGAAGGGCATCGACTTCAAGGAGGACGGCAACATCCTGGGGCACAAGCTG
GAGTACAACTACAACAGCCACAACGTCTATATCATGGCCGACAAGCAGAAGAACGGCATCAAGGTGAACTT
CAAGATCCGCCACAACATCGAGGACGGCAGCGTGCAGCTCGCCGACCACTACCAGCAGAACACCCCCATCG
GCGACGGCCCCGTGCTGCTGCCCGACAACCACTACCTGAGCACCCAGTCCGCCCTGAGCAAAGACCCCAAC
GAGAAGCGCGATCACATGGTCCTGCTGGAGTTCGTGACCGCCGCCGGGATCACTCTCGGCATGGACGAGCT
GTACAAGTAA
eGFP protein
(SEQ ID NO: 397)
MVSKGEELFTGVVPILVELDGDVNGHKFSVSGEGEGDATYGKLTLKFICTTGKLPVPWPTLVTTLTYGVQC
FSRYPDHMKQHDFFKSAMPEGYVQERTIFFKDDGNYKTRAEVKFEGDTLVNRIELKGIDFKEDGNILGHKL
EYNYNSHNVYIMADKQKNGIKVNFKIRHNIEDGSVQLADHYQQNTPIGDGPVLLPDNHYLSTQSALSKDPN
EKRDHMVLLEFVTAAGITLGMDELYK

### 6.8 Example 8—In Vivo Expression of Vectorized mAbs and scFv-Fc Injected ICV or Intrahippocampal in Adult C57BL/6 Mice

Adult C57BL/6 mice were treated in a similar manner described in Example 6. Mice were administered AAV9 containing vectorized constructs as set forth in Table 16, including constructs for expression of the full length 4P3 antibody under control of the CAG, GFAP or hSyn promoter, and the 4P3 HL-L3 scFV-Fc construct under the control of the CAG promoter (administered bilaterally either ICV or intrahippocampal).

TABLE 16
Treatment groups, dosage and application route

| Mouse | Treatment | Dose | Administration | Group | Time |
|---|---|---|---|---|---|
| line | group | [vg/side] | route | size | point |
| C57BL/6 | CAG-4P3 IgG2a | 1.2 × 10¹⁰ | Bilateral, IP- | n = 3 | 4 weeks |
| | GFAP.4P3 IgG2a | | hippocampus | | p.i. |
| | hSyn.4P3 IgG2a | | | | |
| | CAG.4P3.HL-L3 scFv-Fc | | | | |
| | CAG.4P3 HL-L3 scFv-Fc | | Bilateral, ICV | | |
| | Formulation buffer | n/a, 2 ul | Bilateral, IP- | | |
| | | | hippocampus | | |

Adult C57BL/6 mice were anaesthetized using an intraperitoneal (i.p.) injection of a mixture of fentanyl (0.05 mg/kg), midazolam (5.0 mg/kg), and medetomidin (0.5 mg/kg) in saline. A loss of response to nociceptive stimulation of the tail and between the toes indicated deep sedation. Mice were placed on a heating pad to maintain body temperature during surgery, and eye cream (Viscotears, carbomerum 980, 2.0 mg) was applied onto the eyes to prevent drying out during the surgery. Gene therapy vectors (see Table 15) were administered by bilateral intracerebroventricular (lateral ventricle) or intrahippocampal injections at a dose of 1.2e10 gc/side (n=3 per group, see Table 16). Bilateral stereotaxic injections of 2.0 μl of AAV viral vectors were performed by drilling a hole in the skull at the injection site and placing a Hamilton syringe into either the lateral ventricle (A/P, −0.6 mm from bregma; L, ±1.0 mm; D/V, −2.0 mm), or the hippocampus (A/P, −2.0 mm from bregma; L, ±1.6 mm; D/V, −1.7 mm). The injection speed was 0.2 L/minute and the needle was kept in place for an additional 10 minutes before it was slowly withdrawn. The surgical area was cleaned with sterile saline and the incision was sutured. Immediately after the surgery the antidote was administered intraperitoneally, a mixture containing flumazenil (0.5 mg/kg) and atipamezol (2.5 mg/kg) in saline. Mice were monitored until recovery from anesthesia. In addition to the routine monitoring by animal care takers, operated animals were checked daily for the 3 days following surgery for recovery of the wounds, as well as weekly for signs of abnormal behavior until the time of sacrifice for sample collection. Mice were provided with 200 mg/kg paracetamol in the drinking water starting 24 hours prior to the surgery and over a period of the next 7 days. Four weeks post administration, mice were deeply anesthetized (i.p. injection of ketamine 12.5 mg/ml (1.25%) and xylazine 2.5 mg/ml (0.25%) in PBS with 10 μl/g body weight) and blood was withdrawn from the vena cava before mice were transcardially perfused with cold phosphate-buffered saline/heparin through the left ventricle. Blood samples were collected into BD Microtainer K2E tubes and processed according to manufacturer's protocol for obtaining blood plasma. Brains were then dissected: left hemibrains were fixed in phosphate-buffered 4% paraformaldehyde solution overnight at 4° C. whereas right hemibrains were further dissected for isolating cortex, hippocampus and striatum that were then fresh frozen on dry ice.

Frozen tissues (cortex, hippocampus, and striatum) were homogenized using DEA buffer (50 mM NaCl (Sigma-Aldrich, Buchs, Switzerland) and 0,2% diethylamine (Sigma-Aldrich, Buchs, Switzerland) in ddH₂0) complemented with PhosphoSTOP and cOmplete tablets (Roche, Basel, Switzerland). Soluble fractions were collected after spinning down the homogenates for 60 min at 18213 rpm. Samples were stored at −80° C. until use.

96-well microplates (Corning Incorporated, Corning, USA) were coated with goat anti-mouse IgG2a antibody (Southern Biotech, Birmingham, USA) at a concentration of 1 μg/ml in PBS overnight at 4° C. Non-specific binding sites were blocked for 1 h at RT with PBS/0.1% Tween®-20 containing 2% BSA (Sigma-Aldrich, Buchs, Switzerland). Brain homogenates (hippocampus, 30 μl diluted at either 1:200, or 1:100, or 1:500; cortex, 30 μl diluted at either 1:20, or 1:300; striatum, 30 μl diluted at either 1:5, or 1:20 in PBS) were added and incubated for 2 h at RT, followed by incubation with a goat anti-mouse IgG2a-specific antibody conjugated with HRP (Southern Biotech, Birmingham, USA). Standard curves were prepared by two-fold serial dilutions of recombinant 4P3 IgG2a antibody in PBS with initial antibody concentration at 1 nM or 2 nM. Binding was determined by measurement of HRP activity in a standard colorimetric assay. Brain homogenate concentrations were estimated by linear regression using GraphPad Prism software (San Diego, USA).

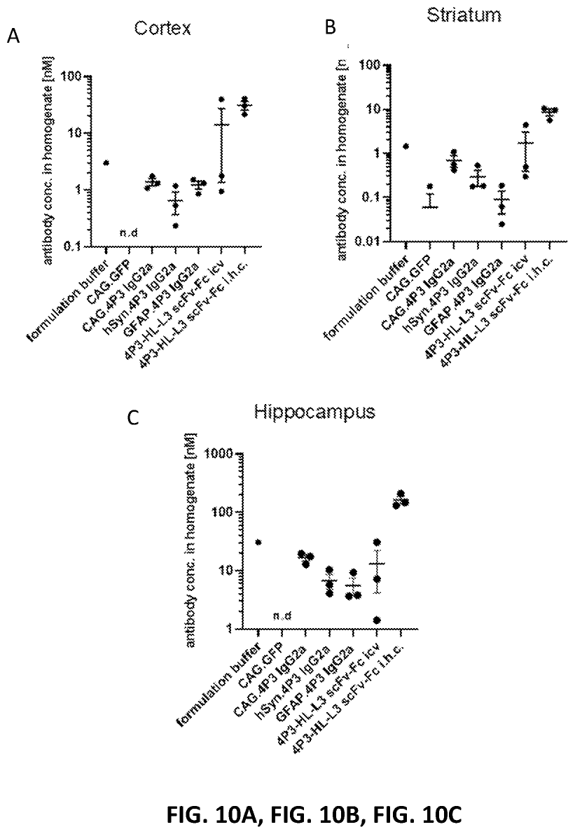

FIGS. 10A-10C show antibody concentration (nM) for the antibodies or scFv-Fcs expressed from the administered vectors in the homogenates of cortex (A), striatum (B), and hippocampus (C). As noted in Table 16 above, all vectors were administered intrahippocampal except for the one treatment group in which the 4P3-HL-L3 scFv-Fc was administered ICV. It is noted that the concentration of scFv-Fc derivative is underestimated due to the use of 4P3 IgG2a as standard curve (⅓ MW difference). Nevertheless, the concentrations are semi-quantitative and accounting for the underestimate, the scFv-Fc constructs, are generally expressed at higher levels in the cortex, striatum and hippocampus than the IgG2a constructs

### 6.9 Example 9—Longer Term In Vivo Expression of Vectorized Antibody Injected in Adult C57BL/6 Mice

Adult C57BL/6 mice were treated in a similar manner described in Example 6. AAV9 constructs encoding 4P3 IgG2a (including hSyn-4P3 IgG2a and GFAP-4P3 IgG2a) and the 4P3 IgG2a antibody were administered as described in Table 17, which shows the treatment groups, dosage and application route.

TABLE 17

| Mouse | Treatment | Dose | Administration | |
|---|---|---|---|---|
| line | group | [vg/side] | route | Timepoints |
| C57BL/6 | AAV9-null | 1.2 × 10¹⁰ | intrahippocampal; | 1 month |
| | hSyn-4P3 | 1.2 × 10¹⁰ | bilateral | 3 months |
| | IgG2a | | | |
| | GFAP-4P3 | 1.2 × 10¹⁰ | | 6 months |
| | IgG2a | | | |
| | 4P3 Ig2a | 30 mg/kg | Intraperitoneal; | 1 month |
| | (antibody) | | weekly | |

Adult C57BL/6 mice were anaesthetized using an intraperitoneal (i.p.) injection of a mixture of fentanyl (0.05 mg/kg), midazolam (5.0 mg/kg), and medetomidin (0.5 mg/kg) in saline. A loss of response to nociceptive stimulation of the tail and between the toes indicated deep sedation. Mice were placed on a heating pad to maintain body temperature during surgery, and eye cream (Viscotears, carbomerum 980, 2.0 mg) was applied onto the eyes to prevent drying out during the surgery. Gene therapy vectors were administered by bilateral intrahippocampal injections at a dose of 1.2e10 gc/side (n=3 per group, see Table 17). Bilateral stereotaxic injections of 2.0 μl of AAV viral vectors were performed by drilling a hole in the skull at the injection site and placing a Hamilton syringe into the hippocampus (A/P, −2.0 mm from bregma; L, ±1.6 mm; D/V, −1.7 mm). The injection speed was 0.2 μL/minute and the needle was kept in place for an additional 10 minutes before it was slowly withdrawn. The surgical area was cleaned with sterile saline and the incision was sutured. Immediately after the surgery the antidote was administered intraperitoneally, a mixture containing flumazenil (0.5 mg/kg) and atipamezol (2.5 mg/kg) in saline. Mice were monitored until recovery from anesthesia. In addition to the routine monitoring by animal care takers, operated animals were checked daily for the 3 days following surgery for recovery of the wounds, as well as weekly for signs of abnormal behavior until the time of sacrifice for sample collection. Mice were provided with 200 mg/kg paracetamol in the drinking water starting 24 hours prior to the surgery and over a period of the next 7 days. For chronic peripheral antibody treatment, mice were weekly dosed intraperitoneally with the recombinant 4P3 IgG2a antibody at 30 mg/kg. 1 month, 3 months or 6 months post administration, mice were deeply anesthetized (i.p. injection of ketamine 12.5 mg/ml (1.25%) and xylazine 2.5 mg/ml (0.25%) in PBS with 10 μl/g body weight) and blood was withdrawn from the vena cava before mice were transcardially perfused with cold phosphate-buffered saline/heparin through the left ventricle. Blood samples were collected into BD Microtainer K2E tubes and processed according to manufacturer's protocol for obtaining blood plasma. Brains were then dissected: left hemibrains were fixed in phosphate-buffered 4% paraformaldehyde solution overnight at 4° C. whereas right hemibrains were further dissected for isolating cortex, hippocampus and striatum that were then fresh frozen on dry ice. Paraformaldehyde-fixed hemibrains were then processed on a Shandon Citadel 2000 Tissue Processor (Thermo Scientific), paraffin-embedded, and cut into 3-μm sagittal sections.

Frozen tissues (cortex, hippocampus, and striatum) were homogenized using DEA buffer (50 mM NaCl (Sigma-Aldrich, Buchs, Switzerland) and 0,2% diethylamine (Sigma-Aldrich, Buchs, Switzerland) in ddH₂0) complemented with PhosphoSTOP and cOmplete tablets (Roche, Basel, Switzerland). Soluble fractions were collected after spinning down the homogenates for 60 min at 18213 rpm. Samples were stored at −80° C. until use.

96-well microplates (Corning Incorporated, Corning, USA) were coated with goat anti-mouse IgG2a antibody (Southern Biotech, Birmingham, USA) at a concentration of 1 μg/ml in PBS overnight at 4° C. Non-specific binding sites were blocked for 1 h at RT with PBS/0.1% Tween®-20 containing 2% BSA (Sigma-Aldrich, Buchs, Switzerland). Brain homogenates (hippocampus, 30 μl diluted at either 1:50, or 1:200; cortex, 30 μl diluted at 1:50; striatum, 30 μl diluted at either 1:20 in PBS) were added and incubated for 2 h at RT, followed by incubation with a goat anti-mouse IgG2a-specific antibody conjugated with HRP (Southern Biotech, Birmingham, USA). Standard curves were prepared by two-fold serial dilutions of recombinant 4P3 IgG2a antibody in PBS with initial antibody concentration at 1 nM or 2 nM. Binding was determined by measurement of HRP activity in a standard colorimetric assay. Drug plasma and brain levels were estimated by linear regression using GraphPad Prism software (San Diego, USA).

Formalin fixed, paraffin-embedded sections (3 μm) were deparaffinized in xylene and rehydrated through graded ethanol. Quenching of endogenous peroxidase activity was achieved by treatment with 3% H₂O₂ in methanol for 10 min at RT. Non-specific binding sites were blocked for 1 h at RT with PBS/5% serum (horse/goat)/4% BSA. After the blocking step, sections were incubated with biotinylated goat anti-mouse IgG2a antibody (1:50 dilution, Southern Biotech, Birmingham, USA) for 3 h at RT. Antibody signal was amplified with the Vectastain Elite ABC kit (Vector Laboratories, Burlingame, USA) and detected with diaminobenzidine (DAB, Thermo Scientific, Rockford, USA). Slides were mounted using Eukitt® mounting medium (O. Kindler GmbH; Freiburg, Germany). Bright-field imaging was performed using a Dotslide VS120 slide scanner (Olympus Schweiz AG, Switzerland).

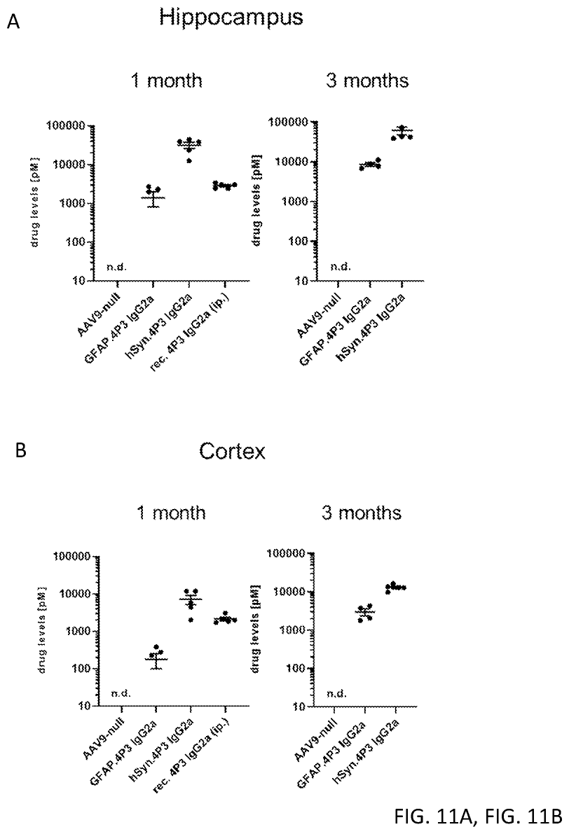

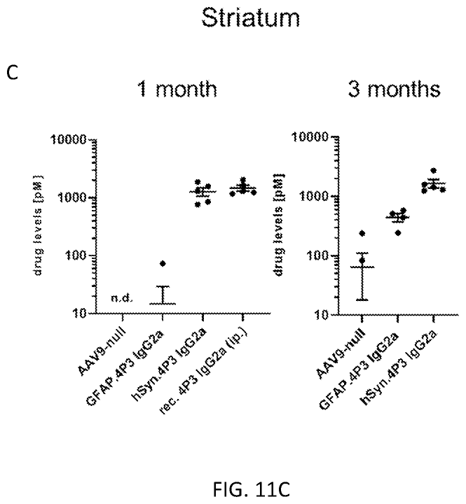

FIGS. 11A-11C show the results at 1 month and 3 months post treatment. Intraparenchymal administration of hSyn-4P3 IgG2a as well as GFAP-4P3 IgG2a AAV vectors resulted in sustained antibody expression in the brain. hSyn-4P3 IgG2a hippocampal drug levels increased 10-fold as compared to peripheral recombinant antibody dosing (30 mg/kg, once weekly). Brain drug levels increased over time, mainly in the GFAP-4P3 IgG2a-administered animals. hSyn-4P3 IgG2a- and GFAP-4P3 IgG2a-mediated cell type-specific antibody expression was confirmed by IHC (data not shown).

### 6.10 Example 10—Tau Seeding Depletion Assay: Antibodies NI-502.4P3, NI-502.31B6 and NI-502.8H1 Deplete Seeding-Competent Tau from AD Homogenates

### Brain Homogenate Preparation

Alzheimer's Disease brain tissue (NBB 194-037), inferior frontal gyrus) was procured from the Netherlands Brain Bank (NBB). Tissue was weighed and homogenized in 3× mass/volume of PBS containing protease (cOmplete Tablets, Mini EDTA-free, Roche, Switzerland) and phosphatase (PhosSTOP Tablets, Roche, Switzerland) inhibitors. Tissue was homogenized using FastPrep-24 Homogenizer (Lucerna Chem AG) twice with 6.0 m/s for 40 seconds. After homogenization, homogenates were cleared by centrifugation (Microcentrifuge 5430 R (Vaudaux-Eppendorf AG, Switzerland), full speed for 1.5 hours, 4° C.). Protein concentration in the cleared brain homogenate was determined by BCA protein assay (Pierce™ BCA Protein Assay Kit, Thermo Fischer Scientific, USA) according to the manufacturer's instructions. Total Tau concentration was determined using INNOTEST hTAU Ag ELISA (Fujirebio Europe N.V., Belgium) according to the manufacturer's instructions.

### Immunodepletion Studies

Brain homogenates containing 10 ng of tau were mixed with 2-fold serially diluted NI-502.4P3, NI-502.31B6 and NI-502.8H1 (final concentrations of 0.31-80 g/mL) in 150 μL of Opti-MEM (Invitrogen, Thermo Fisher Scientific, USA) containing protease inhibitors (cOmplete Tablets, Mini EDTA-free, Roche, Switzerland) and allowed to incubate overnight at 4° C. The next day, 50 μL of protein A magnetic bead slurry (Dynabeads™ ProteinA Immunoprecipitation Kit, Thermo Fisher Scientific, USA) was added to each sample to isolate immune complexes. Immunodepleted supernatants were transferred to clean low binding tubes (Vaudaux-Eppendorf AG, Switzerland). Each immunodepletion reaction was performed in duplicate.

### Tau Seeding Assay in HEK293T Biosensor Cells

The HEK293T tau biosensor cell line (HEK293T tau RD-CFP/YFP, ATCC® CRL-3275™) was previously described (Holmes et al., Proc. Natl. Acad. Sci. USA 111 (2014), E4376-85, doi: 10.1073/pnas.1411649111). The cells stably express the repeat domains (RD) of tau protein with a P301S mutation fused to either CFP or YFP. Although at baseline the tau reporter proteins exist in a stable, soluble form within the cell, exposure to exogenous tau seeds leads to tau reporter protein aggregation, which generates a fluorescence resonance energy transfer (FRET signal). Tau aggregation was measured by CFP to YFP FRET signal, detected with fluorescence-activated cell sorting (FACS).

HEK293T tau biosensor cells were plated in 24-well plates (TPP, Switzerland) at 50′000 cells per well in complete HEK Cell culture medium (DMEM/10% FBS/PenStrep/L-Glutamine, Gibco, Thermo Fisher Scientific, USA) and incubated at 37° C., 5% CO2 for 24 or 48 hours. Immunodepleted brain homogenates (200 μL) were mixed with 6 μL Lipofectamine 2000 (Invitrogen, Thermo Fisher Scientific, USA), gently mixed, incubated for 20 min at RT and then added to the cell media. Cells were cultured for another 24 hours, trypsinized, washed, and subjected to FRET analysis of tau aggregation by FACS. Aggregation was evaluated using a CFP-YFP FRET pair. Signals were measured on an LSR II Fortessa 4L flow cytometer (BD Biosciences, Switzerland). Forward scatter signal generated by a 488-nm laser line was used as trigger signal. CFP was excited at 405 nm and fluorescence detected with a 450/50-nm bandpass filter indicated no aggregation. YFP excited by CFP emission (FRET), detected with a 525/50-nm bandpass filter, indicated aggregation. The YFP signal was detected using a 530/30-nm bandpass filter. FCS 3.0 files were analyzed using Flowing Software version 2.5.1 (Turku Centre for Biotechnology). Data were reported as integrated FRET density, which was calculated as previously described (Holmes et al., (2014), supra): Integrated FRET density=number of FRET-positive cells×mean FRET signal intensity.

As shown in FIGS. 12A-12C, subjecting human brain homogenates derived from Alzheimer's Disease brain tissue with increasing concentrations of antibodies NI-502.4P3, NI-502.31B6 and NI-502.8H1 resulted in a concentration-dependent reduction of Tau aggregation in Tau biosensor cells, which is reflected by the reduction in the integrated FRET density.

### Equivalents

Although the invention is described in detail with reference to specific embodiments thereof, it will be understood that variations which are functionally equivalent are within the scope of this invention. Indeed, various modifications of the invention in addition to those shown and described herein will become apparent to those skilled in the art from the foregoing description and accompanying drawings. Such modifications are intended to fall within the scope of the appended claims. Those skilled in the art will recognize, or be able to ascertain using no more than routine experimentation, many equivalents to the specific embodiments of the invention described herein. Such equivalents are intended to be encompassed by the following claims. The discussion herein provides a better understanding of the nature of the problems confronting the art and should not be construed in any way as an admission as to prior art nor should the citation of any reference herein be construed as an admission that such reference constitutes “prior art” to the instant application.

All references including patent applications and publications cited herein are incorporated herein by reference in their entirety and for all purposes to the same extent as if each individual publication or patent or patent application was specifically and individually indicated to be incorporated by reference in its entirety for all purposes.

1. A recombinant AAV genome comprising a nucleic acid expression cassette, wherein the expression cassette comprises a nucleotide sequence encoding a variable heavy chain (VH) chain and a variable light (VL) chain domain of an anti-Tau monoclonal antibody (mAb) or antigen binding fragment thereof, wherein the mAb or the antigen-binding fragment thereof,
(i) binds pathological hyperphosphorylated Tau filaments in dystrophic neurites, neurofibrillary tangles and neuropil threads in an immunohistochemical (HC) assay with brain tissue of patients with Alzheimer's Disease (AD), Progressive supranuclear palsy (PSP) and/or Pick's Disease (PiD); and/or
(ii) captures Tau and AD-associated Tau in an immunoprecipitation (IP) assay with brain extracts of patients with AD; and/or
(iii) recognizes an epitope comprising the amino acid sequence 217-TPPTREPKKVA-227 (SEQ ID NO: 120) and 249-PMPDLKN-255 (SEQ ID NO: 121) or the phosphorylated Tau peptide pS202/pT205 having the amino acid sequence SGYSSPG(pS)PG(pT)PGSRSRT (SEQ ID NO: 122) or the phosphorylated Tau peptide pT212/pS214 having the amino acid sequence GTPGSRSR(pT)P(pS)LPTPPTR (SEQ ID NO: 123);
wherein (1) AAV inverted terminal repeats flank the expression cassette; and (2) the expression cassette comprises one or more regulatory elements operably linked to the nucleotide sequence encoding the mAb or antigen binding fragment thereof and wherein the one or more regulatory elements control expression of the mAb or antigen binding fragment thereof in human CNS tissue or liver cells.

2. The recombinant AAV genome of claim 1, wherein the variable heavy (VH) chain domain comprises VH complementary determining regions (CDRs) 1, 2, and 3, and the variable light (VL) chain domain comprises VL CDRs 1, 2, and 3, wherein
(a) VH-CDR1 comprises the amino acid sequence of SEQ ID NO: 154 or a variant thereof, wherein the variant comprises one or two amino acid substitutions,
(b) VH-CDR2 comprises the amino acid sequence of SEQ ID NO: 155 or a variant thereof, wherein the variant comprises one or two amino acid substitutions,
(c) VH-CDR3 comprises the amino acid sequence of SEQ ID NO: 156 or a variant thereof, wherein the variant comprises one or two amino acid substitutions,
(d) VL-CDR1 comprises the amino acid sequence of SEQ ID NO: 157 or a variant thereof, wherein the variant comprises one or two amino acid substitutions,
(e) VL-CDR2 comprises the amino acid sequence of SEQ ID NO: 158 or a variant thereof, wherein the variant comprises one or two amino acid substitutions, and
(f) VL-CDR3 comprises the amino acid sequence of SEQ ID NO: 159 or a variant thereof, wherein the variant comprises one or two amino acid substitutions;
or wherein
(g) VH-CDR1 comprises the amino acid sequence of SEQ ID NO: 160 or a variant thereof, wherein the variant comprises one or two amino acid substitutions,
(h) VH-CDR2 comprises the amino acid sequence of SEQ ID NO: 161 or a variant thereof, wherein the variant comprises one or two amino acid substitutions,
(i) VH-CDR3 comprises the amino acid sequence of SEQ ID NO: 162 or a variant thereof, wherein the variant comprises one or two amino acid substitutions,
(j) VL-CDR1 comprises the amino acid sequence of SEQ ID NO: 163 or a variant thereof, wherein the variant comprises one or two amino acid substitutions,
(k) VL-CDR2 comprises the amino acid sequence of SEQ ID NO: 164 or a variant thereof, wherein the variant comprises one or two amino acid substitutions, and
(l) VL-CDR3 comprises the amino acid sequence of SEQ ID NO: 165 or a variant thereof, wherein the variant comprises one or two amino acid substitutions;
or wherein
(m) VH-CDR1 comprises the amino acid sequence of SEQ ID NO: 166 or a variant thereof, wherein the variant comprises one or two amino acid substitutions,
(n) VH-CDR2 comprises the amino acid sequence of SEQ ID NO: 167 or a variant thereof, wherein the variant comprises one or two amino acid substitutions,
(o) VH-CDR3 comprises the amino acid sequence of SEQ ID NO: 168 or a variant thereof, wherein the variant comprises one or two amino acid substitutions,
(p) VL-CDR1 comprises the amino acid sequence of SEQ ID NO: 169 or a variant thereof, wherein the variant comprises one or two amino acid substitutions,
(q) VL-CDR2 comprises the amino acid sequence of SEQ ID NO: 170 or a variant thereof, wherein the variant comprises one or two amino acid substitutions, and
(r) VL-CDR3 comprises the amino acid sequence of SEQ ID NO: 171 or a variant thereof, wherein the variant comprises one or two amino acid substitutions.

3. The recombinant AAV genome of claim 2, wherein the VH domain comprises an amino acid sequence of SEQ ID NO: 97 or a variant thereof having at least 90% sequence identity to the amino acid sequence of SEQ ID NO: 97 and the VL domain comprises an amino acid sequence of SEQ ID NO: 98 or a variant thereof having at least 90% sequence identity to the amino acid sequence of SEQ ID NO: 98; or the VH domain comprises an amino acid sequence of SEQ ID NO: 99 or a variant thereof having at least 90% sequence identity to the amino acid sequence of SEQ ID NO: 99 and the VL domain comprises an amino acid sequence of SEQ ID NO: 100 or a variant thereof having at least 90% sequence identity to the amino acid sequence of SEQ ID NO: 100; or the VH domain comprises an amino acid sequence of SEQ ID NO: 101 or a variant thereof having at least 90% sequence identity to the amino acid sequence of SEQ ID NO: 101 and the VL domain comprises an amino acid sequence of SEQ ID NO: 102 or a variant thereof having at least 90% sequence identity to the amino acid sequence of SEQ ID NO: 102.

4. The recombinant AAV genome claim 2, wherein the anti-Tau mAb or antigen-binding fragment thereof comprises a heavy chain comprising or consisting of the VH domain and a C_{H}1 domain comprising or consisting of an amino acid sequence of SEQ ID NO: 193, 194, or 195 or a heavy chain constant region having an amino acid sequence of SEQ ID NO: 103, 105, 107, 109 or 110 and comprising a light chain comprising a light chain constant region comprising an amino acid sequence of SEQ ID NO: 116 or 118.

5. The recombinant AAV genome claim 4, wherein a nucleotide sequence encoding a furin 2A linker is in between the nucleotide sequence encoding the heavy chain and the nucleotide sequence encoding the light chain sequences, resulting in a construct with the structure: Leader—Heavy chain—Furin site—2A site—Leader—Light chain—PolyA.

6. The recombinant AAV genome of claim 2, wherein the VH domain and the VL domain are linked via a flexible, non-cleavable linker to form a single chain (scFv).

7. The recombinant AAV genome of claim 3, wherein the VH domain and the VL domain are linked via a flexible, non-cleavable linker to form a single chain (scFv).

8. The recombinant AAV genome of claim 6, wherein the scFv has an amino acid sequence of one of SEQ ID Nos: 297 to 305.

9. The recombinant AAV genome of claim 6, wherein the scFv comprises an Fc domain or a C_{H}3 domain fused to the VH or VL domain by a hinge/linker peptide.

10. The recombinant AAV genome of claim 1, wherein the expression cassette comprises a nucleic acid encoding a signal sequence at the N-terminus of each of the heavy chain and the light chain and wherein said signal sequence is MYRMQLLLLIALSLALVTNS (SEQ ID NO: 87), MNFLLSWVHWSLALLLYLHHAKWSQA (SEQ ID NO: 88), MERAAPSRRVPLPLLLLGGLALLAAGVDA (SEQ ID NO: 89), MAPLRPLLILALLAWVALA (SEQ ID NO: 90), MRLLAKIICLMLWAICVA (SEQ ID NO: 91), MRLLAFLSLLALVLQETGT (SEQ ID NO: 92), MKWVTFISLLFLFSSAYS (SEQ ID NO: 93), MAFLWLLSCWALLGTTFG (SEQ ID NO: 94), MYRMQLLSCIALILALVTNS (SEQ ID NO: 95), or MNLLLILTFVAAAVA (SEQ ID NO: 96).

11. A recombinant viral vector, which comprises:
(a) a viral capsid that is at least 95% identical to the amino acid sequence of an AAV8 capsid (SEQ ID NO: 131), AAV9 capsid (SEQ ID NO: 132), AAVrh10 capsid (SEQ ID NO: 133), an AAV.PHP.B capsid (SEQ ID NO: 220), an AAV.PHP.eB capsid (SEQ ID NO: 219), an AAVrh20 capsid (SEQ ID NO: 134), an AAVrh39 capsid (SEQ ID NO: 341) or an AAVcy5 capsid; and
(b) recombinant AAV genome of claim 1.

12. A method of prophylactic or therapeutic treatment of a neurodegenerative tauopathy in a human subject in need thereof, comprising an AAV vector having:
(a) a viral capsid that is at least 95% identical to the amino acid sequence of an AAV8 capsid (SEQ ID NO: 131), an AAV9 capsid (SEQ ID NO: 132), an AAV.PHP.B capsid (SEQ ID NO: 220), an AAV.PHP.eB capsid (SEQ ID NO: 219), AAVrh10 capsid (SEQ ID NO: 133), an AAVrh20 capsid (SEQ ID NO: 134), an AAVrh39 capsid (SEQ ID NO: 341) or an AAVcy5 capsid; and
(b) the recombinant AAV genome of claim 1 flanked by AAV inverted terminal repeats (ITRs),
wherein said AAV vector is formulated for administration to the subject, optionally wherein administration is intrathecal, intravenous, subcutaneous, intranasal, intraparenchymal, or intramuscular or to the cisterna magna.

13. A method of producing recombinant AAVs comprising:
(a) culturing a host cell comprising:
(i) a nucleic acid comprising the recombinant AAV genome of claim 2;
(ii) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans; and
(iii) sufficient adenovirus helper functions to permit replication and packaging of the artificial genome by the AAV capsid protein; and
(b) recovering recombinant AAV encapsidating the artificial genome from the cell culture.

14. A host cell comprising
(a) a nucleic acid comprising the recombinant AAV genome of claim 2;
(b) a trans expression cassette lacking AAV ITRs, wherein the trans expression cassette encodes an AAV rep and an AAV capsid protein operably linked to expression control elements that drive expression of the AAV rep and the AAV capsid protein in the host cell in culture and supply the AAV rep and the AAV capsid protein in trans; and
(c) sufficient adenovirus helper functions to permit replication and packaging of the artificial genome by the AAV capsid protein.

15. A recombinant AAV produced by the method comprising:
(a) culturing the host cell of claim 14; and
(b) recovering recombinant AAV encapsidating the artificial genome from the cell culture.

16. The recombinant AAV genome of claim 6, wherein the expression cassette comprises a nucleic acid encoding a signal sequence at the N-terminus of the scFv, wherein said signal sequence is appropriate for expression and secretion in human cells and wherein said signal sequence is MYRMQLLLLIALSLALVTNS (SEQ ID NO: 87), MNFLLSWVHWSLALLLYLHHAKWSQA (SEQ ID NO: 88), MERAAPSRRVPLPLLLLGGLALLAAGVDA (SEQ ID NO: 89), MAPLRPLLILALLAWVALA (SEQ ID NO: 90), MRLLAKIICLMLWAICVA (SEQ ID NO: 91), MRLLAFLSLLALVLQETGT (SEQ ID NO: 92), MKWVTFISLLFLFSSAYS (SEQ ID NO: 93), MAFLWLLSCWALLGTTFG (SEQ ID NO: 94), MYRMQLLSCIALILALVTNS (SEQ ID NO: 95), or MNLLLILTFVAAAVA (SEQ ID NO: 96).

17. The recombinant AAV genome of claim 10, wherein the recombinant AAV genome is packaged in a viral capsid.

18. The recombinant AAV genome of claim 17, wherein the viral capsid comprises a capsid protein at least 95% identical to the VP1 sequence of an AAV capsid serotype selected from AAV1, AAV1, AAV2, rAAV3, AAV4, AAV5, AAV6, AAV7, AAV8, AAV9, AAV10, AAV11, AAV-12, AAV13, AAV14, AAV15, AAV16, AAV.rh8, AAV.rh10, AAV.rh20, AAV.rh39, AAV.Rh74v1, AAV.Rh74v2, AAV.RHM4-1, AAV.hu37, AAV.Anc80, AAV.Anc80L65, AAV.7m8, AAV.PHP.B, AAV.PHP.eB, AAV2.5, AAV2tYF, AAV3B, AAV.LK03, AAV.HSC1, AAV.HSC2, AAV.HSC3, AAV.HSC4, AAV.HSC5, AAV.HSC6, AAV.HSC7, AAV.HSC8, AAV.HSC9, AAV.HSC10, AAV.HSC11, AAV.HSC12, AAV.HSC13, AAV.HSC14, AAV.HSC15, or AAV.HSC16 or a derivative, modification, or pseudotype thereof.

19. The recombinant AAV genome of claim 1, wherein the expression cassette comprises SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, or SEQ ID NO: 15.

20. The recombinant AAV genome of claim 6, wherein the scFv comprises SEQ ID NO: 317, SEQ ID NO: 318, SEQ ID NO: 319, SEQ ID NO: 320, SEQ ID NO: 321, SEQ ID NO: 322, SEQ ID NO: 323, SEQ ID NO: 324, SEQ ID NO: 325, SEQ ID NO: 326, SEQ ID NO: 327, SEQ ID NO: 328, SEQ ID NO: 329, SEQ ID NO: 330, SEQ ID NO: 331, SEQ ID NO: 332, SEQ ID NO: 333, SEQ ID NO: 334, SEQ ID NO: 335, SEQ ID NO: 336, SEQ ID NO: 337, SEQ ID NO: 338, SEQ ID NO: 339, SEQ ID NO: 340, SEQ ID NO: 345, SEQ ID NO: 346, SEQ ID NO: 347, SEQ ID NO: 348, SEQ ID NO: 349, or SEQ ID NO: 350.
